(19) United States
(12) Patent Application Publication
(10) Pub. No.: US 2018/0171027 A1
(43) Pub. Date: Jun. 21, 2018

(54) Biomarkers Related to Treatment of Cancer with HER3 and EGFR Inhibitors

(71) Applicant: Celldex Therapeutics, Inc., Hampton, NJ (US)

(72) Inventors: Theresa Marie Lavallee, Rockville, MD (US); Diego Alvarado, Madison, CT (US)

(73) Assignee: Celldex Therapeutics, Inc., Hampton, NJ (US)

(21) Appl. No.: 15/566,989

(22) PCT Filed: Apr. 15, 2016
(86) PCT No.: PCT/US16/27802

§ 371 Date: Oct. 16, 2017

Related U.S. Application Data
(60) Provisional application No. 62/149,474, filed on Apr. 17, 2015.
(60) Provisional application No. 62/175,135, filed on Jun. 12, 2015.
(60) Provisional application No. 62/251,076, filed on Nov. 4, 2015.

(51) Int. Cl. C07K 16/32; A61P 35/00; A61K 39/395; C12Q 1/6886; G01N 33/574; A61K 47/68; A61K 31/519; A61K 31/4184; A61K 31/437; A61K 31/506; A61P 35/02; C07K 16/28

(52) U.S. Cl. CPC C07K 16/32; G01N 2333/71; A61K 39/39558; A61K 39/3955; C12Q 1/6886; G01N 33/57492; A61K 47/6851; A61K 47/6855; A61K 47/6859; A61K 47/6861; A61K 47/6863; A61K 47/6867; A61K 47/6869; A61K 31/519; A61K 31/4184; A61K 31/437; A61K 31/506; A61P 35/02; C07K 16/2863; C07K 2317/92; C07K 2317/56; C07K 2317/565; C07K 2317/52; C07K 2317/94; C07K 2317/21; C07K 2317/76; C07K 2317/732; C12Q 2600/158; C12Q 2600/106; G01N 2333/495; G01N 2333/485; A61P 35/00

(57) ABSTRACT

Provided herein are methods of treating cancers expressing specific biomarkers with HER3 and/or EGFR inhibitors, and provided herein are also biomarkers and uses thereof in determining likelihood of effective cancer treatment with HER3 and/or EGFR inhibitors. In one aspect, the disclosure provides methods for treating a cancer, comprising administering to a patient diagnosed with said cancer a therapeutically effective amount of a HER3 inhibitor, an EGFR inhibitor, or a combination of a HER3 inhibitor and an EGFR inhibitor. Also disclosed herein are kits comprising components for performing the methods for determining whether a patient diagnosed with cancer is indicated as likely to be responsive to treatment with a HER3 inhibitor and/or an EGFR inhibitor.

This application claims the benefit of U.S. Provisional Application No. 62/149,474, filed Apr. 17, 2015, U.S. Provisional Application No. 62/175,135, filed Jun. 12, 2015, and U.S. Provisional Application No. 62/251,076, filed Nov. 4, 2015, each of which is hereby incorporated by reference in its entirety.

### REFERENCE TO SEQUENCE LISTING SUBMITTED ELECTRONICALLY

This application incorporates by reference a Sequence Listing submitted with this application as text file entitled “Sequence_Listing_12638-131-228.txt” created on Apr. 14, 2016 and having a size of 32,768 bytes.

### FIELD

Provided herein are methods of treating cancers expressing specific biomarkers with HER3 and/or EGFR inhibitors, and provided herein are also biomarkers and uses thereof in determining likelihood of effective cancer treatment with HER3 and/or EGFR inhibitors.

### BACKGROUND

The human epidermal growth factor receptor 3 (HER3, also known as Erbb3) is a receptor protein tyrosine and belongs to the epidermal growth factor receptor (EGFR) EGFR/HER subfamily of receptor protein tyrosine kinases (RTK), consisting of EGFR (HER1/Erbb1), HER2/Erbb2, HER3/Erbb3 and HER4/Erbb4. EGFR and HER2 are among the most well-established oncogenic RTKs driving the tumorigenesis of multiple types of solid tumors, including major categories such as breast, colorectal, and lung cancers. The tyrosine kinase activities of EGFR and HER2 have been shown to be essential for their oncogenic activities.

Like the prototypical EGFR, the transmembrane receptor HER3 consists of an extracellular ligand-binding domain (ECD), a dimerization domain within the ECD, an transmembrane domain, and intracellular protein tyrosine kinase domain (TKD) and a C-terminal phosphorylation domain (see, e.g., Kim et al. (1998), Biochem. J. 334, 189-195; Roepstorff et al. (2008) Histochem. Cell Biol. 129, 563-578).

The ligand neuregulin (NRG, also known as heregulin, HRG) binds to the extracellular domain of HER3 and activates the receptor-mediated signaling pathway by promoting dimerization with other EGFR family members (e.g., other HER receptors) and transphosphorylation of its intracellular domain. HER3 has been shown to lack detectable tyrosine kinase activity, likely due to a non-conservative replacement of certain key residues in the tyrosine kinase domain. Therefore, a consequence of this kinase-deficiency, HER3 needs to form hetero-dimers with other RTKs, especially EGFR and HER2, to undergo phosphorylation and be functionally active.

The central role for HER3 in oncogenesis is acting as a scaffolding protein to enable the maximum induction of the PI3K/AKT pathway. HER3 promotes tumor growth in unstressed conditions, and has also been found to be highly involved in conferring therapeutic resistances to many targeted drugs, including EGFR tyrosine kinase inhibitors, HER2 monoclonal antibodies such as trastuzumab, as well as small molecule inhibitors of PI3K or AKT or MEK.

In addition, HER3 has been found to be overexpressed and/or overactivated in several types of cancers such as breast cancer, ovarian cancer, prostate cancer, liver cancer, kidney and urinary bladder cancers, pancreatic cancers, brain cancers, hematopoietic neoplasms, retinoblastomas, melanomas, colorectal cancers, gastric cancers, head and neck cancers, lung cancer, etc. (see, e.g., Sithanandam & Anderson (2008) Cancer Gene Ther. 15, 413-448). In general, HER3 is frequently activated in EGFR, HER2, C-Met, and FGFRII-expressing cancers.

There is a need for improved diagnosis, prognosis prediction, and treatment of many of these cancers, especially wherein HER3 is activated alongside other receptor tyrosine kinases, such as EGFR.

### BRIEF SUMMARY

In one aspect, the disclosure provides methods for treating a cancer (e.g., squamous cell carcinoma of the head and neck (SCCHN)), comprising administering to a patient diagnosed with said cancer a therapeutically effective amount of a HER3 inhibitor, an EGFR inhibitor, or a combination of a HER3 inhibitor and an EGFR inhibitor, wherein the patient has a tumor that has been characterized in that cells from the tumor: i) express a neuregulin, and ii) express high levels of one or more of amphiregulin (AREG), TGF-α, and EGFR homodimer. In certain aspects, a patient diagnosed with a cancer treated by a method herein has a tumor that has been characterized as comprising cells that express high levels of a neuregulin and one or more of AREG, TGF-α, and EGFR homodimer. In certain embodiments the neuregulin is neuregulin 1 (NRG1) (e.g., NRG1α and/or NRG1β). In certain embodiments the neuregulin is neuregulin 2 (NRG2) (e.g., NRG2α and/or NRG2β). In specific embodiments, the HER3 inhibitor is an anti-HER3 antibody or antigen-binding fragment thereof (e.g. 2C2-YTE). In specific embodiments, the EGFR inhibitor is an anti-EGFR antibody or antigen-binding fragment thereof (e.g., cetuximab). In particular embodiments, the HER3 inhibitor is a 2C2-YTE anti-HER3 antibody and the EGFR inhibitor is cetuximab. In particular embodiments, the anti-HER3 antibody is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E) (2C2-YTE) and the EGFR inhibitor is cetuximab.

In another aspect, the disclosure provides methods for treating a cancer (e.g., squamous cell carcinoma of the head and neck (SCCHN)), comprising administering to a patient diagnosed with said cancer a therapeutically effective amount of a combination of a HER3 inhibitor and an EGFR inhibitor, wherein the patient has a tumor that has been characterized in that cells from the tumor: i) express a neuregulin, and ii) express high levels of one or more of AREG, TGF-α, and EGFR homodimer. In certain aspects, a patient diagnosed with a cancer treated by a method herein has a tumor that has been characterized as comprising cells that express high levels of a neuregulin and one or more of AREG, TGF-α, and EGFR homodimer. In certain embodiments the neuregulin is neuregulin 1 (NRG1) (e.g., NRG1α and/or NRG1β). In certain embodiments the neuregulin is NRG2 (e.g., NRG2α and/or NRG2β). In specific embodiments, the HER3 inhibitor is an anti-HER3 antibody or antigen-binding fragment thereof (e.g. 2C2-YTE). In specific embodiments, the EGFR inhibitor is an anti-EGFR antibody or antigen-binding fragment thereof (e.g., cetuximab). In particular embodiments, the HER3 inhibitor is a 2C2-YTE anti-HER3 antibody and the EGFR inhibitor is cetuximab. In particular embodiments, the HER3 inhibitor is a 2C2-YTE anti-HER3 antibody and the EGFR inhibitor is cetuximab. In particular embodiments, the anti-HER3 antibody is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E) and the EGFR inhibitor is cetuximab.

In yet another aspect, the disclosure provides methods for treating a cancer (e.g., squamous cell carcinoma of the head and neck (SCCHN)), comprising administering to a patient diagnosed with said cancer a therapeutically effective amount of a HER3 inhibitor, wherein the patient has a tumor that has been characterized in that cells from the tumor: i) express a neuregulin, and ii) express high levels of one or more of AREG, TGF-α, and EGFR homodimer. In certain aspects, a patient diagnosed with a cancer treated by a method herein has a tumor that has been characterized as comprising cells that express high levels of a neuregulin and one or more of amphiregulin, TGF-α, and EGFR homodimer. In certain embodiments the neuregulin is neuregulin 1 (NRG1) (e.g., NRG1α and/or NRG1β). In certain embodiments the neuregulin is NRG2 (e.g., NRG2α and/or NRG2β). In specific embodiments, the HER3 inhibitor is an anti-HER3 antibody or antigen-binding fragment thereof (e.g. 2C2-YTE). In particular embodiments, the HER3 inhibitor is a 2C2-YTE anti-HER3 antibody and the EGFR inhibitor is cetuximab. In particular embodiments, the HER3 inhibitor is a 2C2-YTE anti-HER3 antibody and the EGFR inhibitor is cetuximab. In particular embodiments, the anti-HER3 antibody is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E).

In another aspect, the disclosure provides methods for treating a cancer (e.g., head and neck cancer, for example, squamous cell carcinoma of the head and neck (SCCHN)), comprising administering to a patient diagnosed with said cancer a therapeutically effective amount of a EGFR inhibitor, wherein the patient has a tumor that has been characterized in that cells from the tumor: i) express a neuregulin, and ii) express high levels of one or more of AREG, TGF-α, and EGFR homodimer. In certain aspects, a patient diagnosed with a cancer treated by a method herein has a tumor that has been characterized as comprising cells that express high levels of a neuregulin and one or more of amphiregulin, TGF-α, and EGFR homodimer. In certain embodiments the neuregulin is neuregulin 1 (NRG1) (e.g., NRG1α and/or NRG1β). In certain embodiments the neuregulin is NRG2 (e.g., NRG2α and/or NRG2β). In specific embodiments, the EGFR inhibitor is an anti-EGFR antibody or an antigen-binding fragment thereof. In more specific embodiments, the anti-EGFR antibody is cetuximab.

In another aspect, the disclosure provides methods for treating a cancer (e.g., squamous cell carcinoma of the head and neck (SCCHN)), comprising administering to a patient diagnosed with said cancer a therapeutically effective amount of a HER3 inhibitor, an EGFR inhibitor, or a combination of a HER3 inhibitor and an EGFR inhibitor, wherein the patient has a tumor that has been characterized in that cells from the tumor: i) express high levels of EGFR homodimer, and ii) express high levels of one or more of AREG and TGF-α. In certain aspects, a patient diagnosed with a cancer treated by a method herein has a tumor that has been characterized as comprising cells that express high levels of EGFR homodimer and express high levels of one or more of amphiregulin and TGF-α. In specific embodiments, the HER3 inhibitor is an anti-HER3 antibody or antigen-binding fragment thereof (e.g. 2C2-YTE). In specific embodiments, the EGFR inhibitor is an anti-EGFR antibody or antigen-binding fragment thereof (e.g., cetuximab). In particular embodiments, the HER3 inhibitor is a 2C2-YTE anti-HER3 antibody and the EGFR inhibitor is cetuximab. In particular embodiments, the HER3 inhibitor is a 2C2-YTE anti-HER3 antibody and the EGFR inhibitor is cetuximab. In particular embodiments, the anti-HER3 antibody is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E) and the EGFR inhibitor is cetuximab.

In yet another aspect, the disclosure provides methods for treating a cancer (e.g., squamous cell carcinoma of the head and neck (SCCHN)), comprising administering to a patient diagnosed with said cancer a therapeutically effective amount of a combination of a HER3 inhibitor and an EGFR inhibitor, wherein the patient has a tumor has been characterized in that cells from the tumor: i) express high levels of EGFR homodimer, and ii) express high levels of one or more of AREG and TGF-α. In certain aspects, a patients diagnosed with a cancer treated by a method herein has a tumor characterized as comprising cells that express high levels of EGFR homodimer and express high levels of one or more of amphiregulin and TGF-α. In specific embodiments, the HER3 inhibitor is an anti-HER3 antibody or antigen-binding fragment thereof (e.g. 2C2-YTE). In specific embodiments, the EGFR inhibitor is an anti-EGFR antibody or antigen-binding fragment thereof (e.g., cetuximab). In particular embodiments, the HER3 inhibitor is a 2C2-YTE anti-HER3 antibody and the EGFR inhibitor is cetuximab. In particular embodiments, the HER3 inhibitor is a 2C2-YTE anti-HER3 antibody and the EGFR inhibitor is cetuximab. In particular embodiments, the anti-HER3 antibody is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E) and the EGFR inhibitor is cetuximab.

In another aspect, the disclosure provides methods for treating a cancer (e.g., squamous cell carcinoma of the head and neck (SCCHN)), comprising administering to a patient diagnosed with said cancer a therapeutically effective amount of a HER3 inhibitor, wherein the patient has a tumor that has been characterized in that cells from the tumor: i) express high levels of EGFR homodimer, and ii) express high levels of one or more of AREG and TGF-α. In certain aspects, a patient diagnosed with a cancer treated by a method herein has a tumor that has been characterized as comprising cells that express high levels of EGFR homodimer and express high levels of one or more of amphiregulin and TGF-α. In specific embodiments, the HER3 inhibitor is an anti-HER3 antibody or antigen-binding fragment thereof (e.g. 2C2-YTE). In particular embodiments, the HER3 inhibitor is a 2C2-YTE anti-HER3 antibody. In particular embodiments, the HER3 inhibitor is a 2C2-YTE anti-HER3 antibody and the EGFR inhibitor is cetuximab. In particular embodiments, the anti-HER3 antibody is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E).

In another aspect, the disclosure provides methods for treating a cancer (e.g., squamous cell carcinoma of the head and neck (SCCHN)), comprising administering to a patient diagnosed with said cancer a therapeutically effective amount of a EGFR inhibitor, wherein the patient has a tumor that has been characterized in that cells from the tumor: i) express high levels of EGFR homodimer, and ii) express high levels of one or more of AREG and TGF-α. In certain aspects, a patients diagnosed with a cancer treated by a method herein has a tumor that has been characterized as comprising cells that express high levels of EGFR homodimer and express high levels of one or more of amphiregulin and TGF-α. In specific embodiments, the EGFR inhibitor is an anti-EGFR antibody or antigen-binding fragment thereof (e.g., cetuximab). In particular embodiments, the HER3 inhibitor is a 2C2-YTE anti-HER3 antibody and the EGFR inhibitor is cetuximab.

In specific aspects, disclosed herein is a method of treating a human papillomavirus (HPV) positive head and neck cancer (e.g., SCCHN) in a human subject in need thereof, comprising administering to the human subject a therapeutically effective amount of an anti-HER3 antibody (e.g., 2C2-YTE) or an antigen-binding fragment thereof. In a specific aspect, disclosed herein is a method of treating an HPV positive head and neck cancer (e.g., SCCHN) in a human subject in need thereof, comprising administering to the human subject a therapeutically effective amount of an anti-HER3 antibody (e.g., 2C2-YTE). In specific aspects, the HPV positive head and neck cancer (e.g. SCCHN) is a cancer of the oral cavity, hypopharynx, oropharynx, rynopharynx, or larynx. In more specific embodiments, the HPV positive head and neck cancer (e.g., SCCHN) is an oropharyngeal cancer. In a specific aspect, a method of treating HPV positive head and neck cancer (e.g., SCCHN) described herein with an anti-HER3 antibody (e.g., 2C2-YTE) or an antigen-binding fragment thereof further comprises administering to the human subject a therapeutically effective amount of an EGFR inhibitor. In one aspect, the EGFR inhibitor is an anti-EGFR antibody such as cetuximab. In a particular aspect, the human subject has been diagnosed with an HPV positive head and neck cancer (e.g., SCCHN). In a certain aspect, the head and neck cancer (e.g., SCCHN) is EGFR expressing head and neck cancer.

In specific aspects, disclosed herein is a method of treating an HPV negative head and neck cancer (e.g., SCCHN) in a human subject in need thereof, comprising administering to the human subject a therapeutically effective amount of anti-HER3 antibody (e.g., 2C2-YTE) or an antigen-binding fragment thereof. In a specific aspect, disclosed herein is a method of treating an HPV negative head and neck cancer (e.g., SCCHN) in a human subject in need thereof, comprising administering to the human subject a therapeutically effective amount of an anti-HER3 antibody (e.g., 2C2-YTE). In specific aspects, the HPV positive head and neck cancer (e.g. SCCHN) is a cancer of the oral cavity, hypopharynx, oropharynx, rynopharynx, or larynx. In a specific aspect, a method of treating HPV negative head and neck cancer (e.g., SCCHN) described herein with an anti-HER3 antibody (e.g., 2C2-YTE) or an antigen-binding fragment thereof further comprises administering to the human subject a therapeutically effective amount of an EGFR inhibitor. In one aspect, the EGFR inhibitor is an anti-EGFR antibody such as cetuximab. In a particular aspect, the human subject has been diagnosed with an HPV negative head and neck cancer (e.g., SCCHN). In a certain aspect, the head and neck cancer (e.g., SCCHN) is EGFR expressing head and neck cancer.

In one aspect, the disclosure provides methods for treating a cancer (e.g., thyroid cancer or melanoma), comprising administering to a patient diagnosed with said cancer a therapeutically effective amount of a HER3 inhibitor or a combination of a HER3 inhibitor and a B-Raf inhibitor, wherein the patient has a tumor that has been characterized in that cells from the tumor express a neuregulin. In certain aspects, a patient diagnosed with a cancer treated by a method herein has a tumor that has been characterized as comprising cells that express high levels of a neuregulin. In certain embodiments the neuregulin is neuregulin 1 (NRG1) (e.g., NRG1α and/or NRG1β). In certain embodiments the neuregulin is neuregulin 2 (NRG2) (e.g., NRG2α and/or NRG2β). In particular embodiments, the tumor has been characterized as comprising cells that express high levels of NRG1 and NRG2. In specific embodiments, the HER3 inhibitor is an anti-HER3 antibody or antigen-binding fragment thereof (e.g. 2C2-YTE). In specific embodiments, the B-Raf inhibitor is vemurafenib. In specific embodiments, the B-Raf inhibitor is dabrafenib. In particular embodiments, the HER3 inhibitor is a 2C2-YTE anti-HER3 antibody and the B-Raf inhibitor is vemurafenib. In particular embodiments, the HER3 inhibitor is a 2C2-YTE anti-HER3 antibody and the B-Raf inhibitor is dabrafenib. In particular embodiments, the anti-HER3 antibody is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E) and the B-Raf inhibitor is vemurafenib or dabrafenib. In certain embodiments, the cancer is characterized by a BRAF mutation (e.g., V600E or V600K). In certain embodiments, the cancer is resistant to treatment with a BRAF inhibitor (e.g., vemurafenib or dabrafenib). In another embodiment, the cancer is resistant to treatment with a MEK inhibitor (e.g., trametinib). In other embodiments, the cancer is resistant to treatment with a BRAF inhibitor (e.g., vemurafenib or dabrafenib) and a MEK inhibitor (e.g., trametinib). In certain embodiments, the cancer is melanoma. In specific embodiments, the cancer is B-Raf mutated melanoma. In certain embodiments, the cancer is thyroid cancer. In specific embodiments, the cancer is B-Raf mutated thyroid cancer. In certain embodiments, the cancer is colorectal cancer. In specific embodiments, the cancer is B-Raf mutated colorectal cancer. In certain embodiments, the cancer is lung cancer. In specific embodiments, the cancer is B-Raf mutated lung cancer. In certain embodiments, the cancer is hairy cell leukemia. In specific embodiments, the cancer is B-Raf mutated hairy cell leukemia. In particular embodiments, the lung cancer is non-small cell lung carcinoma. In certain embodiments, the cancer is a squamous cell carcinoma of the head and neck (SCCHN). In certain embodiments, the cancer is ovarian cancer. In certain embodiments, the cancer is pancreatic cancer. In certain embodiments, the cancer is bladder cancer. In certain embodiments, the cancer is testicular cancer. In certain embodiments, the cancer is endometrial cancer. In certain embodiments, the cancer is hepatocellular carcinoma. In certain embodiments, the cancer is breast cancer. In certain embodiments, the cancer is gastric cancer. In certain embodiments, the cancer is prostate cancer. In certain embodiments, the method of treatment comprises a step of measuring the expression of a neuregulin (e.g. NRG1 and/or NRG2) in the cells of the tumor. In certain embodiments, said measuring is performed in vitro.

In one aspect, the disclosure provides methods for treating a cancer (e.g., thyroid cancer or melanoma), comprising administering to a patient diagnosed with said cancer a therapeutically effective amount of a HER3 inhibitor, wherein the patient has a tumor that has been characterized in that cells from the tumor express a neuregulin. In certain aspects, a patient diagnosed with a cancer treated by a method herein has a tumor that has been characterized as comprising cells that express high levels of a neuregulin. In certain embodiments the neuregulin is neuregulin 1 (NRG1) (e.g., NRG1α and/or NRG1β). In certain embodiments the neuregulin is neuregulin 2 (NRG2) (e.g., NRG2α and/or NRG2β). In particular embodiments, the tumor has been characterized as comprising cells that express high levels of NRG1 and NRG2. In specific embodiments, the HER3 inhibitor is an anti-HER3 antibody or antigen-binding fragment thereof (e.g. 2C2-YTE). In particular embodiments, the HER3 inhibitor is a 2C2-YTE anti-HER3 antibody. In particular embodiments, the anti-HER3 antibody is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E). In certain embodiments, the cancer is characterized by a BRAF mutation (e.g., V600E or V600K). In certain embodiments, the cancer is resistant to treatment with a BRAF inhibitor (e.g., vemurafenib or dabrafenib). In another embodiment, the cancer is resistant to treatment with a MEK inhibitor (e.g., trametinib). In other embodiments, the cancer is resistant to treatment with a BRAF inhibitor (e.g., vemurafenib or dabrafenib) and a MEK inhibitor (e.g., trametinib). In certain embodiments, the cancer is melanoma. In specific embodiments, the cancer is B-Raf mutated melanoma. In certain embodiments, the cancer is thyroid cancer. In specific embodiments, the cancer is B-Raf mutated thyroid cancer. In certain embodiments, the cancer is colorectal cancer. In specific embodiments, the cancer is B-Raf mutated colorectal cancer. In certain embodiments, the cancer is lung cancer. In specific embodiments, the cancer is B-Raf mutated lung cancer. In particular embodiments, the lung cancer is non-small cell lung carcinoma. In certain embodiments, the cancer is a squamous cell carcinoma of the head and neck (SCCHN). In certain embodiments, the cancer is ovarian cancer. In certain embodiments, the cancer is pancreatic cancer. In certain embodiments, the cancer is bladder cancer. In certain embodiments, the cancer is testicular cancer. In certain embodiments, the cancer is endometrial cancer. In certain embodiments, the cancer is hepatocellular carcinoma. In certain embodiments, the cancer is breast cancer. In certain embodiments, the cancer is gastric cancer. In certain embodiments, the cancer is prostate cancer. In certain embodiments, the method of treatment comprises a step of measuring the expression of a neuregulin (e.g. NRG1 and/or NRG2) in the cells of the tumor. In certain embodiments, said measuring is performed in vitro.

In one aspect, the disclosure provides methods for treating a cancer (e.g., thyroid cancer or melanoma), comprising administering to a patient diagnosed with said cancer a therapeutically effective amount of a combination of a HER3 inhibitor and a MEK inhibitor, wherein the patient has a tumor that has been characterized in that cells from the tumor express a neuregulin. In certain aspects, a patient diagnosed with a cancer treated by a method herein has a tumor that has been characterized as comprising cells that express high levels of a neuregulin. In certain embodiments the neuregulin is neuregulin 1 (NRG1) (e.g., NRG1α and/or NRG1β). In certain embodiments the neuregulin is neuregulin 2 (NRG2) (e.g., NRG2α and/or NRG2β). In particular embodiments, the tumor has been characterized as comprising cells that express high levels of NRG1 and NRG2. In specific embodiments, the HER3 inhibitor is an anti-HER3 antibody or antigen-binding fragment thereof (e.g. 2C2-YTE). In specific embodiments, MEK inhibitor is selumetinib. In specific embodiments, the MEK inhibitor is trametinib. In particular embodiments, the HER3 inhibitor is a 2C2-YTE anti-HER3 antibody and the B-Raf inhibitor is selumetinib. In particular embodiments, the HER3 inhibitor is a 2C2-YTE anti-HER3 antibody and the MEK inhibitor is trametinib. In particular embodiments, the anti-HER3 antibody is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E) and the MEK inhibitor is selumetinib or trametinib. In certain embodiments, the cancer is characterized by a BRAF mutation (e.g., V600E or V600K). In certain embodiments, the cancer is resistant to treatment with a BRAF inhibitor (e.g., vemurafenib or dabrafenib). In another embodiment, the cancer is resistant to treatment with a MEK inhibitor (e.g., trametinib). In other embodiments, the cancer is resistant to treatment with a BRAF inhibitor (e.g., vemurafenib or dabrafenib) and a MEK inhibitor (e.g., trametinib). In certain embodiments, the cancer is melanoma. In specific embodiments, the cancer is B-Raf mutated melanoma. In certain embodiments, the cancer is thyroid cancer. In specific embodiments, the cancer is B-Raf mutated thyroid cancer. In certain embodiments, the cancer is colorectal cancer. In specific embodiments, the cancer is B-Raf mutated colorectal cancer. In certain embodiments, the cancer is lung cancer. In specific embodiments, the cancer is B-Raf mutated lung cancer. In certain embodiments, the cancer is hairy cell leukemia. In specific embodiments, the cancer is B-Raf mutated hairy cell leukemia. In particular embodiments, the lung cancer is non-small cell lung carcinoma. In certain embodiments, the cancer is a squamous cell carcinoma of the head and neck (SCCHN). In certain embodiments, the cancer is ovarian cancer. In certain embodiments, the cancer is pancreatic cancer. In certain embodiments, the cancer is bladder cancer. In certain embodiments, the cancer is testicular cancer. In certain embodiments, the cancer is endometrial cancer. In certain embodiments, the cancer is hepatocellular carcinoma. In certain embodiments, the cancer is breast cancer. In certain embodiments, the cancer is gastric cancer. In certain embodiments, the cancer is prostate cancer. In certain embodiments, the method of treatment comprises a step of measuring the expression of a neuregulin (e.g. NRG1 and/or NRG2) in the cells of the tumor. In certain embodiments, said measuring is performed in vitro.

In one aspect, the disclosure provides methods for treating a cancer (e.g., thyroid cancer or melanoma), comprising administering to a patient diagnosed with said cancer a therapeutically effective amount of a combination of a HER3 inhibitor, a B-Raf inhibitor, and a MEK inhibitor, wherein the patient has a tumor that has been characterized in that cells from the tumor express a neuregulin. In certain aspects, a patient diagnosed with a cancer treated by a method herein has a tumor that has been characterized as comprising cells that express high levels of a neuregulin. In certain embodiments the neuregulin is neuregulin 1 (NRG1) (e.g., NRG1α and/or NRG1β). In certain embodiments the neuregulin is neuregulin 2 (NRG2) (e.g., NRG2α and/or NRG2β). In particular embodiments, the tumor has been characterized as comprising cells that express high levels of NRG1 and NRG2. In specific embodiments, the HER3 inhibitor is an anti-HER3 antibody or antigen-binding fragment thereof (e.g. 2C2-YTE). In specific embodiments, the B-Raf inhibitor is vemurafenib. In specific embodiments, the B-Raf inhibitor is dabrafenib. In specific embodiments, MEK inhibitor is selumetinib. In specific embodiments, the MEK inhibitor is trametinib. In particular embodiments, the HER3 inhibitor is a 2C2-YTE anti-HER3 antibody, the B-Raf inhibitor is vemurafenib, and the MEK inhibitor is selumetinib or trametinib. In particular embodiments, the HER3 inhibitor is a 2C2-YTE anti-HER3 antibody, the B-Raf inhibitor is dabrafenib, and the MEK inhibitor is selumetinib or trametinib. In particular embodiments, the anti-HER3 antibody is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E), the B-Raf inhibitor is vemurafenib or dabrafenib, and the MEK inhibitor is selumetinib or trametinib. In certain embodiments, the cancer is characterized by a BRAF mutation (e.g., V600E or V600K). In certain embodiments, the cancer is resistant to treatment with a BRAF inhibitor (e.g., vemurafenib or dabrafenib). In another embodiment, the cancer is resistant to treatment with a MEK inhibitor (e.g., trametinib). In other embodiments, the cancer is resistant to treatment with a BRAF inhibitor (e.g., vemurafenib or dabrafenib) and a MEK inhibitor (e.g., trametinib). In certain embodiments, the cancer is melanoma. In specific embodiments, the cancer is B-Raf mutated melanoma. In certain embodiments, the cancer is thyroid cancer. In specific embodiments, the cancer is B-Raf mutated thyroid cancer. In certain embodiments, the cancer is colorectal cancer. In specific embodiments, the cancer is B-Raf mutated colorectal cancer. In certain embodiments, the cancer is lung cancer. In specific embodiments, the cancer is B-Raf mutated lung cancer. In certain embodiments, the cancer is hairy cell leukemia. In specific embodiments, the cancer is B-Raf mutated hairy cell leukemia. In particular embodiments, the lung cancer is non-small cell lung carcinoma. In certain embodiments, the cancer is a squamous cell carcinoma of the head and neck (SCCHN). In certain embodiments, the cancer is ovarian cancer. In certain embodiments, the cancer is pancreatic cancer. In certain embodiments, the cancer is bladder cancer. In certain embodiments, the cancer is testicular cancer. In certain embodiments, the cancer is endometrial cancer. In certain embodiments, the cancer is hepatocellular carcinoma. In certain embodiments, the cancer is breast cancer. In certain embodiments, the cancer is gastric cancer. In certain embodiments, the cancer is prostate cancer. In certain embodiments, the method of treatment comprises a step of measuring the expression of a neuregulin (e.g. NRG1 and/or NRG2) in the cells of the tumor. In certain embodiments, said measuring is performed in vitro.

In one aspect, the disclosure provides methods for treating a cancer (e.g., thyroid cancer or melanoma), comprising administering to a patient diagnosed with said cancer a therapeutically effective amount of a HER3 inhibitor or a combination of a HER3 inhibitor and an EGFR inhibitor, wherein the patient has a tumor that has been characterized in that cells from the tumor express a neuregulin. In certain aspects, a patient diagnosed with a cancer treated by a method herein has a tumor that has been characterized as comprising cells that express high levels of a neuregulin. In certain embodiments the neuregulin is neuregulin 1 (NRG1) (e.g., NRG1α and/or NRG1β). In certain embodiments the neuregulin is neuregulin 2 (NRG2) (e.g., NRG2α and/or NRG2β). In particular embodiments, the tumor has been characterized as comprising cells that express high levels of NRG1 and NRG2. In specific embodiments, the HER3 inhibitor is an anti-HER3 antibody or antigen-binding fragment thereof (e.g. 2C2-YTE). In specific embodiments, the EGFR inhibitor is cetuximab. In particular embodiments, the HER3 inhibitor is a 2C2-YTE anti-HER3 antibody and the EGFR inhibitor is cetuximab. In particular embodiments, the anti-HER3 antibody is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E) and the EGFR inhibitor is cetuximab. In certain embodiments, the cancer is characterized by a BRAF mutation (e.g., V600E or V600K). In certain embodiments, the cancer is resistant to treatment with a BRAF inhibitor (e.g., vemurafenib or dabrafenib). In another embodiment, the cancer is resistant to treatment with a MEK inhibitor (e.g., trametinib). In other embodiments, the cancer is resistant to treatment with a BRAF inhibitor (e.g., vemurafenib or dabrafenib) and a MEK inhibitor (e.g., trametinib). In certain embodiments, the cancer is melanoma. In specific embodiments, the cancer is B-Raf mutated melanoma. In certain embodiments, the cancer is thyroid cancer. In specific embodiments, the cancer is B-Raf mutated thyroid cancer. In certain embodiments, the cancer is colorectal cancer. In specific embodiments, the cancer is B-Raf mutated colorectal cancer. In certain embodiments, the cancer is lung cancer. In specific embodiments, the cancer is B-Raf mutated lung cancer. In certain embodiments, the cancer is hairy cell leukemia. In specific embodiments, the cancer is B-Raf mutated hairy cell leukemia. In particular embodiments, the lung cancer is non-small cell lung carcinoma. In certain embodiments, the cancer is a squamous cell carcinoma of the head and neck (SCCHN). In certain embodiments, the cancer is ovarian cancer. In certain embodiments, the cancer is pancreatic cancer. In certain embodiments, the cancer is bladder cancer. In certain embodiments, the cancer is testicular cancer. In certain embodiments, the cancer is endometrial cancer. In certain embodiments, the cancer is hepatocellular carcinoma. In certain embodiments, the cancer is breast cancer. In certain embodiments, the cancer is gastric cancer. In certain embodiments, the cancer is prostate cancer. In certain embodiments, the method of treatment comprises a step of measuring the expression of a neuregulin (e.g. NRG1 and/or NRG2) in the cells of the tumor. In certain embodiments, said measuring is performed in vitro.

In yet another aspect, the disclosure provides methods for determining whether a patient diagnosed with cancer (e.g., head and neck cancer, for example, SCCHN) is indicated as likely to be responsive to treatment with a HER3 inhibitor, an EGFR inhibitor, or a combination of a HER3 inhibitor and an EGFR inhibitor, comprising measuring the expression of one or more of AREG, TGF-α, and EGFR homodimer in a sample from the patient, wherein the sample comprises tumor cells and a neuregulin, and wherein high levels of one or more of AREG, TGF-α, and EGFR homodimer in the sample indicates that the patient is likely to be responsive to treatment with a HER3 inhibitor, an EGFR inhibitor, or a combination of a HER3 inhibitor and an EGFR inhibitor. In specific embodiments, the neuregulin is NRG1 (e.g., NRG1α and/or NRG1β). In specific embodiments, the neuregulin is NRG2 (e.g., NRG2α and/or NRG2β). In certain embodiments, the neuregulin is expressed at a high level in the sample. In certain embodiments, said measuring is performed in vitro.

In certain embodiments, such methods for determining whether a patient diagnosed with cancer (e.g., head and neck cancer, for example, SCCHN) is indicated as likely to be responsive to treatment with a HER3 inhibitor, an EGFR inhibitor, or a combination of a HER3 inhibitor and an EGFR inhibitor comprise measuring a level of one or more of AREG, TGF-α, and EGFR homodimer in the sample. In certain embodiments, the method comprises measuring a level of two or more of AREG, TGF-α, and EGFR homodimer in the sample. In certain embodiments, the method comprises measuring a level of AREG and TGF-α in the sample. In certain embodiments, the method comprises measuring a level of AREG, TGF-α, and EGFR homodimer in the sample. In certain embodiments, the method further comprises measuring a level of neuregulin (e.g., NRG1 and/or NRG2) in the sample. In certain embodiments, the step of measuring a level of AREG, TGF-α and/or neuregulin (e.g., NRG1 and/or NRG2) comprises measuring the level of AREG, TGF-α and/or neuregulin (e.g., NRG1 and/or NRG2) RNA in the sample. In certain embodiments, the step of measuring the expression of EGFR homodimer comprises measuring the level of protein-protein interaction between EGFR monomers in the sample. In particular embodiments, the expression of EGFR homodimer is measured using a protein proximity assay. In certain embodiments, said measuring is performed in vitro.

In certain embodiments, such methods for determining whether a patient diagnosed with cancer (e.g., head and neck cancer, for example, SCCHN) is indicated as likely to be responsive to treatment with a HER3 inhibitor, an EGFR inhibitor, or a combination of a HER3 inhibitor and an EGFR inhibitor comprise a first step of obtaining the sample from a tumor from the patient. In certain embodiments, the method comprises an additional step of administering to the patient a therapeutically effective amount of a HER3 inhibitor. In certain embodiments, the method comprises an additional step of administering to the patient a therapeutically effective amount of an EGFR inhibitor. In certain embodiments, the method comprises an additional step of administering to the patient a therapeutically effective amount of a HER3 inhibitor and a therapeutically effective amount of an EGFR inhibitor. In specific embodiments, the HER3 inhibitor is an anti-HER3 antibody or antigen-binding fragment thereof (e.g. 2C2-YTE). In specific embodiments, the EGFR inhibitor is an anti-EGFR antibody or antigen-binding fragment thereof (e.g., cetuximab). In particular embodiments, the HER3 inhibitor is a 2C2-YTE anti-HER3 antibody and the EGFR inhibitor is cetuximab. In particular embodiments, the HER3 inhibitor is a 2C2-YTE anti-HER3 antibody and the EGFR inhibitor is cetuximab. In particular embodiments, the anti-HER3 antibody is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E) and the EGFR inhibitor is cetuximab.

In yet another aspect, the disclosure provides methods for determining whether a patient diagnosed with cancer (e.g., head and neck cancer, for example, SCCHN) is indicated as likely to be responsive to treatment with a combination of a HER3 inhibitor and an EGFR inhibitor, comprising measuring the expression of one or more of AREG, TGF-α, and EGFR homodimer in a sample from the patient, wherein the sample comprises tumor cells and a neuregulin, and wherein high levels of one or more of AREG, TGF-α, and EGFR homodimer in the sample indicates that the patient is likely to be responsive to treatment with a combination of a HER3 inhibitor and an EGFR inhibitor. In specific embodiments, the neuregulin is NRG1 (e.g., NRG1α and/or NRG1β). In specific embodiments, the neuregulin is NRG2 (e.g., NRG2α and/or NRG2β). In certain embodiments, the neuregulin is expressed at a high level in the sample. In certain embodiments, said measuring is performed in vitro.

In certain embodiments, such methods for determining whether a patient diagnosed with cancer (e.g., head and neck cancer, for example, SCCHN) is indicated as likely to be responsive to treatment with a combination of a HER3 inhibitor and an EGFR inhibitor, comprise measuring a level of one or more of AREG, TGF-α, and EGFR homodimer in the sample. In certain embodiments, the method comprises measuring a level of two or more of AREG, TGF-α, and EGFR homodimer in the sample. In certain embodiments, the method comprises measuring a level of AREG, TGF-α, and EGFR homodimer in the sample. In certain embodiments, the method further comprises measuring a level of neuregulin (e.g., NRG1 and/or NRG2) in the sample. In certain embodiments, the step of measuring a level of AREG, TGF-α and/or neuregulin (e.g., NRG1 and/or NRG2) comprises measuring the level of AREG, TGF-α and/or neuregulin (e.g., NRG1 and/or NRG2) RNA in the sample. In certain embodiments, the step of measuring the expression of EGFR homodimer comprises measuring the level of protein-protein interaction between EGFR monomers in the sample. In particular embodiments, the expression of EGFR homodimer is measured using a protein proximity assay. In certain embodiments, said measuring is performed in vitro.

In certain embodiments, such methods for determining whether a patient diagnosed with cancer (e.g., head and neck cancer, for example, SCCHN) is indicated as likely to be responsive to treatment with a combination of a HER3 inhibitor and an EGFR inhibitor, comprise a first step of obtaining the sample from a tumor from the patient. In certain embodiments, the method comprises an additional step of administering to the patient a therapeutically effective amount of a HER3 inhibitor and a therapeutically effective amount of an EGFR inhibitor. In specific embodiments, the HER3 inhibitor is an anti-HER3 antibody or antigen-binding fragment thereof (e.g. 2C2-YTE). In specific embodiments, the EGFR inhibitor is an anti-EGFR antibody or antigen-binding fragment thereof (e.g., cetuximab). In particular embodiments, the HER3 inhibitor is a 2C2-YTE anti-HER3 antibody and the EGFR inhibitor is cetuximab. In particular embodiments, the HER3 inhibitor is a 2C2-YTE anti-HER3 antibody and the EGFR inhibitor is cetuximab. In particular embodiments, the anti-HER3 antibody is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E) and the EGFR inhibitor is cetuximab.

In yet another aspect, the disclosure provides methods for determining whether a patient diagnosed with cancer (e.g., head and neck cancer, for example, SCCHN) is indicated as likely to be responsive to treatment with a HER3 inhibitor, comprising measuring the expression of one or more of AREG, TGF-α, and EGFR homodimer in a sample from the patient, wherein the sample comprises tumor cells and a neuregulin, and wherein high levels of one or more of AREG, TGF-α, and EGFR homodimer in the sample indicates that the patient is likely to be responsive to treatment with a HER3 inhibitor. In specific embodiments, the neuregulin is NRG1 (e.g., NRG1α and/or NRG1β). In specific embodiments, the neuregulin is NRG2 (e.g., NRG2α and/or NRG2β). In certain embodiments, the neuregulin is expressed at a high level in the sample. In certain embodiments, said measuring is performed in vitro.

In certain embodiments, such methods for determining whether a patient diagnosed with cancer (e.g., head and neck cancer, for example, SCCHN) is indicated as likely to be responsive to treatment with a HER3 inhibitor comprise measuring a level of one or more of AREG, TGF-α, and EGFR homodimer in the sample. In certain embodiments, the method comprises measuring a level of two or more of AREG, TGF-α, and EGFR homodimer in the sample. In certain embodiments, the method comprises measuring a level of AREG, TGF-α, and EGFR homodimer in the sample. In certain embodiments, the method further comprises measuring a level of neuregulin (e.g., NRG1 and/or NRG2) in the sample. In certain embodiments, the step of measuring a level of AREG, TGF-α and/or neuregulin (e.g., NRG1 and/or NRG2) comprises measuring the level of AREG, TGF-α and/or neuregulin (e.g., NRG1 and/or NRG2) RNA in the sample. In certain embodiments, the step of measuring the expression of EGFR homodimer comprises measuring the level of protein-protein interaction between EGFR monomers in the sample. In particular embodiments, the expression of EGFR homodimer is measured using a protein proximity assay. In certain embodiments, said measuring is performed in vitro.

In certain embodiments, such methods for determining whether a patient diagnosed with cancer (e.g., head and neck cancer, for example, SCCHN) is indicated as likely to be responsive to treatment with a HER3 inhibitor comprise a first step of obtaining the sample from a tumor from the patient. In certain embodiments, the method comprises an additional step of administering to the patient a therapeutically effective amount of a HER3 inhibitor. In specific embodiments, the HER3 inhibitor is an anti-HER3 antibody or antigen-binding fragment thereof (e.g. 2C2-YTE). In particular embodiments, the HER3 inhibitor is a 2C2-YTE anti-HER3 antibody. In particular embodiments, the HER3 inhibitor is a 2C2-YTE anti-HER3 antibody and the EGFR inhibitor is cetuximab. In particular embodiments, the anti-HER3 antibody is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E).

In yet another aspect, the disclosure provides methods for determining whether a patient diagnosed with cancer (e.g., head and neck cancer, for example, SCCHN) is indicated as likely to be responsive to treatment with an EGFR inhibitor, comprising measuring the expression of one or more of AREG, TGF-α, and EGFR homodimer in a sample from the patient, wherein the sample comprises tumor cells and a neuregulin, and wherein high levels of one or more of AREG, TGF-α, and EGFR homodimer in the sample indicates that the patient is likely to be responsive to treatment with an EGFR inhibitor. In specific embodiments, the neuregulin is NRG1 (e.g., NRG1α and/or NRG1β). In specific embodiments, the neuregulin is NRG2 (e.g., NRG2α and/or NRG2β). In certain embodiments, the neuregulin is expressed at a high level in the sample. In certain embodiments, said measuring is performed in vitro.

In certain embodiments, such methods for determining whether a patient diagnosed with cancer (e.g., head and neck cancer, for example, SCCHN) is indicated as likely to be responsive to treatment with an EGFR inhibitor comprise measuring a level of one or more of AREG, TGF-α, and EGFR homodimer in the sample. In certain embodiments, the method comprises measuring a level of two or more of AREG, TGF-α, and EGFR homodimer in the sample. In certain embodiments, the method comprises measuring a level of AREG, TGF-α, and EGFR homodimer in the sample. In certain embodiments, the method further comprises measuring a level of neuregulin (e.g., NRG1 and/or NRG2) in the sample. In certain embodiments, the step of measuring a level of AREG, TGF-α and/or neuregulin (e.g., NRG1 and/or NRG2) comprises measuring the level of AREG, TGF-α and/or neuregulin (e.g., NRG1 and/or NRG2) RNA in the sample. In certain embodiments, the step of measuring the expression of EGFR homodimer comprises measuring the level of protein-protein interaction between EGFR monomers in the sample. In particular embodiments, the expression of EGFR homodimer is measured using a protein proximity assay. In certain embodiments, said measuring is performed in vitro.

In certain embodiments, such methods for determining whether a patient diagnosed with cancer (e.g., head and neck cancer, for example, SCCHN) is indicated as likely to be responsive to treatment with an EGFR inhibitor comprise a first step of obtaining the sample from a tumor from the patient. In certain embodiments, the method comprises an additional step of administering to the patient a therapeutically effective amount of an EGFR inhibitor. In specific embodiments, the EGFR inhibitor is an anti-EGFR antibody or antigen-binding fragment thereof (e.g. cetuximab). In particular embodiments, the EGFR inhibitor is cetuximab.

In another aspect, the disclosure provides methods for determining whether a patient diagnosed with cancer (e.g., head and neck cancer, for example, SCCHN) is indicated as likely to be responsive to treatment with a HER3 inhibitor, an EGFR inhibitor, or a combination of a HER3 inhibitor and an EGFR inhibitor, comprising measuring the expression of a neuregulin, and at least one of AREG, TGF-α, and EGFR homodimer in a sample from the patient, wherein the sample comprises tumor cells, and wherein the presence of the neuregulin and high levels of one or more of AREG, TGF-α, and EGFR homodimer in the sample indicates that the patient is likely to be responsive to treatment with a HER3 inhibitor, an EGFR inhibitor, or a combination of a HER3 inhibitor and an EGFR inhibitor. In certain embodiments, the neuregulin is expressed at a high level in the sample. In specific embodiments, the neuregulin is NRG1 (e.g., NRG1α and/or NRG1β). In specific embodiments, the neuregulin is NRG2 (e.g., NRG2α and/or NRG2β). In certain embodiments, said measuring is performed in vitro.

In certain embodiments, such methods for determining whether a patient diagnosed with cancer (e.g., head and neck cancer, for example, SCCHN) is indicated as likely to be responsive to treatment with a HER3 inhibitor, an EGFR inhibitor, or a combination of a HER3 inhibitor and an EGFR inhibitor comprise measuring a level of one or more of AREG, TGF-α, and EGFR homodimer in the sample. In certain embodiments, the method comprises measuring a level of two or more of AREG, TGF-α, and EGFR homodimer in the sample. In certain embodiments, the method comprises measuring a level of AREG and TGF-α in the sample. In certain embodiments, the method comprises measuring a level of AREG, TGF-α, and EGFR homodimer in the sample. In certain embodiments, the step of measuring expression of neuregulin (e.g., NRG1 and/or NRG2) and AREG and/or TGF-α comprises measuring the level of neuregulin (e.g., NRG1 and/or NRG2) and AREG and/or TGF-α RNA in the sample. In certain embodiments, the step of measuring the expression of EGFR homodimer comprises measuring the level of protein-protein interaction between EGFR monomers in the sample. In particular embodiments, the expression of EGFR homodimer is measured using a protein proximity assay. In certain embodiments, said measuring is performed in vitro.

In certain embodiments, such methods for determining whether a patient diagnosed with cancer (e.g., head and neck cancer, for example, SCCHN) is indicated as likely to be responsive to treatment with a HER3 inhibitor, an EGFR inhibitor, or a combination of a HER3 inhibitor and an EGFR inhibitor comprise a first step of obtaining the sample from a tumor from the patient. In certain embodiments, the method comprises an additional step of administering to the patient a therapeutically effective amount of a HER3 inhibitor. In certain embodiments, the method comprises an additional step of administering to the patient a therapeutically effective amount of an EGFR inhibitor. In certain embodiments, the method comprises an additional step of administering to the patient a therapeutically effective amount of a HER3 inhibitor and a therapeutically effective amount of an EGFR inhibitor. In specific embodiments, the HER3 inhibitor is an anti-HER3 antibody or antigen-binding fragment thereof (e.g. 2C2-YTE). In specific embodiments, the EGFR inhibitor is an anti-EGFR antibody or antigen-binding fragment thereof (e.g., cetuximab). In particular embodiments, the HER3 inhibitor is a 2C2-YTE anti-HER3 antibody and the EGFR inhibitor is cetuximab. In particular embodiments, the HER3 inhibitor is a 2C2-YTE anti-HER3 antibody and the EGFR inhibitor is cetuximab. In particular embodiments, the anti-HER3 antibody is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E) and the EGFR inhibitor is cetuximab.

In another aspect, the disclosure provides methods for determining whether a patient diagnosed with cancer (e.g., head and neck cancer, for example, SCCHN) is indicated as likely to be responsive to treatment with a combination of a HER3 inhibitor and an EGFR inhibitor, comprising measuring the expression of a neuregulin, and at least one of AREG, TGF-α, and EGFR homodimer in a sample from the patient, wherein the sample comprises tumor cells, and wherein the presence of the neuregulin and high levels of one or more of AREG, TGF-α, and EGFR homodimer in the sample indicates that the patient is likely to be responsive to treatment with a combination of a HER3 inhibitor and an EGFR inhibitor. In certain embodiments, the neuregulin is expressed at a high level in the sample. In specific embodiments, the neuregulin is NRG1 (e.g., NRG1α and/or NRG1β). In specific embodiments, the neuregulin is NRG2 (e.g., NRG2α and/or NRG2β). In certain embodiments, said measuring is performed in vitro.

In certain embodiments, such methods for determining whether a patient diagnosed with cancer (e.g., head and neck cancer, for example, SCCHN) is indicated as likely to be responsive to treatment with a combination of a HER3 inhibitor and an EGFR inhibitor comprise measuring a level of one or more of AREG, TGF-α, and EGFR homodimer in the sample. In certain embodiments, the method comprises measuring a level of two or more of AREG, TGF-α, and EGFR homodimer in the sample. In certain embodiments, the method comprises measuring a level of AREG and TGF-α in the sample. In certain embodiments, the method comprises measuring a level of AREG, TGF-α, and EGFR homodimer in the sample. In certain embodiments, the step of measuring expression of neuregulin (e.g., NRG1 and/or NRG2) and AREG and/or TGF-α comprises measuring the level of neuregulin (e.g., NRG1 and/or NRG2) and AREG and/or TGF-α RNA in the sample. In certain embodiments, the step of measuring the expression of EGFR homodimer comprises measuring the level of protein-protein interaction between EGFR monomers in the sample. In particular embodiments, the expression of EGFR homodimer is measured using a protein proximity assay. In certain embodiments, said measuring is performed in vitro.

In certain embodiments, such methods for determining whether a patient diagnosed with cancer (e.g., head and neck cancer, for example, SCCHN) is indicated as likely to be responsive to treatment with a combination of a HER3 inhibitor and an EGFR inhibitor comprise a first step of obtaining the sample from a tumor from the patient. In certain embodiments, the method comprises an additional step of administering to the patient a therapeutically effective amount of a HER3 inhibitor and a therapeutically effective amount of an EGFR inhibitor. In specific embodiments, the HER3 inhibitor is an anti-HER3 antibody or antigen-binding fragment thereof (e.g. 2C2-YTE). In specific embodiments, the EGFR inhibitor is an anti-EGFR antibody or antigen-binding fragment thereof (e.g., cetuximab). In particular embodiments, the HER3 inhibitor is a 2C2-YTE anti-HER3 antibody and the EGFR inhibitor is cetuximab. In particular embodiments, the HER3 inhibitor is a 2C2-YTE anti-HER3 antibody and the EGFR inhibitor is cetuximab. In particular embodiments, the anti-HER3 antibody is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E) and the EGFR inhibitor is cetuximab.

In another aspect, the disclosure provides methods for determining whether a patient diagnosed with cancer (e.g., head and neck cancer, for example, SCCHN) is indicated as likely to be responsive to treatment with a HER3 inhibitor, comprising measuring the expression of a neuregulin, and at least one of AREG, TGF-α, and EGFR homodimer in a sample from the patient, wherein the sample comprises tumor cells, and wherein the presence of the neuregulin and high levels of one or more of AREG, TGF-α, and EGFR homodimer in the sample indicates that the patient is likely to be responsive to treatment with a HER3 inhibitor. In certain embodiments, the neuregulin is expressed at a high level in the sample. In specific embodiments, the neuregulin is NRG1 (e.g., NRG1α and/or NRG1β). In specific embodiments, the neuregulin is NRG2 (e.g., NRG2α and/or NRG2β). In certain embodiments, said measuring is performed in vitro.

In certain embodiments, such methods for determining whether a patient diagnosed with cancer (e.g., head and neck cancer, for example, SCCHN) is indicated as likely to be responsive to treatment with a HER3 inhibitor comprise measuring a level of one or more of AREG, TGF-α, and EGFR homodimer in the sample. In certain embodiments, the method comprises measuring a level of two or more of AREG, TGF-α, and EGFR homodimer in the sample. In certain embodiments, the method comprises measuring a level of AREG and TGF-α in the sample. In certain embodiments, the method comprises measuring a level of AREG, TGF-α, and EGFR homodimer in the sample. In certain embodiments, the step of measuring expression of neuregulin (e.g., NRG1 and/or NRG2) and AREG and/or TGF-α comprises measuring the level of neuregulin (e.g., NRG1 and/or NRG2) and AREG and/or TGF-α RNA in the sample. In certain embodiments, the step of measuring the expression of EGFR homodimer comprises measuring the level of protein-protein interaction between EGFR monomers in the sample. In particular embodiments, the expression of EGFR homodimer is measured using a protein proximity assay. In certain embodiments, said measuring is performed in vitro.

In certain embodiments, such methods for determining whether a patient diagnosed with cancer (e.g., head and neck cancer, for example, SCCHN) is indicated as likely to be responsive to treatment with a HER3 inhibitor comprise a first step of obtaining the sample from a tumor from the patient. In certain embodiments, the method comprises an additional step of administering to the patient a therapeutically effective amount of a HER3 inhibitor. In specific embodiments, the HER3 inhibitor is an anti-HER3 antibody or antigen-binding fragment thereof (e.g. 2C2-YTE). In particular embodiments, the HER3 inhibitor is a 2C2-YTE anti-HER3 antibody. In particular embodiments, the HER3 inhibitor is a 2C2-YTE anti-HER3 antibody and the EGFR inhibitor is cetuximab. In particular embodiments, the anti-HER3 antibody is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E).

In another aspect, the disclosure provides methods for determining whether a patient diagnosed with cancer (e.g., head and neck cancer, for example, SCCHN) is indicated as likely to be responsive to treatment with an EGFR inhibitor, comprising measuring the expression of a neuregulin, and at least one of AREG, TGF-α, and EGFR homodimer in a sample from the patient, wherein the sample comprises tumor cells, and wherein the presence of the neuregulin and high levels of one or more of AREG, TGF-α, and EGFR homodimer in the sample indicates that the patient is likely to be responsive to treatment with an EGFR inhibitor. In certain embodiments, the neuregulin is expressed at a high level in the sample. In specific embodiments, the neuregulin is NRG1 (e.g., NRG1α and/or NRG1β). In specific embodiments, the neuregulin is NRG2 (e.g., NRG2α and/or NRG2β). In certain embodiments, said measuring is performed in vitro.

In certain embodiments, such methods for determining whether a patient diagnosed with cancer (e.g., head and neck cancer, for example, SCCHN) is indicated as likely to be responsive to treatment with an EGFR inhibitor comprise measuring a level of one or more of AREG, TGF-α, and EGFR homodimer in the sample. In certain embodiments, the method comprises measuring a level of two or more of AREG, TGF-α, and EGFR homodimer in the sample. In certain embodiments, the method comprises measuring a level of AREG and TGF-α in the sample. In certain embodiments, the method comprises measuring a level of AREG, TGF-α, and EGFR homodimer in the sample. In certain embodiments, the step of measuring expression of neuregulin (e.g., NRG1 and/or NRG2) and AREG and/or TGF-α comprises measuring the level of neuregulin (e.g., NRG1 and/or NRG2) and AREG and/or TGF-α RNA in the sample. In certain embodiments, the step of measuring the expression of EGFR homodimer comprises measuring the level of protein-protein interaction between EGFR monomers in the sample. In particular embodiments, the expression of EGFR homodimer is measured using a protein proximity assay. In certain embodiments, said measuring is performed in vitro.

In certain embodiments, such methods for determining whether a patient diagnosed with cancer (e.g., head and neck cancer, for example, SCCHN) is indicated as likely to be responsive to treatment with an EGFR inhibitor comprise a first step of obtaining the sample from a tumor from the patient. In certain embodiments, the method comprises an additional step of administering to the patient a therapeutically effective amount of an EGFR inhibitor. In specific embodiments, the EGFR inhibitor is an anti-EGFR antibody or antigen-binding fragment thereof (e.g. cetuximab). In particular embodiments, the EGFR inhibitor is cetuximab.

In certain embodiments of such methods for determining whether a patient diagnosed with cancer (e.g., head and neck cancer, for example, SCCHN) is indicated as likely to be responsive to treatment with a HER3 inhibitor, an EGFR inhibitor, or a combination of a HER 3 inhibitor and an EGFR inhibitor, and expression of neuregulin is determined to present in the sample, the method comprises an additional step, e.g., a subsequent step, of measuring the expression of AREG in the sample, wherein high expression of AREG in the sample indicates that the patient is likely to be responsive to treatment with a HER3 inhibitor, an EGFR inhibitor, or a combination of a HER 3 inhibitor and an EGFR inhibitor. In certain embodiments, said measuring is performed in vitro.

In certain embodiments of such methods for determining whether a patient diagnosed with cancer (e.g., head and neck cancer, for example, SCCHN) is indicated as likely to be responsive to treatment with a HER3 inhibitor, an EGFR inhibitor, or a combination of a HER 3 inhibitor and an EGFR inhibitor, and expression of neuregulin is determined to present in the sample, the method comprises an additional step, e.g., a subsequent step, of measuring the expression of TGF-α in the sample, wherein high expression of TGF-α in the sample indicates that the patient is likely to be responsive to treatment with a HER3 inhibitor, an EGFR inhibitor, or a combination of a HER 3 inhibitor and an EGFR inhibitor. In certain embodiments, said measuring is performed in vitro.

In certain embodiments of such methods for determining whether a patient diagnosed with cancer (e.g., head and neck cancer, for example, SCCHN) is indicated as likely to be responsive to treatment with a HER3 inhibitor, an EGFR inhibitor, or a combination of a HER 3 inhibitor and an EGFR inhibitor, and expression of neuregulin is determined to present in the sample, the method comprises an additional step, e.g., a subsequent step, of measuring the expression of EGFR homodimer in the sample, wherein high expression of EGFR homodimer in the sample indicates that the patient is likely to be responsive to treatment with a HER3 inhibitor, an EGFR inhibitor, or a combination of a HER 3 inhibitor and an EGFR inhibitor. In certain embodiments, said measuring is performed in vitro.

In certain embodiments of such methods for determining whether a patient diagnosed with cancer (e.g., head and neck cancer, for example, SCCHN) is indicated as likely to be responsive to treatment with a HER3 inhibitor, an EGFR inhibitor, or a combination of a HER 3 inhibitor and an EGFR inhibitor, and expression of neuregulin is determined to present in the sample, the method comprises an additional step, e.g., a subsequent step, or additional steps, e.g., subsequent steps, of measuring the expression of one, two, or each of AREG, TGF-α, and EGFR homodimer in the sample, wherein high expression of the one, two or each of AREG, TGF-α, and EGFR homodimer in the sample indicates that the patient is likely to be responsive to treatment with a HER3 inhibitor or an EGFR inhibitor. In certain embodiments, said measuring is performed in vitro.

In yet another aspect, the disclosure provides methods for determining whether a patient diagnosed with cancer (e.g., head and neck cancer, for example, SCCHN) is indicated as likely to be responsive to treatment with a HER3 inhibitor, an EGFR inhibitor, or a combination of a HER3 inhibitor and an EGFR inhibitor, comprising measuring the expression of EGFR homodimer in a sample from the patient, wherein the sample comprises tumor cells, wherein a high level of EGFR homodimer in the sample indicates that the patient is likely to be responsive to treatment with a HER3 inhibitor, an EGFR inhibitor, or a combination of a HER3 inhibitor and an EGFR inhibitor. In certain embodiments, the step of measuring the expression of EGFR homodimer comprises measuring the level of protein-protein interaction between EGFR monomers in the sample. In particular embodiments, the expression of EGFR homodimer is measured using a protein proximity assay. In certain embodiments, said measuring is performed in vitro.

In certain embodiments, such methods for determining whether a patient diagnosed with cancer (e.g., head and neck cancer, for example, SCCHN) is indicated as likely to be responsive to treatment with a combination of a HER3 inhibitor and an EGFR inhibitor comprise a first step of obtaining the sample from a tumor from the patient. In certain embodiments, the method comprises an additional step of administering to the patient a therapeutically effective amount of a HER3 inhibitor and a therapeutically effective amount of an EGFR inhibitor. In specific embodiments, the HER3 inhibitor is an anti-HER3 antibody or antigen-binding fragment thereof (e.g. 2C2-YTE). In specific embodiments, the EGFR inhibitor is an anti-EGFR antibody or antigen-binding fragment thereof (e.g., cetuximab). In particular embodiments, the HER3 inhibitor is a 2C2-YTE anti-HER3 antibody and the EGFR inhibitor is cetuximab. In particular embodiments, the HER3 inhibitor is a 2C2-YTE anti-HER3 antibody and the EGFR inhibitor is cetuximab. In particular embodiments, the anti-HER3 antibody is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E) and the EGFR inhibitor is cetuximab.

In another aspect, the disclosure provides methods for determining whether a patient diagnosed with cancer (e.g., head and neck cancer, for example, SCCHN) is indicated as likely to be responsive to treatment with a HER3 inhibitor, comprising measuring the expression of EGFR homodimer in a sample from the patient, wherein the sample comprises tumor cells, wherein a high level of EGFR homodimer in the sample indicates that the patient is likely to be responsive to treatment with a HER3 inhibitor. In certain embodiments, the step of measuring the expression of EGFR homodimer comprises measuring the level of protein-protein interaction between EGFR monomers in the sample. In particular embodiments, the expression of EGFR homodimer is measured using a protein proximity assay. In certain embodiments, said measuring is performed in vitro.

In certain embodiments, such methods for determining whether a patient diagnosed with cancer (e.g., head and neck cancer, for example, SCCHN) is indicated as likely to be responsive to treatment with a combination of a HER3 inhibitor comprise a first step of obtaining the sample from a tumor from the patient. In certain embodiments, the method comprises an additional step of administering to the patient a therapeutically effective amount of a HER3 inhibitor. In specific embodiments, the HER3 inhibitor is an anti-HER3 antibody or antigen-binding fragment thereof (e.g. 2C2-YTE). In particular embodiments, the HER3 inhibitor is a 2C2-YTE anti-HER3 antibody. In particular embodiments, the HER3 inhibitor is a 2C2-YTE anti-HER3 antibody and the EGFR inhibitor is cetuximab. In particular embodiments, the anti-HER3 antibody is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E).

In another aspect, the disclosure provides methods for determining whether a patient diagnosed with cancer (e.g., head and neck cancer, for example, SCCHN) is indicated as likely to be responsive to treatment with an EGFR inhibitor, comprising measuring the expression of EGFR homodimer in a sample from the patient, wherein the sample comprises tumor cells, wherein a high level of EGFR homodimer in the sample indicates that the patient is likely to be responsive to treatment with an EGFR inhibitor. In certain embodiments, the step of measuring the expression of EGFR homodimer comprises measuring the level of protein-protein interaction between EGFR monomers in the sample. In particular embodiments, the expression of EGFR homodimer is measured using a protein proximity assay. In certain embodiments, said measuring is performed in vitro.

In certain embodiments, such methods for determining whether a patient diagnosed with cancer (e.g., head and neck cancer, for example, SCCHN) is indicated as likely to be responsive to treatment with an EGFR inhibitor comprise a first step of obtaining the sample from a tumor from the patient. In certain embodiments, the method comprises an additional step of administering to the patient a therapeutically effective amount of an EGFR inhibitor. In specific embodiments, the EGFR inhibitor is an anti-EGFR antibody or antigen-binding fragment thereof (e.g., cetuximab). In particular embodiments, the EGFR inhibitor is cetuximab.

Also disclosed herein are kits comprising components for performing the methods for determining whether a patient diagnosed with cancer (e.g., head and neck cancer, for example, SCCHN) is indicated as likely to be responsive to treatment with a HER3 inhibitor, an EGFR inhibitor, or a combination of a HER3 inhibitor and an EGFR inhibitor.

In another aspect, the disclosure provides methods for determining whether a patient diagnosed with cancer (e.g., thyroid cancer or melanoma) is indicated as likely to be responsive to treatment with a HER3 inhibitor, comprising measuring the expression of a neuregulin (e.g. NRG1 and/or NRG2) in a sample from the patient, wherein the sample comprises tumor cells, wherein a high level of neuregulin in the sample indicates that the patient is likely to be responsive to treatment with a HER3 inhibitor. In certain embodiments, the neuregulin is NRG1 (e.g., NRG1α and/or NRG1β). In certain embodiments, the neuregulin is NRG2 (e.g., NRG2α and/or NRG2β). In certain embodiments, said measuring is performed in vitro. In certain embodiments, the cancer is characterized by a BRAF mutation (e.g., V600E or V600K). In certain embodiments, the cancer is resistant to treatment with a BRAF inhibitor (e.g., vemurafenib or dabrafenib). In another embodiment, the cancer is resistant to treatment with a MEK inhibitor (e.g., trametinib). In other embodiments, the cancer is resistant to treatment with a BRAF inhibitor (e.g., vemurafenib or dabrafenib) and a MEK inhibitor (e.g., trametinib). In certain embodiments, the cancer is melanoma. In specific embodiments, the cancer is B-Raf mutated melanoma. In certain embodiments, the cancer is thyroid cancer. In specific embodiments, the cancer is B-Raf mutated thyroid cancer. In certain embodiments, the cancer is hairy cell leukemia. In specific embodiments, the cancer is B-Raf mutated hairy cell leukemia. In certain embodiments, the cancer is colorectal cancer. In specific embodiments, the cancer is B-Raf mutated colorectal cancer. In certain embodiments, the cancer is lung cancer. In specific embodiments, the cancer is B-Raf mutated lung cancer. In particular embodiments, the lung cancer is non-small cell lung carcinoma. In certain embodiments, the cancer is a squamous cell carcinoma of the head and neck (SCCHN). In certain embodiments, the cancer is ovarian cancer. In certain embodiments, the cancer is pancreatic cancer. In certain embodiments, the cancer is bladder cancer. In certain embodiments, the cancer is testicular cancer. In certain embodiments, the cancer is endometrial cancer. In certain embodiments, the cancer is hepatocellular carcinoma. In certain embodiments, the cancer is breast cancer. In certain embodiments, the cancer is gastric cancer. In certain embodiments, the cancer is prostate cancer.

In certain embodiments, such methods for determining whether a patient diagnosed with cancer (e.g., thyroid cancer or melanoma) is indicated as likely to be responsive to treatment with a HER3 inhibitor comprise a first step of obtaining the sample from a tumor from the patient. In certain embodiments, the method comprises an additional step of administering to the patient a therapeutically effective amount of a HER3 inhibitor. In specific embodiments, the HER3 inhibitor is an anti-HER3 antibody or antigen-binding fragment thereof (e.g. 2C2-YTE). In particular embodiments, the HER3 inhibitor is a 2C2-YTE anti-HER3 antibody. In particular embodiments, the HER3 inhibitor is a 2C2-YTE anti-HER3. In particular embodiments, the anti-HER3 antibody is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E).

In another aspect, the disclosure provides methods for determining whether a patient diagnosed with cancer (e.g., thyroid cancer or melanoma) is indicated as likely to be responsive to treatment with a combination of a HER3 inhibitor and a B-Raf inhibitor, comprising measuring the expression of a neuregulin (e.g. NRG1 and/or NRG2) in a sample from the patient, wherein the sample comprises tumor cells, wherein a high level of neuregulin in the sample indicates that the patient is likely to be responsive to treatment with a combination of a HER3 inhibitor and a B-Raf inhibitor. In certain embodiments, the neuregulin is NRG1 (e.g., NRG1α and/or NRG1β). In certain embodiments, the neuregulin is NRG2 (e.g., NRG2α and/or NRG2β). In certain embodiments, said measuring is performed in vitro. In certain embodiments, the cancer is characterized by a BRAF mutation (e.g., V600E or V600K). In certain embodiments, the cancer is resistant to treatment with a BRAF inhibitor (e.g., vemurafenib or dabrafenib). In another embodiment, the cancer is resistant to treatment with a MEK inhibitor (e.g., trametinib). In other embodiments, the cancer is resistant to treatment with a BRAF inhibitor (e.g., vemurafenib or dabrafenib) and a MEK inhibitor (e.g., trametinib). In certain embodiments, the cancer is melanoma. In specific embodiments, the cancer is B-Raf mutated melanoma. In certain embodiments, the cancer is thyroid cancer. In specific embodiments, the cancer is B-Raf mutated thyroid cancer. In certain embodiments, the cancer is hairy cell leukemia. In specific embodiments, the cancer is B-Raf mutated hairy cell leukemia. In certain embodiments, the cancer is colorectal cancer. In specific embodiments, the cancer is B-Raf mutated colorectal cancer. In certain embodiments, the cancer is lung cancer. In specific embodiments, the cancer is B-Raf mutated lung cancer. In particular embodiments, the lung cancer is non-small cell lung carcinoma. In certain embodiments, the cancer is a squamous cell carcinoma of the head and neck (SCCHN). In certain embodiments, the cancer is ovarian cancer. In certain embodiments, the cancer is pancreatic cancer. In certain embodiments, the cancer is bladder cancer. In certain embodiments, the cancer is testicular cancer. In certain embodiments, the cancer is endometrial cancer. In certain embodiments, the cancer is hepatocellular carcinoma. In certain embodiments, the cancer is breast cancer. In certain embodiments, the cancer is gastric cancer. In certain embodiments, the cancer is prostate cancer.

In certain embodiments, such methods for determining whether a patient diagnosed with cancer (e.g., thyroid cancer or melanoma) is indicated as likely to be responsive to treatment with a combination of a HER3 inhibitor and a B-Raf inhibitor comprise a first step of obtaining the sample from a tumor from the patient. In certain embodiments, the method comprises an additional step of administering to the patient a therapeutically effective amount of a combination of a HER3 inhibitor and a B-Raf inhibitor. In specific embodiments, the HER3 inhibitor is an anti-HER3 antibody or antigen-binding fragment thereof (e.g. 2C2-YTE) and the B-Raf inhibitor is vemurafenib. In specific embodiments, the HER3 inhibitor is an anti-HER3 antibody or antigen-binding fragment thereof (e.g. 2C2-YTE) and the B-Raf inhibitor is dabrafenib. In particular embodiments, the HER3 inhibitor is a 2C2-YTE anti-HER3 antibody and the B-Raf inhibitor is vemurafenib. In particular embodiments, the HER3 inhibitor is a 2C2-YTE anti-HER3 antibody and the B-Raf inhibitor is dabrafenib. In particular embodiments, the anti-HER3 antibody is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E), and the B-Raf inhibitor is vemurafenib or dabrafenib.

Also disclosed herein are kits comprising components for performing the methods for determining whether a patient diagnosed with cancer (e.g., thyroid cancer or melanoma) is indicated as likely to be responsive to treatment with a HER3 inhibitor or a combination of a HER3 inhibitor and a B-Raf inhibitor.

In another aspect, the disclosure provides methods for determining whether a patient diagnosed with cancer (e.g., thyroid cancer or melanoma) is indicated as likely to be responsive to treatment with a combination of a HER3 inhibitor and a MEK inhibitor, comprising measuring the expression of a neuregulin (e.g. NRG1 and/or NRG2) in a sample from the patient, wherein the sample comprises tumor cells, wherein a high level of neuregulin in the sample indicates that the patient is likely to be responsive to treatment with a combination of a HER3 inhibitor and a MEK inhibitor. In certain embodiments, the neuregulin is NRG1 (e.g., NRG1α and/or NRG1β). In certain embodiments, the neuregulin is NRG2 (e.g., NRG2α and/or NRG2β). In certain embodiments, said measuring is performed in vitro. In certain embodiments, the cancer is characterized by a BRAF mutation (e.g., V600E or V600K). In certain embodiments, the cancer is resistant to treatment with a BRAF inhibitor (e.g., vemurafenib or dabrafenib). In another embodiment, the cancer is resistant to treatment with a MEK inhibitor (e.g., trametinib). In other embodiments, the cancer is resistant to treatment with a BRAF inhibitor (e.g., vemurafenib or dabrafenib) and a MEK inhibitor (e.g., trametinib). In certain embodiments, the cancer is melanoma. In specific embodiments, the cancer is B-Raf mutated melanoma. In certain embodiments, the cancer is thyroid cancer. In specific embodiments, the cancer is B-Raf mutated thyroid cancer. In certain embodiments, the cancer is hairy cell leukemia. In specific embodiments, the cancer is B-Raf mutated hairy cell leukemia. In certain embodiments, the cancer is colorectal cancer. In specific embodiments, the cancer is B-Raf mutated colorectal cancer. In certain embodiments, the cancer is lung cancer. In specific embodiments, the cancer is B-Raf mutated lung cancer. In particular embodiments, the lung cancer is non-small cell lung carcinoma. In certain embodiments, the cancer is a squamous cell carcinoma of the head and neck (SCCHN). In certain embodiments, the cancer is ovarian cancer. In certain embodiments, the cancer is pancreatic cancer. In certain embodiments, the cancer is bladder cancer. In certain embodiments, the cancer is testicular cancer. In certain embodiments, the cancer is endometrial cancer. In certain embodiments, the cancer is hepatocellular carcinoma. In certain embodiments, the cancer is breast cancer. In certain embodiments, the cancer is gastric cancer. In certain embodiments, the cancer is prostate cancer.

In certain embodiments, such methods for determining whether a patient diagnosed with cancer (e.g., thyroid cancer or melanoma) is indicated as likely to be responsive to treatment with a combination of a HER3 inhibitor and a MEK inhibitor comprise a first step of obtaining the sample from a tumor from the patient. In certain embodiments, the method comprises an additional step of administering to the patient a therapeutically effective amount of a combination of a HER3 inhibitor and a MEK inhibitor. In specific embodiments, the HER3 inhibitor is an anti-HER3 antibody or antigen-binding fragment thereof (e.g. 2C2-YTE) and the MEK inhibitor is selumetinib. In specific embodiments, the HER3 inhibitor is an anti-HER3 antibody or antigen-binding fragment thereof (e.g. 2C2-YTE) and the MEK inhibitor is trametinib. In particular embodiments, the HER3 inhibitor is a 2C2-YTE anti-HER3 antibody and the MEK inhibitor is selumetinib. In particular embodiments, the HER3 inhibitor is a 2C2-YTE anti-HER3 antibody and the MEK inhibitor is trametinib. In particular embodiments, the anti-HER3 antibody is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E), and the MEK inhibitor is selumetinib or trametinib.

Also disclosed herein are kits comprising components for performing the methods for determining whether a patient diagnosed with cancer (e.g., thyroid cancer or melanoma) is indicated as likely to be responsive to treatment with a combination of a HER3 inhibitor and a MEK inhibitor.

In another aspect, the disclosure provides methods for determining whether a patient diagnosed with cancer (e.g., thyroid cancer or melanoma) is indicated as likely to be responsive to treatment with a combination of a HER3 inhibitor, a B-Raf inhibitor, and MEK inhibitor, comprising measuring the expression of a neuregulin (e.g. NRG1 and/or NRG2) in a sample from the patient, wherein the sample comprises tumor cells, wherein a high level of neuregulin in the sample indicates that the patient is likely to be responsive to treatment with a combination of a HER3 inhibitor, a B-Raf inhibitor, and a MEK inhibitor. In certain embodiments, the neuregulin is NRG1 (e.g., NRG1α and/or NRG1β). In certain embodiments, the neuregulin is NRG2 (e.g., NRG2α and/or NRG2β). In certain embodiments, said measuring is performed in vitro. In certain embodiments, the cancer is characterized by a BRAF mutation (e.g., V600E or V600K). In certain embodiments, the cancer is resistant to treatment with a BRAF inhibitor (e.g., vemurafenib or dabrafenib). In another embodiment, the cancer is resistant to treatment with a MEK inhibitor (e.g., trametinib). In other embodiments, the cancer is resistant to treatment with a BRAF inhibitor (e.g., vemurafenib or dabrafenib) and a MEK inhibitor (e.g., trametinib). In certain embodiments, the cancer is melanoma. In specific embodiments, the cancer is B-Raf mutated melanoma. In certain embodiments, the cancer is thyroid cancer. In specific embodiments, the cancer is B-Raf mutated thyroid cancer. In certain embodiments, the cancer is hairy cell leukemia. In specific embodiments, the cancer is B-Raf mutated hairy cell leukemia. In certain embodiments, the cancer is colorectal cancer. In specific embodiments, the cancer is B-Raf mutated colorectal cancer. In certain embodiments, the cancer is lung cancer. In specific embodiments, the cancer is B-Raf mutated lung cancer. In particular embodiments, the lung cancer is non-small cell lung carcinoma. In certain embodiments, the cancer is a squamous cell carcinoma of the head and neck (SCCHN). In certain embodiments, the cancer is ovarian cancer. In certain embodiments, the cancer is pancreatic cancer. In certain embodiments, the cancer is bladder cancer. In certain embodiments, the cancer is testicular cancer. In certain embodiments, the cancer is endometrial cancer. In certain embodiments, the cancer is hepatocellular carcinoma. In certain embodiments, the cancer is breast cancer. In certain embodiments, the cancer is gastric cancer. In certain embodiments, the cancer is prostate cancer.

In certain embodiments, such methods for determining whether a patient diagnosed with cancer (e.g., thyroid cancer or melanoma) is indicated as likely to be responsive to treatment with a combination of a HER3 inhibitor, a B-Raf inhibitor, and a MEK inhibitor comprise a first step of obtaining the sample from a tumor from the patient. In certain embodiments, the method comprises an additional step of administering to the patient a therapeutically effective amount of a combination of a HER3 inhibitor, a B-Raf inhibitor, and a MEK inhibitor. In specific embodiments, the HER3 inhibitor is an anti-HER3 antibody or antigen-binding fragment thereof (e.g. 2C2-YTE), the B-Raf inhibitor is vemurafenib, and the MEK inhibitor is selumetinib or trametinib. In specific embodiments, the HER3 inhibitor is an anti-HER3 antibody or antigen-binding fragment thereof (e.g. 2C2-YTE), the B-Raf inhibitor is dabrafenib, and the MEK inhibitor is selumetinib or trametinib. In particular embodiments, the HER3 inhibitor is a 2C2-YTE anti-HER3 antibody, the B-Raf inhibitor is vemurafenib, and the MEK inhibitor is selumetinib or trametinib. In particular embodiments, the HER3 inhibitor is a 2C2-YTE anti-HER3 antibody, the B-Raf inhibitor is dabrafenib, and the MEK inhibitor is selumetinib or trametinib. In particular embodiments, the anti-HER3 antibody is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E), the B-Raf inhibitor is vemurafenib or dabrafenib, and the MEK inhibitor is selumetinib or trametinib.

Also disclosed herein are kits comprising components for performing the methods for determining whether a patient diagnosed with cancer (e.g., thyroid cancer or melanoma) is indicated as likely to be responsive to treatment with a combination of a HER3 inhibitor, a B-Raf inhibitor, and a MEK inhibitor.

In another aspect, the disclosure provides methods for determining whether a patient diagnosed with cancer (e.g., thyroid cancer or melanoma) is indicated as likely to be responsive to treatment with a combination of a HER3 inhibitor and an EGFR inhibitor, comprising measuring the expression of a neuregulin (e.g. NRG1 and/or NRG2) in a sample from the patient, wherein the sample comprises tumor cells, wherein a high level of neuregulin in the sample indicates that the patient is likely to be responsive to treatment with a combination of a HER3 inhibitor and an EGFR inhibitor. In certain embodiments, the neuregulin is NRG1 (e.g., NRG1α and/or NRG1β). In certain embodiments, the neuregulin is NRG2 (e.g., NRG2α and/or NRG2β). In certain embodiments, said measuring is performed in vitro. In certain embodiments, the cancer is characterized by a BRAF mutation (e.g., V600E or V600K). In certain embodiments, the cancer is resistant to treatment with a BRAF inhibitor (e.g., vemurafenib or dabrafenib). In another embodiment, the cancer is resistant to treatment with a MEK inhibitor (e.g., trametinib). In other embodiments, the cancer is resistant to treatment with a BRAF inhibitor (e.g., vemurafenib or dabrafenib) and a MEK inhibitor (e.g., trametinib). In certain embodiments, the cancer is melanoma. In specific embodiments, the cancer is B-Raf mutated melanoma. In certain embodiments, the cancer is thyroid cancer. In specific embodiments, the cancer is B-Raf mutated thyroid cancer. In certain embodiments, the cancer is hairy cell leukemia. In specific embodiments, the cancer is B-Raf mutated hairy cell leukemia. In certain embodiments, the cancer is colorectal cancer. In specific embodiments, the cancer is B-Raf mutated colorectal cancer. In certain embodiments, the cancer is lung cancer. In specific embodiments, the cancer is B-Raf mutated lung cancer. In particular embodiments, the lung cancer is non-small cell lung carcinoma. In certain embodiments, the cancer is a squamous cell carcinoma of the head and neck (SCCHN). In certain embodiments, the cancer is ovarian cancer. In certain embodiments, the cancer is pancreatic cancer. In certain embodiments, the cancer is bladder cancer. In certain embodiments, the cancer is testicular cancer. In certain embodiments, the cancer is endometrial cancer. In certain embodiments, the cancer is hepatocellular carcinoma. In certain embodiments, the cancer is breast cancer. In certain embodiments, the cancer is gastric cancer. In certain embodiments, the cancer is prostate cancer.

In certain embodiments, such methods for determining whether a patient diagnosed with cancer (e.g., thyroid cancer or melanoma) is indicated as likely to be responsive to treatment with a combination of a HER3 inhibitor and an EGFR inhibitor comprise a first step of obtaining the sample from a tumor from the patient. In certain embodiments, the method comprises an additional step of administering to the patient a therapeutically effective amount of a combination of a HER3 inhibitor and an EGFR inhibitor. In specific embodiments, the HER3 inhibitor is an anti-HER3 antibody or antigen-binding fragment thereof (e.g. 2C2-YTE) and the EGFR inhibitor is cetuximab. In particular embodiments, the anti-HER3 antibody is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E), and the B-Raf inhibitor is vemurafenib or dabrafenib.

Also disclosed herein are kits comprising components for performing the methods for determining whether a patient diagnosed with cancer (e.g., thyroid cancer or melanoma) is indicated as likely to be responsive to treatment with a HER3 inhibitor or a combination of a HER3 inhibitor and an EGFR inhibitor.

In certain embodiments, the anti-HER3 antibodies or antigen-binding fragments thereof used in the methods described herein specifically bind to the same HER3 epitope as an antibody or antigen-binding fragment thereof comprising the heavy chain variable region (VH) and light chain variable region (VL) of CL16 or 2C2. In certain embodiments, the VH and VL of CL16 comprise SEQ ID NOs: 2 and 1, respectively, and the VH and VL of 2C2 comprise SEQ ID NOs: 2 and 3, respectively. In certain embodiments, the anti-HER3 antibody or antigen-binding fragment thereof is affinity matured.

In certain embodiments, the anti-HER3 antibody or antigen-binding fragment thereof comprises an antibody VL and an antibody VH, wherein the VL comprises the amino acid sequence:

[FW1]X₁GSX₂SNIGLNYVS[FW2]RNNQRPS[FW3]AAWDDX₃X₄X₅GEX₆
[FW4]

wherein [FW1], [FW2], [FW3] and [FW4] represent VL framework regions, and wherein
(a) X₁ represents amino acid residues Arginine (R) or Serine (S),
(b) X₂ represents amino acid residues Serine (S) or Leucine (L),
(c) X₃ represents amino acid residues Serine (S) or Glycine (G),
(d) X₄ represents amino acid residues Leucine (L) or Proline (P),
(e) X₅ represents amino acid residues Arginine (R), Isoleucine (I), Proline (P) or Serine (S), and
(f) X₆ represents amino acid residues Valine (V) or Alanine (A), and

wherein the VH comprises the amino acid sequence:

[FW5]YYYMQ[FW6]X₇IGSSGGVTNYADSVKG[FW7]VGLGDAFDI[FW8]

wherein [FW5], [FW6], [FW7] and [FW8] represent VH framework regions, and wherein X₇ represents amino acid residues Tyrosine (Y), Isoleucine (I) or Valine (V). In specific embodiments, FW1 comprises SEQ ID NO: 40 or 44, FW2 comprises SEQ ID NO: 41, FW3 comprises SEQ ID NO: 42, FW4 comprises SEQ ID NO: 43, FW5 comprises SEQ ID NO: 36, FW6 comprises SEQ ID NO: 37, FW7 comprises SEQ ID NO: 38, and FW8 comprises SEQ ID NO: 39.

In certain embodiments the anti-HER3 antibody or antigen-binding fragment thereof comprises a VL and a VH comprising VL-CDR1, VL-CRD2, VL-CDR3, VH-CDR1, VH-CDR2, and VH-CDR3 amino acid sequences identical or identical except for four, three, two, or one amino acid substitutions in one or more CDRs to: SEQ ID NOs: 18, 21, 22, 31, 32, and 35, SEQ ID NOs: 18, 21, 26, 31, 32 and 35, SEQ ID NOs: 18, 21, 27, 31, 32 and 35, SEQ ID NOs: 20, 21, 22, 31, 32 and 35, SEQ ID NOs: 19, 21, 22, 31, 32 and 35, SEQ ID NOs: 18, 21, 25, 31, 32 and 35, SEQ ID NOs: 18, 21, 28, 31, 32 and 35, SEQ ID NOs: 18, 21, 29, 31, 32 and 35, SEQ ID NOs: 18, 21, 30, 31, 32 and 35, SEQ ID NOs: 18, 21, 23, 31, 32 and 35, SEQ ID NOs: 19, 21, 23, 31, 32 and 35, SEQ ID NOs: 20, 21, 23, 31, 32 and 35, SEQ ID NOs: 18, 21, 24, 31, 32 and 35, or SEQ ID NOs: 18, 21, 25, 31, 32 and 35, respectively.

In certain embodiments, the anti-HER3 antibody or antigen-binding fragment thereof comprises an antibody VL and an antibody VH, wherein the VL comprises an amino acid sequence at least about 90% to about 100% identical to a reference amino acid sequence selected from the group consisting of SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 14, SEQ ID NO: 15, SEQ ID NO: 16, and SEQ ID NO: 17.

In certain embodiments, the anti-HER3 antibody or antigen-binding fragment thereof comprises an antibody VL and an antibody VH, wherein the VH comprises an amino acid sequence at least about 90% to about 100% identical to a reference amino acid sequence selected from the group consisting of SEQ ID NO: 2, SEQ ID NO: 12 and SEQ ID NO: 13.

In certain embodiments, the anti-HER3 antibody or antigen-binding fragment thereof comprises a VL comprising a sequence at least about 90% to about 100% identical to a reference amino acid sequence selected from the group consisting of SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 14, SEQ ID NO: 15, SEQ ID NO: 16, and SEQ ID NO: 17, and wherein the anti-HER3 antibody or antigen-binding fragment comprises a VH comprising a sequence at least about 90% to about 100% identical to a reference amino acid sequence selected from the group consisting of SEQ ID NO: 2, SEQ ID NO: 12 and SEQ ID NO: 13.

In certain embodiments, the anti-HER3 antibody or antigen-binding fragment thereof comprises a VL comprising the VL consensus sequence provided in Table 3 and a VH comprising the VH consensus sequence provided in Table 3.

In certain embodiments, the anti-HER3 antibody or antigen-binding fragment thereof comprises a VL-CDR1, a VL-CDR2 and a VL-CDR3 comprising SEQ ID NO: 19, SEQ ID NO: 21 and SEQ ID NO: 23, respectively, and a VH-CDR1, a VH-CDR2, and a VH-CDR3 comprising SEQ ID NO: 31, SEQ ID NO: 32, and SEQ ID NO: 35, respectively.

In certain embodiments, the anti-HER3 antibody or antigen-binding fragment thereof comprises a VL comprising SEQ ID NO: 3 and a VH comprising SEQ ID NO: 2.

In certain embodiments, the anti-HER3 antibody or antigen-binding fragments thereof described herein comprise a human heavy chain constant region or fragment thereof. In specific embodiments, the heavy chain constant region or fragment thereof is an IgG constant region. In certain embodiments, the IgG constant region is selected from an IgG1 constant region, an IgG2 constant region, an IgG3 constant region and an IgG4 constant region. In specific embodiments, the IgG constant region is an IgG1 constant region.

In specific embodiments, the IgG constant domain comprises one or more amino acid substitutions relative to a wild-type IgG constant domain wherein the modified IgG has an increased half-life compared to the half-life of an IgG having the wild-type IgG constant domain. In more specific embodiments, the IgG constant domain comprises one or more amino acid substitutions of amino acid residues at positions 251-257, 285-290, 308-314, 385-389, and 428-436, wherein the numbering is according to the EU index as set forth in Kabat. In even more specific embodiments, at least one IgG constant domain amino acid substitution is selected from the group consisting of:
(a) substitution of the amino acid at position 252 with Tyrosine (Y), Phenylalanine (F), Tryptophan (W), or Threonine (T),
(b) substitution of the amino acid at position 254 with Threonine (T),
(c) substitution of the amino acid at position 256 with Serine (S), Arginine (R), Glutamine (Q), Glutamic acid (E), Aspartic acid (D), or Threonine (T),
(d) substitution of the amino acid at position 257 with Leucine (L),
(e) substitution of the amino acid at position 309 with Proline (P),
(f) substitution of the amino acid at position 311 with Serine (S),
(g) substitution of the amino acid at position 428 with Threonine (T), Leucine (L), Phenylalanine (F), or Serine (S),
(h) substitution of the amino acid at position 433 with Arginine (R), Serine (S), Isoleucine (I), Proline (P), or Glutamine (Q),
(i) substitution of the amino acid at position 434 with Tryptophan (W), Methionine (M), Serine (S), Histidine (H), Phenylalanine (F), or Tyrosine, and
(j) a combination of two or more of said substitutions,
   wherein the numbering is according to the EU index as set forth in Kabat. In further specific embodiments, the human IgG constant domain comprises amino acid substitutions relative to a wild-type human IgG constant domain at positions 252, 254, and 256, wherein
(a) the amino acid at position 252 is substituted with Tyrosine (Y),
(b) the amino acid at position 254 is substituted with Threonine (T), and
(c) the amino acid at position 256 is substituted with Glutamic acid (E),
   wherein the numbering is according to the EU index as set forth in Kabat. In specific embodiments, the amino acid at position 434 is substituted with an amino acid selected from the group consisting of Tryptophan (W), Methionine (M), Tyrosine (Y), and Serine (S), and wherein the numbering is according to the EU index as set forth in Kabat. In even more specific embodiments, wherein the amino acid at position 428 is substituted with an amino acid selected from the group consisting of Threonine (T), Leucine (L), Phenylalanine (F), and Serine (S), and wherein the numbering is according to the EU index as set forth in Kabat. In certain specific embodiments, the amino acid at position 257 is substituted with Leucine (L), and the amino acid at Kabat position 434 is substituted with Tyrosine (Y), and wherein the numbering is according to the EU index as set forth in Kabat. In certain specific embodiments, the amino acid at Kabat position 428 is substituted with Leucine (L), and the amino acid at Kabat position 434 is substituted with Serine (S).

In certain embodiments of the anti-HER3 antibody or antigen-binding fragments thereof described herein comprise a light chain constant region selected from the group consisting of a human kappa constant region and a human lambda constant region. In specific embodiments, the anti-HER3 antibody or antigen-binding fragment comprises a human lambda constant region.

In certain embodiments, the anti-HER3 antibody or antigen-binding fragment thereof comprises an antibody VL of SEQ ID NO:3, an antibody VH of SEQ ID NO: 2, and an IgG1 constant region of SEQ ID 46. In other embodiments, the human IgG constant region, comprises amino acid substitutions relative to a wild-type human IgG constant domain at positions 252, 254, and 256, wherein the numbering is according to the EU index as set forth in Kabat, and wherein
(a) the amino acid at position 252 is substituted with Tyrosine (Y),
(b) the amino acid at position 254 is substituted with Threonine (T), and
(c) the amino acid at position 256 is substituted with Glutamic acid (E).

In certain embodiments, the anti-HER3 antibody described herein is a human antibody, a humanized antibody, a chimeric antibody, a monoclonal antibody, a polyclonal antibody, a recombinant antibody, a multispecific antibody, or an antigen-binding fragment thereof. In a specific embodiment, the anti-HER3 antibody is a human antibody. In specific embodiments, the antigen-binding fragment is Fv, Fab, F(ab′)2, Fab′, dsFv, scFv, and sc(Fv)2. In certain specific embodiments, the anti-HER3 antibody or antigen-binding fragment is conjugated to at least one heterologous agent.

In one embodiment, provided herein is a method of treating a cancer, comprising administering to a patient diagnosed with said cancer a therapeutically effective amount of a HER3 inhibitor and an EGFR inhibitor, wherein the patient has a tumor that has been characterized in that cells from the tumor: i) express a neuregulin, and ii) express high levels of one or more of amphiregulin (AREG), TGF-α, and EGFR homodimer; wherein the cancer is a SCCHN, wherein the HER3 inhibitor is an antibody comprising a VL that comprises a VL-CDR1, a VL-CDR2, and a VL-CDR3 comprising SEQ ID NO:19, SEQ ID NO: 21 and SEQ ID NO: 23, respectively, a human lambda constant region, a VH that comprises a VH-CDR1, a VH-CDR2, and a VH-CDR3 comprising SEQ IDNO: 31, SEQ ID NO: 32, and SEQ ID NO: 35, respectively, and a human IgG1 constant region comprising amino acid substitutions relative to a wild-type human IgG constant domain at positions 252, 254, and 256, wherein
(a) the amino acid at position 252 is substituted with Tyrosine (Y),
(b) the amino acid at position 254 is substituted with Threonine (T), and
(c) the amino acid at position 256 is substituted with Glutamic acid (E), wherein the numbering is according to the EU index as set forth in Kabat; and wherein the EGFR inhibitor is cetuximab.

In another embodiment, provided herein is a method of treating a cancer, comprising administering to a patient diagnosed with said cancer a therapeutically effective amount of a HER3 inhibitor and an EGFR inhibitor, wherein the patient has a tumor that has been characterized in that cells from the tumor: i) express a neuregulin, and ii) express high levels of one or more of amphiregulin (AREG), TGF-α, and EGFR homodimer; wherein the cancer is a SCCHN, wherein the HER3 inhibitor is an antibody comprising a VL that comprises SEQ ID NO: 3, a human lambda constant region, a VH that comprises SEQ ID NO: 2, and a human IgG1 constant region comprising amino acid substitutions relative to a wild-type human IgG constant domain at positions 252, 254, and 256, wherein
(a) the amino acid at position 252 is substituted with Tyrosine (Y),
(b) the amino acid at position 254 is substituted with Threonine (T), and
(c) the amino acid at position 256 is substituted with Glutamic acid (E), wherein the numbering is according to the EU index as set forth in Kabat; and wherein the EGFR inhibitor is cetuximab.

### BRIEF DESCRIPTION OF THE DRAWINGS/FIGURES

FIG. 1A depicts the level of NRG mRNA overexpression in the indicated cancer sample types. FIG. 1B depicts the relative cell surface protein expression level of EGFR, ErbB2, and ErbB3 across SCCHN cell lines. Bars 1-6 represent EGFR levels in SCC35, SCC61, UNC7, UNC10, 4213 (non-cancerous skin cell line), and FaDu cell lines, respectively. Bars 7-12 represent ErbB2 levels in SCC35, SCC61, UNC7, UNC10, 4213 (non-cancerous skin cell line), and FaDu cell lines, respectively. Bars 13-18 represent ErbB3 levels in SCC35, SCC61, UNC7, UNC10, 4213 (non-cancerous skin cell line), and FaDu cell lines, respectively. FIG. 1C is an enlarged depiction of bars 13-18 in FIG. 1B.

FIGS. 2A-2I depict the fold activation of EGFR (FIGS. 2A, 2D, and 2G), ErbB2 (FIGS. 2B, 2E, and 2H), or ErbB3 (FIGS. 2C, 2F, and 2I), as determined by the level of EGFR phosphorylation (“pEGFR”), ErbB2 phosphorylation (“pErbB2”), or ErbB3 phosphorylation (“pErbB3”), respectively. FIGS. 2A-2C: cells were treated with control, cetuximab (“Cetux”), pertuzumab (“Pertuz”), or 2C2-YTE. FIGS. 2D-2F: cells were treated with control, epidermal growth factor (“EGF”), or EGF with cetuximab, pertuzumab, or 2C2-YTE. FIGS. 2G-21: cells were treated with control, neuregulin (“NRG”), or NRG with cetuximab, pertuzumab, or 2C2-YTE.

FIGS. 3A-3I depict the percent of maximal cell growth in upon treatment with 2C2-YTE (squares), cetuximab (circles), or 2C2-YTE and cetuximab (diamonds).

FIG. 4 depicts a Western blot of the indicated cell lines mock-treated, or treated with 2C2-YTE, cetuximab, or both 2C2-YTE and cetuximab. The western blots were evaluated for expression of the indicated proteins. The arrows indicate certain samples in which the ERK (row 1) or the AKT (row 3) pathways were inhibited by cetuximab or 2C2-YTE treatment, respectively.

FIG. 5A-5B depict tumor volume in a CTG-0790 (patient-derived xenograft (“PDX”); FIG. 5A) or a FaDu (cell-line-derived xenograft (“CDX”); FIG. 5B) xenograft tumor mouse model. FIG. 5A: CTG-0790 xenograft tumor mouse models were treated with control IgG1 (10 mg/kg, circles), 2C2-YTE (10 mg/kg, squares), cetuximab (10 mg/kg, triangles point up), or 2C2-YTE and cetuximab (10 mg/kg of each, triangles, point down) and tumor volume was measured at the indicated days. FIG. 5B: FaDu xenograft tumor mouse models were treated with control IgG1 (10 mg/kg, triangles), 2C2-YTE (10 mg/kg, squares), cetuximab (10 mg/kg, circles), or 2C2-YTE and cetuximab (10 mg/kg of each, diamonds) and tumor volume was measured at the indicated days.

FIG. 6A-6D depict the association between NRG1 mRNA expression and TGF-α (FIG. 6A) or amphiregulin (AREG, FIG. 6B), in human tumor samples from colorectal adenocarcinoma, colorectal mucinous adenocarcinoma, or head and neck squamous cell carcinoma, and the same data for the association between NRG1 mRNA expression and TGF-α (FIG. 6C) or amphiregulin (AREG, FIG. 6D), in human tumor samples from head and neck squamous cell carcinoma alone.

FIG. 7A-7B depict the association between sensitivity of SCCHN cells to treatment with 2C2-YTE and cetuximab and the secretion of TGF-α (FIG. 7A) and amphiregulin (AREG, FIG. 7B). s-TGFα refers to secreted TGF-α. s-AREG refers to secreted AREG. The dashed lines are regression lines. The r² value for the regression line in FIG. 7A is 0.24. The r² value for the regression line in FIG. 7B is 0.50.

FIG. 8A-8B demonstrate that NRG is overexpressed in SCCHN samples. FIG. 8A depicts the level of NRG mRNA over background mRNA levels in individual SCCHN samples. Each bar represents an individual SCCHN patient sample. Background is defined as AQUA (Automated Quantitative Analysis; Genoptix; Carlsbad Calif.) score of a non-specific probe. FIG. 8B depicts the ratio of NRG mRNA to background in human SCCHN tumor samples grouped by anatomical location.

FIGS. 9A-9H depict the level of total EGFR (“H1T”, FIG. 9A), total ErbB2 (“H2T”, FIG. 9B), total ErbB3 (“H3T”, FIG. 9C), EGFR homodimers (“MD”, FIG. 9D), ErbB2/ErbB3 heterodimers (“H23D”, FIG. 9E), ErbB3 phosphorylated at Y1289 (“H3pY1289”, FIG. 9F), and ErbB3-PI3k heterodimers (“H3-PI3k”, FIG. 9G) as determined by the VeraTag® assay in human SCCHN tumor samples grouped by anatomical location, and the comparison of the total EGFR (H1T), ErbB2 (H2T), ErbB3 (H3T) (FIG. 9H) by the VeraTag® assay in human SCCHN tumor samples from all anatomical locations.

FIGS. 10A-10D depict the level of pY-H3 as a function of NRG mean (FIG. 10A), the level of pY-H3 as a function of NRG quartiles (FIG. 10B), the level of H11D as a function of NRG mean (FIG. 10C), and the level H11D as a function of NRG quartiles (FIG. 10D).

FIGS. 11A-11G depict a comparison between H1T (FIG. 11A), H2T (FIG. 11B), H3T (FIG. 11C), H11D (FIG. 11D), H23D (FIG. 11E), pY-H3 (FIG. 11F), and H3-P13K (FIG. 11G) levels in responder versus non-responder SCCHN cell lines.

FIGS. 12A-12C depicts the collection of all log 2 median-centered data from each of Affymetrix U133A (FIG. 12A), Affymetrix U133 2.0 (FIG. 12B), and Affymetrix U133 Plus 2.0 (FIG. 12C) platforms from ONCOMINE®, a cancer microarray database and web-based data-mining platform aimed at facilitating discovery from genome-wide expression analyses (see, e.g., Rhodes et al., 2004, Neoplasia 6(1):1-6). The collection of all log 2 median-centered data from each was extracted and quantile normalized.

FIGS. 13A-13D depict the frequency of overexpression of NRG1 and NRG2 within cancer types, using the mined data collected in FIG. 12, calculated by counting the number of samples and dividing by the total number of samples for each cancer type, then multiplying by 100. In FIGS. 13A-C, the left-hand bars for NRG1 and NRG2 indicate percentage of samples with expression above the median, and the right-hand bars for NRG1 and NRG2 indicate the percentage of samples with expression above the third quartile. In FIG. 13D, the bars indicate percentage of samples with expression above the median. The expression in the following cancer types was measured: B-Raf mutated melanoma (FIG. 13A), B-Raf mutated thyroid cancer (FIG. 13B), B-Raf mutated colorectal cancer (FIG. 13C), and B-Raf mutated lung cancer (FIG. 13D).

FIGS. 14A-14B depict the level of NRG1 mRNA over background mRNA levels in individual thyroid cancer samples from a thyroid cancer tumor microarray (TMA) (FIG. 14A) and the level of NRG2 mRNA over background mRNA levels in individual melanoma samples from a melanoma TMA (FIG. 14B). Each bar represents an individual patient sample. Background is defined as AQUA score of a non-specific probe, and is indicated by the dotted line.

FIGS. 15A-15B depict a protein gel of the purified NRG isoforms (NRG1α, NRG1β, NRG2α, and NRG2β), recombinantly expressed by HEK293 cells, after NiNTA and size exclusion chromatography (FIG. 15A), and the measurement of ErbB3 phosphorylation with titration of the purified NRG proteins on T47D cells (FIG. 15B).

FIGS. 16A-16B depict the correlation of high levels of EGFR homodimer (H11D) with the percent antiproliferative activity (FIG. 16A), and percent Akt phosphorylation (FIG. 16B) upon treatment with 2C2-YTE. A linear regression was performed on the VeraTag data (x-axis of 16A and B) and antiproliferation data (y-axis of 16A), and pAKT inhibition data from cell lines (y-axis of 16B). The r² value for the regression line in FIG. 16A is 0.48. The r² value for the regression line in FIG. 16B is 0.76.

FIGS. 17A-17B depict the reduction in tumor volume over time (days) for 2C2-YTE in comparison with an IgG1 control antibody in Cal27 cells (FIG. 17A), and CTG-0434 tumor fragments (FIG. 17B). Models were pre-selected for NRG positivity and high H11D, with levels (VeraTag output) indicated in parentheses.

### DETAILED DESCRIPTION

Provided herein are methods of treating a cancer comprising administer a HER3 inhibitor, an EGFR inhibitor, or both, as well as methods of determining whether a patient is likely to be responsive to such a method of treatment. In particular aspects, provided herein are methods of treating cancers expressing specific biomarkers with HER3 and/or EGFR inhibitors, and provided herein are also biomarkers and uses therefor in determining likelihood of effective cancer treatment with HER3 and/or EGFR inhibitors.

### I. Terminology

As used in this specification and the appended claims, the singular forms “a”, “an” and “the” include plural referents unless the context clearly dictates otherwise. The terms “a” (or “an”), as well as the terms “one or more,” and “at least one” can be used interchangeably herein.

Furthermore, “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless defined otherwise, all technical and scientific terms used herein have the same meaning as commonly understood by one of ordinary skill in the art to which this disclosure is related.

Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. The headings provided herein are not limitations of the various aspects, which can be had by reference to the specification as a whole. Accordingly, the terms defined immediately below are more fully defined by reference to the specification in its entirety.

It is understood that wherever aspects are described herein with the language “comprising,” otherwise analogous aspects described in terms of “consisting of” and/or “consisting essentially of” are also provided.

Amino acids are referred to herein by either their commonly known three letter symbols or by the one-letter symbols recommended by the IUPAC-IUB Biochemical Nomenclature Commission. Nucleotides, likewise, are referred to by their commonly accepted single-letter codes.

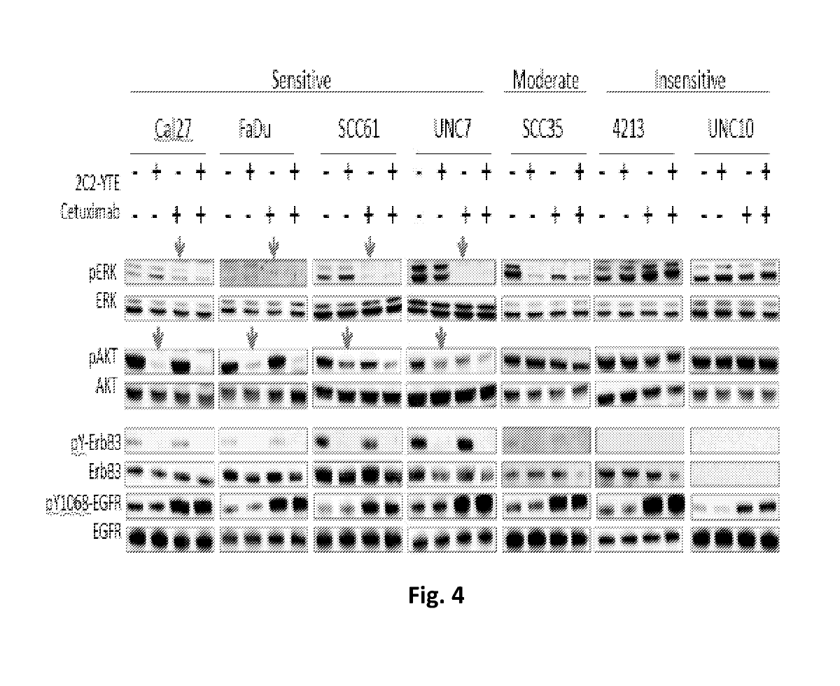

The terms “HER3,” “HER3 receptor,” “ErbB3” and “ErbB3 receptor” are used interchangeably herein, and refer to the ErbB3 protein (also referred to as HER3, ErbB3 receptor in the literature), which is well known in the art; see, e.g., U.S. Pat. No. 5,480,968 Plowman et al. (1990) Proc. Natl. Acad. Sci. USA 87, 4905-4909; see also, Kani et al. (2005) Biochemistry 44, 15842-15857, and Cho & Leahy (2002) Science 297, 1330-1333. A representative full-length, mature HER3 protein sequence (without leader sequence) corresponds to the sequence shown in FIG. 4 and SEQ ID NO: 4 of U.S. Pat. No. 5,480,968 minus the 19 amino acid leader sequence that is cleaved from the mature protein.

The terms “HER” and “HER receptor” are used interchangeably herein, and refer to one or more, or all, members of the epidermal growth factor receptor (EGFR) EGFR/HER subfamily of receptor protein tyrosine kinases (RTK), consisting of EGFR (HER1/Erbb1), HER2/Erbb2, HER3/Erbb3 and HER4/Erbb4. In a particular aspect, the terms “HER” and “HER receptor” are used interchangeably herein, and refer to EGFR, HER2, HER3, or HER4.

The terms “EGFR,” “HER1,” “HER1 receptor” and “EGFR receptor” are used interchangeably herein, and refer to the EGFR protein (also referred to as HER1, or ErbB1 receptor in the literature), for example, as described in Lin et al., Science, 1984, 224:843-848. A non-limiting example of an amino acid sequence of human EGFR is provided with GenBank Accession No. NP_005219.2. A non-limiting example of a nucleotide sequence encoding human EGFR is provided with GenBank Accession No. NM_005228.3.

The terms “B-Raf,” “BRAF,” “B-raf,” “B-Raf1,” “BRAF1,” and “RAFB1” are used interchangeably herein, and refer to the B-Raf protein, for example, as described in Stephens et al., 1992, Mol. Cell. Biol. 12(9):3733-3742. A non-limiting example of an amino acid sequence of human B-Raf is provided with GenBank Accession No. NP_004324.2. A non-limiting example of a nucleotide sequence encoding human B-Raf is provided with GenBank Accession No. NM_004333.4.

The terms MEK, “mitogen-activated protein kinase (MAPK) kinase” and “MAPKK” are used interchangeably herein, and refer to the MEK protein, for example, as described in Zheng and Guan, 1994, EMBO J. 13(5):1123-31. A non-limiting example of an amino acid sequence of human MEK is provided with GenBank Accession No. AAI37460.1. A non-limiting example of a nucleotide sequence encoding human MEK is provided with GenBank Accession No. BC137459.1.

The terms “neuregulin (NRG)” and “heregulin (HRG)” are used interchangeably herein, and refer to the family of neuregulin proteins, including isoforms type I to type III and subtypes (including, for example, NRG1, NRG2, NRG3, NRG4, and the subtypes or isoforms thereof, such as NRG1α, NRG1β, NRG2α, and NRG2β). Other synonyms for neuregulin include acetylcholine receptor inducing activity (ARIA), breast cancer cell differentiation factor p45, glial growth factor, Neu differentiation factor, and sensory and motor neuron-derived factor. A non-limiting example of an amino acid sequence of a human neuregulin, human NRG1, is provided with GenBank Accession No. AAI50610.1. A non-limiting example of a nucleotide sequence encoding a human neuregulin, human NRG1, is provided with GenBank Accession No. NM_013958.3. A non-limiting example of an amino acid sequence of a human neuregulin, human NRG2, is provided with GenBank Accession No. AAF28848.1. A non-limiting example of a nucleotide sequence encoding a human neuregulin, human NRG2, is provided with GenBank Accession No. NM_004883.2. A non-limiting example of an amino acid sequence of a human neuregulin, human NRG1α, is provided with GenBank Accession No. DAA00048.1. A non-limiting example of an amino acid sequence encoding a human neuregulin, human NRG1β, is provided with GenBank Accession No. AAA58639.1. A non-limiting example of an amino acid sequence of a human neuregulin, human NRG2α, is provided with GenBank Accession No. AAF28848.1. A non-limiting example of an amino acid sequence encoding a human neuregulin, human NRG2β, is provided with GenBank Accession No. AAF28849.1. Levels of NRG isoform expression in a cell or tumor sample may be assayed, for example, by using RNAscope® technology (see Section VI) using an RNAscope® probe developed for the specific isoform.

The term “amphiregulin (AREG)” used herein may be used interchangeably with “colorectum cell-derived growth factor (CRDGF)” and “Schwannoma-derived growth factor (SDGF),” and is a protein of the epidermal growth factor family. A non-limiting example of an amino acid sequence of human AREG is provided with GenBank Accession No. AAA51781.1. A non-limiting example of a nucleotide sequence encoding human AREG is provided with GenBank Accession No. NM_001657.3.

“Transforming growth factor alpha (TGF-α)” is a protein of the epidermal growth factor family. A non-limiting example of an amino acid sequence of human TGF-α is provided with GenBank Accession No. CAA49806.1. A non-limiting example of a nucleotide sequence encoding human TGF-α is provided with GenBank Accession No. NM_003236.3.

“Expressed” or “expression” may refer to: the transcription from a genetic sequence to give an RNA nucleic acid, the translation from an RNA molecule to give a protein, a polypeptide, or portion thereof, or the detectable presence or manifestation of a biomolecule or biomolecular complex, for example, a protein-protein complex. For example, the “expression” of an EGFR-homodimer may refer to the detectable level of EGFR-homodimer as measured, for example, by measuring the level of protein-protein interaction amongst EGFR monomers in a sample. Likewise, the “expression” of AREG may refer to the detectable level of AREG RNA or protein in a sample.

A “high” level refers to a level (e.g., of expression) that is greater than normal, for example, greater than a level in a “reference sample,” or a level that is greater than a particular standard. The reference sample may be normal/healthy cells, or may be all cancer cells, or may be a particular subset of cancer cells. For example, a reference sample may be a subset of cancer cells that do not express a neuregulin (e.g., colorectal cancer cells, for example the colorectal cancer cells used in the examples herein). A “high” level may also refer to a level that is higher than a predetermined amount or measure, such as a predetermined cutoff amount. A group of samples may be divided around a particularly determined value (for example, a median, a mean, or a quartile), or a particularly determined threshold (such as, for example, a line on a graph), such that two subgroups exist, one that is considered to have a “high” level (e.g., of expression) and one that is considered to have a “low” level (or, that does not have a “high” level). In certain instances, the phrase “high level of expression” might be used interchangeably with “overexpression.”

A “reference population” refers to a representative group of reference samples, and can be 1, 5, 10, 25, 50, 75, 100, 200, 250, 300, 400, 500, 1000, 2000, 5000, 10,000, 20,000, 30,000 or more samples. For example, the reference population may be a group of tumor samples, across cancer types (such as those found in Table 4). The reference population might also be tissue samples from a population of healthy individuals. A mean, median, quartile, or a line on a graph as determined from a reference population or a reference sample may determine a value or threshold against which an expression level may be “high.” For example, a reference population may be the Affymetrix U133A, Affymetrix U133 2.0, and Affymetrix U133 Plus 2.0 platforms from ONCOMINE®.

The terms “inhibition” and “suppression” are used interchangeably herein and refer to any statistically significant decrease in biological activity, including full blocking of the activity. For example, “inhibition” can refer to a decrease of about 10%, 20%, 30%, 40%, 50%, 60%, 70%, 80%, 90% or 100% in biological activity. Accordingly, when the terms “inhibition” or “suppression” are applied to describe, e.g., an effect on ligand-mediated HER3 phosphorylation, the term refers to the ability of an antibody or antigen binding fragment thereof to statistically significantly decrease the phosphorylation of HER3 induced by an EGF-like ligand, relative to the phosphorylation in an untreated (control) cell. The cell which expresses HER3 can be a naturally occurring cell or cell line (e.g., a cancer cell) or can be recombinantly produced by introducing a nucleic acid encoding HER3 into a host cell. In one aspect, the anti-HER3 binding molecule, e.g., an antibody or antigen binding fragment thereof inhibits ligand mediated phosphorylation of HER3 by at least 10%, or at least 20%, or at least 30%, or at least 40%, or at least 50%, or at least 60%, or at least 70%, or at least 80%, or at least 905, or about 100%, as determined, for example, by Western blotting followed by probing with an anti-phosphotyrosine antibody or by ELISA.

The terms “antibody” or “immunoglobulin,” as used interchangeably herein, include whole antibodies and any antigen binding fragment or single chains thereof.

A typical antibody comprises at least two heavy (H) chains and two light (L) chains interconnected by disulfide bonds. Each heavy chain is comprised of a heavy chain variable region (abbreviated herein as VH) and a heavy chain constant region. The heavy chain constant region is comprised of three domains, CH1, CH2, and CH3. Each light chain is comprised of a light chain variable region (abbreviated herein as VL) and a light chain constant region. The light chain constant region is comprised of one domain, CL. The VH and VL regions can be further subdivided into regions of hypervariability, termed Complementarity Determining Regions (CDR), interspersed with regions that are more conserved, termed framework regions (FW). Each VH and VL is composed of three CDRs and four FWs, arranged from amino-terminus to carboxy-terminus in the following order: FW1, CDR1, FW2, CDR2, FW3, CDR3, FW4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system. Exemplary antibodies of the present disclosure include the Clone 16 (CL16) anti-HER3 antibodies (original and germlined), affinity optimized clones including for example, the anti-HER3 2C2 antibody, and serum half-life-optimized anti-HER3 antibodies including for example the anti-HER3 2C2-YTE antibody.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, antibody fragments (such as Fab, Fab′, F(ab′)2, and Fv fragments), single chain Fv (scFv) mutants, multispecific antibodies such as bispecific antibodies generated from at least two intact antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

A “blocking” antibody or an “antagonist” antibody is one which inhibits or reduces biological activity of the antigen it binds, such as HER3. In a certain aspect blocking antibodies or antagonist antibodies substantially or completely inhibit the biological activity of the antigen. Desirably, the biological activity is reduced by 10%, 20%, 30%, 50%, 70%, 80%, 90%, 95%, or even 100%.

The term “HER3 antibody” or “an antibody that binds to HER3” or “anti-HER3” refers to an antibody that is capable of binding HER3 with sufficient affinity such that the antibody is useful as a therapeutic agent or diagnostic reagent in targeting HER3. The extent of binding of an anti-HER3 antibody to an unrelated, non-HER3 protein is less than about 10% of the binding of the antibody to HER3 as measured, e.g., by a radioimmunoassay (MA), BIACORE™ (using recombinant HER3 as the analyte and antibody as the ligand, or vice versa), or other binding assays known in the art. In certain aspects, an antibody that binds to HER3 has a dissociation constant (K_{D}) of ≤1 μM, ≤100 nM, ≤10 nM, ≤1 nM, ≤0.1 nM, ≤10 pM, ≤1 pM, or ≤0.1 pM.

The terms “antigen binding fragment” refers to a portion of an intact antibody and refers to the antigenic determining variable regions of an intact antibody. It is known in the art that the antigen binding function of an antibody can be performed by fragments of a full-length antibody. Examples of antibody fragments include, but are not limited to Fab, Fab′, F(ab′)2, and Fv fragments, linear antibodies, single chain antibodies, and multispecific antibodies formed from antibody fragments.

A “monoclonal antibody” refers to a homogeneous antibody population involved in the highly specific recognition and binding of a single antigenic determinant, or epitope. This is in contrast to polyclonal antibodies that typically include different antibodies directed against different antigenic determinants. The term “monoclonal antibody” encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal antibody” refers to such antibodies made in any number of ways including, but not limited to, by hybridoma, phage selection, recombinant expression, and transgenic animals.

The term “humanized antibody” refers to an antibody derived from a non-human (e.g., murine) immunoglobulin, which has been engineered to contain minimal non-human (e.g., murine) sequences. Typically, humanized antibodies are human immunoglobulins in which residues from the complementary determining region (CDR) are replaced by residues from the CDR of a non-human species (e.g., mouse, rat, rabbit, or hamster) that have the desired specificity, affinity, and capability (Jones et al., 1986, Nature, 321:522-525; Riechmann et al., 1988, Nature, 332:323-327; Verhoeyen et al., 1988, Science, 239:1534-1536). In some instances, the Fv framework region (FW) residues of a human immunoglobulin are replaced with the corresponding residues in an antibody from a non-human species that has the desired specificity, affinity, and capability.

The humanized antibody can be further modified by the substitution of additional residues either in the Fv framework region and/or within the replaced non-human residues to refine and optimize antibody specificity, affinity, and/or capability. In general, the humanized antibody will comprise substantially all of at least one, and typically two or three, variable domains containing all or substantially all of the CDR regions that correspond to the non-human immunoglobulin whereas all or substantially all of the FR regions are those of a human immunoglobulin consensus sequence. The humanized antibody can also comprise at least a portion of an immunoglobulin constant region or domain (Fc), typically that of a human immunoglobulin. Examples of methods used to generate humanized antibodies are described in U.S. Pat. No. 5,225,539 or 5,639,641.

A “variable region” of an antibody refers to the variable region of the antibody light chain or the variable region of the antibody heavy chain, either alone or in combination. The variable regions of the heavy and light chain each consist of four framework regions (FW) connected by three complementarity determining regions (CDRs) also known as hypervariable regions. The CDRs in each chain are held together in close proximity by the FW regions and, with the CDRs from the other chain, contribute to the formation of the antigen-binding site of antibodies. There are at least two techniques for determining CDRs: (1) an approach based on cross-species sequence variability (i.e., Kabat et al. Sequences of Proteins of Immunological Interest, (5th ed., 1991, National Institutes of Health, Bethesda Md.)); and (2) an approach based on crystallographic studies of antigen-antibody complexes (Al-lazikani et al. (1997) J. Molec. Biol. 273:927-948)). In addition, combinations of these two approaches are sometimes used in the art to determine CDRs.

The Kabat numbering system is generally used when referring to a residue in the variable domain (approximately residues 1-107 of the light chain and residues 1-113 of the heavy chain) (e.g., Kabat et al., Sequences of Immunological Interest, 5th Ed. Public Health Service, National Institutes of Health, Bethesda, Md. (1991)).

The amino acid position numbering as in Kabat, refers to the numbering system used for heavy chain variable domains or light chain variable domains of the compilation of antibodies in Kabat et al., Sequences of Proteins of Immunological Interest, 5th Ed. Public Health Service, National Institutes of Health, Bethesda, Md. (1991). Using this numbering system, the actual linear amino acid sequence can contain fewer or additional amino acids corresponding to a shortening of, or insertion into, a FW or CDR of the variable domain. For example, a heavy chain variable domain can include a single amino acid insert (residue 52a according to Kabat) after residue 52 of H2 and inserted residues (e.g., residues 82a, 82b, and 82c, etc. according to Kabat) after heavy chain FW residue 82.

TABLE 1

| Loop | Kabat | AbM | Chothia |
|---|---|---|---|
| L1 | L24-L34 | L24-L34 | L24-L34 |
| L2 | L50-L56 | L50-L56 | L50-L56 |
| L3 | L89-L97 | L89-L97 | L89-L97 |
| H1 | H31-H35B | H26-H35B | H26-H32..34 |

(Kabat Numbering)

| | | | |
|---|---|---|---|
| H1 | H31-H35 | H26-H35 | H26-H32 |

(Chothia Numbering)

| | | | |
|---|---|---|---|
| H2 | H50-H65 | H50-H58 | H52-H56 |
| H3 | H95-H102 | H95-H102 | H95-H102 |

The Kabat numbering of residues can be determined for a given antibody by alignment at regions of homology of the sequence of the antibody with a “standard” Kabat numbered sequence. Chothia refers instead to the location of the structural loops (Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software.

IMGT (ImMunoGeneTics) also provides a numbering system for the immunoglobulin variable regions, including the CDRs. See e.g., Lefranc, M. P. et al., Dev. Comp. Immunol. 27: 55-77(2003), which is herein incorporated by reference. The IMGT numbering system was based on an alignment of more than 5,000 sequences, structural data, and characterization of hypervariable loops and allows for easy comparison of the variable and CDR regions for all species. According to the IMGT numbering schema VH-CDR1 is at positions 26 to 35, VH-CDR2 is at positions 51 to 57, VH-CDR3 is at positions 93 to 102, VL-CDR1 is at positions 27 to 32, VL-CDR2 is at positions 50 to 52, and VL-CDR3 is at positions 89 to 97.

As used throughout the specification the VH CDRs sequences described correspond to the classical Kabat numbering locations, namely Kabat VH-CDR1 is at positions 31-35, VH-CDR2 is a positions 50-65, and VH-CDR3 is at positions 95-102. VL-CDR2 and VL-CDR3 also correspond to classical Kabat numbering locations, namely positions 50-56 and 89-97, respectively. As used herein, the terms “VL-CDR1” or “light chain CDR1” correspond to sequences located at Kabat positions 23-34 in the VL (in contrast, the classical VL-CDR1 location according to the Kabat numbering schema corresponds to positions 24-34).

As used herein the Fc region includes the polypeptides comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM Fc may include the J chain. For IgG, Fc comprises immunoglobulin domains Cgamma2 and Cgamma3 (Cγ2 and Cγ3) and the hinge between Cgamma1 (Cγ1) and Cgamma2 (Cγ2). Although the boundaries of the Fc region may vary, the human IgG heavy chain Fc region is usually defined to comprise residues C226 or P230 to its carboxyl-terminus, wherein the numbering is according to the EU index as set forth in Kabat (Kabat et al., Sequences of Proteins of Immunological Interest, 5th Ed. Public Health Service, National Institutes of Health, Bethesda, Md. (1991)). Fc may refer to this region in isolation, or this region in the context of an antibody, antibody fragment, or Fc fusion protein. Polymorphisms have been observed at a number of different Fc positions, including but not limited to positions 270, 272, 312, 315, 356, and 358 as numbered by the EU index, and thus slight differences between the presented sequence and sequences in the prior art may exist.

The term “human antibody” means an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide such as, for example, an antibody comprising murine light chain and human heavy chain polypeptides.

The term “chimeric antibodies” refers to antibodies wherein the amino acid sequence of the immunoglobulin molecule is derived from two or more species. Typically, the variable region of both light and heavy chains corresponds to the variable region of antibodies derived from one species of mammals (e.g., mouse, rat, rabbit, etc) with the desired specificity, affinity, and capability while the constant regions are homologous to the sequences in antibodies derived from another (usually human) to avoid eliciting an immune response in that species.

The terms “YTE” or “YTE mutant” refer to a mutation in IgG1 Fc that results in an increase in the binding to human FcRn and improves the serum half-life of the antibody having the mutation. A YTE mutant comprises a combination of three mutations, M252Y/S254T/T256E (EU numbering Kabat et al. (1991) Sequences of Proteins of Immunological Interest, U.S. Public Health Service, National Institutes of Health, Washington, D.C.), introduced into the heavy chain of an IgG1. See U.S. Pat. No. 7,658,921, which is incorporated by reference herein. The YTE mutant has been shown to increase the serum half-life of antibodies approximately four-times as compared to wild-type versions of the same antibody (Dall'Acqua et al., J. Biol. Chem. 281:23514-24 (2006)). See also U.S. Pat. No. 7,083,784, which is hereby incorporated by reference in its entirety.

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes described herein.

“Potency” is normally expressed as an IC₅₀ value, in nM unless otherwise stated. IC₅₀ is the median inhibitory concentration of an antibody molecule. In functional assays, IC₅₀ is the concentration that reduces a biological response by 50% of its maximum. In ligand-binding studies, IC₅₀ is the concentration that reduces receptor binding by 50% of maximal specific binding level. IC₅₀ can be calculated by any number of means known in the art. Improvement in potency can be determined by measuring, e.g., against the parent CL16 (Clone 16) monoclonal antibody.

The fold improvement in potency for the antibodies or polypeptides described herein as compared to a Clone 16 antibody can be at least about 2-fold, at least about 4-fold, at least about 6-fold, at least about 8-fold, at least about 10-fold, at least about 20-fold, at least about 30-fold, at least about 40-fold, at least about 50-fold, at least about 60-fold, at least about 70-fold, at least about 80-fold, at least about 90-fold, at least about 100-fold, at least about 110-fold, at least about 120-fold, at least about 130-fold, at least about 140-fold, at least about 150-fold, at least about 160-fold, at least about 170-fold, or at least about 180-fold or more.

“Antibody-dependent cell-mediated cytotoxicity” or “ADCC” refers to a form of cytotoxicity in which secreted Ig bound onto Fc receptors (FcRs) present on certain cytotoxic cells (e.g., Natural Killer (NK) cells, neutrophils, and macrophages) enables these cytotoxic effector cells to bind specifically to an antigen-bearing target cell and subsequently kill the target cell with cytotoxins. Specific high-affinity IgG antibodies directed to the surface of target cells “arm” the cytotoxic cells and are absolutely required for such killing. Lysis of the target cell is extracellular, requires direct cell-to-cell contact, and does not involve complement. It is contemplated that, in addition to antibodies, other proteins comprising Fc regions, specifically Fc fusion proteins, having the capacity to bind specifically to an antigen-bearing target cell will be able to effect cell-mediated cytotoxicity. For simplicity, the cell-mediated cytotoxicity resulting from the activity of an Fc fusion protein is also referred to herein as ADCC activity.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cells or compositions include those which have been purified to a degree that they are no longer in a form in which they are found in nature. In some aspects, an antibody, polynucleotide, vector, cell, or composition which is isolated is substantially pure.

The term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

The term “pharmaceutical composition” refers to a preparation which is in such form as to permit the biological activity of the active ingredient to be effective, and which contains no additional components which are unacceptably toxic to a subject to which the composition would be administered. Such composition can be sterile.

An “effective amount” of an antibody as disclosed herein is an amount sufficient to carry out a specifically stated purpose. An “effective amount” can be determined empirically and in a routine manner, in relation to the stated purpose.

The term “therapeutically effective amount” refers to an amount of an antibody or other drug effective to “treat” a disease or disorder in a subject or mammal.

The word “label” when used herein refers to a detectable compound or composition which is conjugated directly or indirectly to the antibody so as to generate a “labeled” antibody. The label can be detectable by itself (e.g., radioisotope labels or fluorescent labels) or, in the case of an enzymatic label, can catalyze chemical alteration of a substrate compound or composition which is detectable.

Terms such as “treating” or “treatment” or “to treat” or “alleviating” or “to alleviate” refer to therapeutic measures that cure, slow down, lessen symptoms of, and/or halt progression of a diagnosed pathologic condition or disorder and slow the development of a targeted pathologic condition or disorder. Thus, those in need of treatment include those already with the disorder; those prone to have the disorder; and those in whom the disorder is to be prevented. In certain aspects, a subject is successfully “treated” for cancer according to the methods described herein if the patient shows, e.g., total, partial, or transient remission of a certain type of cancer.

The terms “cancer”, “tumor”, “cancerous”, and “malignant” refer to or describe the physiological condition in mammals that is typically characterized by unregulated cell growth. Examples of cancers include but are not limited to, carcinoma including adenocarcinomas, lymphomas, blastomas, melanomas, sarcomas, and leukemias. More particular examples of such cancers include brain cancer, CNS cancer, squamous cell cancer, small-cell lung cancer, non-small cell lung cancer, gastrointestinal cancer, Hodgkin's and non-Hodgkin's lymphoma, pancreatic cancer, glioblastoma, glioma, cervical cancer, ovarian cancer, liver cancer such as hepatic carcinoma and hepatoma, bladder cancer, breast cancer (including hormonally mediated breast cancer, see, e.g., Innes et al. (2006) Br. J. Cancer 94:1057-1065), colon cancer, colorectal cancer, endometrial carcinoma, myeloma (such as multiple myeloma), salivary gland carcinoma, kidney cancer such as renal cell carcinoma and Wilms' tumors, basal cell carcinoma, melanoma, prostate cancer, vulval cancer, thyroid cancer, testicular cancer, esophageal cancer, various types of head and neck cancer and cancers of mucinous origins, such as, mucinous ovarian cancer, cholangiocarcinoma (liver) and renal papillary carcinoma.

As used herein, the term “carcinomas” refers to cancers of epithelial cells, which are cells that cover the surface of the body, produce hormones, and make up glands. Examples of carcinomas are cancers of the skin, lung, colon, stomach, breast, prostate and thyroid gland.

By “assaying the activity level of HER3 protein” is intended qualitatively or quantitatively measuring or estimating the activity of HER3 protein in a first biological sample either directly (e.g., by determining or estimating absolute activity level) or relatively (e.g., by comparing to the activity level in a second biological sample). HER3 protein activity level in the first biological sample can be measured or estimated and compared to a standard HER3 protein activity, the standard being taken from a second biological sample obtained from an individual not having the disorder or being determined by averaging levels from a population of individuals not having the disorder or from an individual prior to treatment. As will be appreciated in the art, once the “standard” HER3 protein activity level is known, it can be used repeatedly as a standard for comparison. In certain aspects, the activity level of HER3 in a biological sample is measured or estimated or compared by detecting phosphorylated HER3 in a biological sample. In a specific aspect, the activity level of HER3 in a biological sample is measured or estimated or compared by detecting phosphorylated HER3 in a skin biopsy, wherein the skin is stimulated with HRG prior to or after biopsy.

The term “positive therapeutic response” with respect to cancer treatment refers to an improvement in the disease in association with the activity of these anti-HER3 binding molecules, e.g., antibodies or antigen-binding fragments, variants, or derivatives thereof, and/or an improvement in the symptoms associated with the disease. Thus, for example, an improvement in the disease can be characterized as a complete response. By “complete response” is intended an absence of clinically detectable disease with normalization of any previously test results. Alternatively, an improvement in the disease can be categorized as being a partial response. A “positive therapeutic response” encompasses a reduction or inhibition of the progression and/or duration of cancer, the reduction or amelioration of the severity of cancer, and/or the amelioration of one or more symptoms thereof resulting from the administration of an anti-HER3 binding molecule described herein. In specific aspects, such terms refer to one, two or three or more results following the administration of anti-HER3 binding molecules described herein: (1) a stabilization, reduction or elimination of the cancer cell population; (2) a stabilization or reduction in cancer growth; (3) an impairment in the formation of cancer; (4) eradication, removal, or control of primary, regional and/or metastatic cancer; (5) a reduction in mortality; (6) an increase in disease-free, relapse-free, progression-free, and/or overall survival, duration, or rate; (7) an increase in the response rate, the durability of response, or number of patients who respond or are in remission; (8) a decrease in hospitalization rate, (9) a decrease in hospitalization lengths, (10) the size of the cancer is maintained and does not increase or increases by less than 10%, preferably less than 5%, preferably less than 4%, preferably less than 2%, and (12) an increase in the number of patients in remission.

By “sample from a patient” or “biological sample” is intended any biological sample obtained from an individual, cell line, tissue culture, or other source of cells potentially expressing HER3. Methods for obtaining tissue biopsies and body fluids from mammals are well known in the art.

A combination therapy can provide “synergy” and prove “synergistic”, i.e., the effect achieved when the active ingredients used together is greater than the sum of the effects that results from using the compounds separately. A synergistic effect can be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect can be attained when the compounds are administered or delivered sequentially, e.g., by different injections in separate syringes. In general, during alternation therapy, an effective dosage of each active ingredient is administered sequentially, i.e., serially, whereas in combination therapy, effective dosages of two or more active ingredients are administered together.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide can comprise modified nucleotides, such as methylated nucleotides and their analogs. The preceding description applies to all polynucleotides referred to herein, including RNA and DNA.

The term “vector” means a construct, which is capable of delivering, and in some aspects, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides described herein are based upon antibodies, in certain aspects, the polypeptides can occur as single chains or associated chains.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences.

One such non-limiting example of a sequence alignment algorithm is the algorithm described in Karlin et al., 1990, Proc. Natl. Acad. Sci., 87:2264-2268, as modified in Karlin et al., 1993, Proc. Natl. Acad. Sci., 90:5873-5877, and incorporated into the NBLAST and) XBLAST programs (Altschul et al., 1991, Nucleic Acids Res., 25:3389-3402). In certain aspects, Gapped BLAST can be used as described in Altschul et al., 1997, Nucleic Acids Res. 25:3389-3402. BLAST-2, WU-BLAST-2 (Altschul et al., 1996, Methods in Enzymology, 266:460-480), ALIGN, ALIGN-2 (Genentech, South San Francisco, Calif.) or Megalign (DNASTAR) are additional publicly available software programs that can be used to align sequences. In certain aspects, the percent identity between two nucleotide sequences is determined using the GAP program in the GCG software package (e.g., using a NWSgapdna.CMP matrix and a gap weight of 40, 50, 60, 70, or 90 and a length weight of 1, 2, 3, 4, 5, or 6). In certain alternative aspects, the GAP program in the GCG software package, which incorporates the algorithm of Needleman and Wunsch (J. Mol. Biol. (48):444-453 (1970)) can be used to determine the percent identity between two amino acid sequences (e.g., using either a BLOSUM 62 matrix or a PAM250 matrix, and a gap weight of 16, 14, 12, 10, 8, 6, or 4 and a length weight of 1, 2, 3, 4, 5). Alternatively, in certain aspects, the percent identity between nucleotide or amino acid sequences is determined using the algorithm of Myers and Miller (CABIOS, 4:11-17 (1989)). For example, the percent identity can be determined using the ALIGN program (version 2.0) and using a PAM120 with residue table, a gap length penalty of 12 and a gap penalty of 4. Appropriate parameters for maximal alignment by particular alignment software can be determined by one skilled in the art. In certain aspects, the default parameters of the alignment software are used.

In certain aspects, the percentage identity “X” of a first amino acid sequence to a second sequence amino acid is calculated as 100×(Y/Z), where Y is the number of amino acid residues scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

A “conservative amino acid substitution” is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. In certain aspects, conservative substitutions in the sequences of the polypeptides and antibodies described herein do not abrogate the binding of the polypeptide or antibody containing the amino acid sequence, to the antigen(s), i.e., the HER3 to which the polypeptide or antibody binds. Methods of identifying nucleotide and amino acid conservative substitutions which do not eliminate antigen binding are well-known in the art (see, e.g., Brummell et al., Biochem. 32: 1180-1187 (1993); Kobayashi et al., Protein Eng. 12(10):879-884 (1999); and Burks et al., Proc. Natl. Acad. Sci. USA 94:412-417 (1997)).

The term “consensus sequence,” as used herein with respect to light chain (VL) and heavy chain (VH) variable regions, refers to a composite or genericized VL or VH sequence defined based on information as to which amino acid residues within the VL or VH chain are amenable to modification without detriment to antigen binding. Thus, in a “consensus sequence” for a VL or VH chain, certain amino acid positions are occupied by one of multiple possible amino acid residues at that position. For example, if an arginine (R) or a serine (S) occur at a particular position, then that particular position within the consensus sequence can be either arginine or serine (R or S). Consensus sequences for VH and VL chain can be defined, for example, by in vitro affinity maturation (e.g., randomizing every amino acid position in a certain CDR using degenerate coding primers), by scanning mutagenesis (e.g., alanine scanning mutagenesis) of amino acid residues within the antibody CDRs, or any other methods known in the art, followed by evaluation of the binding of the mutants to the antigen to determine whether the mutated amino acid position affects antigen binding. In some aspects, mutations are introduced in the CDR regions. In other aspects, mutations are introduced in framework regions. In some other aspects, mutations are introduced in CDR and framework regions.

### II. Methods of Treating Cancer

In one embodiment, disclosed herein is a method for treating a cancer (e.g., squamous cell carcinoma of the head and neck (SCCHN)), comprising administering to a patient diagnosed with said cancer a therapeutically effective amount of a combination of a HER3 inhibitor, for example, an anti-HER3 antibody (e.g., 2C2-YTE), and an EGFR inhibitor, for example, an anti-EGFR antibody (e.g. cetuximab), wherein the patient has a tumor that has been characterized in that cells from the tumor: i) express a neuregulin (e.g. NRG1 and/or NRG2), and ii) express high levels of AREG and TGF-α, for example, cells from the tumor express AREG and TGF-α protein or mRNA at levels above the median level for that cancer type. In specific embodiments, cells from the tumor express a neuregulin (e.g. NRG1 and/or NRG2) at high levels. In another embodiment, high levels of AREG and TGF-α, refer to expression in comparison to a reference sample, e.g., AREG and TGF-α are expressed at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells that do not express a neuregulin, or a healthy tissue sample, or the UNC10 cell line (Coleman et al., Arch Otolaryngol Head Neck Surg. 2002; 128(2):167-176), see Examples), or the mean or median levels expressed by a reference population (e.g., a representative group of tumor samples, across cancer types, such as those found in Table 4). In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E).

In one embodiment, disclosed herein is a method for treating a cancer (e.g., squamous cell carcinoma of the head and neck (SCCHN)), comprising administering to a patient diagnosed with said cancer a therapeutically effective amount of a HER3 inhibitor, for example, an anti-HER3 antibody (e.g., 2C2-YTE), wherein the patient has a tumor that has been characterized in that cells from the tumor: i) express a neuregulin (e.g. NRG1 and/or NRG2), and ii) express high levels of AREG and TGF-α, for example, cells from the tumor express AREG and TGF-α protein or mRNA at levels above the median level for that cancer type. In specific embodiments, cells from the tumor express a neuregulin (e.g. NRG1 and/or NRG2) at high levels. In another embodiment, high levels of AREG and TGF-α, refer to expression in comparison to a reference sample, e.g., AREG and TGF-α are expressed at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4). In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E).

In one embodiment, disclosed herein is a method for treating a cancer (e.g., squamous cell carcinoma of the head and neck (SCCHN)), comprising administering to a patient diagnosed with said cancer a therapeutically effective amount of an EGFR inhibitor, for example, an anti-EGFR antibody (e.g. cetuximab), wherein the patient has a tumor that has been characterized in that cells from the tumor: i) express a neuregulin (e.g. NRG1 and/or NRG2), and ii) express high levels of AREG and TGF-α, for example, cells from the tumor express AREG and TGF-α protein or mRNA at levels above the median level for that cancer type. In specific embodiments, cells from the tumor express a neuregulin (e.g. NRG1 and/or NRG2) at high levels. In another embodiment, high levels of AREG and TGF-α, refer to expression in comparison to a reference sample, e.g., AREG and TGF-α are expressed at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4).

In one embodiment, disclosed herein is a method for treating a cancer (e.g., squamous cell carcinoma of the head and neck (SCCHN)), comprising administering to a patient diagnosed with said cancer a therapeutically effective amount of a combination of a HER3 inhibitor, for example, an anti-HER3 antibody (e.g., 2C2-YTE), and an EGFR inhibitor, for example, an anti-EGFR antibody (e.g. cetuximab), wherein the patient has a tumor that has been characterized in that cells from the tumor: i) express a neuregulin (e.g. NRG1 and/or NRG2), and ii) express a high level of AREG, for example, cells from the tumor express AREG protein or mRNA at levels above the median level for that cancer type. In specific embodiments, cells from the tumor express a neuregulin (e.g. NRG1 and/or NRG2) at high levels. In another embodiment, a high levels of AREG refers to expression in comparison to a reference sample, e.g., AREG is expressed at a level 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the level expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4). In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E).

In one embodiment, disclosed herein is a method for treating a cancer (e.g., squamous cell carcinoma of the head and neck (SCCHN)), comprising administering to a patient diagnosed with said cancer a therapeutically effective amount of a HER3 inhibitor, for example, an anti-HER3 antibody (e.g., 2C2-YTE), wherein the patient has a tumor that has been characterized in that cells from the tumor: i) express a neuregulin (e.g. NRG1 and/or NRG2), and ii) express a high level of AREG, for example, cells from the tumor express AREG protein or mRNA at levels above the median level for that cancer type. In specific embodiments, cells from the tumor express a neuregulin (e.g. NRG1 and/or NRG2) at high levels. In another embodiment, a high levels of AREG refers to expression in comparison to a reference sample, e.g., AREG is expressed at a level 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the level expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4). In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E).

In one embodiment, disclosed herein is a method for treating a cancer (e.g., squamous cell carcinoma of the head and neck (SCCHN)), comprising administering to a patient diagnosed with said cancer a therapeutically effective amount of an EGFR inhibitor, for example, an anti-EGFR antibody (e.g. cetuximab), wherein the patient has a tumor that has been characterized in that cells from the tumor: i) express a neuregulin (e.g. NRG1 and/or NRG2), and ii) express a high level of AREG, for example, cells from the tumor express AREG protein or mRNA at levels above the median level for that cancer type. In specific embodiments, cells from the tumor express a neuregulin (e.g. NRG1 and/or NRG2) at high levels. In another embodiment, a high levels of AREG refers to expression in comparison to a reference sample, e.g., AREG is expressed at a level 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the level expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4).

In one embodiment, disclosed herein is a method for treating a cancer (e.g., squamous cell carcinoma of the head and neck (SCCHN)), comprising administering to a patient diagnosed with said cancer a therapeutically effective amount of a combination of a HER3 inhibitor, for example, an anti-HER3 antibody (e.g., 2C2-YTE), and an EGFR inhibitor, for example, an anti-EGFR antibody (e.g. cetuximab), wherein the patient has a tumor that has been characterized in that cells from the tumor: i) express a neuregulin (e.g. NRG1 and/or NRG2), and ii) express a high level of TGF-α, for example, cells from the tumor express TGF-α protein or mRNA at levels above the median level for that cancer type. In specific embodiments, cells from the tumor express a neuregulin (e.g. NRG1 and/or NRG2) at high levels. In another embodiment, a high levels of TGF-α refers to expression in comparison to a reference sample, e.g., TGF-α is expressed at a level 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the level expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4). In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E).

In one embodiment, disclosed herein is a method for treating a cancer (e.g., squamous cell carcinoma of the head and neck (SCCHN)), comprising administering to a patient diagnosed with said cancer a therapeutically effective amount of a HER3 inhibitor, for example, an anti-HER3 antibody (e.g., 2C2-YTE), wherein the patient has a tumor that has been characterized in that cells from the tumor: i) express a neuregulin (e.g. NRG1 and/or NRG2), and ii) express a high level of TGF-α, for example, cells from the tumor express TGF-α protein or mRNA at levels above the median level for that cancer type. In specific embodiments, cells from the tumor express a neuregulin (e.g. NRG1 and/or NRG2) at high levels. In another embodiment, a high levels of AREG refers to expression in comparison to a reference sample, e.g., TGF-α is expressed at a level 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the level expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4). In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E).

In one embodiment, disclosed herein is a method for treating a cancer (e.g., squamous cell carcinoma of the head and neck (SCCHN)), comprising administering to a patient diagnosed with said cancer a therapeutically effective amount of an EGFR inhibitor, for example, an anti-EGFR antibody (e.g. cetuximab), wherein the patient has a tumor that has been characterized in that cells from the tumor: i) express a neuregulin (e.g. NRG1 and/or NRG2), and ii) express a high level of TGF-α, for example, cells from the tumor express TGF-α protein or mRNA at levels above the median level for that cancer type. In specific embodiments, cells from the tumor express a neuregulin (e.g. NRG1 and/or NRG2) at high levels. In another embodiment, a high levels of TGF-α refers to expression in comparison to a reference sample, e.g., TGF-α is expressed at a level 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the level expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4).

In one embodiment, disclosed herein is a method for treating a cancer (e.g., squamous cell carcinoma of the head and neck (SCCHN)), comprising administering to a patient diagnosed with said cancer a therapeutically effective amount of a combination of a HER3 inhibitor, for example, an anti-HER3 antibody (e.g., 2C2-YTE), and an EGFR inhibitor, for example, an anti-EGFR antibody (e.g. cetuximab), wherein the patient has a tumor that has been characterized in that cells from the tumor: i) express a neuregulin (e.g. NRG1 and/or NRG2), and ii) express high levels of EGFR homodimer, AREG and TGF-α, for example, cells from the tumor express EGFR homodimer, AREG and TGF-α at levels above the median level for that cancer type. In specific embodiments, cells from the tumor express a neuregulin (e.g. NRG1 and/or NRG2) at high levels. In another embodiment, high levels of EGFR homodimer, AREG and TGF-α, refer to expression in comparison to a reference sample, e.g., EGFR homodimer, AREG and TGF-α are expressed at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4). In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E).

In one embodiment, disclosed herein is a method for treating a cancer (e.g., squamous cell carcinoma of the head and neck (SCCHN)), comprising administering to a patient diagnosed with said cancer a therapeutically effective amount of a HER3 inhibitor, for example, an anti-HER3 antibody (e.g., 2C2-YTE), wherein the patient has a tumor that has been characterized in that cells from the tumor: i) express a neuregulin (e.g. NRG1 and/or NRG2), and ii) express high levels of EGFR homodimer, AREG and TGF-α, for example, cells from the tumor express EGFR homodimer, AREG and TGF-α at levels above the median level for that cancer type. In specific embodiments, cells from the tumor express a neuregulin (e.g. NRG1 and/or NRG2) at high levels. In another embodiment, high levels of EGFR homodimer, AREG and TGF-α, refer to expression in comparison to a reference sample, e.g., EGFR homodimer, AREG and TGF-α are expressed at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4). In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E).

In one embodiment, disclosed herein is a method for treating a cancer (e.g., squamous cell carcinoma of the head and neck (SCCHN)), comprising administering to a patient diagnosed with said cancer a therapeutically effective amount of an EGFR inhibitor, for example, an anti-EGFR antibody (e.g. cetuximab), wherein the patient has a tumor that has been characterized in that cells from the tumor: i) express a neuregulin (e.g. NRG1 and/or NRG2), and ii) express high levels of EGFR homodimer, AREG and TGF-α, for example, cells from the tumor express EGFR homodimer, AREG and TGF-α at levels above the median level for that cancer type. In specific embodiments, cells from the tumor express a neuregulin (e.g. NRG1 and/or NRG2) at high levels. In another embodiment, high levels of EGFR homodimer, AREG and TGF-α, refer to expression in comparison to a reference sample, e.g., EGFR homodimer, AREG and TGF-α are expressed at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4).

In one embodiment, disclosed herein is a method for treating a cancer (e.g., squamous cell carcinoma of the head and neck (SCCHN)), comprising administering to a patient diagnosed with said cancer a therapeutically effective amount of a combination of a HER3 inhibitor, for example, an anti-HER3 antibody (e.g., 2C2-YTE), and an EGFR inhibitor, for example, an anti-EGFR antibody (e.g. cetuximab), wherein the patient has a tumor that has been characterized in that cells from the tumor: i) express a neuregulin (e.g. NRG1 and/or NRG2), and ii) express high levels of EGFR homodimer and AREG, for example, cells from the tumor express EGFR homodimer and AREG at levels above the median level for that cancer type. In specific embodiments, cells from the tumor express a neuregulin (e.g. NRG1 and/or NRG2) at high levels. In another embodiment, high levels of EGFR homodimer and AREG refer to expression in comparison to a reference sample, e.g., EGFR homodimer and AREG are expressed at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4). In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E).

In one embodiment, disclosed herein is a method for treating a cancer (e.g., squamous cell carcinoma of the head and neck (SCCHN)), comprising administering to a patient diagnosed with said cancer a therapeutically effective amount of a HER3 inhibitor, for example, an anti-HER3 antibody (e.g., 2C2-YTE), wherein the patient has a tumor that has been characterized in that cells from the tumor: i) express a neuregulin (e.g. NRG1 and/or NRG2), and ii) express high levels of EGFR homodimer and AREG, for example, cells from the tumor express EGFR homodimer and AREG at levels above the median level for that cancer type. In specific embodiments, cells from the tumor express a neuregulin (e.g. NRG1 and/or NRG2) at high levels. In another embodiment, high levels of EGFR homodimer and AREG refer to expression in comparison to a reference sample, e.g., EGFR homodimer and AREG are expressed at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4). In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E).

In one embodiment, disclosed herein is a method for treating a cancer (e.g., squamous cell carcinoma of the head and neck (SCCHN)), comprising administering to a patient diagnosed with said cancer a therapeutically effective amount of an EGFR inhibitor, for example, an anti-EGFR antibody (e.g. cetuximab), wherein the patient has a tumor that has been characterized in that cells from the tumor: i) express a neuregulin (e.g. NRG1 and/or NRG2), and ii) express high levels of EGFR homodimer and AREG, for example, cells from the tumor express EGFR homodimer and AREG at levels above the median level for that cancer type. In specific embodiments, cells from the tumor express a neuregulin (e.g. NRG1 and/or NRG2) at high levels. In another embodiment, high levels of EGFR homodimer and AREG refer to expression in comparison to a reference sample, e.g., EGFR homodimer and AREG are expressed at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4).

In one embodiment, disclosed herein is a method for treating a cancer (e.g., squamous cell carcinoma of the head and neck (SCCHN)), comprising administering to a patient diagnosed with said cancer a therapeutically effective amount of a combination of a HER3 inhibitor, for example, an anti-HER3 antibody (e.g., 2C2-YTE), and an EGFR inhibitor, for example, an anti-EGFR antibody (e.g. cetuximab), wherein the patient has a tumor that has been characterized in that cells from the tumor: i) express a neuregulin (e.g. NRG1 and/or NRG2), and ii) express high levels of EGFR homodimer and TGF-α, for example, cells from the tumor express EGFR homodimer and TGF-α at levels above the median level for that cancer type. In specific embodiments, cells from the tumor express a neuregulin (e.g. NRG1 and/or NRG2) at high levels. In another embodiment, high levels of EGFR homodimer and TGF-α refer to expression in comparison to a reference sample, e.g., EGFR homodimer and TGF-α are expressed at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4). In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E).

In one embodiment, disclosed herein is a method for treating a cancer (e.g., squamous cell carcinoma of the head and neck (SCCHN)), comprising administering to a patient diagnosed with said cancer a therapeutically effective amount of a HER3 inhibitor, for example, an anti-HER3 antibody (e.g., 2C2-YTE), wherein the patient has a tumor that has been characterized in that cells from the tumor: i) express a neuregulin (e.g. NRG1 and/or NRG2), and ii) express high levels of EGFR homodimer and TGF-α, for example, cells from the tumor express EGFR homodimer and TGF-α at levels above the median level for that cancer type. In specific embodiments, cells from the tumor express a neuregulin (e.g. NRG1 and/or NRG2) at high levels. In another embodiment, high levels of EGFR homodimer and TGF-α refer to expression in comparison to a reference sample, e.g., EGFR homodimer and TGF-α are expressed at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4). In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E).

In one embodiment, disclosed herein is a method for treating a cancer (e.g., squamous cell carcinoma of the head and neck (SCCHN)), comprising administering to a patient diagnosed with said cancer a therapeutically effective amount of an EGFR inhibitor, for example, an anti-EGFR antibody (e.g. cetuximab), wherein the patient has a tumor that has been characterized in that cells from the tumor: i) express a neuregulin (e.g. NRG1 and/or NRG2), and ii) express high levels of EGFR homodimer and TGF-α, for example, cells from the tumor express EGFR homodimer and TGF-α at levels above the median level for that cancer type. In specific embodiments, cells from the tumor express a neuregulin (e.g. NRG1 and/or NRG2) at high levels. In another embodiment, high levels of EGFR homodimer and TGF-α refer to expression in comparison to a reference sample, e.g., EGFR homodimer and TGF-α are expressed at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4).

In one embodiment, disclosed herein is a method for treating a cancer (e.g., squamous cell carcinoma of the head and neck (SCCHN)), comprising administering to a patient diagnosed with said cancer a therapeutically effective amount of a combination of a HER3 inhibitor, for example, an anti-HER3 antibody (e.g., 2C2-YTE), and an EGFR inhibitor, for example, an anti-EGFR antibody (e.g. cetuximab), wherein the patient has a tumor that has been characterized in that cells from the tumor: i) express a neuregulin (e.g. NRG1 and/or NRG2), and ii) express high levels of EGFR homodimer, for example, cells from the tumor express EGFR homodimer at a level above the median level for that cancer type. In specific embodiments, cells from the tumor express a neuregulin (e.g. NRG1 and/or NRG2) at high levels. In another embodiment, a high level of EGFR homodimer refers to expression in comparison to a reference sample, e.g., EGFR homodimer is expressed at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the level expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4). In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E).

In one embodiment, disclosed herein is a method for treating a cancer (e.g., squamous cell carcinoma of the head and neck (SCCHN)), comprising administering to a patient diagnosed with said cancer a therapeutically effective amount of a HER3 inhibitor, for example, an anti-HER3 antibody (e.g., 2C2-YTE), wherein the patient has a tumor that has been characterized in that cells from the tumor: i) express a neuregulin (e.g. NRG1 and/or NRG2), and ii) express high levels of EGFR homodimer, for example, cells from the tumor express EGFR homodimer at a level above the median level for that cancer type. In specific embodiments, cells from the tumor express a neuregulin (e.g. NRG1 and/or NRG2) at high levels. In another embodiment, a high level of EGFR homodimer refers to expression in comparison to a reference sample, e.g., EGFR homodimer is expressed at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the level expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4). In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E).

In one embodiment, disclosed herein is a method for treating a cancer (e.g., squamous cell carcinoma of the head and neck (SCCHN)), comprising administering to a patient diagnosed with said cancer a therapeutically effective amount of an EGFR inhibitor, for example, an anti-EGFR antibody (e.g. cetuximab), wherein the patient has a tumor that has been characterized in that cells from the tumor: i) express a neuregulin (e.g. NRG1 and/or NRG2), and ii) express high levels of EGFR homodimer, for example, cells from the tumor express EGFR homodimer at a level above the median level for that cancer type. In specific embodiments, cells from the tumor express a neuregulin (e.g. NRG1 and/or NRG2) at high levels. In another embodiment, a high level of EGFR homodimer refers to expression in comparison to a reference sample, e.g., EGFR homodimer is expressed at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the level expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4).

In one embodiment, disclosed herein is a method for treating a cancer (e.g., squamous cell carcinoma of the head and neck (SCCHN)), comprising administering to a patient diagnosed with said cancer a therapeutically effective amount of a combination of a HER3 inhibitor, for example, an anti-HER3 antibody (e.g., 2C2-YTE), and an EGFR inhibitor, for example, an anti-EGFR antibody (e.g. cetuximab), wherein the patient has a tumor that has been characterized in that cells from the tumor express high levels of EGFR homodimer, AREG and TGF-α, for example, cells from the tumor express EGFR homodimer, AREG and TGF-α at levels above the median level for that cancer type. In another embodiment, high levels of EGFR homodimer, AREG and TGF-α, refer to expression in comparison to a reference sample, e.g., EGFR homodimer, AREG and TGF-α are expressed at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells known to be unresponsive to treatment, or a healthy tissue sample), or the mean or median levels expressed by a reference population (e.g., a representative group of tumor samples, across cancer types, such as those found in Table 4). In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E).

In one embodiment, disclosed herein is a method for treating a cancer (e.g., squamous cell carcinoma of the head and neck (SCCHN)), comprising administering to a patient diagnosed with said cancer a therapeutically effective amount of a HER3 inhibitor, for example, an anti-HER3 antibody (e.g., 2C2-YTE), wherein the patient has a tumor that has been characterized in that cells from the tumor express high levels of EGFR homodimer, AREG and TGF-α, for example, cells from the tumor express EGFR homodimer, AREG and TGF-α at levels above the median level for that cancer type. In another embodiment, high levels of EGFR homodimer, AREG and TGF-α, refer to expression in comparison to a reference sample, e.g., EGFR homodimer, AREG and TGF-α are expressed at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells known to be unresponsive to treatment, or a healthy tissue sample), or the mean or median levels expressed by a reference population (e.g., a representative group of tumor samples, across cancer types, such as those found in Table 4). In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E).

In one embodiment, disclosed herein is a method for treating a cancer (e.g., squamous cell carcinoma of the head and neck (SCCHN)), comprising administering to a patient diagnosed with said cancer a therapeutically effective amount of an EGFR inhibitor, for example, an anti-EGFR antibody (e.g. cetuximab), wherein the patient has a tumor that has been characterized in that cells from the tumor express high levels of EGFR homodimer, AREG and TGF-α, for example, cells from the tumor express EGFR homodimer, AREG and TGF-α at levels above the median level for that cancer type. In another embodiment, high levels of EGFR homodimer, AREG and TGF-α, refer to expression in comparison to a reference sample, e.g., EGFR homodimer, AREG and TGF-α are expressed at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells known to be unresponsive to treatment, or a healthy tissue sample), or the mean or median levels expressed by a reference population (e.g., a representative group of tumor samples, across cancer types, such as those found in Table 4).

In one embodiment, disclosed herein is a method for treating a cancer (e.g., squamous cell carcinoma of the head and neck (SCCHN)), comprising administering to a patient diagnosed with said cancer a therapeutically effective amount of a combination of a HER3 inhibitor, for example, an anti-HER3 antibody (e.g., 2C2-YTE), and an EGFR inhibitor, for example, an anti-EGFR antibody (e.g. cetuximab), wherein the patient has a tumor that has been characterized in that cells from the tumor express high levels of EGFR homodimer and AREG, for example, cells from the tumor express EGFR homodimer and AREG at levels above the median level for that cancer type. In another embodiment, high levels of EGFR homodimer and AREG refer to expression in comparison to a reference sample, e.g., EGFR homodimer and AREG are expressed at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells known to be unresponsive to treatment, or a healthy tissue sample), or the mean or median levels expressed by a reference population (e.g., a representative group of tumor samples, across cancer types, such as those found in Table 4). In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E).

In one embodiment, disclosed herein is a method for treating a cancer (e.g., squamous cell carcinoma of the head and neck (SCCHN)), comprising administering to a patient diagnosed with said cancer a therapeutically effective amount of a HER3 inhibitor, for example, an anti-HER3 antibody (e.g., 2C2-YTE), wherein the patient has a tumor that has been characterized in that cells from the tumor express high levels of EGFR homodimer and AREG, for example, cells from the tumor express EGFR homodimer and AREG at levels above the median level for that cancer type. In another embodiment, high levels of EGFR homodimer and AREG refer to expression in comparison to a reference sample, e.g., EGFR homodimer and AREG are expressed at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells known to be unresponsive to treatment, or a healthy tissue sample), or the mean or median levels expressed by a reference population (e.g., a representative group of tumor samples, across cancer types, such as those found in Table 4). In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E).

In one embodiment, disclosed herein is a method for treating a cancer (e.g., squamous cell carcinoma of the head and neck (SCCHN)), comprising administering to a patient diagnosed with said cancer a therapeutically effective amount of an EGFR inhibitor, for example, an anti-EGFR antibody (e.g. cetuximab), wherein the patient has a tumor that has been characterized in that cells from the tumor express high levels of EGFR homodimer and AREG, for example, cells from the tumor express EGFR homodimer and AREG at levels above the median level for that cancer type. In another embodiment, high levels of EGFR homodimer and AREG refer to expression in comparison to a reference sample, e.g., EGFR homodimer and AREG are expressed at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells known to be unresponsive to treatment, or a healthy tissue sample), or the mean or median levels expressed by a reference population (e.g., a representative group of tumor samples, across cancer types, such as those found in Table 4).

In one embodiment, disclosed herein is a method for treating a cancer (e.g., squamous cell carcinoma of the head and neck (SCCHN)), comprising administering to a patient diagnosed with said cancer a therapeutically effective amount of a combination of a HER3 inhibitor, for example, an anti-HER3 antibody (e.g., 2C2-YTE), and an EGFR inhibitor, for example, an anti-EGFR antibody (e.g. cetuximab), wherein the patient has a tumor that has been characterized in that cells from the tumor express high levels of EGFR homodimer and TGF-α, for example, cells from the tumor express EGFR homodimer and TGF-α at levels above the median level for that cancer type. In another embodiment, high levels of EGFR homodimer and TGF-α refer to expression in comparison to a reference sample, e.g., EGFR homodimer and TGF-α are expressed at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells known to be unresponsive to treatment, or a healthy tissue sample), or the mean or median levels expressed by a reference population (e.g., a representative group of tumor samples, across cancer types, such as those found in Table 4). In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E).

In one embodiment, disclosed herein is a method for treating a cancer (e.g., squamous cell carcinoma of the head and neck (SCCHN)), comprising administering to a patient diagnosed with said cancer a therapeutically effective amount of a combination of a HER3 inhibitor, for example, an anti-HER3 antibody (e.g., 2C2-YTE), wherein the patient has a tumor that has been characterized in that cells from the tumor express high levels of EGFR homodimer and TGF-α, for example, cells from the tumor express EGFR homodimer and TGF-α at levels above the median level for that cancer type. In another embodiment, high levels of EGFR homodimer and TGF-α refer to expression in comparison to a reference sample, e.g., EGFR homodimer and TGF-α are expressed at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells known to be unresponsive to treatment, or a healthy tissue sample), or the mean or median levels expressed by a reference population (e.g., a representative group of tumor samples, across cancer types, such as those found in Table 4). In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E).

In one embodiment, disclosed herein is a method for treating a cancer (e.g., squamous cell carcinoma of the head and neck (SCCHN)), comprising administering to a patient diagnosed with said cancer a therapeutically effective amount of an EGFR inhibitor, for example, an anti-EGFR antibody (e.g. cetuximab), wherein the patient has a tumor that has been characterized in that cells from the tumor express high levels of EGFR homodimer and TGF-α, for example, cells from the tumor express EGFR homodimer and TGF-α at levels above the median level for that cancer type. In another embodiment, high levels of EGFR homodimer and TGF-α refer to expression in comparison to a reference sample, e.g., EGFR homodimer and TGF-α are expressed at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells known to be unresponsive to treatment, or a healthy tissue sample), or the mean or median levels expressed by a reference population (e.g., a representative group of tumor samples, across cancer types, such as those found in Table 4).

In one embodiment, disclosed herein is a method for treating a cancer (e.g., squamous cell carcinoma of the head and neck (SCCHN)), comprising administering to a patient diagnosed with said cancer a therapeutically effective amount of a combination of a HER3 inhibitor, for example, an anti-HER3 antibody (e.g., 2C2-YTE), and an EGFR inhibitor, for example, an anti-EGFR antibody (e.g. cetuximab), wherein the patient has a tumor that has been characterized in that cells from the tumor express high levels of EGFR homodimer, for example, cells from the tumor express EGFR homodimer at a level above the median level for that cancer type. In another embodiment, a high level of EGFR homodimer refers to expression in comparison to a reference sample, e.g., EGFR homodimer is expressed at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the level expressed by a reference sample, (e.g. a sample comprised of tumor cells known to be unresponsive to treatment, or a healthy tissue sample), or the mean or median levels expressed by a reference population (e.g., a representative group of tumor samples, across cancer types, such as those found in Table 4). In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E).

In one embodiment, disclosed herein is a method for treating a cancer (e.g., squamous cell carcinoma of the head and neck (SCCHN)), comprising administering to a patient diagnosed with said cancer a therapeutically effective amount of a HER3 inhibitor, for example, an anti-HER3 antibody (e.g., 2C2-YTE), wherein the patient has a tumor that has been characterized in that cells from the tumor express high levels of EGFR homodimer, for example, cells from the tumor express EGFR homodimer at a level above the median level for that cancer type. In another embodiment, a high level of EGFR homodimer refers to expression in comparison to a reference sample, e.g., EGFR homodimer is expressed at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the level expressed by a reference sample, (e.g. a sample comprised of tumor cells known to be unresponsive to treatment, or a healthy tissue sample), or the mean or median levels expressed by a reference population (e.g., a representative group of tumor samples, across cancer types, such as those found in Table 4). In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E).

In one embodiment, disclosed herein is a method for treating a cancer (e.g., squamous cell carcinoma of the head and neck (SCCHN)), comprising administering to a patient diagnosed with said cancer a therapeutically effective amount of an EGFR inhibitor, for example, an anti-EGFR antibody (e.g. cetuximab), wherein the patient has a tumor that has been characterized in that cells from the tumor express high levels of EGFR homodimer, for example, cells from the tumor express EGFR homodimer at a level above the median level for that cancer type. In another embodiment, a high level of EGFR homodimer refers to expression in comparison to a reference sample, e.g., EGFR homodimer is expressed at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the level expressed by a reference sample, (e.g. a sample comprised of tumor cells known to be unresponsive to treatment, or a healthy tissue sample), or the mean or median levels expressed by a reference population (e.g., a representative group of tumor samples, across cancer types, such as those found in Table 4).

In specific aspects, disclosed herein is a method of treating an HPV positive head and neck cancer (e.g., SCCHN) in a human subject in need thereof, comprising administering to the human subject a therapeutically effective amount of an anti-HER3 antibody (e.g., 2C2-YTE) or an antigen-binding fragment thereof. In a specific aspect, disclosed herein is a method of treating an HPV positive head and neck cancer (e.g., SCCHN) in a human subject in need thereof, comprising administering to the human subject a therapeutically effective amount of an anti-HER3 antibody (e.g., 2C2-YTE). In specific aspects, the HPV positive head and neck cancer (e.g. SCCHN) is a cancer of the oral cavity, hypopharynx, oropharynx, rynopharynx, or larynx. In more specific embodiments, the HPV positive head and neck cancer (e.g., SCCHN) is an oropharyngeal cancer. In a specific aspect, a method of treating HPV positive head and neck cancer (e.g., SCCHN) described herein with an anti-HER3 antibody (e.g., 2C2-YTE) or an antigen-binding fragment thereof further comprises administering to the human subject a therapeutically effective amount of an EGFR inhibitor. In one aspect, the EGFR inhibitor is an anti-EGFR antibody such as cetuximab. In a particular aspect, the human subject has been diagnosed with an HPV positive head and neck cancer (e.g., SCCHN). In a certain aspect, the head and neck cancer (e.g., SCCHN) is EGFR expressing head and neck cancer.

In specific aspects, disclosed herein is a method of treating an HPV negative head and neck cancer (e.g., SCCHN) in a human subject in need thereof, comprising administering to the human subject a therapeutically effective amount of an anti-HER3 antibody (e.g., 2C2-YTE) or an antigen-binding fragment thereof. In a specific aspect, disclosed herein is a method of treating an HPV negative head and neck cancer (e.g., SCCHN) in a human subject in need thereof, comprising administering to the human subject a therapeutically effective amount of an anti-HER3 antibody (e.g., 2C2-YTE). In specific aspects, the HPV positive head and neck cancer (e.g. SCCHN) is a cancer of the oral cavity, hypopharynx, oropharynx, rynopharynx, or larynx. In a specific aspect, a method of treating HPV negative head and neck cancer (e.g., SCCHN) described herein with an anti-HER3 antibody (e.g., 2C2-YTE) or an antigen-binding fragment thereof further comprises administering to the human subject a therapeutically effective amount of an EGFR inhibitor. In one aspect, the EGFR inhibitor is an anti-EGFR antibody such as cetuximab. In a particular aspect, the human subject has been diagnosed with an HPV negative head and neck cancer (e.g., SCCHN). In a certain aspect, the head and neck cancer (e.g., SCCHN) is EGFR expressing head and neck cancer.

A cancer is considered an HPV positive cancer when evidence of HPV or a marker indicating the presence of HPV (e.g., HPV DNA or p16) is detected in the cancer or cells of the cancer. The HPV status (i.e., positive or negative) of a head and neck cancer (e.g., SCCHN) from a human subject may be determined, for example, using various methods, such as but not limited to type-specific polymerase chain reaction (PCR), real-time PCR (RT-PCR), immunohistochemical detection of surrogate markers such as p16, HPV deoxyribonucleic acid (DNA) in situ hybridization (ISH), southern blot hybridization (SBH), dot blot hybridization, or a hybrid capture-2 assay (see, e.g., Smith et al., 2014, Oral Oncol. 50(6):600-604). The specimen type for determination of HPV status using such methods may be, for example, a biopsy, a scrape, a brushing, or a mouth rinse. The specimen may be stored, for example, as a fresh frozen (FF) or formalin fixed paraffin-embedded (PE) biopsy.

In one embodiment, disclosed herein are methods for treating a cancer (e.g., thyroid cancer or melanoma), comprising administering to a patient diagnosed with said cancer a therapeutically effective amount of a HER3 inhibitor or a combination of a HER3 inhibitor and a B-Raf inhibitor, wherein the patient has a tumor that has been characterized in that cells from the tumor express a neuregulin. In certain aspects, a patient diagnosed with a cancer treated by a method herein has a tumor that has been characterized as comprising cells that express high levels of a neuregulin. In one embodiment, a high level of a neuregulin refers to expression in comparison to a reference sample, e.g., NRG1 or NRG2 is expressed at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the level expressed by a reference sample, (e.g. a sample comprised of tumor cells known to be unresponsive to treatment, or a healthy tissue sample), or the mean or median levels expressed by a reference population (e.g., a representative group of tumor samples, across cancer types, such as those found in Table 4). In certain embodiments the neuregulin is neuregulin 1 (NRG1) (e.g., NRG1α and/or NRG1β). In certain embodiments the neuregulin is neuregulin 2 (NRG2) (e.g., NRG2α and/or NRG2β). In specific embodiments, the HER3 inhibitor is an anti-HER3 antibody or antigen-binding fragment thereof (e.g. 2C2-YTE). In specific embodiments, the B-Raf inhibitor is vemurafenib. In specific embodiments, the B-Raf inhibitor is dabrafenib. In particular embodiments, the HER3 inhibitor is a 2C2-YTE anti-HER3 antibody and the B-Raf inhibitor is vemurafenib. In particular embodiments, the HER3 inhibitor is a 2C2-YTE anti-HER3 antibody and the B-Raf inhibitor is dabrafenib. In particular embodiments, the anti-HER3 antibody is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E) and the B-Raf inhibitor is vemurafenib or dabrafenib. In certain embodiments, the cancer is characterized by a BRAF mutation (e.g., V600E or V600K). In certain embodiments, the cancer is resistant to treatment with a BRAF inhibitor (e.g., vemurafenib or dabrafenib). In another embodiment, the cancer is resistant to treatment with a MEK inhibitor (e.g., trametinib). In other embodiments, the cancer is resistant to treatment with a BRAF inhibitor (e.g., vemurafenib or dabrafenib) and a MEK inhibitor (e.g., trametinib). In certain embodiments, the cancer is melanoma. In specific embodiments, the cancer is B-Raf mutated melanoma. In certain embodiments, the cancer is thyroid cancer. In specific embodiments, the cancer is B-Raf mutated thyroid cancer. In certain embodiments, the cancer is hairy cell leukemia. In specific embodiments, the cancer is B-Raf mutated hairy cell leukemia. In certain embodiments, the cancer is colorectal cancer. In specific embodiments, the cancer is B-Raf mutated colorectal cancer. In certain embodiments, the cancer is lung cancer. In specific embodiments, the cancer is B-Raf mutated lung cancer. In particular embodiments, the lung cancer is non-small cell lung carcinoma. In certain embodiments, the cancer is a squamous cell carcinoma of the head and neck (SCCHN). In certain embodiments, the cancer is ovarian cancer. In certain embodiments, the cancer is pancreatic cancer. In certain embodiments, the cancer is bladder cancer. In certain embodiments, the cancer is testicular cancer. In certain embodiments, the cancer is endometrial cancer. In certain embodiments, the cancer is hepatocellular carcinoma. In certain embodiments, the cancer is breast cancer. In certain embodiments, the cancer is gastric cancer. In certain embodiments, the cancer is prostate cancer. In certain embodiments, the method of treatment comprises a step of measuring the expression of a neuregulin (e.g. NRG1 and/or NRG2) in the cells of the tumor. In certain embodiments, said measuring is performed in vitro.

In one embodiment, disclosed herein are methods for treating a cancer (e.g., thyroid cancer or melanoma), comprising administering to a patient diagnosed with said cancer a therapeutically effective amount of a HER3 inhibitor, wherein the patient has a tumor that has been characterized in that cells from the tumor express a neuregulin. In certain aspects, a patient diagnosed with a cancer treated by a method herein has a tumor that has been characterized as comprising cells that express high levels of a neuregulin. In one embodiment, a high level of a neuregulin refers to expression in comparison to a reference sample, e.g., NRG1 or NRG2 is expressed at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the level expressed by a reference sample, (e.g. a sample comprised of tumor cells known to be unresponsive to treatment, or a healthy tissue sample), or the mean or median levels expressed by a reference population (e.g., a representative group of tumor samples, across cancer types, such as those found in Table 4). In certain embodiments the neuregulin is neuregulin 1 (NRG1) (e.g., NRG1α and/or NRG1β). In certain embodiments the neuregulin is neuregulin 2 (NRG2) (e.g., NRG2α and/or NRG2β). In specific embodiments, the HER3 inhibitor is an anti-HER3 antibody or antigen-binding fragment thereof (e.g. 2C2-YTE). In particular embodiments, the HER3 inhibitor is a 2C2-YTE anti-HER3 antibody. In particular embodiments, the anti-HER3 antibody is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E). In certain embodiments, the cancer is characterized by a BRAF mutation (e.g., V600E or V600K). In certain embodiments, the cancer is resistant to treatment with a BRAF inhibitor (e.g., vemurafenib or dabrafenib). In another embodiment, the cancer is resistant to treatment with a MEK inhibitor (e.g., trametinib). In other embodiments, the cancer is resistant to treatment with a BRAF inhibitor (e.g., vemurafenib or dabrafenib) and a MEK inhibitor (e.g., trametinib). In certain embodiments, the cancer is melanoma. In specific embodiments, the cancer is B-Raf mutated melanoma. In certain embodiments, the cancer is thyroid cancer. In specific embodiments, the cancer is B-Raf mutated thyroid cancer. In certain embodiments, the cancer is hairy cell leukemia. In specific embodiments, the cancer is B-Raf mutated hairy cell leukemia. In certain embodiments, the cancer is colorectal cancer. In specific embodiments, the cancer is B-Raf mutated colorectal cancer. In certain embodiments, the cancer is lung cancer. In specific embodiments, the cancer is B-Raf mutated lung cancer. In particular embodiments, the lung cancer is non-small cell lung carcinoma. In certain embodiments, the cancer is a squamous cell carcinoma of the head and neck (SCCHN). In certain embodiments, the cancer is ovarian cancer. In certain embodiments, the cancer is pancreatic cancer. In certain embodiments, the cancer is bladder cancer. In certain embodiments, the cancer is testicular cancer. In certain embodiments, the cancer is endometrial cancer. In certain embodiments, the cancer is hepatocellular carcinoma. In certain embodiments, the cancer is breast cancer. In certain embodiments, the cancer is gastric cancer. In certain embodiments, the cancer is prostate cancer. In certain embodiments, the method of treatment comprises a step of measuring the expression of a neuregulin (e.g. NRG1 and/or NRG2) in the cells of the tumor. In certain embodiments, said measuring is performed in vitro.

In one embodiment, disclosed herein are methods for treating a cancer (e.g., thyroid cancer or melanoma), comprising administering to a patient diagnosed with said cancer a therapeutically effective amount of a HER3 inhibitor and a MEK inhibitor, wherein the patient has a tumor that has been characterized in that cells from the tumor express a neuregulin. In certain aspects, a patient diagnosed with a cancer treated by a method herein has a tumor that has been characterized as comprising cells that express high levels of a neuregulin. In one embodiment, a high level of a neuregulin refers to expression in comparison to a reference sample, e.g., NRG1 or NRG2 is expressed at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the level expressed by a reference sample, (e.g. a sample comprised of tumor cells known to be unresponsive to treatment, or a healthy tissue sample), or the mean or median levels expressed by a reference population (e.g., a representative group of tumor samples, across cancer types, such as those found in Table 4). In certain embodiments the neuregulin is neuregulin 1 (NRG1) (e.g., NRG1α and/or NRG1β). In certain embodiments the neuregulin is neuregulin 2 (NRG2) (e.g., NRG2α and/or NRG2β). In specific embodiments, the HER3 inhibitor is an anti-HER3 antibody or antigen-binding fragment thereof (e.g. 2C2-YTE). In specific embodiments, the MEK inhibitor is selumetinib. In specific embodiments, the MEK inhibitor is trametinib. In particular embodiments, the HER3 inhibitor is a 2C2-YTE anti-HER3 antibody and the MEK inhibitor is selumetinib. In particular embodiments, the HER3 inhibitor is a 2C2-YTE anti-HER3 antibody and the MEK inhibitor is trametinib. In particular embodiments, the anti-HER3 antibody is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E) and the B-Raf inhibitor is selumetinib or trametinib. In certain embodiments, the cancer is characterized by a BRAF mutation (e.g., V600E or V600K). In certain embodiments, the cancer is resistant to treatment with a BRAF inhibitor (e.g., vemurafenib or dabrafenib). In another embodiment, the cancer is resistant to treatment with a MEK inhibitor (e.g., trametinib). In other embodiments, the cancer is resistant to treatment with a BRAF inhibitor (e.g., vemurafenib or dabrafenib) and a MEK inhibitor (e.g., trametinib). In certain embodiments, the cancer is melanoma. In specific embodiments, the cancer is B-Raf mutated melanoma. In certain embodiments, the cancer is thyroid cancer. In specific embodiments, the cancer is B-Raf mutated thyroid cancer. In certain embodiments, the cancer is hairy cell leukemia. In specific embodiments, the cancer is B-Raf mutated hairy cell leukemia. In certain embodiments, the cancer is colorectal cancer. In specific embodiments, the cancer is B-Raf mutated colorectal cancer. In certain embodiments, the cancer is lung cancer. In specific embodiments, the cancer is B-Raf mutated lung cancer. In particular embodiments, the lung cancer is non-small cell lung carcinoma. In certain embodiments, the cancer is a squamous cell carcinoma of the head and neck (SCCHN). In certain embodiments, the cancer is ovarian cancer. In certain embodiments, the cancer is pancreatic cancer. In certain embodiments, the cancer is bladder cancer. In certain embodiments, the cancer is testicular cancer. In certain embodiments, the cancer is endometrial cancer. In certain embodiments, the cancer is hepatocellular carcinoma. In certain embodiments, the cancer is breast cancer. In certain embodiments, the cancer is gastric cancer. In certain embodiments, the cancer is prostate cancer. In certain embodiments, the method of treatment comprises a step of measuring the expression of a neuregulin (e.g. NRG1 and/or NRG2) in the cells of the tumor. In certain embodiments, said measuring is performed in vitro.

In one embodiment, disclosed herein are methods for treating a cancer (e.g., thyroid cancer or melanoma), comprising administering to a patient diagnosed with said cancer a therapeutically effective amount of a combination of a HER3 inhibitor, a B-Raf inhibitor, and a MEK inhibitor, wherein the patient has a tumor that has been characterized in that cells from the tumor express a neuregulin. In certain aspects, a patient diagnosed with a cancer treated by a method herein has a tumor that has been characterized as comprising cells that express high levels of a neuregulin. In one embodiment, a high level of a neuregulin refers to expression in comparison to a reference sample, e.g., NRG1 or NRG2 is expressed at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the level expressed by a reference sample, (e.g. a sample comprised of tumor cells known to be unresponsive to treatment, or a healthy tissue sample), or the mean or median levels expressed by a reference population (e.g., a representative group of tumor samples, across cancer types, such as those found in Table 4). In certain embodiments the neuregulin is neuregulin 1 (NRG1) (e.g., NRG1α and/or NRG1β). In certain embodiments the neuregulin is neuregulin 2 (NRG2) (e.g., NRG2α and/or NRG2β). In specific embodiments, the HER3 inhibitor is an anti-HER3 antibody or antigen-binding fragment thereof (e.g. 2C2-YTE). In specific embodiments, the B-Raf inhibitor is vemurafenib. In specific embodiments, the B-Raf inhibitor is dabrafenib. In specific embodiments, the MEK inhibitor is selumetinib. In specific embodiments, the MEK inhibitor is trametinib. In particular embodiments, the HER3 inhibitor is a 2C2-YTE anti-HER3 antibody, the B-Raf inhibitor is vemurafenib, and the MEK inhibitor is selumetinib or trametinib. In particular embodiments, the HER3 inhibitor is a 2C2-YTE anti-HER3 antibody, the B-Raf inhibitor is dabrafenib, and the MEK inhibitor is selumetinib or trametinib. In particular embodiments, the anti-HER3 antibody is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E), the B-Raf inhibitor is vemurafenib or dabrafenib, and the MEK inhibitor is selumetinib or trametinib. In certain embodiments, the cancer is characterized by a BRAF mutation (e.g., V600E or V600K). In certain embodiments, the cancer is resistant to treatment with a BRAF inhibitor (e.g., vemurafenib or dabrafenib). In another embodiment, the cancer is resistant to treatment with a MEK inhibitor (e.g., trametinib). In other embodiments, the cancer is resistant to treatment with a BRAF inhibitor (e.g., vemurafenib or dabrafenib) and a MEK inhibitor (e.g., trametinib). In certain embodiments, the cancer is melanoma. In specific embodiments, the cancer is B-Raf mutated melanoma. In certain embodiments, the cancer is thyroid cancer. In specific embodiments, the cancer is B-Raf mutated thyroid cancer. In certain embodiments, the cancer is hairy cell leukemia. In specific embodiments, the cancer is B-Raf mutated hairy cell leukemia. In certain embodiments, the cancer is colorectal cancer. In specific embodiments, the cancer is B-Raf mutated colorectal cancer. In certain embodiments, the cancer is lung cancer. In specific embodiments, the cancer is B-Raf mutated lung cancer. In particular embodiments, the lung cancer is non-small cell lung carcinoma. In certain embodiments, the cancer is a squamous cell carcinoma of the head and neck (SCCHN). In certain embodiments, the cancer is ovarian cancer. In certain embodiments, the cancer is pancreatic cancer. In certain embodiments, the cancer is bladder cancer. In certain embodiments, the cancer is testicular cancer. In certain embodiments, the cancer is endometrial cancer. In certain embodiments, the cancer is hepatocellular carcinoma. In certain embodiments, the cancer is breast cancer. In certain embodiments, the cancer is gastric cancer. In certain embodiments, the cancer is prostate cancer. In certain embodiments, the method of treatment comprises a step of measuring the expression of a neuregulin (e.g. NRG1 and/or NRG2) in the cells of the tumor. In certain embodiments, said measuring is performed in vitro.

### III. Methods for Determining if a Patient is Likely to be Responsive to Treatment

In one embodiment is a method for determining whether a patient diagnosed with cancer (e.g., head and neck cancer, for example, SCCHN) is indicated as likely to be responsive to treatment with a combination of a HER3 inhibitor, for example an anti-HER3 antibody (e.g. 2C2-YTE) and an EGFR inhibitor, for example an anti-EGFR antibody (e.g. cetuximab), comprising measuring the expression of AREG, TGF-α, and EGFR homodimer in a sample from the patient, wherein the sample comprises tumor cells and a neuregulin (e.g. NRG1 and/or NRG2), and wherein high levels of AREG, TGF-α, and EGFR homodimer in the sample indicates that the patient is likely to be responsive to treatment with a combination of a HER3 inhibitor and an EGFR inhibitor. In a specific embodiment, the method comprises a first step of obtaining the sample from a tumor from the patient. In a further specific embodiment, the method comprises an additional step of administering to the patient a therapeutically effective amount of a combination of a HER3 inhibitor and an EGFR inhibitor. In another specific embodiment, the sample comprises high levels of a neuregulin (e.g. NRG1 and/or NRG2). In certain embodiments, the step of measuring the expression of EGFR homodimer comprises measuring the level of protein-protein interaction between EGFR monomers in the sample. In specific embodiments, expression of EGFR homodimer is measured using a protein proximity assay. In certain embodiments, the step of measuring the expression of AREG and TGF-α in the sample comprises measuring the level of AREG and TGF-α protein or mRNA. In certain embodiments, expression of EGFR homodimer, AREG and TGF-α at levels above the median level for that cancer type indicates a patient is likely to be responsive to treatment with a combination of a HER3 inhibitor and an EGFR inhibitor. In another embodiment, high levels of EGFR homodimer, AREG and TGF-α in comparison to a reference sample, e.g., EGFR homodimer, AREG and TGF-α, for example, at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4), indicates a patient is likely to be responsive to treatment with a combination of a HER3 inhibitor and an EGFR inhibitor. In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E). In certain embodiments, one or more measuring steps are performed in vitro.

In one embodiment is a method for determining whether a patient diagnosed with cancer (e.g., head and neck cancer, for example, SCCHN) is indicated as likely to be responsive to treatment with a HER3 inhibitor, for example an anti-HER3 antibody (e.g. 2C2-YTE), comprising measuring the expression of AREG, TGF-α, and EGFR homodimer in a sample from the patient, wherein the sample comprises tumor cells and a neuregulin (e.g. NRG1 and/or NRG2), and wherein high levels of AREG, TGF-α, and EGFR homodimer in the sample indicates that the patient is likely to be responsive to treatment with a HER3 inhibitor. In a specific embodiment, the method comprises a first step of obtaining the sample from a tumor from the patient. In a further specific embodiment, the method comprises an additional step of administering to the patient a therapeutically effective amount of a HER3 inhibitor. In another specific embodiment, the sample comprises high levels of a neuregulin (e.g. NRG1 and/or NRG2). In certain embodiments, the step of measuring the expression of EGFR homodimer comprises measuring the level of protein-protein interaction between EGFR monomers in the sample. In specific embodiments, expression of EGFR homodimer is measured using a protein proximity assay. In certain embodiments, the step of measuring the expression of AREG and TGF-α in the sample comprises measuring the level of AREG and TGF-α protein or mRNA. In certain embodiments, expression of EGFR homodimer, AREG and TGF-α at levels above the median level for that cancer type indicates a patient is likely to be responsive to treatment with a HER3 inhibitor. In another embodiment, high levels of EGFR homodimer, AREG and TGF-α in comparison to a reference sample, e.g., EGFR homodimer, AREG and TGF-α, for example, at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4), indicates a patient is likely to be responsive to treatment with a HER3 inhibitor. In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E). In certain embodiments, one or more measuring steps are performed in vitro.

In one embodiment is a method for determining whether a patient diagnosed with cancer (e.g., head and neck cancer, for example, SCCHN) is indicated as likely to be responsive to treatment with a an EGFR inhibitor, for example an anti-EGFR antibody (e.g. cetuximab), comprising measuring the expression of AREG, TGF-α, and EGFR homodimer in a sample from the patient, wherein the sample comprises tumor cells and a neuregulin (e.g. NRG1 and/or NRG2), and wherein high levels of AREG, TGF-α, and EGFR homodimer in the sample indicates that the patient is likely to be responsive to treatment with an EGFR inhibitor. In a specific embodiment, the method comprises a first step of obtaining the sample from a tumor from the patient. In a further specific embodiment, the method comprises an additional step of administering to the patient a therapeutically effective amount of an EGFR inhibitor. In another specific embodiment, the sample comprises high levels of a neuregulin (e.g. NRG1 and/or NRG2). In certain embodiments, the step of measuring the expression of EGFR homodimer comprises measuring the level of protein-protein interaction between EGFR monomers in the sample. In specific embodiments, expression of EGFR homodimer is measured using a protein proximity assay. In certain embodiments, the step of measuring the expression of AREG and TGF-α in the sample comprises measuring the level of AREG and TGF-α protein or mRNA. In certain embodiments, expression of EGFR homodimer, AREG and TGF-α at levels above the median level for that cancer type indicates a patient is likely to be responsive to treatment with an EGFR inhibitor. In another embodiment, high levels of EGFR homodimer, AREG and TGF-α in comparison to a reference sample, e.g., EGFR homodimer, AREG and TGF-α, for example, at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4), indicates a patient is likely to be responsive to treatment with an EGFR inhibitor. In certain embodiments, one or more measuring steps are performed in vitro.

In one embodiment is a method for determining whether a patient diagnosed with cancer (e.g., head and neck cancer, for example, SCCHN) is indicated as likely to be responsive to treatment with a combination of a HER3 inhibitor, for example an anti-HER3 antibody (e.g. 2C2-YTE) and an EGFR inhibitor, for example an anti-EGFR antibody (e.g. cetuximab), comprising measuring the expression of AREG and TGF-α in a sample from the patient, wherein the sample comprises tumor cells and a neuregulin (e.g. NRG1 and/or NRG2), and wherein high levels of AREG and TGF-α in the sample indicates that the patient is likely to be responsive to treatment with a combination of a HER3 inhibitor and an EGFR inhibitor. In a specific embodiment, the method comprises a first step of obtaining the sample from a tumor from the patient. In a further specific embodiment, the method comprises an additional step of administering to the patient a therapeutically effective amount of a combination of a HER3 inhibitor and an EGFR inhibitor. In another specific embodiment, the sample comprises high levels of a neuregulin (e.g. NRG1 and/or NRG2). In certain embodiments, the step of measuring the expression of AREG and TGF-α in the sample comprises measuring the level of AREG and TGF-α protein or mRNA. In certain embodiments, expression of AREG and TGF-α at levels above the median level for that cancer type indicates a patient is likely to be responsive to treatment with a combination of a HER3 inhibitor and an EGFR inhibitor. In another embodiment, high levels of AREG and TGF-α in comparison to a reference sample, e.g., AREG and TGF-α, for example, at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4), indicates a patient is likely to be responsive to treatment with a combination of a HER3 inhibitor and an EGFR inhibitor. In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E). In certain embodiments, one or more measuring steps are performed in vitro.

In one embodiment is a method for determining whether a patient diagnosed with cancer (e.g., head and neck cancer, for example, SCCHN) is indicated as likely to be responsive to treatment with a HER3 inhibitor, for example an anti-HER3 antibody (e.g. 2C2-YTE), for example an anti-EGFR antibody (e.g. cetuximab), comprising measuring the expression of AREG and TGF-α in a sample from the patient, wherein the sample comprises tumor cells and a neuregulin (e.g. NRG1 and/or NRG2), and wherein high levels of AREG and TGF-α in the sample indicates that the patient is likely to be responsive to treatment with a HER3 inhibitor. In a specific embodiment, the method comprises a first step of obtaining the sample from a tumor from the patient. In a further specific embodiment, the method comprises an additional step of administering to the patient a therapeutically effective amount of a HER3 inhibitor. In another specific embodiment, the sample comprises high levels of a neuregulin (e.g. NRG1 and/or NRG2). In certain embodiments, the step of measuring the expression of AREG and TGF-α in the sample comprises measuring the level of AREG and TGF-α protein or mRNA. In certain embodiments, expression of AREG and TGF-α at levels above the median level for that cancer type indicates a patient is likely to be responsive to treatment with a HER3 inhibitor. In another embodiment, high levels of AREG and TGF-α in comparison to a reference sample, e.g., AREG and TGF-α, for example, at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4), indicates a patient is likely to be responsive to treatment with a HER3 inhibitor. In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E). In certain embodiments, one or more measuring steps are performed in vitro.

In one embodiment is a method for determining whether a patient diagnosed with cancer (e.g., head and neck cancer, for example, SCCHN) is indicated as likely to be responsive to treatment with an EGFR inhibitor, for example an anti-EGFR antibody (e.g. cetuximab), comprising measuring the expression of AREG and TGF-α in a sample from the patient, wherein the sample comprises tumor cells and a neuregulin (e.g. NRG1 and/or NRG2), and wherein high levels of AREG and TGF-α in the sample indicates that the patient is likely to be responsive to treatment with an EGFR inhibitor. In a specific embodiment, the method comprises a first step of obtaining the sample from a tumor from the patient. In a further specific embodiment, the method comprises an additional step of administering to the patient a therapeutically effective amount of an EGFR inhibitor. In another specific embodiment, the sample comprises high levels of a neuregulin (e.g. NRG1 and/or NRG2). In certain embodiments, the step of measuring the expression of AREG and TGF-α in the sample comprises measuring the level of AREG and TGF-α protein or mRNA. In certain embodiments, expression of AREG and TGF-α at levels above the median level for that cancer type indicates a patient is likely to be responsive to treatment with an EGFR inhibitor. In another embodiment, high levels of AREG and TGF-α in comparison to a reference sample, e.g., AREG and TGF-α, for example, at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4), indicates a patient is likely to be responsive to treatment with an EGFR inhibitor. In certain embodiments, one or more measuring steps are performed in vitro.

In one embodiment is a method for determining whether a patient diagnosed with cancer (e.g., head and neck cancer, for example, SCCHN) is indicated as likely to be responsive to treatment with a combination of a HER3 inhibitor, for example an anti-HER3 antibody (e.g. 2C2-YTE) and an EGFR inhibitor, for example an anti-EGFR antibody (e.g. cetuximab), comprising measuring the expression of AREG and EGFR homodimer in a sample from the patient, wherein the sample comprises tumor cells and a neuregulin (e.g. NRG1 and/or NRG2), and wherein high levels of AREG and EGFR homodimer in the sample indicates that the patient is likely to be responsive to treatment with a combination of a HER3 inhibitor and an EGFR inhibitor. In a specific embodiment, the method comprises a first step of obtaining the sample from a tumor from the patient. In a further specific embodiment, the method comprises an additional step of administering to the patient a therapeutically effective amount of a combination of a HER3 inhibitor and an EGFR inhibitor. In another specific embodiment, the sample comprises high levels of a neuregulin (e.g. NRG1 and/or NRG2). In certain embodiments, the step of measuring the expression of EGFR homodimer comprises measuring the level of protein-protein interaction between EGFR monomers in the sample. In specific embodiments, expression of EGFR homodimer is measured using a protein proximity assay. In certain embodiments, the step of measuring the expression of AREG in the sample comprises measuring the level of AREG protein or mRNA. In certain embodiments, expression of AREG and EGFR homodimer at levels above the median level for that cancer type indicates a patient is likely to be responsive to treatment with a combination of a HER3 inhibitor and an EGFR inhibitor. In another embodiment, high levels of AREG and EGFR homodimer in comparison to a reference sample, e.g., AREG and EGFR homodimer, for example, at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4), indicates a patient is likely to be responsive to treatment with a combination of a HER3 inhibitor and an EGFR inhibitor. In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E). In certain embodiments, one or more measuring steps are performed in vitro.

In one embodiment is a method for determining whether a patient diagnosed with cancer (e.g., head and neck cancer, for example, SCCHN) is indicated as likely to be responsive to treatment with a HER3 inhibitor, for example an anti-HER3 antibody (e.g. 2C2-YTE), for example an anti-EGFR antibody (e.g. cetuximab), comprising measuring the expression of AREG and EGFR homodimer in a sample from the patient, wherein the sample comprises tumor cells and a neuregulin (e.g. NRG1 and/or NRG2), and wherein high levels of AREG and EGFR homodimer in the sample indicates that the patient is likely to be responsive to treatment with a HER3 inhibitor. In a specific embodiment, the method comprises a first step of obtaining the sample from a tumor from the patient. In a further specific embodiment, the method comprises an additional step of administering to the patient a therapeutically effective amount of a HER3 inhibitor. In another specific embodiment, the sample comprises high levels of a neuregulin (e.g. NRG1 and/or NRG2). In certain embodiments, the step of measuring the expression of EGFR homodimer comprises measuring the level of protein-protein interaction between EGFR monomers in the sample. In specific embodiments, expression of EGFR homodimer is measured using a protein proximity assay. In certain embodiments, the step of measuring the expression of AREG in the sample comprises measuring the level of AREG protein or mRNA. In certain embodiments, expression of AREG and EGFR homodimer at levels above the median level for that cancer type indicates a patient is likely to be responsive to treatment with a HER3 inhibitor. In another embodiment, high levels of AREG and EGFR homodimer in comparison to a reference sample, e.g., AREG and EGFR homodimer, for example, at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4), indicates a patient is likely to be responsive to treatment with a HER3 inhibitor. In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E). In certain embodiments, one or more measuring steps are performed in vitro.

In one embodiment is a method for determining whether a patient diagnosed with cancer (e.g., head and neck cancer, for example, SCCHN) is indicated as likely to be responsive to treatment with an EGFR inhibitor, for example an anti-EGFR antibody (e.g. cetuximab), comprising measuring the expression of AREG and EGFR homodimer in a sample from the patient, wherein the sample comprises tumor cells and a neuregulin (e.g. NRG1 and/or NRG2), and wherein high levels of AREG and EGFR homodimer in the sample indicates that the patient is likely to be responsive to treatment with an EGFR inhibitor. In a specific embodiment, the method comprises a first step of obtaining the sample from a tumor from the patient. In a further specific embodiment, the method comprises an additional step of administering to the patient a therapeutically effective amount of an EGFR inhibitor. In another specific embodiment, the sample comprises high levels of a neuregulin (e.g. NRG1 and/or NRG2). In certain embodiments, the step of measuring the expression of EGFR homodimer comprises measuring the level of protein-protein interaction between EGFR monomers in the sample. In specific embodiments, expression of EGFR homodimer is measured using a protein proximity assay. In certain embodiments, the step of measuring the expression of AREG in the sample comprises measuring the level of AREG protein or mRNA. In certain embodiments, expression of AREG and EGFR homodimer at levels above the median level for that cancer type indicates a patient is likely to be responsive to treatment with an EGFR inhibitor. In another embodiment, high levels of AREG and EGFR homodimer in comparison to a reference sample, e.g., AREG and EGFR homodimer, for example, at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4), indicates a patient is likely to be responsive to treatment with an EGFR inhibitor. In certain embodiments, one or more measuring steps are performed in vitro.

In one embodiment is a method for determining whether a patient diagnosed with cancer (e.g., head and neck cancer, for example, SCCHN) is indicated as likely to be responsive to treatment with a combination of a HER3 inhibitor, for example an anti-HER3 antibody (e.g. 2C2-YTE) and an EGFR inhibitor, for example an anti-EGFR antibody (e.g. cetuximab), comprising measuring the expression of TGF-α and EGFR homodimer in a sample from the patient, wherein the sample comprises tumor cells and a neuregulin (e.g. NRG1 and/or NRG2), and wherein high levels of TGF-α and EGFR homodimer in the sample indicates that the patient is likely to be responsive to treatment with a combination of a HER3 inhibitor and an EGFR inhibitor. In a specific embodiment, the method comprises a first step of obtaining the sample from a tumor from the patient. In a further specific embodiment, the method comprises an additional step of administering to the patient a therapeutically effective amount of a combination of a HER3 inhibitor and an EGFR inhibitor. In another specific embodiment, the sample comprises high levels of a neuregulin (e.g. NRG1 and/or NRG2). In certain embodiments, the step of measuring the expression of EGFR homodimer comprises measuring the level of protein-protein interaction between EGFR monomers in the sample. In specific embodiments, expression of EGFR homodimer is measured using a protein proximity assay. In certain embodiments, the step of measuring the expression of TGF-α in the sample comprises measuring the level of TGF-α protein or mRNA. In certain embodiments, expression of TGF-α and EGFR homodimer at levels above the median level for that cancer type indicates a patient is likely to be responsive to treatment with a combination of a HER3 inhibitor and an EGFR inhibitor. In another embodiment, high levels of TGF-α and EGFR homodimer in comparison to a reference sample, e.g., TGF-α and EGFR homodimer, for example, at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4), indicates a patient is likely to be responsive to treatment with a combination of a HER3 inhibitor and an EGFR inhibitor. In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E). In certain embodiments, one or more measuring steps are performed in vitro.

In one embodiment is a method for determining whether a patient diagnosed with cancer (e.g., head and neck cancer, for example, SCCHN) is indicated as likely to be responsive to treatment with a HER3 inhibitor, for example an anti-HER3 antibody (e.g. 2C2-YTE), for example an anti-EGFR antibody (e.g. cetuximab), comprising measuring the expression of TGF-α and EGFR homodimer in a sample from the patient, wherein the sample comprises tumor cells and a neuregulin (e.g. NRG1 and/or NRG2), and wherein high levels of TGF-α and EGFR homodimer in the sample indicates that the patient is likely to be responsive to treatment with a HER3 inhibitor. In a specific embodiment, the method comprises a first step of obtaining the sample from a tumor from the patient. In a further specific embodiment, the method comprises an additional step of administering to the patient a therapeutically effective amount of a HER3 inhibitor. In another specific embodiment, the sample comprises high levels of a neuregulin (e.g. NRG1 and/or NRG2). In certain embodiments, the step of measuring the expression of EGFR homodimer comprises measuring the level of protein-protein interaction between EGFR monomers in the sample. In specific embodiments, expression of EGFR homodimer is measured using a protein proximity assay. In certain embodiments, the step of measuring the expression of TGF-α in the sample comprises measuring the level of TGF-α protein or mRNA. In certain embodiments, expression of TGF-α and EGFR homodimer at levels above the median level for that cancer type indicates a patient is likely to be responsive to treatment with a HER3 inhibitor. In another embodiment, high levels of TGF-α and EGFR homodimer in comparison to a reference sample, e.g., TGF-α and EGFR homodimer, for example, at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4), indicates a patient is likely to be responsive to treatment with a HER3 inhibitor. In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E). In certain embodiments, one or more measuring steps are performed in vitro.

In one embodiment is a method for determining whether a patient diagnosed with cancer (e.g., head and neck cancer, for example, SCCHN) is indicated as likely to be responsive to treatment with an EGFR inhibitor, for example an anti-EGFR antibody (e.g. cetuximab), comprising measuring the expression of TGF-α and EGFR homodimer in a sample from the patient, wherein the sample comprises tumor cells and a neuregulin (e.g. NRG1 and/or NRG2), and wherein high levels of TGF-α and EGFR homodimer in the sample indicates that the patient is likely to be responsive to treatment with an EGFR inhibitor. In a specific embodiment, the method comprises a first step of obtaining the sample from a tumor from the patient. In a further specific embodiment, the method comprises an additional step of administering to the patient a therapeutically effective amount of an EGFR inhibitor. In another specific embodiment, the sample comprises high levels of a neuregulin (e.g. NRG1 and/or NRG2). In certain embodiments, the step of measuring the expression of EGFR homodimer comprises measuring the level of protein-protein interaction between EGFR monomers in the sample. In specific embodiments, expression of EGFR homodimer is measured using a protein proximity assay. In certain embodiments, the step of measuring the expression of AREG in the sample comprises measuring the level of TGF-α protein or mRNA. In certain embodiments, expression of TGF-α and EGFR homodimer at levels above the median level for that cancer type indicates a patient is likely to be responsive to treatment with an EGFR inhibitor. In another embodiment, high levels of TGF-α and EGFR homodimer in comparison to a reference sample, e.g., TGF-α and EGFR homodimer, for example, at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4), indicates a patient is likely to be responsive to treatment with an EGFR inhibitor. In certain embodiments, one or more measuring steps are performed in vitro.

In one embodiment is a method for determining whether a patient diagnosed with cancer (e.g., head and neck cancer, for example, SCCHN) is indicated as likely to be responsive to treatment with a combination of a HER3 inhibitor, for example an anti-HER3 antibody (e.g. 2C2-YTE) and an EGFR inhibitor, for example an anti-EGFR antibody (e.g. cetuximab), comprising measuring the expression of EGFR homodimer in a sample from the patient, wherein the sample comprises tumor cells and a neuregulin (e.g. NRG1 and/or NRG2), and wherein high levels of EGFR homodimer in the sample indicates that the patient is likely to be responsive to treatment with a combination of a HER3 inhibitor and an EGFR inhibitor. In a specific embodiment, the method comprises a first step of obtaining the sample from a tumor from the patient. In a further specific embodiment, the method comprises an additional step of administering to the patient a therapeutically effective amount of a combination of a HER3 inhibitor and an EGFR inhibitor. In another specific embodiment, the sample comprises high levels of a neuregulin (e.g. NRG1 and/or NRG2). In certain embodiments, the step of measuring the expression of EGFR homodimer comprises measuring the level of protein-protein interaction between EGFR monomers in the sample. In specific embodiments, expression of EGFR homodimer is measured using a protein proximity assay. In certain embodiments, expression of EGFR homodimer at levels above the median level for that cancer type indicates a patient is likely to be responsive to treatment with a combination of a HER3 inhibitor and an EGFR inhibitor. In another embodiment, high levels of EGFR homodimer in comparison to a reference sample, e.g., EGFR homodimer, for example, at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4), indicates a patient is likely to be responsive to treatment with a combination of a HER3 inhibitor and an EGFR inhibitor. In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E). In certain embodiments, one or more measuring steps are performed in vitro.

In one embodiment is a method for determining whether a patient diagnosed with cancer (e.g., head and neck cancer, for example, SCCHN) is indicated as likely to be responsive to treatment with a HER3 inhibitor, for example an anti-HER3 antibody (e.g. 2C2-YTE), for example an anti-EGFR antibody (e.g. cetuximab), comprising measuring the expression of EGFR homodimer in a sample from the patient, wherein the sample comprises tumor cells and a neuregulin (e.g. NRG1 and/or NRG2), and wherein high levels of EGFR homodimer in the sample indicates that the patient is likely to be responsive to treatment with a HER3 inhibitor. In a specific embodiment, the method comprises a first step of obtaining the sample from a tumor from the patient. In a further specific embodiment, the method comprises an additional step of administering to the patient a therapeutically effective amount of a HER3 inhibitor. In another specific embodiment, the sample comprises high levels of a neuregulin (e.g. NRG1 and/or NRG2). In certain embodiments, the step of measuring the expression of EGFR homodimer comprises measuring the level of protein-protein interaction between EGFR monomers in the sample. In specific embodiments, expression of EGFR homodimer is measured using a protein proximity assay. In certain embodiments, expression of EGFR homodimer at levels above the median level for that cancer type indicates a patient is likely to be responsive to treatment with a HER3 inhibitor. In another embodiment, high levels of EGFR homodimer in comparison to a reference sample, e.g., EGFR homodimer, for example, at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4), indicates a patient is likely to be responsive to treatment with a HER3 inhibitor. In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E). In certain embodiments, one or more measuring steps are performed in vitro.

In one embodiment is a method for determining whether a patient diagnosed with cancer (e.g., head and neck cancer, for example, SCCHN) is indicated as likely to be responsive to treatment with an EGFR inhibitor, for example an anti-EGFR antibody (e.g. cetuximab), comprising measuring the expression of EGFR homodimer in a sample from the patient, wherein the sample comprises tumor cells and a neuregulin (e.g. NRG1 and/or NRG2), and wherein high levels of EGFR homodimer in the sample indicates that the patient is likely to be responsive to treatment with an EGFR inhibitor. In a specific embodiment, the method comprises a first step of obtaining the sample from a tumor from the patient. In a further specific embodiment, the method comprises an additional step of administering to the patient a therapeutically effective amount of an EGFR inhibitor. In another specific embodiment, the sample comprises high levels of a neuregulin (e.g. NRG1 and/or NRG2). In certain embodiments, the step of measuring the expression of EGFR homodimer comprises measuring the level of protein-protein interaction between EGFR monomers in the sample. In specific embodiments, expression of EGFR homodimer is measured using a protein proximity assay. In certain embodiments, expression of EGFR homodimer at levels above the median level for that cancer type indicates a patient is likely to be responsive to treatment with an EGFR inhibitor. In another embodiment, high levels of EGFR homodimer in comparison to a reference sample, e.g., EGFR homodimer, for example, at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4), indicates a patient is likely to be responsive to treatment with an EGFR inhibitor. In certain embodiments, one or more measuring steps are performed in vitro.

In one embodiment is a method for determining whether a patient diagnosed with cancer (e.g., head and neck cancer, for example, SCCHN) is indicated as likely to be responsive to treatment with a combination of a HER3 inhibitor, for example an anti-HER3 antibody (e.g. 2C2-YTE) and an EGFR inhibitor, for example an anti-EGFR antibody (e.g. cetuximab), wherein the sample comprises tumor cells, comprising measuring the expression of a neuregulin (e.g. NRG1 and/or NRG2), AREG, TGF-α, and EGFR homodimer in a sample from the patient, wherein expression of a neuregulin (e.g. NRG1 and/or NRG2), and high levels of AREG, TGF-α, and EGFR homodimer in the sample indicates that the patient is likely to be responsive to treatment with a combination of a HER3 inhibitor and an EGFR inhibitor. In a specific embodiment, the method comprises a first step of obtaining the sample from a tumor from the patient. In a further specific embodiment, the method comprises an additional step of administering to the patient a therapeutically effective amount of a combination of a HER3 inhibitor and an EGFR inhibitor. In another specific embodiment, expression of a neuregulin (e.g. NRG1 and/or NRG2) is at high levels. In certain embodiments, the step of measuring the expression of EGFR homodimer comprises measuring the level of protein-protein interaction between EGFR monomers in the sample. In specific embodiments, expression of EGFR homodimer is measured using a protein proximity assay. In certain embodiments, the step of measuring the expression of AREG and TGF-α in the sample comprises measuring the level of AREG and TGF-α protein or mRNA. In certain embodiments, expression of EGFR homodimer, AREG and TGF-α at levels above the median level for that cancer type indicates a patient is likely to be responsive to treatment with a combination of a HER3 inhibitor and an EGFR inhibitor. In another embodiment, high levels of EGFR homodimer, AREG and TGF-α in comparison to a reference sample, e.g., EGFR homodimer, AREG and TGF-α, for example, at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4), indicates a patient is likely to be responsive to treatment with a combination of a HER3 inhibitor and an EGFR inhibitor. In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E). In certain embodiments, one or more measuring steps are performed in vitro.

In one embodiment is a method for determining whether a patient diagnosed with cancer (e.g., head and neck cancer, for example, SCCHN) is indicated as likely to be responsive to treatment with a HER3 inhibitor, for example an anti-HER3 antibody (e.g. 2C2-YTE), wherein the sample comprises tumor cells, comprising measuring the expression of a neuregulin (e.g. NRG1 and/or NRG2), AREG, TGF-α, and EGFR homodimer in a sample from the patient, wherein expression of a neuregulin (e.g. NRG1 and/or NRG2), and high levels of AREG, TGF-α, and EGFR homodimer in the sample indicates that the patient is likely to be responsive to treatment with a HER3 inhibitor. In a specific embodiment, the method comprises a first step of obtaining the sample from a tumor from the patient. In a further specific embodiment, the method comprises an additional step of administering to the patient a therapeutically effective amount of a HER3 inhibitor. In another specific embodiment, expression of a neuregulin (e.g. NRG1 and/or NRG2) is at high levels. In certain embodiments, the step of measuring the expression of EGFR homodimer comprises measuring the level of protein-protein interaction between EGFR monomers in the sample. In specific embodiments, expression of EGFR homodimer is measured using a protein proximity assay. In certain embodiments, the step of measuring the expression of AREG and TGF-α in the sample comprises measuring the level of AREG and TGF-α protein or mRNA. In certain embodiments, expression of EGFR homodimer, AREG and TGF-α at levels above the median level for that cancer type indicates a patient is likely to be responsive to treatment with a HER3 inhibitor. In another embodiment, high levels of EGFR homodimer, AREG and TGF-α in comparison to a reference sample, e.g., EGFR homodimer, AREG and TGF-α, for example, at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4), indicates a patient is likely to be responsive to treatment with a HER3 inhibitor. In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E). In certain embodiments, one or more measuring steps are performed in vitro.

In one embodiment is a method for determining whether a patient diagnosed with cancer (e.g., head and neck cancer, for example, SCCHN) is indicated as likely to be responsive to treatment with a an EGFR inhibitor, for example an anti-EGFR antibody (e.g. cetuximab), wherein the sample comprises tumor cells, comprising measuring the expression of a neuregulin (e.g. NRG1 and/or NRG2), AREG, TGF-α, and EGFR homodimer in a sample from the patient, wherein expression of a neuregulin (e.g. NRG1 and/or NRG2), and high levels of AREG, TGF-α, and EGFR homodimer in the sample indicates that the patient is likely to be responsive to treatment with an EGFR inhibitor. In a specific embodiment, the method comprises a first step of obtaining the sample from a tumor from the patient. In a further specific embodiment, the method comprises an additional step of administering to the patient a therapeutically effective amount of an EGFR inhibitor. In another specific embodiment, expression of a neuregulin (e.g. NRG1 and/or NRG2) is at high levels. In certain embodiments, the step of measuring the expression of EGFR homodimer comprises measuring the level of protein-protein interaction between EGFR monomers in the sample. In specific embodiments, expression of EGFR homodimer is measured using a protein proximity assay. In certain embodiments, the step of measuring the expression of AREG and TGF-α in the sample comprises measuring the level of AREG and TGF-α protein or mRNA. In certain embodiments, expression of EGFR homodimer, AREG and TGF-α at levels above the median level for that cancer type indicates a patient is likely to be responsive to treatment with an EGFR inhibitor. In another embodiment, high levels of EGFR homodimer, AREG and TGF-α in comparison to a reference sample, e.g., EGFR homodimer, AREG and TGF-α, for example, at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4), indicates a patient is likely to be responsive to treatment with an EGFR inhibitor. In certain embodiments, one or more measuring steps are performed in vitro.

In one embodiment is a method for determining whether a patient diagnosed with cancer (e.g., head and neck cancer, for example, SCCHN) is indicated as likely to be responsive to treatment with a combination of a HER3 inhibitor, for example an anti-HER3 antibody (e.g. 2C2-YTE) and an EGFR inhibitor, for example an anti-EGFR antibody (e.g. cetuximab), wherein the sample comprises tumor cells, comprising measuring the expression of a neuregulin (e.g. NRG1 and/or NRG2), AREG, and TGF-α in a sample from the patient, wherein expression of a neuregulin (e.g. NRG1 and/or NRG2), and high levels of AREG and TGF-α in the sample indicates that the patient is likely to be responsive to treatment with a combination of a HER3 inhibitor and an EGFR inhibitor. In a specific embodiment, the method comprises a first step of obtaining the sample from a tumor from the patient. In a further specific embodiment, the method comprises an additional step of administering to the patient a therapeutically effective amount of a combination of a HER3 inhibitor and an EGFR inhibitor. In another specific embodiment, expression of a neuregulin (e.g. NRG1 and/or NRG2) is at high levels. In certain embodiments, the step of measuring the expression of AREG and TGF-α in the sample comprises measuring the level of AREG and TGF-α protein or mRNA. In certain embodiments, expression of AREG and TGF-α at levels above the median level for that cancer type indicates a patient is likely to be responsive to treatment with a combination of a HER3 inhibitor and an EGFR inhibitor. In another embodiment, high levels of AREG and TGF-α in comparison to a reference sample, e.g., AREG and TGF-α, for example, at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4), indicates a patient is likely to be responsive to treatment with a combination of a HER3 inhibitor and an EGFR inhibitor. In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E). In certain embodiments, one or more measuring steps are performed in vitro.

In one embodiment is a method for determining whether a patient diagnosed with cancer (e.g., head and neck cancer, for example, SCCHN) is indicated as likely to be responsive to treatment with a HER3 inhibitor, for example an anti-HER3 antibody (e.g. 2C2-YTE), wherein the sample comprises tumor cells, comprising measuring the expression of a neuregulin (e.g. NRG1 and/or NRG2), AREG, and TGF-α in a sample from the patient, wherein expression of a neuregulin (e.g. NRG1 and/or NRG2), and high levels of AREG and TGF-α in the sample indicates that the patient is likely to be responsive to treatment with a HER3 inhibitor. In a specific embodiment, the method comprises a first step of obtaining the sample from a tumor from the patient. In a further specific embodiment, the method comprises an additional step of administering to the patient a therapeutically effective amount of a HER3 inhibitor. In another specific embodiment, expression of a neuregulin (e.g. NRG1 and/or NRG2) is at high levels. In certain embodiments, the step of measuring the expression of AREG and TGF-α in the sample comprises measuring the level of AREG and TGF-α protein or mRNA. In certain embodiments, expression of AREG and TGF-α at levels above the median level for that cancer type indicates a patient is likely to be responsive to treatment with a HER3 inhibitor. In another embodiment, high levels of AREG and TGF-α in comparison to a reference sample, e.g., AREG and TGF-α, for example, at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4), indicates a patient is likely to be responsive to treatment with a HER3 inhibitor. In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E). In certain embodiments, one or more measuring steps are performed in vitro.

In one embodiment is a method for determining whether a patient diagnosed with cancer (e.g., head and neck cancer, for example, SCCHN) is indicated as likely to be responsive to treatment with an EGFR inhibitor, for example an anti-EGFR antibody (e.g. cetuximab), wherein the sample comprises tumor cells, comprising measuring the expression of a neuregulin (e.g. NRG1 and/or NRG2), AREG, and TGF-α in a sample from the patient, wherein expression of a neuregulin (e.g. NRG1 and/or NRG2), and high levels of AREG and TGF-α in the sample indicates that the patient is likely to be responsive to treatment with an EGFR inhibitor. In a specific embodiment, the method comprises a first step of obtaining the sample from a tumor from the patient. In a further specific embodiment, the method comprises an additional step of administering to the patient a therapeutically effective amount of an EGFR inhibitor. In another specific embodiment, expression of a neuregulin (e.g. NRG1 and/or NRG2) is at high levels. In certain embodiments, the step of measuring the expression of AREG and TGF-α in the sample comprises measuring the level of AREG and TGF-α protein or mRNA. In certain embodiments, expression of AREG and TGF-α at levels above the median level for that cancer type indicates a patient is likely to be responsive to treatment with an EGFR inhibitor. In another embodiment, high levels of AREG and TGF-α in comparison to a reference sample, e.g., AREG and TGF-α, for example, at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4), indicates a patient is likely to be responsive to treatment with an EGFR inhibitor. In certain embodiments, one or more measuring steps are performed in vitro.

In one embodiment is a method for determining whether a patient diagnosed with cancer (e.g., head and neck cancer, for example, SCCHN) is indicated as likely to be responsive to treatment with a combination of a HER3 inhibitor, for example an anti-HER3 antibody (e.g. 2C2-YTE) and an EGFR inhibitor, for example an anti-EGFR antibody (e.g. cetuximab), wherein the sample comprises tumor cells, comprising measuring the expression of a neuregulin (e.g. NRG1 and/or NRG2), AREG, and EGFR homodimer in a sample from the patient, wherein expression of a neuregulin (e.g. NRG1 and/or NRG2), and high levels of AREG and EGFR homodimer in the sample indicates that the patient is likely to be responsive to treatment with a combination of a HER3 inhibitor and an EGFR inhibitor. In a specific embodiment, the method comprises a first step of obtaining the sample from a tumor from the patient. In a further specific embodiment, the method comprises an additional step of administering to the patient a therapeutically effective amount of a combination of a HER3 inhibitor and an EGFR inhibitor. In another specific embodiment, expression of a neuregulin (e.g. NRG1 and/or NRG2) is at high levels. In certain embodiments, the step of measuring the expression of EGFR homodimer comprises measuring the level of protein-protein interaction between EGFR monomers in the sample. In specific embodiments, expression of EGFR homodimer is measured using a protein proximity assay. In certain embodiments, the step of measuring the expression of AREG in the sample comprises measuring the level of AREG protein or mRNA. In certain embodiments, expression of AREG and EGFR homodimer at levels above the median level for that cancer type indicates a patient is likely to be responsive to treatment with a combination of a HER3 inhibitor and an EGFR inhibitor. In another embodiment, high levels of AREG and EGFR homodimer in comparison to a reference sample, e.g., AREG and EGFR homodimer, for example, at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4), indicates a patient is likely to be responsive to treatment with a combination of a HER3 inhibitor and an EGFR inhibitor. In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E). In certain embodiments, one or more measuring steps are performed in vitro.

In one embodiment is a method for determining whether a patient diagnosed with cancer (e.g., head and neck cancer, for example, SCCHN) is indicated as likely to be responsive to treatment with a HER3 inhibitor, for example an anti-HER3 antibody (e.g. 2C2-YTE), wherein the sample comprises tumor cells, comprising measuring the expression of a neuregulin (e.g. NRG1 and/or NRG2), AREG, and EGFR homodimer in a sample from the patient, wherein expression of a neuregulin (e.g. NRG1 and/or NRG2), and high levels of AREG and EGFR homodimer in the sample indicates that the patient is likely to be responsive to treatment with a HER3 inhibitor. In a specific embodiment, the method comprises a first step of obtaining the sample from a tumor from the patient. In a further specific embodiment, the method comprises an additional step of administering to the patient a therapeutically effective amount of a HER3 inhibitor. In certain embodiments, the step of measuring the expression of EGFR homodimer comprises measuring the level of protein-protein interaction between EGFR monomers in the sample. In specific embodiments, expression of EGFR homodimer is measured using a protein proximity assay. In certain embodiments, the step of measuring the expression of AREG in the sample comprises measuring the level of AREG protein or mRNA. In certain embodiments, expression of AREG and EGFR homodimer at levels above the median level for that cancer type indicates a patient is likely to be responsive to treatment with a HER3 inhibitor. In another embodiment, high levels of AREG and EGFR homodimer in comparison to a reference sample, e.g., AREG and EGFR homodimer, for example, at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4), indicates a patient is likely to be responsive to treatment with a HER3 inhibitor. In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E). In certain embodiments, one or more measuring steps are performed in vitro.

In one embodiment is a method for determining whether a patient diagnosed with cancer (e.g., head and neck cancer, for example, SCCHN) is indicated as likely to be responsive to treatment with an EGFR inhibitor, for example an anti-EGFR antibody (e.g. cetuximab), wherein the sample comprises tumor cells, comprising measuring the expression of a neuregulin (e.g. NRG1 and/or NRG2), AREG, and EGFR homodimer in a sample from the patient, wherein expression of a neuregulin (e.g. NRG1 and/or NRG2), and high levels of AREG and EGFR homodimer in the sample indicates that the patient is likely to be responsive to treatment with an EGFR inhibitor. In a specific embodiment, the method comprises a first step of obtaining the sample from a tumor from the patient. In a further specific embodiment, the method comprises an additional step of administering to the patient a therapeutically effective amount of an EGFR inhibitor. In another specific embodiment, expression of a neuregulin (e.g. NRG1 and/or NRG2) is at high levels. In certain embodiments, the step of measuring the expression of EGFR homodimer comprises measuring the level of protein-protein interaction between EGFR monomers in the sample. In specific embodiments, expression of EGFR homodimer is measured using a protein proximity assay. In certain embodiments, the step of measuring the expression of AREG in the sample comprises measuring the level of AREG protein or mRNA. In certain embodiments, expression of AREG and EGFR homodimer at levels above the median level for that cancer type indicates a patient is likely to be responsive to treatment with an EGFR inhibitor. In another embodiment, high levels of AREG and EGFR homodimer in comparison to a reference sample, e.g., AREG and EGFR homodimer, for example, at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4), indicates a patient is likely to be responsive to treatment with an EGFR inhibitor. In certain embodiments, one or more measuring steps are performed in vitro.

In one embodiment is a method for determining whether a patient diagnosed with cancer (e.g., head and neck cancer, for example, SCCHN) is indicated as likely to be responsive to treatment with a combination of a HER3 inhibitor, for example an anti-HER3 antibody (e.g. 2C2-YTE) and an EGFR inhibitor, for example an anti-EGFR antibody (e.g. cetuximab), wherein the sample comprises tumor cells, comprising measuring the expression of a neuregulin (e.g. NRG1 and/or NRG2), TGF-α, and EGFR homodimer in a sample from the patient, wherein expression of a neuregulin (e.g. NRG1 and/or NRG2), and high levels of TGF-α and EGFR homodimer in the sample indicates that the patient is likely to be responsive to treatment with a combination of a HER3 inhibitor and an EGFR inhibitor. In a specific embodiment, the method comprises a first step of obtaining the sample from a tumor from the patient. In a further specific embodiment, the method comprises an additional step of administering to the patient a therapeutically effective amount of a combination of a HER3 inhibitor and an EGFR inhibitor. In another specific embodiment, expression of a neuregulin (e.g. NRG1 and/or NRG2) is at high levels. In certain embodiments, the step of measuring the expression of EGFR homodimer comprises measuring the level of protein-protein interaction between EGFR monomers in the sample. In specific embodiments, expression of EGFR homodimer is measured using a protein proximity assay. In certain embodiments, the step of measuring the expression of TGF-α in the sample comprises measuring the level of TGF-α protein or mRNA. In certain embodiments, expression of TGF-α and EGFR homodimer at levels above the median level for that cancer type indicates a patient is likely to be responsive to treatment with a combination of a HER3 inhibitor and an EGFR inhibitor. In another embodiment, high levels of TGF-α and EGFR homodimer in comparison to a reference sample, e.g., TGF-α and EGFR homodimer, for example, at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4), indicates a patient is likely to be responsive to treatment with a combination of a HER3 inhibitor and an EGFR inhibitor. In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E). In certain embodiments, one or more measuring steps are performed in vitro.

In one embodiment is a method for determining whether a patient diagnosed with cancer (e.g., head and neck cancer, for example, SCCHN) is indicated as likely to be responsive to treatment with a HER3 inhibitor, for example an anti-HER3 antibody (e.g. 2C2-YTE), for example an anti-EGFR antibody (e.g. cetuximab), wherein the sample comprises tumor cells, comprising measuring the expression of a neuregulin (e.g. NRG1 and/or NRG2), TGF-α, and EGFR homodimer in a sample from the patient, wherein expression of a neuregulin (e.g. NRG1 and/or NRG2), and high levels of TGF-α and EGFR homodimer in the sample indicates that the patient is likely to be responsive to treatment with a HER3 inhibitor. In a specific embodiment, the method comprises a first step of obtaining the sample from a tumor from the patient. In a further specific embodiment, the method comprises an additional step of administering to the patient a therapeutically effective amount of a HER3 inhibitor. In another specific embodiment, expression of a neuregulin (e.g. NRG1 and/or NRG2) is at high levels. In certain embodiments, the step of measuring the expression of EGFR homodimer comprises measuring the level of protein-protein interaction between EGFR monomers in the sample. In specific embodiments, expression of EGFR homodimer is measured using a protein proximity assay. In certain embodiments, the step of measuring the expression of TGF-α in the sample comprises measuring the level of TGF-α protein or mRNA. In certain embodiments, expression of TGF-α and EGFR homodimer at levels above the median level for that cancer type indicates a patient is likely to be responsive to treatment with a HER3 inhibitor. In another embodiment, high levels of TGF-α and EGFR homodimer in comparison to a reference sample, e.g., TGF-α and EGFR homodimer, for example, at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4), indicates a patient is likely to be responsive to treatment with a HER3 inhibitor. In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E). In certain embodiments, one or more measuring steps are performed in vitro.

In one embodiment is a method for determining whether a patient diagnosed with cancer (e.g., head and neck cancer, for example, SCCHN) is indicated as likely to be responsive to treatment with an EGFR inhibitor, for example an anti-EGFR antibody (e.g. cetuximab), wherein the sample comprises tumor cells, comprising measuring the expression of a neuregulin (e.g. NRG1 and/or NRG2), TGF-α, and EGFR homodimer in a sample from the patient, wherein expression of a neuregulin (e.g. NRG1 and/or NRG2), and high levels of TGF-α and EGFR homodimer in the sample indicates that the patient is likely to be responsive to treatment with an EGFR inhibitor. In a specific embodiment, the method comprises a first step of obtaining the sample from a tumor from the patient. In a further specific embodiment, the method comprises an additional step of administering to the patient a therapeutically effective amount of an EGFR inhibitor. In another specific embodiment, expression of a neuregulin (e.g. NRG1 and/or NRG2) is at high levels. In certain embodiments, the step of measuring the expression of EGFR homodimer comprises measuring the level of protein-protein interaction between EGFR monomers in the sample. In specific embodiments, expression of EGFR homodimer is measured using a protein proximity assay. In certain embodiments, the step of measuring the expression of AREG in the sample comprises measuring the level of TGF-α protein or mRNA. In certain embodiments, expression of TGF-α and EGFR homodimer at levels above the median level for that cancer type indicates a patient is likely to be responsive to treatment with an EGFR inhibitor. In another embodiment, high levels of TGF-α and EGFR homodimer in comparison to a reference sample, e.g., TGF-α and EGFR homodimer, for example, at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4), indicates a patient is likely to be responsive to treatment with an EGFR inhibitor. In certain embodiments, one or more measuring steps are performed in vitro.

In one embodiment is a method for determining whether a patient diagnosed with cancer (e.g., head and neck cancer, for example, SCCHN) is indicated as likely to be responsive to treatment with a combination of a HER3 inhibitor, for example an anti-HER3 antibody (e.g. 2C2-YTE) and an EGFR inhibitor, for example an anti-EGFR antibody (e.g. cetuximab), wherein the sample comprises tumor cells, comprising measuring the expression of a neuregulin (e.g. NRG1 and/or NRG2) and EGFR homodimer in a sample from the patient, wherein expression of a neuregulin (e.g. NRG1 and/or NRG2), and high levels of EGFR homodimer in the sample indicates that the patient is likely to be responsive to treatment with a combination of a HER3 inhibitor and an EGFR inhibitor. In a specific embodiment, the method comprises a first step of obtaining the sample from a tumor from the patient. In a further specific embodiment, the method comprises an additional step of administering to the patient a therapeutically effective amount of a combination of a HER3 inhibitor and an EGFR inhibitor. In another specific embodiment, expression of a neuregulin (e.g. NRG1 and/or NRG2) is at high levels. In certain embodiments, the step of measuring the expression of EGFR homodimer comprises measuring the level of protein-protein interaction between EGFR monomers in the sample. In specific embodiments, expression of EGFR homodimer is measured using a protein proximity assay. In certain embodiments, expression of EGFR homodimer at levels above the median level for that cancer type indicates a patient is likely to be responsive to treatment with a combination of a HER3 inhibitor and an EGFR inhibitor. In another embodiment, high levels of EGFR homodimer in comparison to a reference sample, e.g., EGFR homodimer, for example, at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4), indicates a patient is likely to be responsive to treatment with a combination of a HER3 inhibitor and an EGFR inhibitor. In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E). In certain embodiments, one or more measuring steps are performed in vitro.

In one embodiment is a method for determining whether a patient diagnosed with cancer (e.g., head and neck cancer, for example, SCCHN) is indicated as likely to be responsive to treatment with a HER3 inhibitor, for example an anti-HER3 antibody (e.g. 2C2-YTE), wherein the sample comprises tumor cells, comprising measuring the expression of a neuregulin (e.g. NRG1 and/or NRG2) and EGFR homodimer in a sample from the patient, wherein expression of a neuregulin (e.g. NRG1 and/or NRG2), and high levels of EGFR homodimer in the sample indicates that the patient is likely to be responsive to treatment with a HER3 inhibitor. In a specific embodiment, the method comprises a first step of obtaining the sample from a tumor from the patient. In a further specific embodiment, the method comprises an additional step of administering to the patient a therapeutically effective amount of a HER3 inhibitor. In another specific embodiment, expression of a neuregulin (e.g. NRG1 and/or NRG2) is at high levels. In certain embodiments, the step of measuring the expression of EGFR homodimer comprises measuring the level of protein-protein interaction between EGFR monomers in the sample. In specific embodiments, expression of EGFR homodimer is measured using a protein proximity assay. In certain embodiments, expression of EGFR homodimer at levels above the median level for that cancer type indicates a patient is likely to be responsive to treatment with a HER3 inhibitor. In another embodiment, high levels of EGFR homodimer in comparison to a reference sample, e.g., EGFR homodimer, for example, at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4), indicates a patient is likely to be responsive to treatment with a HER3 inhibitor. In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E). In certain embodiments, one or more measuring steps are performed in vitro.

In one embodiment is a method for determining whether a patient diagnosed with cancer (e.g., head and neck cancer, for example, SCCHN) is indicated as likely to be responsive to treatment with an EGFR inhibitor, for example an anti-EGFR antibody (e.g. cetuximab), wherein the sample comprises tumor cells, comprising measuring the expression of a neuregulin (e.g. NRG1 and/or NRG2) and EGFR homodimer in a sample from the patient, wherein expression of a neuregulin (e.g. NRG1 and/or NRG2), and high levels of EGFR homodimer in the sample indicates that the patient is likely to be responsive to treatment with an EGFR inhibitor. In a specific embodiment, the method comprises a first step of obtaining the sample from a tumor from the patient. In a further specific embodiment, the method comprises an additional step of administering to the patient a therapeutically effective amount of an EGFR inhibitor. In another specific embodiment, expression of a neuregulin (e.g. NRG1 and/or NRG2) is at high levels. In certain embodiments, the step of measuring the expression of EGFR homodimer comprises measuring the level of protein-protein interaction between EGFR monomers in the sample. In specific embodiments, expression of EGFR homodimer is measured using a protein proximity assay. In certain embodiments, expression of EGFR homodimer at levels above the median level for that cancer type indicates a patient is likely to be responsive to treatment with an EGFR inhibitor. In another embodiment, high levels of EGFR homodimer in comparison to a reference sample, e.g., EGFR homodimer, for example, at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells that do not express a neuregulin, or a healthy tissue sample, or the UNC10 cell line, see Examples), or the mean or median levels expressed by a reference population (e.g., a representative group of tumor samples, across cancer types, such as those found in Table 4), indicates a patient is likely to be responsive to treatment with an EGFR inhibitor. In certain embodiments, one or more measuring steps are performed in vitro.

In one embodiment is a method for determining whether a patient diagnosed with cancer (e.g., head and neck cancer, for example, SCCHN) is indicated as likely to be responsive to treatment with a combination of a HER3 inhibitor, for example an anti-HER3 antibody (e.g. 2C2-YTE) and an EGFR inhibitor, for example an anti-EGFR antibody (e.g. cetuximab), wherein the sample comprises tumor cells, comprising measuring the expression of AREG, TGF-α, and EGFR homodimer in a sample from the patient, wherein expression of high levels of AREG, TGF-α, and EGFR homodimer in the sample indicates that the patient is likely to be responsive to treatment with a combination of a HER3 inhibitor and an EGFR inhibitor. In a specific embodiment, the method comprises a first step of obtaining the sample from a tumor from the patient. In a further specific embodiment, the method comprises an additional step of administering to the patient a therapeutically effective amount of a combination of a HER3 inhibitor and an EGFR inhibitor. In certain embodiments, the step of measuring the expression of EGFR homodimer comprises measuring the level of protein-protein interaction between EGFR monomers in the sample. In specific embodiments, expression of EGFR homodimer is measured using a protein proximity assay. In certain embodiments, the step of measuring the expression of AREG and TGF-α in the sample comprises measuring the level of AREG and TGF-α protein or mRNA. In certain embodiments, expression of EGFR homodimer, AREG and TGF-α at levels above the median level for that cancer type indicates a patient is likely to be responsive to treatment with a combination of a HER3 inhibitor and an EGFR inhibitor. In another embodiment, high levels of EGFR homodimer, AREG and TGF-α in comparison to a reference sample, e.g., EGFR homodimer, AREG and TGF-α, for example, at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells known to be unresponsive to treatment, or a healthy tissue sample), or the mean or median levels expressed by a reference population (e.g., a representative group of tumor samples, across cancer types, such as those found in Table 4), indicates a patient is likely to be responsive to treatment with a combination of a HER3 inhibitor and an EGFR inhibitor. In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E). In certain embodiments, one or more measuring steps are performed in vitro.

In one embodiment is a method for determining whether a patient diagnosed with cancer (e.g., head and neck cancer, for example, SCCHN) is indicated as likely to be responsive to treatment with a HER3 inhibitor, for example an anti-HER3 antibody (e.g. 2C2-YTE), wherein the sample comprises tumor cells, comprising measuring the expression of AREG, TGF-α, and EGFR homodimer in a sample from the patient, wherein high levels of AREG, TGF-α, and EGFR homodimer in the sample indicates that the patient is likely to be responsive to treatment with a HER3 inhibitor. In a specific embodiment, the method comprises a first step of obtaining the sample from a tumor from the patient. In a further specific embodiment, the method comprises an additional step of administering to the patient a therapeutically effective amount of a HER3 inhibitor. In certain embodiments, the step of measuring the expression of EGFR homodimer comprises measuring the level of protein-protein interaction between EGFR monomers in the sample. In specific embodiments, expression of EGFR homodimer is measured using a protein proximity assay. In certain embodiments, the step of measuring the expression of AREG and TGF-α in the sample comprises measuring the level of AREG and TGF-α protein or mRNA. In certain embodiments, expression of EGFR homodimer, AREG and TGF-α at levels above the median level for that cancer type indicates a patient is likely to be responsive to treatment with a HER3 inhibitor. In another embodiment, high levels of EGFR homodimer, AREG and TGF-α in comparison to a reference sample, e.g., EGFR homodimer, AREG and TGF-α, for example, at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells known to be unresponsive to treatment, or a healthy tissue sample), or the mean or median levels expressed by a reference population (e.g., a representative group of tumor samples, across cancer types, such as those found in Table 4), indicates a patient is likely to be responsive to treatment with a HER3 inhibitor. In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E). In certain embodiments, one or more measuring steps are performed in vitro.

In one embodiment is a method for determining whether a patient diagnosed with cancer (e.g., head and neck cancer, for example, SCCHN) is indicated as likely to be responsive to treatment with a an EGFR inhibitor, for example an anti-EGFR antibody (e.g. cetuximab), wherein the sample comprises tumor cells, comprising measuring the expression of AREG, TGF-α, and EGFR homodimer in a sample from the patient, wherein expression of high levels of AREG, TGF-α, and EGFR homodimer in the sample indicates that the patient is likely to be responsive to treatment with an EGFR inhibitor. In a specific embodiment, the method comprises a first step of obtaining the sample from a tumor from the patient. In a further specific embodiment, the method comprises an additional step of administering to the patient a therapeutically effective amount of an EGFR inhibitor. In certain embodiments, the step of measuring the expression of EGFR homodimer comprises measuring the level of protein-protein interaction between EGFR monomers in the sample. In specific embodiments, expression of EGFR homodimer is measured using a protein proximity assay. In certain embodiments, the step of measuring the expression of AREG and TGF-α in the sample comprises measuring the level of AREG and TGF-α protein or mRNA. In certain embodiments, expression of EGFR homodimer, AREG and TGF-α at levels above the median level for that cancer type indicates a patient is likely to be responsive to treatment with an EGFR inhibitor. In another embodiment, high levels of EGFR homodimer, AREG and TGF-α in comparison to a reference sample, e.g., EGFR homodimer, AREG and TGF-α, for example, at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells known to be unresponsive to treatment, or a healthy tissue sample), or the mean or median levels expressed by a reference population (e.g., a representative group of tumor samples, across cancer types, such as those found in Table 4), indicates a patient is likely to be responsive to treatment with an EGFR inhibitor. In certain embodiments, one or more measuring steps are performed in vitro.

In one embodiment is a method for determining whether a patient diagnosed with cancer (e.g., head and neck cancer, for example, SCCHN) is indicated as likely to be responsive to treatment with a combination of a HER3 inhibitor, for example an anti-HER3 antibody (e.g. 2C2-YTE) and an EGFR inhibitor, for example an anti-EGFR antibody (e.g. cetuximab), wherein the sample comprises tumor cells, comprising measuring the expression of AREG and EGFR homodimer in a sample from the patient, wherein expression of high levels of AREG and EGFR homodimer in the sample indicates that the patient is likely to be responsive to treatment with a combination of a HER3 inhibitor and an EGFR inhibitor. In a specific embodiment, the method comprises a first step of obtaining the sample from a tumor from the patient. In a further specific embodiment, the method comprises an additional step of administering to the patient a therapeutically effective amount of a combination of a HER3 inhibitor and an EGFR inhibitor. In certain embodiments, the step of measuring the expression of EGFR homodimer comprises measuring the level of protein-protein interaction between EGFR monomers in the sample. In specific embodiments, expression of EGFR homodimer is measured using a protein proximity assay. In certain embodiments, the step of measuring the expression of AREG in the sample comprises measuring the level of AREG protein or mRNA. In certain embodiments, expression of AREG and EGFR homodimer at levels above the median level for that cancer type indicates a patient is likely to be responsive to treatment with a combination of a HER3 inhibitor and an EGFR inhibitor. In another embodiment, high levels of AREG and EGFR homodimer in comparison to a reference sample, e.g., AREG and EGFR homodimer, for example, at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells known to be unresponsive to treatment, or a healthy tissue sample), or the mean or median levels expressed by a reference population (e.g., a representative group of tumor samples, across cancer types, such as those found in Table 4), indicates a patient is likely to be responsive to treatment with a combination of a HER3 inhibitor and an EGFR inhibitor. In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E). In certain embodiments, one or more measuring steps are performed in vitro.

In one embodiment is a method for determining whether a patient diagnosed with cancer (e.g., head and neck cancer, for example, SCCHN) is indicated as likely to be responsive to treatment with a HER3 inhibitor, for example an anti-HER3 antibody (e.g. 2C2-YTE), wherein the sample comprises tumor cells, comprising measuring the expression of AREG and EGFR homodimer in a sample from the patient, wherein expression of high levels of AREG and EGFR homodimer in the sample indicates that the patient is likely to be responsive to treatment with a HER3 inhibitor. In a specific embodiment, the method comprises a first step of obtaining the sample from a tumor from the patient. In a further specific embodiment, the method comprises an additional step of administering to the patient a therapeutically effective amount of a HER3 inhibitor. In certain embodiments, the step of measuring the expression of EGFR homodimer comprises measuring the level of protein-protein interaction between EGFR monomers in the sample. In specific embodiments, expression of EGFR homodimer is measured using a protein proximity assay. In certain embodiments, the step of measuring the expression of AREG in the sample comprises measuring the level of AREG protein or mRNA. In certain embodiments, expression of AREG and EGFR homodimer at levels above the median level for that cancer type indicates a patient is likely to be responsive to treatment with a HER3 inhibitor. In another embodiment, high levels of AREG and EGFR homodimer in comparison to a reference sample, e.g., AREG and EGFR homodimer, for example, at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells known to be unresponsive to treatment, or a healthy tissue sample), or the mean or median levels expressed by a reference population (e.g., a representative group of tumor samples, across cancer types, such as those found in Table 4), indicates a patient is likely to be responsive to treatment with a HER3 inhibitor. In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E). In certain embodiments, one or more measuring steps are performed in vitro.

In one embodiment is a method for determining whether a patient diagnosed with cancer (e.g., head and neck cancer, for example, SCCHN) is indicated as likely to be responsive to treatment with an EGFR inhibitor, for example an anti-EGFR antibody (e.g. cetuximab), wherein the sample comprises tumor cells, comprising measuring the expression of AREG and EGFR homodimer in a sample from the patient, wherein expression of high levels of AREG and EGFR homodimer in the sample indicates that the patient is likely to be responsive to treatment with an EGFR inhibitor. In a specific embodiment, the method comprises a first step of obtaining the sample from a tumor from the patient. In a further specific embodiment, the method comprises an additional step of administering to the patient a therapeutically effective amount of an EGFR inhibitor. In certain embodiments, the step of measuring the expression of EGFR homodimer comprises measuring the level of protein-protein interaction between EGFR monomers in the sample. In specific embodiments, expression of EGFR homodimer is measured using a protein proximity assay. In certain embodiments, the step of measuring the expression of AREG in the sample comprises measuring the level of AREG protein or mRNA. In certain embodiments, expression of AREG and EGFR homodimer at levels above the median level for that cancer type indicates a patient is likely to be responsive to treatment with an EGFR inhibitor. In another embodiment, high levels of AREG and EGFR homodimer in comparison to a reference sample, e.g., AREG and EGFR homodimer, for example, at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells known to be unresponsive to treatment, or a healthy tissue sample), or the mean or median levels expressed by a reference population (e.g., a representative group of tumor samples, across cancer types, such as those found in Table 4), indicates a patient is likely to be responsive to treatment with an EGFR inhibitor. In certain embodiments, one or more measuring steps are performed in vitro.

In one embodiment is a method for determining whether a patient diagnosed with cancer (e.g., head and neck cancer, for example, SCCHN) is indicated as likely to be responsive to treatment with a combination of a HER3 inhibitor, for example an anti-HER3 antibody (e.g. 2C2-YTE) and an EGFR inhibitor, for example an anti-EGFR antibody (e.g. cetuximab), wherein the sample comprises tumor cells, comprising measuring the expression of TGF-α and EGFR homodimer in a sample from the patient, wherein expression of high levels of TGF-α and EGFR homodimer in the sample indicates that the patient is likely to be responsive to treatment with a combination of a HER3 inhibitor and an EGFR inhibitor. In a specific embodiment, the method comprises a first step of obtaining the sample from a tumor from the patient. In a further specific embodiment, the method comprises an additional step of administering to the patient a therapeutically effective amount of a combination of a HER3 inhibitor and an EGFR inhibitor. In certain embodiments, the step of measuring the expression of EGFR homodimer comprises measuring the level of protein-protein interaction between EGFR monomers in the sample. In specific embodiments, expression of EGFR homodimer is measured using a protein proximity assay. In certain embodiments, the step of measuring the expression of TGF-α in the sample comprises measuring the level of TGF-α protein or mRNA. In certain embodiments, expression of TGF-α and EGFR homodimer at levels above the median level for that cancer type indicates a patient is likely to be responsive to treatment with a combination of a HER3 inhibitor and an EGFR inhibitor. In another embodiment, high levels of TGF-α and EGFR homodimer in comparison to a reference sample, e.g., TGF-α and EGFR homodimer, for example, at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells known to be unresponsive to treatment, or a healthy tissue sample), or the mean or median levels expressed by a reference population (e.g., a representative group of tumor samples, across cancer types, such as those found in Table 4), indicates a patient is likely to be responsive to treatment with a combination of a HER3 inhibitor and an EGFR inhibitor. In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E). In certain embodiments, one or more measuring steps are performed in vitro.

In one embodiment is a method for determining whether a patient diagnosed with cancer (e.g., head and neck cancer, for example, SCCHN) is indicated as likely to be responsive to treatment with a HER3 inhibitor, for example an anti-HER3 antibody (e.g. 2C2-YTE), wherein the sample comprises tumor cells, comprising measuring the expression of TGF-α and EGFR homodimer in a sample from the patient, wherein expression of high levels of TGF-α and EGFR homodimer in the sample indicates that the patient is likely to be responsive to treatment with a HER3 inhibitor. In a specific embodiment, the method comprises a first step of obtaining the sample from a tumor from the patient. In a further specific embodiment, the method comprises an additional step of administering to the patient a therapeutically effective amount of a HER3 inhibitor. In certain embodiments, the step of measuring the expression of EGFR homodimer comprises measuring the level of protein-protein interaction between EGFR monomers in the sample. In specific embodiments, expression of EGFR homodimer is measured using a protein proximity assay. In certain embodiments, the step of measuring the expression of TGF-α in the sample comprises measuring the level of TGF-α protein or mRNA. In certain embodiments, expression of TGF-α and EGFR homodimer at levels above the median level for that cancer type indicates a patient is likely to be responsive to treatment with a HER3 inhibitor. In another embodiment, high levels of TGF-α and EGFR homodimer in comparison to a reference sample, e.g., TGF-α and EGFR homodimer, for example, at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells known to be unresponsive to treatment, or a healthy tissue sample), or the mean or median levels expressed by a reference population (e.g., a representative group of tumor samples, across cancer types, such as those found in Table 4), indicates a patient is likely to be responsive to treatment with a HER3 inhibitor. In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E). In certain embodiments, one or more measuring steps are performed in vitro.

In one embodiment is a method for determining whether a patient diagnosed with cancer (e.g., head and neck cancer, for example, SCCHN) is indicated as likely to be responsive to treatment with an EGFR inhibitor, for example an anti-EGFR antibody (e.g. cetuximab), wherein the sample comprises tumor cells, comprising measuring the expression of TGF-α and EGFR homodimer in a sample from the patient, wherein expression high levels of TGF-α and EGFR homodimer in the sample indicates that the patient is likely to be responsive to treatment with an EGFR inhibitor. In a specific embodiment, the method comprises a first step of obtaining the sample from a tumor from the patient. In a further specific embodiment, the method comprises an additional step of administering to the patient a therapeutically effective amount of an EGFR inhibitor. In certain embodiments, the step of measuring the expression of EGFR homodimer comprises measuring the level of protein-protein interaction between EGFR monomers in the sample. In specific embodiments, expression of EGFR homodimer is measured using a protein proximity assay. In certain embodiments, the step of measuring the expression of AREG in the sample comprises measuring the level of TGF-α protein or mRNA. In certain embodiments, expression of TGF-α and EGFR homodimer at levels above the median level for that cancer type indicates a patient is likely to be responsive to treatment with an EGFR inhibitor. In another embodiment, high levels of TGF-α and EGFR homodimer in comparison to a reference sample, e.g., TGF-α and EGFR homodimer, for example, at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells known to be unresponsive to treatment, or a healthy tissue sample), or the mean or median levels expressed by a reference population (e.g., a representative group of tumor samples, across cancer types, such as those found in Table 4), indicates a patient is likely to be responsive to treatment with an EGFR inhibitor. In certain embodiments, one or more measuring steps are performed in vitro.

In one embodiment is a method for determining whether a patient diagnosed with cancer (e.g., head and neck cancer, for example, SCCHN) is indicated as likely to be responsive to treatment with a combination of a HER3 inhibitor, for example an anti-HER3 antibody (e.g. 2C2-YTE) and an EGFR inhibitor, for example an anti-EGFR antibody (e.g. cetuximab), wherein the sample comprises tumor cells, comprising measuring the expression of EGFR homodimer in a sample from the patient, wherein expression of high levels of EGFR homodimer in the sample indicates that the patient is likely to be responsive to treatment with a combination of a HER3 inhibitor and an EGFR inhibitor. In a specific embodiment, the method comprises a first step of obtaining the sample from a tumor from the patient. In a further specific embodiment, the method comprises an additional step of administering to the patient a therapeutically effective amount of a combination of a HER3 inhibitor and an EGFR inhibitor. In certain embodiments, the step of measuring the expression of EGFR homodimer comprises measuring the level of protein-protein interaction between EGFR monomers in the sample. In specific embodiments, expression of EGFR homodimer is measured using a protein proximity assay. In certain embodiments, expression of EGFR homodimer at levels above the median level for that cancer type indicates a patient is likely to be responsive to treatment with a combination of a HER3 inhibitor and an EGFR inhibitor. In another embodiment, high levels of EGFR homodimer in comparison to a reference sample, e.g., EGFR homodimer, for example, at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells known to be unresponsive to treatment, or a healthy tissue sample), or the mean or median levels expressed by a reference population (e.g., a representative group of tumor samples, across cancer types, such as those found in Table 4), indicates a patient is likely to be responsive to treatment with a combination of a HER3 inhibitor and an EGFR inhibitor. In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E). In certain embodiments, one or more measuring steps are performed in vitro.

In one embodiment is a method for determining whether a patient diagnosed with cancer (e.g., head and neck cancer, for example, SCCHN) is indicated as likely to be responsive to treatment with a HER3 inhibitor, for example an anti-HER3 antibody (e.g. 2C2-YTE), wherein the sample comprises tumor cells, comprising measuring the expression of EGFR homodimer in a sample from the patient, wherein expression of high levels of EGFR homodimer in the sample indicates that the patient is likely to be responsive to treatment with a HER3 inhibitor. In a specific embodiment, the method comprises a first step of obtaining the sample from a tumor from the patient. In a further specific embodiment, the method comprises an additional step of administering to the patient a therapeutically effective amount of a HER3 inhibitor. In certain embodiments, the step of measuring the expression of EGFR homodimer comprises measuring the level of protein-protein interaction between EGFR monomers in the sample. In specific embodiments, expression of EGFR homodimer is measured using a protein proximity assay. In certain embodiments, expression of EGFR homodimer at levels above the median level for that cancer type indicates a patient is likely to be responsive to treatment with a HER3 inhibitor. In another embodiment, high levels of EGFR homodimer in comparison to a reference sample, e.g., EGFR homodimer, for example, at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells known to be unresponsive to treatment, or a healthy tissue sample), or the mean or median levels expressed by a reference population (e.g., a representative group of tumor samples, across cancer types, such as those found in Table 4), indicates a patient is likely to be responsive to treatment with a HER3 inhibitor. In particular embodiments, the HER3 inhibitor is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E). In certain embodiments, one or more measuring steps are performed in vitro.

In one embodiment is a method for determining whether a patient diagnosed with cancer (e.g., head and neck cancer, for example, SCCHN) is indicated as likely to be responsive to treatment with an EGFR inhibitor, for example an anti-EGFR antibody (e.g. cetuximab), wherein the sample comprises tumor cells, comprising measuring the expression of EGFR homodimer in a sample from the patient, wherein expression of high levels of EGFR homodimer in the sample indicates that the patient is likely to be responsive to treatment with an EGFR inhibitor. In a specific embodiment, the method comprises a first step of obtaining the sample from a tumor from the patient. In a further specific embodiment, the method comprises an additional step of administering to the patient a therapeutically effective amount of an EGFR inhibitor. In certain embodiments, the step of measuring the expression of EGFR homodimer comprises measuring the level of protein-protein interaction between EGFR monomers in the sample. In specific embodiments, expression of EGFR homodimer is measured using a protein proximity assay. In certain embodiments, expression of EGFR homodimer at levels above the median level for that cancer type indicates a patient is likely to be responsive to treatment with an EGFR inhibitor. In another embodiment, high levels of EGFR homodimer in comparison to a reference sample, e.g., EGFR homodimer, for example, at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells known to be unresponsive to treatment, or a healthy tissue sample), or the mean or median levels expressed by a reference population (e.g., a representative group of tumor samples, across cancer types, such as those found in Table 4), indicates a patient is likely to be responsive to treatment with an EGFR inhibitor. In certain embodiments, one or more measuring steps are performed in vitro.

In another aspect, the disclosure provides methods for determining whether a patient diagnosed with cancer (e.g., thyroid cancer or melanoma) is indicated as likely to be responsive to treatment with a HER3 inhibitor, comprising measuring the expression of a neuregulin (e.g. NRG1 and/or NRG2) in a sample from the patient, wherein the sample comprises tumor cells, wherein a high level of neuregulin in the sample, e.g., NRG1 or NRG2, for example, at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells known to be unresponsive to treatment, or a healthy tissue sample), or the mean or median levels expressed by a reference population (e.g., a representative group of tumor samples, across cancer types, such as those found in Table 4), indicates that the patient is likely to be responsive to treatment with a HER3 inhibitor. In certain embodiments, the neuregulin is NRG1. In certain embodiments, the neuregulin is NRG2 (E.G., NRG2A AND/OR NRG2B). In certain embodiments, the cancer is characterized by a BRAF mutation (e.g., V600E or V600K). In certain embodiments, the cancer is resistant to treatment with a BRAF inhibitor (e.g., vemurafenib or dabrafenib). In another embodiment, the cancer is resistant to treatment with a MEK inhibitor (e.g., trametinib). In other embodiments, the cancer is resistant to treatment with a BRAF inhibitor (e.g., vemurafenib or dabrafenib) and a MEK inhibitor (e.g., trametinib). In certain embodiments, the cancer is melanoma. In specific embodiments, the cancer is B-Raf mutated melanoma. In certain embodiments, the cancer is thyroid cancer. In specific embodiments, the cancer is B-Raf mutated thyroid cancer. In certain embodiments, the cancer is colorectal cancer. In specific embodiments, the cancer is B-Raf mutated colorectal cancer. In certain embodiments, the cancer is lung cancer. In specific embodiments, the cancer is B-Raf mutated lung cancer. In particular embodiments, the lung cancer is non-small cell lung carcinoma. In certain embodiments, the cancer is a squamous cell carcinoma of the head and neck (SCCHN). In certain embodiments, the cancer is ovarian cancer. In certain embodiments, the cancer is pancreatic cancer. In certain embodiments, the cancer is bladder cancer. In certain embodiments, the cancer is testicular cancer. In certain embodiments, the cancer is endometrial cancer. In certain embodiments, the cancer is hepatocellular carcinoma. In certain embodiments, the cancer is breast cancer. In certain embodiments, the cancer is gastric cancer. In certain embodiments, the cancer is prostate cancer. In certain embodiments, one or more measuring steps are performed in vitro.

In certain embodiments, such methods for determining whether a patient diagnosed with cancer (e.g., thyroid cancer or melanoma) is indicated as likely to be responsive to treatment with a HER3 inhibitor comprise a first step of obtaining the sample from a tumor from the patient. In certain embodiments, the method comprises an additional step of administering to the patient a therapeutically effective amount of a HER3 inhibitor. In specific embodiments, the HER3 inhibitor is an anti-HER3 antibody or antigen-binding fragment thereof (e.g. 2C2-YTE). In particular embodiments, the HER3 inhibitor is a 2C2-YTE anti-HER3 antibody. In particular embodiments, the HER3 inhibitor is a 2C2-YTE anti-HER3. In particular embodiments, the anti-HER3 antibody is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E).

In another aspect, the disclosure provides methods for determining whether a patient diagnosed with cancer (e.g., thyroid cancer or melanoma) is indicated as likely to be responsive to treatment with a combination of a HER3 inhibitor and a B-Raf inhibitor, comprising measuring the expression of a neuregulin (e.g. NRG1 and/or NRG2) in a sample from the patient, wherein the sample comprises tumor cells, wherein a high level of neuregulin in the sample, e.g., NRG1 or NRG2, for example, at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells known to be unresponsive to treatment, or a healthy tissue sample), or the mean or median levels expressed by a reference population (e.g., a representative group of tumor samples, across cancer types, such as those found in Table 4), indicates that the patient is likely to be responsive to treatment with a combination of a HER3 inhibitor and a B-Raf inhibitor. In certain embodiments, the neuregulin is NRG1. In certain embodiments, the neuregulin is NRG2 (e.g., NRG2A AND/OR NRG2B). In certain embodiments, the cancer is characterized by a BRAF mutation (e.g., V600E or V600K). In certain embodiments, the cancer is resistant to treatment with a BRAF inhibitor (e.g., vemurafenib or dabrafenib). In another embodiment, the cancer is resistant to treatment with a MEK inhibitor (e.g., trametinib). In other embodiments, the cancer is resistant to treatment with a BRAF inhibitor (e.g., vemurafenib or dabrafenib) and a MEK inhibitor (e.g., trametinib). In certain embodiments, the cancer is melanoma. In specific embodiments, the cancer is B-Raf mutated melanoma. In certain embodiments, the cancer is thyroid cancer. In specific embodiments, the cancer is B-Raf mutated thyroid cancer. In certain embodiments, the cancer is colorectal cancer. In specific embodiments, the cancer is B-Raf mutated colorectal cancer. In certain embodiments, the cancer is lung cancer. In specific embodiments, the cancer is B-Raf mutated lung cancer. In particular embodiments, the lung cancer is non-small cell lung carcinoma. In certain embodiments, the cancer is a squamous cell carcinoma of the head and neck (SCCHN). In certain embodiments, the cancer is ovarian cancer. In certain embodiments, the cancer is pancreatic cancer. In certain embodiments, the cancer is bladder cancer. In certain embodiments, the cancer is testicular cancer. In certain embodiments, the cancer is endometrial cancer. In certain embodiments, the cancer is hepatocellular carcinoma. In certain embodiments, the cancer is breast cancer. In certain embodiments, the cancer is gastric cancer. In certain embodiments, the cancer is prostate cancer. In certain embodiments, one or more measuring steps are performed in vitro.

In certain embodiments, such methods for determining whether a patient diagnosed with cancer (e.g., thyroid cancer or melanoma) is indicated as likely to be responsive to treatment with a combination of a HER3 inhibitor and a B-Raf inhibitor comprise a first step of obtaining the sample from a tumor from the patient. In certain embodiments, the method comprises an additional step of administering to the patient a therapeutically effective amount of a combination of a HER3 inhibitor and a B-Raf inhibitor. In specific embodiments, the HER3 inhibitor is an anti-HER3 antibody or antigen-binding fragment thereof (e.g. 2C2-YTE) and the B-Raf inhibitor is vemurafenib. In specific embodiments, the HER3 inhibitor is an anti-HER3 antibody or antigen-binding fragment thereof (e.g. 2C2-YTE) and the B-Raf inhibitor is dabrafenib. In particular embodiments, the HER3 inhibitor is a 2C2-YTE anti-HER3 antibody and the B-Raf inhibitor is vemurafenib. In particular embodiments, the HER3 inhibitor is a 2C2-YTE anti-HER3 antibody and the B-Raf inhibitor is dabrafenib. In particular embodiments, the anti-HER3 antibody is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E), and the B-Raf inhibitor is vemurafenib or dabrafenib.

In another aspect, the disclosure provides methods for determining whether a patient diagnosed with cancer (e.g., thyroid cancer or melanoma) is indicated as likely to be responsive to treatment with a combination of a HER3 inhibitor and a MEK inhibitor, comprising measuring the expression of a neuregulin (e.g. NRG1 and/or NRG2) in a sample from the patient, wherein the sample comprises tumor cells, wherein a high level of neuregulin in the sample, e.g., NRG1 or NRG2, for example, at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells known to be unresponsive to treatment, or a healthy tissue sample), or the mean or median levels expressed by a reference population (e.g., a representative group of tumor samples, across cancer types, such as those found in Table 4), indicates that the patient is likely to be responsive to treatment with a combination of a HER3 inhibitor and a MEK inhibitor. In certain embodiments, the neuregulin is NRG1. In certain embodiments, the neuregulin is NRG2 (E.G., NRG2A AND/OR NRG2B). In certain embodiments, the cancer is characterized by a BRAF mutation (e.g., V600E or V600K). In certain embodiments, the cancer is resistant to treatment with a BRAF inhibitor (e.g., vemurafenib or dabrafenib). In another embodiment, the cancer is resistant to treatment with a MEK inhibitor (e.g., trametinib). In other embodiments, the cancer is resistant to treatment with a BRAF inhibitor (e.g., vemurafenib or dabrafenib) and a MEK inhibitor (e.g., trametinib). In certain embodiments, the cancer is melanoma. In specific embodiments, the cancer is B-Raf mutated melanoma. In certain embodiments, the cancer is thyroid cancer. In specific embodiments, the cancer is B-Raf mutated thyroid cancer. In certain embodiments, the cancer is colorectal cancer. In specific embodiments, the cancer is B-Raf mutated colorectal cancer. In certain embodiments, the cancer is lung cancer. In specific embodiments, the cancer is B-Raf mutated lung cancer. In particular embodiments, the lung cancer is non-small cell lung carcinoma. In certain embodiments, the cancer is a squamous cell carcinoma of the head and neck (SCCHN). In certain embodiments, the cancer is ovarian cancer. In certain embodiments, the cancer is pancreatic cancer. In certain embodiments, the cancer is bladder cancer. In certain embodiments, the cancer is testicular cancer. In certain embodiments, the cancer is endometrial cancer. In certain embodiments, the cancer is hepatocellular carcinoma. In certain embodiments, the cancer is breast cancer. In certain embodiments, the cancer is gastric cancer. In certain embodiments, the cancer is prostate cancer. In certain embodiments, one or more measuring steps are performed in vitro.

In certain embodiments, such methods for determining whether a patient diagnosed with cancer (e.g., thyroid cancer or melanoma) is indicated as likely to be responsive to treatment with a combination of a HER3 inhibitor and a MEK inhibitor comprise a first step of obtaining the sample from a tumor from the patient. In certain embodiments, the method comprises an additional step of administering to the patient a therapeutically effective amount of a combination of a HER3 inhibitor and a MEK inhibitor. In specific embodiments, the HER3 inhibitor is an anti-HER3 antibody or antigen-binding fragment thereof (e.g. 2C2-YTE) and the MEK inhibitor is selumetinib. In specific embodiments, the HER3 inhibitor is an anti-HER3 antibody or antigen-binding fragment thereof (e.g. 2C2-YTE) and the MEK inhibitor is trametinib. In particular embodiments, the HER3 inhibitor is a 2C2-YTE anti-HER3 antibody and the MEK inhibitor is selumetinib. In particular embodiments, the HER3 inhibitor is a 2C2-YTE anti-HER3 antibody and the MEK inhibitor is trametinib. In particular embodiments, the anti-HER3 antibody is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E), and the MEK inhibitor is selumetinib or trametinib.

In another aspect, the disclosure provides methods for determining whether a patient diagnosed with cancer (e.g., thyroid cancer or melanoma) is indicated as likely to be responsive to treatment with a combination of a HER3 inhibitor, B-Raf inhibitor, and a MEK inhibitor, comprising measuring the expression of a neuregulin (e.g. NRG1 and/or NRG2) in a sample from the patient, wherein the sample comprises tumor cells, wherein a high level of neuregulin in the sample, e.g., NRG1 or NRG2, for example, at levels 1.2-fold, 1.5-fold, 1.7-fold, 1.9-fold, 2-fold, 3-fold, 4-fold, 5-fold, 10-fold, 20-fold, 50-fold, 100-fold, 500-fold, 1000-fold, 10,000-fold greater or more than the levels expressed by a reference sample, (e.g. a sample comprised of tumor cells known to be unresponsive to treatment, or a healthy tissue sample), or the mean or median levels expressed by a reference population (e.g., a representative group of tumor samples, across cancer types, such as those found in Table 4), indicates that the patient is likely to be responsive to treatment with a combination of a HER3 inhibitor, a B-Raf inhibitor, and a MEK inhibitor. In certain embodiments, the neuregulin is NRG1. In certain embodiments, the neuregulin is NRG2 (E.G., NRG2A AND/OR NRG2B). In certain embodiments, the cancer is characterized by a BRAF mutation (e.g., V600E or V600K). In certain embodiments, the cancer is resistant to treatment with a BRAF inhibitor (e.g., vemurafenib or dabrafenib). In another embodiment, the cancer is resistant to treatment with a MEK inhibitor (e.g., trametinib). In other embodiments, the cancer is resistant to treatment with a BRAF inhibitor (e.g., vemurafenib or dabrafenib) and a MEK inhibitor (e.g., trametinib). In certain embodiments, the cancer is melanoma. In specific embodiments, the cancer is B-Raf mutated melanoma. In certain embodiments, the cancer is thyroid cancer. In specific embodiments, the cancer is B-Raf mutated thyroid cancer. In certain embodiments, the cancer is colorectal cancer. In specific embodiments, the cancer is B-Raf mutated colorectal cancer. In certain embodiments, the cancer is lung cancer. In specific embodiments, the cancer is B-Raf mutated lung cancer. In particular embodiments, the lung cancer is non-small cell lung carcinoma. In certain embodiments, the cancer is a squamous cell carcinoma of the head and neck (SCCHN). In certain embodiments, the cancer is ovarian cancer. In certain embodiments, the cancer is pancreatic cancer. In certain embodiments, the cancer is bladder cancer. In certain embodiments, the cancer is testicular cancer. In certain embodiments, the cancer is endometrial cancer. In certain embodiments, the cancer is hepatocellular carcinoma. In certain embodiments, the cancer is breast cancer. In certain embodiments, the cancer is gastric cancer. In certain embodiments, the cancer is prostate cancer. In certain embodiments, one or more measuring steps are performed in vitro.

In certain embodiments, such methods for determining whether a patient diagnosed with cancer (e.g., thyroid cancer or melanoma) is indicated as likely to be responsive to treatment with a combination of a HER3 inhibitor, a B-Raf inhibitor, and a MEK inhibitor comprise a first step of obtaining the sample from a tumor from the patient. In certain embodiments, the method comprises an additional step of administering to the patient a therapeutically effective amount of a combination of a HER3 inhibitor, a B-Raf inhibitor, and a MEK inhibitor. In specific embodiments, the HER3 inhibitor is an anti-HER3 antibody or antigen-binding fragment thereof (e.g. 2C2-YTE), the B-Raf inhibitor is vemurafenib, and the MEK inhibitor is selumetinib or trametinib. In specific embodiments, the HER3 inhibitor is an anti-HER3 antibody or antigen-binding fragment thereof (e.g. 2C2-YTE), the B-Raf inhibitor is dabrafenib, and the MEK inhibitor is selumetinib or trametinib. In particular embodiments, the HER3 inhibitor is a 2C2-YTE anti-HER3 antibody, the B-Raf inhibitor is vemurafenib, and the MEK inhibitor is selumetinib or trametinib. In particular embodiments, the HER3 inhibitor is a 2C2-YTE anti-HER3 antibody, the B-Raf inhibitor is dabrafenib, and the MEK inhibitor is selumetinib or trametinib. In particular embodiments, the anti-HER3 antibody is a monoclonal, human anti-HER3 antibody, which comprises an antibody VL of SEQ ID NO:3 and a human lambda light chain constant region, and an antibody VH of SEQ ID NO: 2 and a human IgG1 constant region comprising amino acid substitutions relative to a wild-type human IgG constant domain at positions 252, 254, and 256, wherein the numbering is according to the EU index as set forth in Kabat, and wherein the amino acid at position 252 is substituted with Tyrosine (Y), the amino acid at position 254 is substituted with Threonine (T), and the amino acid at position 256 is substituted with Glutamic acid (E), the B-Raf inhibitor is vemurafenib or dabrafenib, and the MEK inhibitor is selumetinib or trametinib.

### IV. Cancers to be Treated Using the Methods Herein

In certain embodiments, the methods for treating a cancer provided herein are methods for treating a cancer associated with EGFR or HER3 expression or EGFR-expressing and/or HER3-expressing cells, such as head and neck cancer, e.g., squamous cell carcinoma of the head and neck (SCCHN), sarcoma, melanoma, lung cancer such as non-small cell lung cancer, breast cancer, ovarian cancer, cervical cancer, central nervous system cancers such as brain cancer, pancreatic cancer, thyroid cancer, gastric cancer, esophageal cancer, colon cancer, bladder cancer or kidney cancer such as renal carcinoma. By “EGFR-expressing and/or HER3-expressing cell” is meant a cell expressing EGFR and/or HER3 In certain embodiments, the methods for treating a cancer provided herein are methods for treating a cancer associated with EGFR, wherein treatment with an EGFR inhibitor increases the expression of HER3 in the cancer. In specific embodiments, the methods for treating a cancer provided herein are methods for treating squamous cell carcinoma of the head and neck. In even more specific embodiments, the methods for treating a cancer provided herein are methods for treating squamous cell carcinoma of the head and neck that expresses EGFR and/or HER3.

In certain embodiments, the methods for treating a cancer provided herein are methods for treating a cancer associated with B-Raf, MEK, or HER3 expression or B-Raf-expressing and/or MEK expressing and/or HER3-expressing cells, such as thyroid cancer, melanoma, hairy cell leukemia, head and neck cancer, e.g., squamous cell carcinoma of the head and neck (SCCHN), sarcoma, lung cancer such as non-small cell lung cancer, breast cancer, ovarian cancer, cervical cancer, central nervous system cancers such as brain cancer, pancreatic cancer, gastric cancer, esophageal cancer, colon cancer, endometrial cancer, bladder cancer, testicular cancer, prostate cancer, hepatocellular carcinoma, or kidney cancer such as renal carcinoma. By “B-Raf-expressing and/or MEK-expressing and/or HER3-expressing cell” is meant a cell expressing B-Raf and/or MEK and/or HER3. In certain embodiments, the methods for treating a cancer provided herein are methods for treating a cancer associated with B-Raf, wherein treatment with a B-Raf inhibitor increases the expression of HER3 in the cancer. In certain embodiments, the methods for treating a cancer provided herein are methods for treating a cancer associated with MEK, wherein treatment with a MEK inhibitor increases the expression of HER3 in the cancer. In certain embodiments, the methods for treating a cancer provided herein are methods for treating a cancer associated with MEK and B-Raf, wherein treatment with a MEK inhibitor and a B-Raf inhibitor increases the expression of HER3 in the cancer. In specific embodiments, the methods for treating a cancer provided herein are methods for treating thyroid cancer. In specific embodiments, the methods for treating a cancer provided herein are methods for treating melanoma. In even more specific embodiments, the methods for treating a cancer provided herein are methods for treating thyroid cancer or melanoma that expresses HER3 and/or B-Raf. In certain embodiments, the cancer is characterized by a BRAF mutation (e.g., V600E or V600K). In specific embodiments, the BRAF mutation is the BRAF V600E mutation. In specific embodiments, the BRAF mutation is the BRAF V600K mutation. In certain embodiments, the cancer is resistant to treatment with a BRAF inhibitor (e.g., vemurafenib or dabrafenib). In another embodiment, the cancer is resistant to treatment with a MEK inhibitor (e.g., trametinib). In other embodiments, the cancer is resistant to treatment with a BRAF inhibitor (e.g., vemurafenib or dabrafenib) and a MEK inhibitor (e.g., trametinib).

### V. HER3, EGFR, B-Raf, and MEK Inhibitors

a) HER3 Inhibitors

A HER3 inhibitor used in the methods herein may be any HER3 inhibitor known in the art. The HER3 inhibitor may be an anti-HER3 antibody or antigen-binding fragment thereof, for example, 2C2-YTE, or another anti-HER3 antibody or antigen-binding fragment thereof disclosed herein. The HER3 inhibitor may also be a small molecule inhibitor. Examples of anti-HER3 antibodies include, but are not limited to, AMG-888 (U3-1287; Amgen and Daiichi Sankyo), MM-121, (Merrimack and Sanofi-Aventis), GE-huMaB-HER3 (Roche), TK-A3 (Takis and University of Cantanzaro (Italy)), TK-A4 (Takis and University of Cantanzaro (Italy)), AV-203 (Aveo Pharmaceuticals), MP-RM-1 (Mediapharma), LJM716 (Novartis and Sanofi Aventis), REGN1400 (Regeneron), MEHD7945A (a bispecific antibody against HER3 and EGFR; Genentech), Pertuzumab (a bispecific antibody against HER3 and HER2; Genentech), MM-111 (a bispecific antibody against HER3 and HER2; Merrimack), and MM-141 (blocks binding of NRG to HER3 and IGF-1 to IGFR; Merrimack). Examples of HER3 small molecule inhibitors include, but are not limited to MP-470 (Amuvatinib; Astex Pharmaceuticals) and AZD 8931 (AstraZeneca).

b) EGFR Inhibitors

A EGFR inhibitor used in the methods herein may be any EGFR inhibitor known in the art. The EGFR inhibitor may be an anti-EGFR antibody or antigen-binding fragment thereof, for example, cetuximab (ERBITUX®; Bristol-Myers Squibb/Lilly), panitumumab (Amgen), or zalutumumab (Genmab). The EGFR inhibitor may also be a small molecule inhibitor. Examples of EGFR inhibitors include, but are not limited to, reversible and irreversible inhibitors, such as erlotinib (TARCEVA®; Genentech/Astellas Oncology), AZD9291 (AstraZeneca), gefitinib (IRESSA®; AstraZeneca), icotinib (BPI-2009H; Beta Pharma), rociletinib (CO-1686, AVL-301; Clovis Oncology), poziotinib (NOV120101, HM781-36B; Hanmi Pharmaceuticals/Spectrum Pharmaceuticals), afatinib (BIBW2292; Boehringer Ingelheim), pelitinib (EKB-569; Wyeth Pharmaceuticals), ASP8273 (Astellas), Luminespib (AUY922; Vernalis/Novartis), and XL647 (Exelixis).

c) B-Raf Inhibitors

A B-Raf inhibitor used in the methods herein may be any B-Raf inhibitor known in the art. Examples of B-Raf inhibitors include, but are not limited to, vemurafenib (ZELBORAF®), dabrafenib (TAFINLAR®), encorafenib (LGX818, Novartis), PLX-4720, PLX-3603 (RO5212054, Roche/Genentech), PLX-8394 (Daiichi Sankyo), CEP-32496 (Ambit Biosciences), XL281 (BMS-908662, Exelixis), and RAF265 (CHIR-265, Novartis).

d) MEK Inhibitors

A MEK inhibitor used in the methods herein may be any MEK inhibitor known in the art. Examples of MEK inhibitors include, but are not limited to, such as selumetinib (AZD6244, ARRY-142866, AstraZeneca), WX-554 (Wilex), trametinib (MEKINIST®; GlaxoSmithKline), refametinib (Ardea Biosciences), E-6201 (Eisai), MEK-162 (Novartis), cobimetinib (GDC-0973; XL-518; Exelixis, Roche), TAK-733 (Takeda Phamaceuticals), binimetinib (Array BioPharma), PD-0325901 (Pfizer), pimasertib (MSC1936369; EMD Serono), MSC2015103 (EMD Serono), WX-554 (WILEX), MEK162 (ARRY-162, Novartis), and RO48987655 (CH4987655; CIF/RG7167; Chugai Pharmaceuticals).

A MEK inhibitor used in the methods herein may be administered with a B-Raf inhibitor used in the methods herein.

e) Specific Anti-HER3 Antibodies and Antigen-Binding Fragments Thereof

Provided herein, for use in methods of treating cancer or methods or methods of determining whether a patient diagnosed with cancer is indicated as likely to be responsive to treatment with a HER3 inhibitor, are HER3 binding molecules, e.g., antibodies and antigen-binding fragments thereof that specifically bind HER3 (e.g., CL16, 2C2 or 2C2-YTE antibodies). Examples of such anti-HER3 antibodies and antigen binding fragments thereof may be found in International Application Publication No. WO 2013/078191. The full-length amino acid (aa) and nucleotide (nt) sequences for HER3 are known in the art (see, e.g., UniProt Acc. No. P2186 for human HER3, or UniProt Acc. No. 088458 for mouse HER3). In some aspects, the anti-HER3 binding molecules are human antibodies. In certain aspects, the HER3 binding molecules are antibodies or antigen-binding fragments thereof. In some aspects, HER3 binding molecules, e.g., antibodies or antigen-binding fragments thereof comprise a Fab, a Fab′, a F(ab′)₂, a Fd, a single chain Fv or scFv, a disulfide linked Fv, a V-NAR domain, an IgNar, an intrabody, an IgGΔCH2, a minibody, a F(ab′)₃, a tetrabody, a triabody, a diabody, a single-domain antibody, DVD-Ig, Fcab, mAb², a (scFv)₂, or a scFv-Fc. In some aspects, the antibody is of the IgG1 subtype and comprises the triple mutant YTE, as disclosed supra in the Definitions section.

In certain aspects, anti-HER3 antibodies or antigen-binding fragments thereof described herein are modified compared to the parent Clone 16 (CL16) antibody. The modifications can include mutations in the CDR regions and/or in the FW regions as compared to CL16. In certain aspects, an anti-HER3 antibody described herein comprises modifications to CDR1 and/or CDR3 of the light chain of CL16, including, but not limited to:
1) a light chain CDR1 comprising the consensus sequence X₁GSX₂SNIGLNYVS, wherein X₁ is selected from R or S, and X₂ is selected from S or L; and
2) a light chain CDR3 comprising the consensus sequence AAWDDX₃X₄X₅GEX₆, wherein X₃ is selected from S or G, X₄ is selected from L or P, X₅ is selected from R, I, P or S, and X₆ is selected from V or A.

In certain aspects, an anti-HER3 antibody or antigen-binding fragment thereof described herein comprises modifications to CDR2 of the heavy chain of CL16, including, but not limited to a heavy chain CDR1 comprising the consensus sequence X₇IGSSGGVTNYADSVKG, wherein X₇ is selected from Y, I or V.

In one aspect, an anti-HER3 antibody or antigen binding fragment thereof comprises a VL region comprising the consensus amino acid sequence:

[FW₁]X₁GSX₂SNIGLNYVS[FW₂]RNNQRPS[FW₃]AAWDDX₃X₄X₅GEX₆
[FW₄]

wherein [FW₁], [FW₂], [FW₃] and [FW₄] represent the amino acid residues of VL framework region 1 (SEQ ID NO: 40 or 44), VL framework region 2 (SEQ ID NO: 41), VL framework region 3 (SEQ ID NO: 42) and VL framework region 4 (SEQ ID NO: 43), and wherein X₁ represents amino acid residues arginine (R) or serine (S), X₂ represents amino acid residues serine (S) or leucine (L), X₃ represents amino acid residues serine (S) or glutamic acid (E), X₄ represents amino acid residues leucine (L) or proline (P), X₅ represents amino acid residues arginine (R), isoleucine (I), proline (P) or serine (S), and X₆ represents amino acid residues valine (V) or arginine (R).

In one aspect, an anti-HER3 antibody or antigen binding fragment thereof comprises a VH region comprises the consensus amino acid sequence:

[FW₅]YYYMQ[FW₆]X₇IGSSGGVTNYADSVKG[FW₇]VGLGDAFDI[FW₈]

wherein [FW₅], [FW₆], [FW₇] and [FW₈] represent the amino acid residues of VH framework region 1 (SEQ ID NO: 36), VH framework region 2 (SEQ ID NO: 37), VH framework region 3 (SEQ ID NO: 38) and VH framework region 4 (SEQ ID NO: 39), and wherein X₇ represents amino acid residues tyrosine (Y), isoleucine (I) or valine (V).

In one aspect, an anti-HER3 antibody or antigen binding fragment thereof comprises a VL region comprising the consensus amino acid sequence:

[FW₁]X₁GSX₂SNIGLNYVS[FW₂]RNNQRPS[FW₃]AAWDDX₃X₄X₅GEX₆
[FW₄]

wherein [FW₁], [FW₂], [FW₃] and [FW₄] represent the amino acid residues of VL framework region 1 (SEQ ID NO: 40 or 44), VL framework region 2 (SEQ ID NO: 41), VL framework region 3 (SEQ ID NO: 42) and VL framework region 4 (SEQ ID NO: 43), and wherein X₁ represents amino acid residues arginine (R) or serine (S), X₂ represents amino acid residues serine (S) or leucine (L), X₃ represents amino acid residues serine (S) or glutamic acid (E), X₄ represents amino acid residues leucine (L) or proline (P), X₅ represents amino acid residues arginine (R), isoleucine (I), proline (P) or serine (S), and X₆ represents amino acid residues valine (V) or arginine (R); and wherein said anti-HER3 antibody or antigen binding fragment thereof further comprises a VH region which comprises the consensus amino acid sequence:

[FW₅]YYYMQ[FW₆]X₇IGSSGGVTNYADSVKG[FW₇]VGLGDAFDI[FW₈]

wherein [FW₅], [FW₆], [FW₇] and [FW₈] represent the amino acid residues of VH framework region 1 (SEQ ID NO: 36), VH framework region 2 (SEQ ID NO: 37), VH framework region 3 (SEQ ID NO: 38) and VH framework region 4 (SEQ ID NO: 39), and wherein X₇ represents amino acid residues tyrosine (Y), isoleucine (I) or valine (V).

In some aspects, an anti-HER3 antibody or antigen-binding fragment thereof described herein comprises a VL-CDR1 consisting of sequence selected from the group consisting of SEQ ID NOs: 18, 19 and 20. In some aspects, an anti-HER3 antibody or antigen-binding fragment thereof described herein comprises a VL-CDR1 comprising a sequence selected from the group consisting of SEQ ID NOs: 18, 19 and 20. In some aspects, an anti-HER3 antibody or antigen-binding fragment thereof described herein comprises a VL-CDR2 consisting of SEQ ID NO: 21. In some aspects, an anti-HER3 antibody or antigen-binding fragment thereof described herein comprises a VL-CDR2 comprising SEQ ID NO: 21. In some aspects, an anti-HER3 antibody or antigen-binding fragment thereof described herein comprises a VL-CDR3 consisting of a sequence selected from the group consisting of SEQ ID NOs: 22, 23, 24, 25, 26, 27, 28, 29, and 30. In some aspects, an anti-HER3 antibody or antigen-binding fragment thereof described herein comprises a VL-CDR3 comprising a sequence selected from the group consisting of SEQ ID NOs: 22, 23, 24, 25, 26, 27, 28, 29, and 30.

In some aspects, an anti-HER3 antibody or antigen-binding fragment thereof described herein comprises a VH-CDR1 consisting of SEQ ID NO: 31. In some aspects, an anti-HER3 antibody or antigen-binding fragment thereof described herein comprises a VH-CDR1 comprising SEQ ID NO: 31. In some aspects, an anti-HER3 antibody or antigen-binding fragment thereof described herein comprises a VH-CDR2 consisting of a sequence selected from the group consisting of SEQ ID NOs: 32, 33 and 34. In some aspects, an anti-HER3 antibody or antigen-binding fragment thereof described herein comprises a VH-CDR2 comprising a sequence selected from the group consisting of SEQ ID NOs: 32, 33 and 34. In some aspects, an anti-HER3 antibody or antigen-binding fragment thereof described herein comprises a VH-CDR3 consisting of SEQ ID NO: 35. In some aspects, an anti-HER3 antibody or antigen-binding fragment thereof described herein comprises a VH-CDR3 comprising SEQ ID NO: 35.

In some aspects, an anti-HER3 antibody or antigen-binding fragment thereof described herein comprises a VL-CDR1 consisting of a sequence selected from the group consisting of SEQ ID NOs: 18, 19 and 20, except for one, two, three or four amino acid substitutions. In some aspects, an anti-HER3 antibody or antigen-binding fragment thereof described herein comprises a VL-CDR1 comprising a sequence selected from the group consisting of SEQ ID NOs: 18, 19 and 20, except for one, two, three or four amino acid substitutions. In some aspects, an anti-HER3 antibody or antigen-binding fragment thereof described herein comprises a VL-CDR2 consisting of SEQ ID NO: 21, except for one, two, three or four amino acid substitutions. In some aspects, an anti-HER3 antibody or antigen-binding fragment thereof described herein comprises a VL-CDR2 comprising SEQ ID NO: 21, except for one, two, three or four amino acid substitutions. In some aspects, an anti-HER3 antibody or antigen-binding fragment thereof described herein comprises a VL-CDR3 consisting of a sequence selected from the group consisting of SEQ ID NOs: 22, 23, 24, 25, 26, 27, 28, 29, and 30, except for one, two, three or four amino acid substitutions. In some aspects, an anti-HER3 antibody or antigen-binding fragment thereof described herein comprises a VL-CDR3 comprising a sequence selected from the group consisting of SEQ ID NOs: 22, 23, 24, 25, 26, 27, 28, 29, and 30, except for one, two, three or four amino acid substitutions.

In some aspects, an anti-HER3 antibody or antigen-binding fragment thereof described herein comprises a VH-CDR1 consisting of SEQ ID NO: 31, except for one, two, three or four amino acid substitutions. In some aspects, an anti-HER3 antibody or antigen-binding fragment thereof described herein comprises a VH-CDR1 comprising SEQ ID NO: 31, except for one, two, three or four amino acid substitutions. In some aspects, an anti-HER3 antibody or antigen-binding fragment thereof described herein comprises a VH-CDR2 consisting of a sequence selected from the group consisting of SEQ ID NOs: 32, 33 and 34, except for one, two, three or four amino acid substitutions. In some aspects, an anti-HER3 antibody or antigen-binding fragment thereof described herein comprises a VH-CDR2 comprising a sequence selected from the group consisting of SEQ ID NOs: 32, 33 and 34, except for one, two, three or four amino acid substitutions. In some aspects, an anti-HER3 antibody or antigen-binding fragment thereof described herein comprises a VH-CDR3 consisting of SEQ ID NO: 35, except for one, two, three or four amino acid substitutions. In some aspects, an anti-HER3 antibody or antigen-binding fragment thereof described herein comprises a VH-CDR3 comprising SEQ ID NO: 35, except for one, two, three or four amino acid substitutions.

In some aspects, an anti-HER3 antibody or antigen-binding fragment thereof described herein comprises a VL-CDR1 consisting of a sequence selected from the group consisting of SEQ ID NOs: 18, 19 and 20; a VL-CDR2 consisting of SEQ ID NO: 21; and a VL-CDR3 consisting of a sequence selected from the group consisting of SEQ ID NOs: 22, 23, 24, 25, 26, 27, 28, 29, and 30. In some aspects, an anti-HER3 antibody or antigen-binding fragment thereof described herein comprises a VL-CDR1 comprising a sequence selected from the group consisting of SEQ ID NOs: 18, 19 and 20; a VL-CDR2 comprising SEQ ID NO: 21; and a VL-CDR3 comprising a sequence selected from the group consisting of SEQ ID NOs: 22, 23, 24, 25, 26, 27, 28, 29, and 30.

In some aspects, an anti-HER3 antibody or antigen-binding fragment thereof described herein comprises a VH-CDR1 consisting of SEQ ID NO: 31; a VH-CDR2 consisting of a sequence selected from the group consisting of SEQ ID NOs: 32, 33 and 34; and a VH-CDR3 consisting of SEQ ID NO: 35. In some aspects, an anti-HER3 antibody or antigen-binding fragment thereof described herein comprises a VH-CDR1 comprising SEQ ID NO: 31; a VH-CDR2 comprising a sequence selected from the group consisting of SEQ ID NOs: 32, 33 and 34; a VH-CDR3 comprising SEQ ID NO: 35.

In some aspects, an anti-HER3 antibody or antigen-binding fragment thereof described herein comprises a VL-CDR1 consisting of a sequence selected from the group consisting of SEQ ID NOs: 18, 19 and 20, except for one, two, three or four amino acid substitutions; a VL-CDR2 consisting of SEQ ID NO: 21, except for one, two, three or four amino acid substitutions; and a VL-CDR3 consisting of a sequence selected from the group consisting of SEQ ID NOs: 22, 23, 24, 25, 26, 27, 28, 29, and 30, except for one, two, three or four amino acid substitutions. In some aspects, an anti-HER3 antibody or antigen-binding fragment thereof described herein comprises a VL-CDR1 comprising a sequence selected from the group consisting of SEQ ID NOs: 18, 19 and 20, except for one, two, three or four amino acid substitutions; a VL-CDR2 comprising SEQ ID NO: 21, except for one, two, three or four amino acid substitutions; and a VL-CDR3 comprising a sequence selected from the group consisting of SEQ ID NOs: 22, 23, 24, 25, 26, 27, 28, 29, and 30, except for one, two, three or four amino acid substitutions.

In some aspects, an anti-HER3 antibody or antigen-binding fragment thereof described herein comprises a VH-CDR1 consisting of SEQ ID NO: 31, except for one, two, three or four amino acid substitutions; a VH-CDR2 consisting of a sequence selected from the group consisting of SEQ ID NOs: 32, 33 and 34, except for one, two, three or four amino acid substitutions; and a VH-CDR3 consisting of SEQ ID NO: 35, except for one, two, three or four amino acid substitutions. In some aspects, an anti-HER3 antibody or antigen-binding fragment thereof antibody described herein comprises a VH-CDR1 comprising SEQ ID NO: 31, except for one, two, three or four amino acid substitutions; a VH-CDR2 comprising a sequence selected from the group consisting of SEQ ID NOs: 32, 33 and 34, except for one, two, three or four amino acid substitutions; and VH-CDR3 comprising SEQ ID NO: 35, except for one, two, three or four amino acid substitutions.

In certain aspects, an anti-HER3 antibody or antigen-binding fragment thereof described herein comprises modifications to CDR1, CDR2, and/or CDR3 of the heavy and/or light chain, and further comprises modifications to FW1, FW2, FW3, and/or FW4 of the heavy and/or light chain. In some aspects, FW₁ comprises SEQ ID NO: 40 or 44, FW₂ comprises SEQ ID NO: 41, FW₃ comprises SEQ ID NO: 42, FW₄ comprises SEQ ID NO: 43, FW₅ comprises SEQ ID NO: 36, FW₆ comprises SEQ ID NO: 37, FW₇ comprises SEQ ID NO: 38, and FW₈ comprises SEQ ID NO: 39.

In some aspects, FW₁ comprises SEQ ID NO: 40 or 44, except for one, two, three or four amino acid substitutions; FW₂ comprises SEQ ID NO: 41, except for one, two, three or four amino acid substitutions; FW₃ comprises SEQ ID NO: 42, except for one, two, three or four amino acid substitutions; FW₄ comprises SEQ ID NO: 43, except for one, two, three or four amino acid substitutions; FW₅ comprises SEQ ID NO: 36, except for one, two, three or four amino acid substitutions; FW₆ comprises SEQ ID NO: 37, except for one, two, three or four amino acid substitutions; FW₇ comprises SEQ ID NO: 38, except for one, two, three or four amino acid substitutions; and FW₈ comprises SEQ ID NO: 39, except for one, two, three or four amino acid substitutions.

In certain aspects, an anti-HER3 antibody or antigen-binding fragment thereof described herein comprises a VL and a VH comprising VL-CDR1, VL-CRD2, VL-CDR3, VH-CDR1, VH-CDR2, and VH-CDR3 amino acid sequences identical or identical except for four, three, two, or one amino acid substitutions in one or more CDRs to: SEQ ID NOs: 18, 21, 22, 31, 32, and 35, SEQ ID NOs: 18, 21, 26, 31, 32 and 35, SEQ ID NOs: 18, 21, 27, 31, 32 and 35, SEQ ID NOs: 20, 21, 22, 31, 32 and 35, SEQ ID NOs: 19, 21, 22, 31, 32 and 35, SEQ ID NOs: 18, 21, 25, 31, 32 and 35, SEQ ID NOs: 18, 21, 28, 31, 32 and 35, SEQ ID NOs: 18, 21, 29, 31, 32 and 35, SEQ ID NOs: 18, 21, 30, 31, 32 and 35, SEQ ID NOs: 18, 21, 23, 31, 32 and 35, SEQ ID NOs: 19, 21, 23, 31, 32 and 35, SEQ ID NOs: 20, 21, 23, 31, 32 and 35, SEQ ID NOs: 18, 21, 24, 31, 32 and 35, or SEQ ID NOs: 18, 21, 25, 31, 32 and 35, respectively.

Heavy and light chain variable domains of the anti-HER3 antibody or antigen-binding fragment thereof described herein include the sequences listed in TABLE 2.

TABLE 2

| SEQ | | |
|---|---|---|
| ID | | |
| NO. | Description | Sequence |
| 1 | CL16VL | QSVLTQPPSASGTPGQRVTISCSGSSSNIGLNYVSWYQQLPGTAPKLLISRNNQRP |
| | (Germlined) | SGVPDRFSGSKSGTSASLAISGLRSEDEADYYCAAWDDSLSGEVFGGGTKLTVL |
| 17 | CL16VL | QYELTQPPSASGTPGQRVTMSCSGSSSNIGLNYVSWYQQLPGTAPKLLISRNNQR |
| | (original) | PSGVPDRFSGSKSGTSASLAISGLRSEDEADYYCAAWDDSLSGEVFGGGTKLTVL |
| 2 | CL16 VH | EVQLLESGGGLVQPGGSLRLSCAASGFTFSYYYMQWVRQAPGKGLEWVSYIGS |
| | | SGGVTNYADSVKGRFTISRDNSKNTLYLQMNSLRAEDTAVYYCARVGLGDAFD |
| | | IWGQGTMVTVSS |
| 4 | 5H6 VL | QSVLTQPPSASGTPGQRVTISCSGSSSNIGLNYVSWYQQLPGTAPKLLISRNNQRP |
| | | SGVPDRFSGSKSGTSASLAISGLRSEDEADYYCAAWDDGLPGEVFGGGTKLTVL |
| 5 | 8A3 VL | QSVLTQPPSASGTPGQRVTISCSGSSSNIGLNYVSWYQQLPGTAPKLLISRNNQRP |
| | | SGVPDRFSGSKSGTSASLAISGLRSEDEADYYCAAWDDSLIGEVFGGGTKLTVL |
| 6 | 4H6 VL | QSVLTQPPSASGTPGQRVTISCRGSSSNIGLNYVSWYQQLPGTAPKLLISRNNQRP |
| | | SGVPDRFSGSKSGTSASLAISGLRSEDEADYYCAAWDDSLSGEVFGGGTKLTVL |
| 7 | 6E.3 VL | QSVLTQPPSASGTPGQRVTISCSGSLSNIGLNYVSWYQQLPGTAPKLLISRNNQRP |
| | | SGVPDRFSGSKSGTSASLAISGLRSEDEADYYCAAWDDSLSGEVFGGGTKLTVL |
| 8 | 2B11 VL | QSVLTQPPSASGTPGQRVTISCSGSSSNIGLNYVSWYQQLPGTAPKLLISRNNQRP |
| | | SGVPDRFSGSKSGTSASLAISGLRSEDEADYYCAAWDDSLPGEVFGGGTKLTVL |
| 9 | 2D1 VL | QSVLTQPPSASGTPGQRVTISCSGSSSNIGLNYVSWYQQLPGTAPKLLISRNNQRP |
| | | SGVPDRFSGSKSGTSASLAISGLRSEDEADYYCAAWDDSLSGEAFGGGTKLTVL |
| 10 | 3A6 VL | QSVLTQPPSASGTPGQRVTISCSGSSSNIGLNYVSWYQQLPGTAPKLLISRNNQRP |
| | | SGVPDRFSGSKSGTSASLAISGLRSEDEADYYCAAWDDSPSGEVFGGGTKLTVL |
| 11 | 4C4 VL | QSVLTQPPSASGTPGQRVTISCSGSSSNIGLNYVSWYQQLPGTAPKLLISRNNQRP |
| | | SGVPDRFSGSKSGTSASLAISGLRSEDEADYYCAAWDDSLRGEVFGGGTKLTVL |
| 12 | 15D12.1 | EVQLLESGGGLVQPGGSLRLSCAASGFTFSYYYMQWVRQAPGKGLEWVSIIGSS |
| | (15D12.I) | GGVTNYADSVKGRFTISRDNSKNTLYLQMNSLRAEDTAVYYCARVGLGDAFDI |
| | VH | WGQGTMVTVSS |
| 13 | 15D12.2 | EVQLLESGGGLVQPGGSLRLSCAASGFTFSYYYMQWVRQAPGKGLEWVSVIGS |
| | (15D1.V) | SGGVTNYADSVKGRFTISRDNSKNTLYLQMNSLRAEDTAVYYCARVGLGDAFD |
| | VH | IWGQGTMVTVSS |
| 14 | 1A4 VL | QSVLTQPPSASGTPGQRVTISCSGSSSNIGLNYVSWYQQLPGTAPKLLISRNNQRP |
| | | SGVPDRFSGSKSGTSASLAISGLRSEDEADYYCAAWDDSPPGEAFGGGTKLTVL |
| 3 | 2C2 VL | QSVLTQPPSASGTPGQRVTISCSGSLSNIGLNYVSWYQQLPGTAPKLLISRNNQRP |
| | | SGVPDRFSGSKSGTSASLAISGLRSEDEADYYCAAWDDSPPGEAFGGGTKLTVL |
| 15 | 3E.1 VL | QSVLTQPPSASGTPGQRVTISCRGSSSNIGLNYVSWYQQLPGTAPKLLISRNNQRP |
| | | SGVPDRFSGSKSGTSASLAISGLRSEDEADYYCAAWDDSPPGEAFGGGTKLTVL |
| 16 | 2F 10 | QSVLTQPPSASGTPGQRVTISCSGSSSNIGLNYVSWYQQLPGTAPKLLISRNNQRP |
| | | SGVPDRFSGSKSGTSASLAISGLRSEDEADYYCAAWDDSPSGEAFGGGTKLTVL |

In certain aspects, an anti-HER3 antibody or antigen-binding fragment thereof described herein comprises an antibody VL and an antibody VH, wherein the VL comprises an amino acid sequence at least about 80%, about 85%, about 90%, about 95%, about 96%, about 97%, about 98%, about 99%, or about 100% identical to a reference amino acid sequence selected from the group consisting of SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 14, SEQ ID NO: 15, SEQ ID NO: 16, and SEQ ID NO: 17. In a specific embodiment, the VL comprises VL CDRs identical to those of the VL reference amino acid sequence.

In other aspects, an anti-HER3 antibody or antigen-binding fragment thereof described herein comprises an antibody VL and an antibody VH, wherein the VH comprises an amino acid sequence at least about 80%, about 85%, about 90%, about 95%, about 96%, about 97%, about 98%, about 99%, or about 100% identical to a reference amino acid sequence selected from the group consisting of SEQ ID NO: 2, SEQ ID NO: 12 and SEQ ID NO: 13. In a specific embodiment, the VH comprises VH CDRs identical to those of the VL reference amino acid sequence.

In other aspects, an anti-HER3 antibody or antigen-binding fragment thereof described herein comprises a VL comprising a sequence at least about 80%, about 85%, about 90%, about 95%, about 96%, about 97%, about 98%, about 99%, or about 100% identical to a reference amino acid sequence selected from the group consisting of SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 14, SEQ ID NO: 15, SEQ ID NO: 16, and SEQ ID NO: 17, and further comprises a VH comprising a sequence at least about 80%, about 85%, about 90%, about 95%, about 96%, about 97%, about 98%, about 99%, or about 100% identical to a reference amino acid sequence selected from the group consisting of SEQ ID NO: 2, SEQ ID NO: 12 and SEQ ID NO: 13. In a specific embodiment, the VL comprises VL CDRs identical to those of the VL reference amino acid sequence, and the VH comprises VH CDRs identical to those of the VH reference amino acid sequence. In a certain aspect, an anti-HER3 antibody or antigen-binding fragment provided herein comprises a VL comprising a sequence that is at least 80%, at least 85%, at least 90%, or at least 95% identical to SEQ ID NO: 3 and a VH comprising a sequence that is at least 80%, at least 85%, at least 90%, or at least 95% identical to SEQ ID NO: 2. In a certain aspect, an anti-HER3 antibody or antigen-binding fragment provided herein comprises a VL comprising a sequence that is at least 80%, at least 85%, at least 90%, or at least 95% identical SEQ ID NO: 3 and a VH comprising a sequence that is at least 80%, at least 85%, at least 90%, or at least 95% identical to SEQ ID NO: 2, wherein the VL CDRs are identical to those of SEQ ID NO: 3 and the VH CDRs are identical to those of SEQ ID NO: 2.

In some aspects, an anti-HER3 antibody or antigen-binding fragment thereof comprises a VH of TABLE 2 and a VL of TABLE 2. Antibodies are designated throughout the specification according to their VL chains. The heavy chains of the specific antibodies disclosed in the present specification correspond to the CL16 original heavy chain (SEQ ID NO: 2). Thus, the “CL16 antibody” is an IgG1 comprising two original CL16 light chains (SEQ ID NO: 17) and two CL16 original heavy chains (SEQ ID NO: 2), whereas the “2C2 antibody” is an IgG1 comprising two 2C2 light chains (2C2 VL (SEQ ID NO: 3) and two CL16 original heavy chains (SEQ ID NO: 2).

In some aspects, the anti-HER3 antibody or antigen-binding fragment thereof comprises a heavy chain constant region or fragment thereof. In some specific aspects, the heavy chain constant region is an IgG constant region. The IgG constant region can comprise a light chain constant region selected from the group consisting of a kappa constant region and a lambda constant region.

In certain aspects, an anti-HER3 antibody or antigen-binding fragment thereof described herein binds HER3 with substantially the same or better affinity as the CL16 antibody, comprising the CL16 original heavy chain (SEQ ID NO: 2) and the original CL16 light chain (SEQ ID NO: 17). In certain aspects, an anti-HER3 antibody or antigen-binding fragment thereof described herein binds HER3 with substantially the same or better affinity as the 2C2 antibody, comprising the 2C2 light chain (2C2 VL (SEQ ID NO: 3) and the CL16 original heavy chain (SEQ ID NO: 2).

In one aspect provided herein, an anti-HER3 antibody or antigen-binding fragment thereof specifically binds HER3 and antigenic fragments thereof with a dissociation constant of k_{d} (k_{off}/kₒₙ) of less than 10⁻⁶ M, or of less than 10⁻⁷ M, or of less than 10⁻⁸ M, or of less than 10⁻⁹ M, or of less than 10⁻¹⁰ M, or of less than 10⁻¹¹ M, or of less than 10⁻¹² M, or of less than 10⁻¹³ M. In a particular aspect provided herein, an anti-HER3 antibody or antigen-binding fragment thereof specifically binds HER3 and antigenic fragments thereof with a dissociation constant between 2×10⁻¹⁰ M and 6×10⁻¹⁰ M.

In another aspect, an anti-HER3 antibody or antigen-binding fragment thereof described herein binds to HER3 and/or antigenic fragments thereof with a K_{off} of less than 1×10⁻³ s⁻¹, or less than 2×10⁻³ s⁻¹. In other aspects, an anti-HER3 antibody or antigen-binding fragment thereof binds to HER3 and antigenic fragments thereof with a K_{off} of less than 10⁻³ s⁻¹, less than 5×10³s⁻¹, less than 10⁻⁴ s⁻¹, less than 5×10⁻⁴ s⁻¹, less than 10⁻⁵ s⁻¹, less than 5×10⁻⁵ s⁻¹, less than 10⁻⁶ s⁻¹, less than 5×10⁻⁶ s⁻¹, less than less than 5×10⁻⁷ s⁻¹, less than 10⁻⁸ s⁻¹, less than 5×10⁻⁸ s⁻¹, less than 10⁻⁹ s⁻¹, less than 5×10⁻⁹ s⁻¹, or less than 10⁻¹⁰ s⁻¹. In a particular aspect, an anti-HER3 antibody or antigen-binding fragment thereof described herein binds to HER3 and/or antigenic fragments thereof with a K_{off} of between 0.5×10⁻⁴ s⁻¹ and 2.0×10⁻⁴ s⁻¹.

In another aspect, an anti-HER3 antibody or antigen-binding fragment thereof described herein binds to HER3 and/or antigenic fragments thereof with an association rate constant or kₒₙ rate of at least 10⁵ M⁻¹ s⁻¹, at least 5×10⁵ M⁻¹ s⁻¹, at least 10⁻⁶ M⁻¹ s⁻¹, at least 5×10⁻⁶ M⁻¹ s⁻¹ at least 10⁷ M⁻¹ s⁻¹ at least 5×10⁷ M⁻¹ s⁻¹ or at least 10⁸ M⁻¹ s⁻¹ or at least 10⁻⁹ M⁻¹ s⁻¹. In another aspect, an anti-HER3 antibody or antigen-binding fragment thereof described herein binds to HER3 and/or antigenic fragments thereof with an association rate constant or kon rate of between 1×10⁵ M⁻¹ s⁻¹ and 6×10⁵ M⁻¹s⁻¹.

The VH and VL sequences disclosed in TABLE 1 can be “mixed and matched” to create other anti-HER3 binding molecules described herein. In certain aspects, the VH sequences of 15D12.I and 15D12.V are mixed and matched. Additionally or alternatively, the VL sequences of 5H6, 8A3, 4H6, 6E.3, 2B11, 2D1, 3A6, 4C4, 1A4, 2C2, 3E.1 can be mixed and matched.

In certain aspects, an anti-HER3 antibody or antigen-binding fragment thereof described herein comprises mutations that improve the binding to human FcRn and improve the half-life of the anti-HER3 antibody or antigen-binding fragment thereof. In some aspects, such mutations are a methionine (M) to tyrosine (Y) mutation in position 252, a serine (S) to threonine (T) mutation in position 254, and a threonine (T) to glutamic acid (E) mutation in position 256, numbered according to the EU index as in Kabat (Kabat, et al. (1991) Sequences of Proteins of Immunological Interest, U.S. Public Health Service, National Institutes of Health, Washington, D.C.), introduced into the constant domain of an IgG1. See U.S. Pat. No. 7,658,921, which is incorporated by reference herein. This type of mutant IgG, referred to as a “YTE mutant” has been shown display approximately four-times increased half-life as compared to wild-type versions of the same antibody (Dall'Acqua et al., J. Biol. Chem. 281:23514-24 (2006)). In some aspects, an anti-HER3 antibody or antigen-binding fragment thereof comprising an IgG constant domain comprises one or more amino acid substitutions of amino acid residues at positions 251-257, 285-290, 308-314, 385-389, and 428-436, numbered according to the EU index as in Kabat, wherein such mutations increase the serum half-life of the anti-HER3 antibody or antigen-binding fragment thereof.

In some aspects, a YTE mutant further comprises a substitution at position 434 of the IgG constant domain, numbered according to the EU index as in Kabat, with an amino acid selected from the group consisting of tryptophan (W), methionine (M), tyrosine (Y), and serine (S). In other aspects, a YTE mutant further comprises a substitution at position 434 of the IgG constant domain, numbered according to the EU index as in Kabat, with an amino acid selected from the group consisting of tryptophan (W), methionine (M), tyrosine (Y), and serine (S), and substitution at position 428 of the IgG constant domain, numbered according to the EU index as in Kabat, with an amino acid selected from the group consisting of threonine (T), leucine (L), phenylalanine (F), and serine (S).

In yet other aspect, a YTE mutant further comprises a substitution at position 434 of the IgG constant domain, numbered according to the EU index as in Kabat, with tyrosine (Y), and a substitution at position 257 of the IgG constant domain, numbered according to the EU index as in Kabat, with leucine (L). In some aspects, a YTE mutant further comprises a substitution at position 434 of the IgG constant domain, numbered according to the EU index as in Kabat, with serine (S), and a substitution at position 428 of the IgG constant domain, numbered according to the EU index as in Kabat, with leucine (L).

In a specific aspect, an anti-HER3 antibody or antigen-binding fragment thereof comprises a 2C2 light chain variable region (2C2 VL; SEQ ID NO: 3), an original CL16 heavy chain variable region (SEQ ID NO: 2), and an IgG1 constant domain comprising a methionine (M) to tyrosine (Y) mutation in position 252, a serine (S) to threonine (T) mutation in position 254, and a threonine (T) to glutamic acid (E) mutation in position 256 of the IgG1 constant domain, numbered according to the EU index as in Kabat.

In a specific aspect, an anti-HER3 antibody or antigen-binding fragment thereof comprises a light chain variable region and a heavy chain variable region as presented in Table 3. TABLE 3 provides the SEQ ID NOs for each clone.

TABLE 3

| SEQ | | SEQ | |
|---|---|---|---|
| ID | DESCRIPTION | ID | DESCRIPTION |
| 17 | Clone 16 V_{L} aa | 21 | Clone 4H6 V_{L} CDR2 aa |
| 1 | Clone 16-germlined V_{L} aa | 22 | Clone 4H6 V_{L} CDR3 aa |
| 2 | Clone 16 V_{H} aa | 7 | Clone 6E.3 V_{L} aa |
| 18 | Clone 16 V_{L} CDR1 aa | 19 | Clone 6E.3 V_{L} CDR1 aa |
| 21 | Clone 16 V_{L} CDR2 aa | 21 | Clone 6E.3 V_{L} CDR2 aa |
| 22 | Clone 16 V_{L} CDR3 aa | 22 | Clone 6E.3 V_{L} CDR3 aa |
| 31 | Clone 16 V_{H} CDR1 aa | 9 | Clone 2D1 V_{L} aa |
| 32 | Clone 16 V_{H} CDR2 aa | 18 | Clone 2D1 V_{L} CDR1 aa |
| 35 | Clone 16 V_{H} CDR3 aa | 21 | Clone 2D1 V_{L} CDR2 aa |
| 8 | Clone 2B11 V_{L} aa | 28 | Clone 2D1 V_{L} CDR3 aa |
| 18 | Clone 2B11 V_{L} CDR1 aa | 10 | Clone 3A6 V_{L} aa |
| 21 | Clone 2B11 V_{L} CDR2 aa | 18 | Clone 3A6 V_{L} CDR1 aa |
| 25 | Clone 2B11 V_{L} CDR3 aa | 21 | Clone 3A6 V_{L} CDR2 aa |
| 14 | Clone 1A4 V_{L} aa | 29 | Clone 3A6 V_{L} CDR3 aa |
| 18 | Clone 1A4 V_{L} CDR1 aa | 11 | Clone 4C4 V_{L} aa |
| 21 | Clone 1A4 V_{L} CDR2 aa | 18 | Clone 4C4 V_{L} CDR1 aa |
| 22 | Clone 1A4 V_{L} CDR3 aa | 21 | Clone 4C4 V_{L} CDR2 aa |
| 3 | Clone 2C2 V_{L} aa | 30 | Clone 4C4 V_{L} CDR3 aa |
| 19 | Clone 2C2 V_{L} CDR1 aa | 12 | Clone 15D12.1 V_{H} aa |
| 21 | Clone 2C2 V_{L} CDR2 aa | 31 | Clone 15D12.1 V_{H} CDR1 aa |
| 23 | Clone 2C2 V_{L} CDR3 aa | 33 | Clone 15D12.1 V_{H} CDR2 aa |
| 16 | Clone 2F10 V_{L} aa | 35 | Clone 15D12.1 V_{H} CDR3 aa |
| 18 | Clone 2F10 V_{L} CDR1 aa | 13 | Clone 15D12.2 V_{H} aa |
| 21 | Clone 2F10 V_{L} CDR2 aa | 31 | Clone 15D12.2 V_{H} CDR1 aa |
| 24 | Clone 2F10 V_{L} CDR3 aa | 34 | Clone 15D12.2 V_{H} CDR2 aa |
| 15 | Clone 3E.1 V_{L} aa | 35 | Clone 15D12.2 V_{H} CDR3 aa |
| 20 | Clone 3E.1 V_{L} CDR1 aa | 36 | V_{H} FW1 aa |
| 21 | Clone 3E.1 V_{L} CDR2 aa | 37 | V_{H} FW2 aa |
| 23 | Clone 3E.1 V_{L} CDR3 aa | 38 | V_{H} FW3 aa |
| 4 | Clone 5H6 V_{L} aa | 39 | V_{H} FW4 aa |
| 18 | Clone 5H6 V_{L} CDR1 aa | 40 | V_{L} FW1 germlined aa |
| 21 | Clone 5H6 V_{L} CDR2 aa | 41 | V_{L} FW2 aa |
| 26 | Clone 5H6 V_{L} CDR3 aa | 42 | V_{L} FW3 aa |
| 5 | Clone 8A3 V_{L} aa | 43 | V_{L} FW4 aa |
| 18 | Clone 8A3 V_{L} CDR1 aa | 44 | V_{L} FW1 original aa |
| 21 | Clone 8A3 V_{L} CDR2 aa | 45 | IgG1 constant region* |
| 27 | Clone 8A3 V_{L} CDR3 aa | 46 | IgG1 constant region* - YTE |
| 6 | Clone 4H6 V_{L} aa | 47 | Clone 16 V_{L} nt |
| 20 | Clone 4H6 V_{L} CDR1 aa | 48 | Clone 16 V_{H} nt |

*allotype differences are provided
V_{L} aa consensus:
[FW₁]X₁GSX₂SNIGLNYVS[FW₂]RNNQRPS[FW₃]AAWDDX₃X₄X₅GEX₆[FW₄] wherein [FW₁], [FW₂], [FW₃] and [FW₄] represent VL framework regions, wherein
(a) X₁ represents amino acid residues Arginine (R) or Serine (S),
(b) X₂ represents amino acid residues Serine (S) or Leucine (L),
(c) X₃ represents amino acid residues Serine (S) or Glycine (G),
(d) X₄ represents amino acid residues Leucine (L) or Proline (P),
(e) X₅ represents amino acid residues Arginine (R), Isoleucine (I), Proline (P) or Serine (S), and
(f) X₆ represents amino acid residues Valine (V) or Alanine (A).
V_{H} aa consensus:
[FW₅]YYYMQ[FW₆]X₇IGSSGGVTNYADSVKG[FW₇]VGLGDAFDI[FW₈] wherein [FW₅], [FW₆], [FW₇] and [FW₈] represent VH framework regions, wherein X₇ represents amino acid residues Tyrosine (Y), Isoleucine (I) or Valine (V)

In a specific aspect, an anti-HER3 antibody or antigen-binding fragment thereof comprises a light chain variable region and a heavy chain variable region as described in PCT International Publication No. WO 2013/078191 A1, which is hereby incorporated by reference in its entirety.

In certain aspects, an anti-HER3 antibody or antigen-binding fragment thereof described herein comprise at least one IgG constant domain amino acid substitution selected from the group consisting of:
(a) substitution of the amino acid at position 252 with tyrosine (Y), phenylalanine (F), tryptophan (W), or threonine (T),
(b) substitution of the amino acid at position 254 with threonine (T),
(c) substitution of the amino acid at position 256 with serine (S), arginine (R), glutamine (Q), glutamic acid (E), aspartic acid (D), or threonine (T),
(d) substitution of the amino acid at position 257 with leucine (L),
(e) substitution of the amino acid at position 309 with proline (P),
(f) substitution of the amino acid at position 311 with serine (S),
(g) substitution of the amino acid at position 428 with threonine (T), leucine (L), phenylalanine (F), or serine (S),
(h) substitution of the amino acid at position 433 with arginine (R), serine (S), isoleucine (I), proline (P), or glutamine (Q),
(i) substitution of the amino acid at position 434 with tryptophan (W), methionine (M), serine (S), histidine (H), phenylalanine (F), or tyrosine, and
(j) a combination of two or more of said substitutions, wherein the positions are numbered according to the EU index as in Kabat, and wherein the modified IgG has an increased serum half-life compared to the serum half-life of an IgG having the wild-type IgG constant domain.

In other aspects, the VH and/or VL amino acid sequences can be at least 85%, 90%, 95%, 96%, 97%, 98% or 99% identical to the sequences set forth above, and comprise 1, 2, 3, 4, 5 or more conservative substitutions. In certain aspects, the VH and/or VL amino acid sequences can be at least 85%, 90%, 95%, 96%, 97%, 98% or 99% identical to the sequences set forth above, and comprise 1, 2, 3, 4, 5 or more conservative substitutions which are not within the VH CDRs or VL CDRs. In other aspects, the VH and/or VL amino acid sequences can be at least 80%, 85%, 90%, or 95% identical to the sequences set forth above, and comprise 1, 2, 3, 4, 5 or more conservative substitutions. In certain aspects, the VH and/or VL amino acid sequences can be at least 80%, 85%, 90%, or 95% identical to the sequences set forth above, and comprise 1, 2, 3, 4, 5 or more conservative substitutions which are not within the VH CDRs or VL CDRs. A HER3 antibody having VH and VL regions having high (i.e., 80% or greater) similarity to the VH regions of SEQ ID NOs: 2, 12 or 13 and/or VL regions of SEQ ID NOs: 1, 3, 4, 5, 6, 7, 8, 9, 10, 11, 14, 15, 16, or 17, respectively, can be obtained by mutagenesis (e.g., site-directed or PCR-mediated mutagenesis) of nucleic acid molecules encoding SEQ ID NOs: 1-17, followed by testing of the encoded altered antibody for retained function using the functional assays described herein.

The affinity or avidity of an antibody for an antigen can be determined experimentally using any suitable method well known in the art, e.g., flow cytometry, enzyme-linked immunosorbent assay (ELISA), or radioimmunoassay (RIA), or kinetics (e.g., BIACORE™ analysis). Direct binding assays as well as competitive binding assay formats can be readily employed. (See, for example, Berzofsky et al., “Antibody-Antigen Interactions,” In Fundamental Immunology, Paul, W. E., Ed., Raven Press: New York, N.Y. (1984); Kuby, Immunology, W. H. Freeman and Company: New York, N.Y. (1992); and methods described herein. The measured affinity of a particular antibody-antigen interaction can vary if measured under different conditions (e.g., salt concentration, pH, temperature). Thus, measurements of affinity and other antigen-binding parameters (e.g., K_{D} or Kd, Kₒₙ, K_{off}) are made with standardized solutions of antibody and antigen, and a standardized buffer, as known in the art and such as the buffer described herein.

It also known in the art that affinities measured using BIACORE™ analysis can vary depending on which one of the reactants is bound to the chip. In this respect, affinity can be measured using a format in which the targeting antibody (e.g., the 2C2 monoclonal antibody) is immobilized onto the chip (referred to as an “IgG down” format) or using a format in which the target protein (e.g., HER3) is immobilized onto the chip (referred to as, e.g., a “HER3 down” format).

In another aspect, provided herein are HER3-binding molecules that bind to the same epitope as do the various anti-HER3 antibodies described herein. The term “epitope” as used herein refers to a protein determinant capable of binding to an antibody described herein. Epitopes usually consist of chemically active surface groupings of molecules such as amino acids or sugar side chains and usually have specific three dimensional structural characteristics, as well as specific charge characteristics. Conformational and non-conformational epitopes are distinguished in that the binding to the former but not the latter is lost in the presence of denaturing solvents. Such antibodies can be identified based on their ability to cross-compete (e.g., to competitively inhibit the binding of, in a statistically significant manner) with antibodies such as the CL16 antibody, the 2C2 antibody, or the 2C2-YTE mutant, in standard HER3 binding assays. Accordingly, in one aspect, provided herein are anti-HER3 antibodies and antigen-binding fragments thereof, e.g., human monoclonal antibodies that compete for binding to HER3 with another anti-HER3 antibody or antigen-binding fragment thereof described herein, such as the CL16 antibody or the 2C2 antibody. The ability of a test antibody to inhibit the binding of, e.g., the CL16 antibody or the 2C2 antibody demonstrates that the test antibody can compete with that antibody for binding to HER3; such an antibody can, according to non-limiting theory, bind to the same or a related (e.g., a structurally similar or spatially proximal) epitope on HER3 as the anti-HER3 antibody or antigen-binding fragment thereof with which it competes. In one aspect, the anti-HER3 antibody or antigen-binding fragment thereof that binds to the same epitope on HER3 as, e.g., the CL16 antibody or the 2C2 antibody, is a human monoclonal antibody.

### VI. Methods of Determining Expression Levels and Clinical Response

RNA expression may be assayed by detecting or quantitating mRNA levels by any method known in the art. These methods include, but are not limited to northern blots, ribonuclease protection assays, in situ hybridization, for example, RNAscope® technology, ILLUMINA® RNASeq, ILLUMINA® next generation sequencing (NGS), ION TORRENT™ RNA next generation sequencing, 454™ pyrosequencing, or Sequencing by Oligo Ligation Detection (SOLID™), PCR-based methods, and the like. PCR-based methods include RT-PCR and Real-Time (or quantitative) RT-PCR (qRT-PCR).

Protein expression may be assayed in a particular sample using a variety of methods. Any suitable protein quantification method can be used. In some embodiments, antibody-based/immunospecific methods are used. The immunoassays that can be used include techniques such as immunohistochemistry assays, Western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. ELISA methods include, for example, direct ELISA, indirect ELISA, and sandwich ELISA. Such assays are routine and well known in the art (see, e.g., Ausubel et al., eds, (1994) Current Protocols in Molecular Biology (John Wiley & Sons, Inc., NY) Vol. 1, which is incorporated by reference herein in its entirety). Methods such as flow cytometry and cytometric bead array may be used to measure expression of cell-surface proteins. Mass spectroscopic methods may also be used to measure protein expression levels in a sample.

Levels of expression of protein dimers or other complexes, such as, for example, EGFR homodimers, may be assayed using well-known technques, for example, can be assayed by measuring the protein-protein interactions between members of the dimers or other complex, e.g., between EGFR monomers. These protein-protein interactions may be determined, for example, through VeraTag® formalin-fixed paraffin embedded (FFPE) or lysate assays (International Patent Application Publication No. WO 2014/165855; DeFazio et al., 2011, Breast Cancer Research, 2011, 13:R44; Mukherjee et al., 2011, PLoS One 6:e16443), Fluorescence Resonance Energy Transfer (FRET) (Kong et al., 2006, Cancer Res 66:2834-2843), or in situ proximity ligation assays (Koos et al., 2009, Am J Pathol 175:1631-1637; Zieba et al., 2010, Clin Chem 56:99-110).

Clinical response can be assessed using screening techniques such as magnetic resonance imaging (MM) scan, x-radiographic imaging, computed tomographic (CT) scan, flow cytometry or fluorescence-activated cell sorter (FACS) analysis, histology, gross pathology, and blood chemistry, including but not limited to changes detectable by ELISA, RIA, chromatography, and the like. In addition to these positive therapeutic responses, the subject undergoing therapy with the HER3 inhibitor, EGFR inhibitor, and/or B-Raf inhibitor, can experience the beneficial effect of an improvement in the symptoms associated with the disease.

### EXAMPLES

### Example 1: Dual ErbB Blockade with 2C2-YTE and Cetuximab Yields Enhanced Antitumor Activity in SCCHN by Inhibiting Parallel Signaling Pathways

EGFR homodimers are activated upon ligand (TGF-α and AREG) binding, stimulating downstream signaling, and ultimately, cellular proliferation. HER3 (ErbB3) and HER2 (ErbB2) heterodimers are activated upon ligand (NRG) binding, stimulating downstream signaling, and ultimately, cellular survival. 2C2-YTE is an IgG1 monoclonal antibody that binds to ErbB3 and potently inhibits ErbB3 ligand-(NRG-)dependent and independent ErbB3 activation. This Example demonstrates that ErbB3 acts in concert with EGFR in squamous cell carcinoma of the head and neck (SCCHN) cancer.

### HER3 Ligand (NRG), but not HER3, is Overexpressed in SCCHN Cancer Samples.

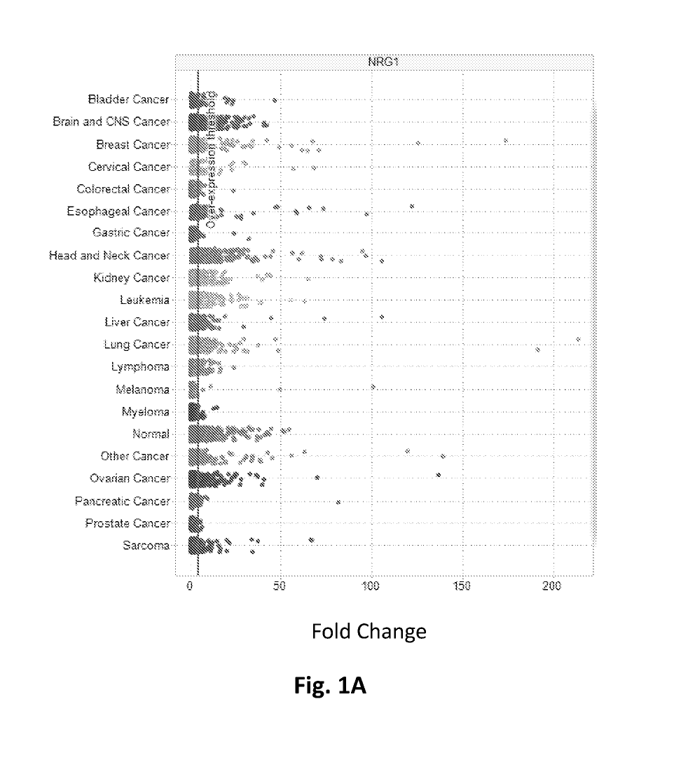

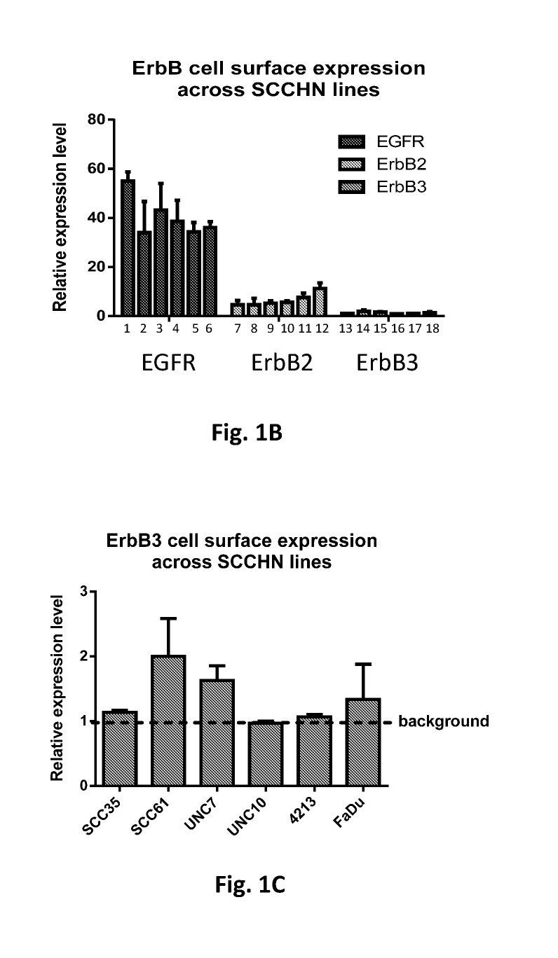

To elucidate the role of HER3 (ErbB3) and its ligand NRG in cancer, samples from different cancers were evaluated for HER3 (ErbB3) and NRG mRNA expression level (see, Tables 4 and 5, and FIG. 1). NRG mRNA is frequently overexpressed in head and neck cancer (Table 4 and FIG. 1A). In contrast, NRG receptor ErbB3 mRNA is not overexpressed in SCCHN samples (Table 5).

TABLE 4
NRG mRNA expression levels in cancer. Overexpression is defined
as >4-fold greater NRG mRNA relative to NRG
mRNA across all tumor types.

| | NRG mRNA | | |
|---|---|---|---|
| | | Number | Frequency of |
| | Total | of samples | samples exhibiting |
| General Cancer | Number of | with NRG mRNA | NRG mRNA |
| Type | samples | overexpressed | overexpression |
| Head and Neck | 506 | 228 | 45.1% |
| Esophageal | 149 | 54 | 36.2% |
| Cervical | 153 | 38 | 24.8% |
| Kidney | 758 | 183 | 24.1% |
| Brain and CNS | 1431 | 277 | 19.4% |
| Bladder | 227 | 27 | 11.9% |
| Normal, non-cancer | 2657 | 314 | 11.8% |
| Liver | 669 | 74 | 11.1% |
| Lung | 1567 | 169 | 10.8% |
| Ovarian | 1775 | 148 | 8.3% |
| Other | 878 | 69 | 7.9% |
| Pancreatic | 171 | 10 | 5.8% |
| Sarcoma | 1198 | 67 | 5.6% |
| Leukemia | 5371 | 264 | 4.9% |
| Lymphoma | 2024 | 70 | 3.5% |
| Breast | 4339 | 111 | 2.6% |
| Gastric | 416 | 8 | 1.9% |
| Melanoma | 403 | 5 | 1.2% |
| Prostate | 330 | 4 | 1.2% |
| Myeloma | 1823 | 15 | 0.8% |
| Colorectal | 2204 | 18 | 0.8% |
| Grand Total | 29049 | 2153 | 7.4% |

TABLE 5
ErbB3 mRNA expression levels in cancer. Overexpression is
defined as >4-fold greater ErbB3 mRNA relative to
ErbB3 mRNA across all tumor types.

| | ErbB3 mRNA | | |
|---|---|---|---|
| | | Number of | Frequency of |
| | Total | samples with | samples exhibiting |
| | number | ErbB3 mRNA | ErbB3 mRNA |
| General Cancer Type | of samples | overexpressed | overexpression |
| Colorectal | 2204 | 1646 | 74.7% |
| Melanoma | 403 | 277 | 68.7% |
| Breast | 4339 | 2139 | 49.3% |
| Gastric | 416 | 189 | 45.4% |
| Bladder | 227 | 75 | 33.0% |
| Prostate | 330 | 86 | 26.1% |
| Lung | 1567 | 377 | 24.1% |
| Pancreatic | 171 | 38 | 22.2% |
| Kidney | 758 | 159 | 21.0% |
| Normal, non-cancer | 2657 | 543 | 20.4% |
| Ovarian | 1775 | 346 | 19.5% |
| Cervical | 153 | 25 | 16.3% |
| Liver | 669 | 109 | 16.3% |
| Other | 878 | 63 | 7.2% |
| Esophageal | 149 | 10 | 6.7% |
| Brain and CNS | 1431 | 35 | 2.4% |
| Head and Neck | 506 | 10 | 2.0% |
| Sarcoma | 1198 | 7 | 0.6% |
| Lymphoma | 2024 | 5 | 0.2% |
| Leukemia | 5371 | 0 | 0.0% |
| Myeloma | 1823 | 0 | 0.0% |
| Grand Total | 29049 | 6139 | 21.1% |

Given the disparity between the high expression of ErbB3 and ErbB3 ligand (NRG) in SCCHN samples, the expression level of related ErbBs, EGFR (also referred to as ErbB1 or HER1) and ErbB2, were evaluated in SCCHN cell lines. While EGFR is highly expressed on the cell surface across SCCHN lines, ErbB2 and ErbB3 are relatively underexpressed (FIG. 1B and FIG. 1C).

The prevalence of HPV and PI3K mutations in SCCHN samples was also determined and compared with NRG expression in those samples, as shown in Table 6. The Cancer Genome Atlas head and neck squamous cell carcinoma mRNASeq gene expression data for NRG were sourced from the Broad Institute's Firehose analysis infrastructure (HPV+/HPV− and PI3K wild-type/PI3K mutant) and plotted using TIBCO Spotfire. Units on both axes were upper-quartile normalized and log 2 transformed RSEM (RNASeq by Expectation Maximization) values. Linear regression was used to quantify the relationship between genes and p-values were generated using an F-test to determine if the independent variable (X) predicted a significant proportion of the variance of the dependent variable (Y). The p-value was then calculated from the F-distribution where the F-statistic is calculated with the sum of squares between the estimated line and the total mean of the y,'s having one degree of freedom as numerator and the residual sum of squares divided by the number of degrees of freedom (n−2) as denominator. The median NRG expression was higher in HPV negative SCCHN samples than in HPV positive samples. The median NRG expression was also higher in PI3K wildtype SCCHN samples than in PI3K mutant samples.

TABLE 6
Prevalence of HPV positive and negative, and PI3K mutations
in SCCHN samples, compared with median NRG expression.

| | HPV− | HPV+ | PI3K wt | PI3K mut |
|---|---|---|---|---|
| Count | 84 | 25 | 258 | 45 |
| Median NRG | 9.004 | 7.453 | 8.751 | 7.886 |
| expression | | | | |
| Outliers | 4 | 0 | 9 | 0 |

| | | |
|---|---|---|
| P-value | 4.95E−8 | 0.0013 |

### NRG-Dependent ErbB3 Phosphorylation is Chiefly Dependent on ErbB2

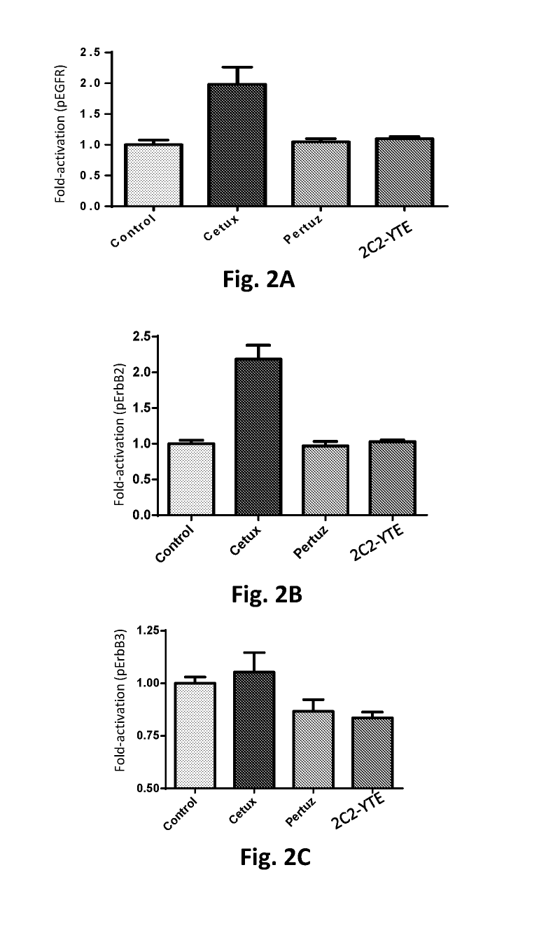

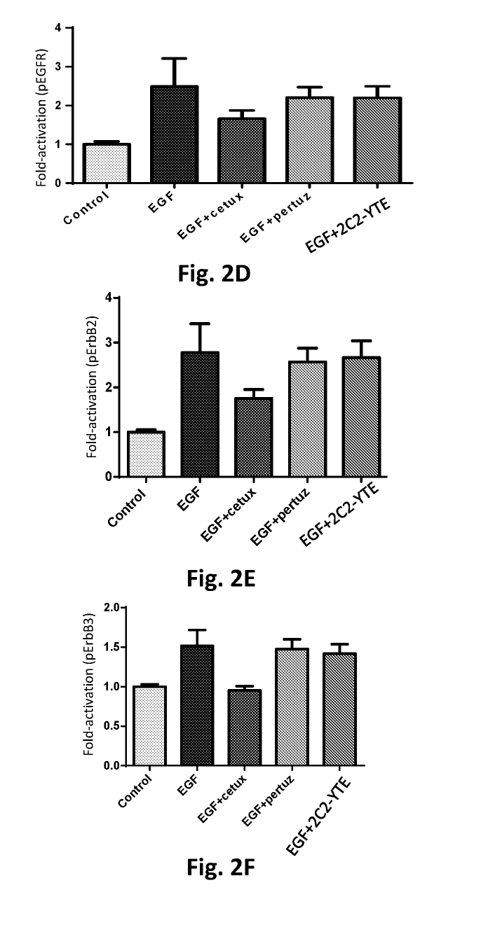

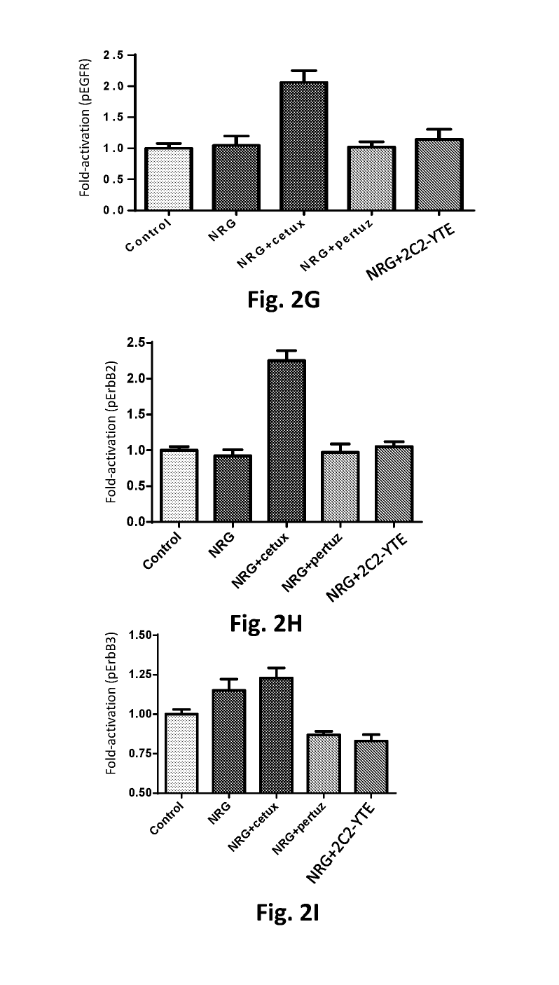

Cal27, a SCCHN cell line, was treated with a control (IgG1), the anti-EGFR monoclonal antibody cetuximab (“Cetux”), the anti-ErbB2 monoclonal antibody pertuzumab (“Pertuz”), or with 2C2-YTE, and activation of EGFR, ErbB2, and ErbB3 was analyzed (FIG. 2A, FIG. 2B, and FIG. 2C, respectively). In the absence of ligand stimulation, cetuximab, but neither pertuzumab nor 2C2-YTE, stimulates activation of EGFR and ErbB2, but not ErbB3, as assayed by ErbB phosphorylation. ErbB phosphorylation was measured using the Theranostics Health platform. Similar results were observed in the SCCHN FaDu cell line.

Next, activation of the ErbBs was evaluated in the presence of the EGFR ligand, EGF (FIG. 2D, FIG. 2E, and FIG. 2F). While incubation of the cells with EGF activated EGFR, ErbB2, and ErbB3, cetuximab, but neither pertuzumab nor 2C2-YTE were capable of inhibiting or blocking the EGF-stimulated activation (FIG. 2D, FIG. 2E, and FIG. 2F). Similar results were observed in the SCCHN FaDu cell line.

Finally, activation of the ErbBs was evaluated in the presence of the ErbB2/ErbB3 ligand, NRG (FIG. 2G, FIG. 2H, and FIG. 2I). Incubation with NRG and cetuximab resulted in activation of EGFR, ErbB2, and ErbB3. There was no effect on activation of EGFR or ErbB2 in cells treated with NRG alone, or treated with NRG together with either with pertuzumab or 2C2-YTE (FIG. 2G and FIG. 2H). However, treatment with NRG and pertuzumab or 2C2-YTE, but not cetuximab, blocked activation of ErbB3 (FIG. 2I). Similar results were observed in the SCCHN FaDu cell line.

Taken together, these data indicate that ErbB2 is the major kinase responsible for responsible for activating ErbB3 in a NRG-dependent fashion in SCCHN. Thus, EGFR and ErbB3-targeting agents are required to achieve maximum receptor shutdown.

SCCHN cell lines exhibit differential anti-proliferative sensitivity to 2C2-YTE and cetuximab in vitro and inhibit parallel signaling pathways in drug-sensitive SCCHN cell lines

The ability of cetuximab and 2C2-YTE to exhibit anti-proliferative activity on a panel of SCCHN cell lines was evaluated. SCCHN cells were seeded in 96 well-plates at 3-5,000 cells/well in media containing reduced (1-3)% fetal bovine serum. 2C2-YTE, cetuximab, and their combination were titrated on cells, which were allowed to grow until the control-treated cells reached near-confluency (3-7 days). Cells were treated with CellTiter-Glo® (Promega), and luminescence was measured in a BioTek plate reader. Normalized luminescence were plotted as a function of log-transformed drug concentration, and data were fit to a 4-parameter nonlinear regression algorithm using GraphPad Prism.

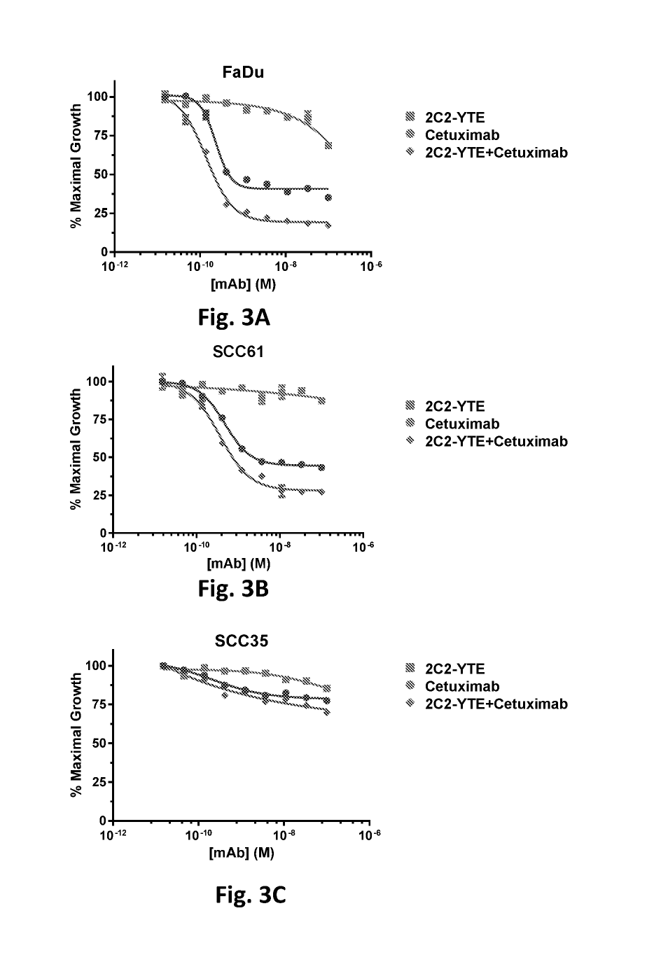

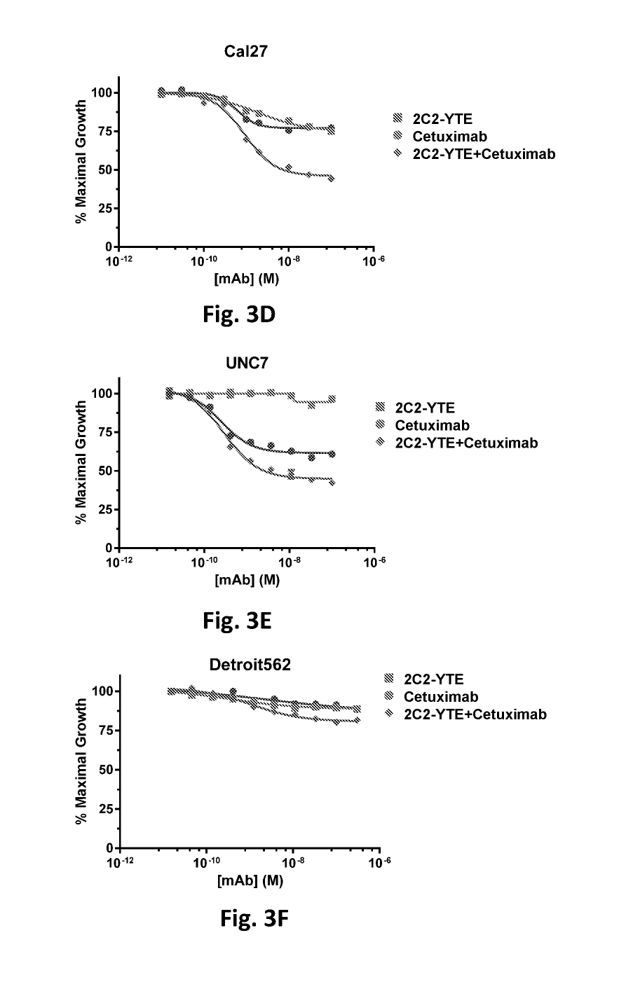

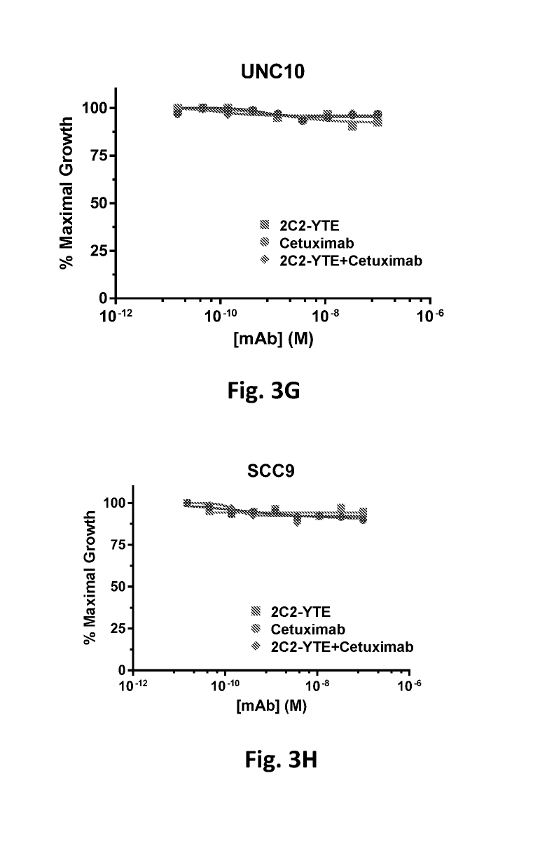

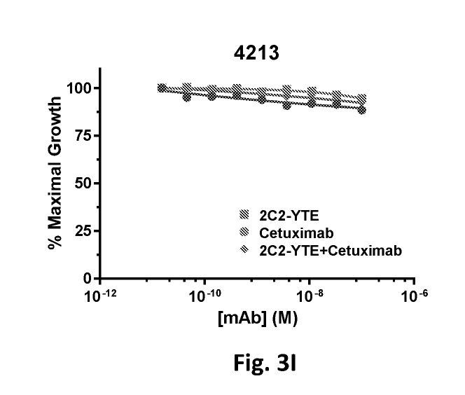

Either cetuximab or 2C2-YTE was capable of impairing cellular proliferation in certain SCCHN cell lines (FIGS. 3A-3E), yet had minimal impact in other SCCHN cell lines (FIGS. 3F-3H), and in a non-cancerous skin cell line (FIG. 31). Moreover, in certain SCCHN cell lines, incubation with both cetuximab and 2C2-YTE has an enhanced effect on the inhibition of cellular proliferation (FIGS. 3A-3E).

Next, the interplay between ErbB activation and activation of ERK and AKT pathways was assessed by analyzing activation of ErbB, ERK, and AKT, respectively, in the absence of exogenous ligand (FIG. 4).

Treatment of drug-sensitive cells with cetuximab, but not 2C2-YTE, inhibited activation of the ERK pathway, but not activation of the AKT pathway. In contrast, treatment of drug-sensitive cells with 2C2-YTE inhibited activation of ErbB3 and AKT, but had no impact of activation of EGFR and ERK. Thus, EGFR primarily activates the ERK pathway, while ErbB3 primarily activates the AKT pathway. Moreover, treatment of drug-sensitive cells with both cetuximab and 2C2-YTE yielded maximum inhibition of ErbB-dependent activation of ERK and AKT signaling.

2C2-YTE Potentiates Cetuximab Antitumor Activity in Xenograft Models of SCCHN

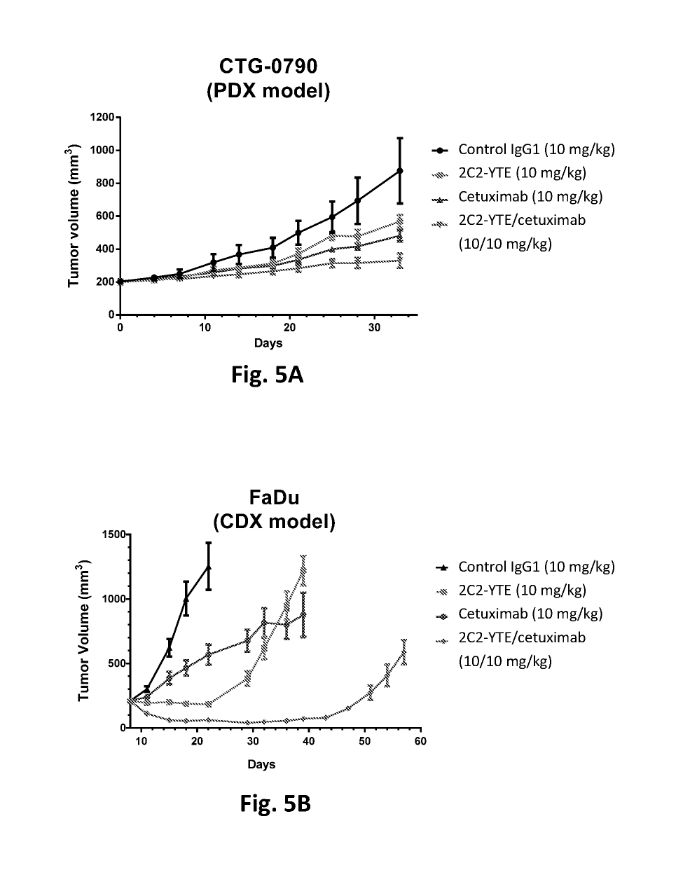

In both CAL27 and UNC7 SCCHN cell lines, treatment with cetuximab resulted in an upregulation of cell surface ErbB3. Given that cetuximab induces ErbB3 upregulation in SCCHN cell lines, it is possible that such upregulation impairs cetuximab's antitumor activity. Thus, the ability of 2C2-YTE to enhance cetuximab's antitumor activity by counteracting cetuximab's ErbB3 upregulation was evaluated. Patient-derived (PDX, FIG. 5A) and cell-line-derived (CDX, FIG. 5B) xenograft tumor mouse models were treated with 10 mg/kg of either control, 2C2-YTE, cetuximab, or 2C2-YTE and cetuximab and tumor volume was evaluated.

CTG-0790 cells were a primary patient-derived SCCHN tumor. Human FADU Head and Neck cells (ATCC No. HTB-43) were maintained at 37° C. in a 5% CO₂ incubator in RPMI 1640 medium containing 4.5 g/L glucose, L-glutamine, sodium pyruvate and 10% fetal bovine serum. Xenografts were established by subcutaneously injecting 5×10⁻⁶ cells per mouse (suspended in 50% matrigel) into the right flanks of 4- to 6-week-old athymic nu/nu mice. Tumors were allowed to grow up to 200 mm³ before randomization for efficacy studies. 2C2-YTE (10 mg/kg), cetuximab (10 mg/kg), control IgG1 or the combination of 2C2-YTE with cetuximab (10/10 mg/kg) monoclonal antibodies were administered intraperitoneally. Caliper measurements were used to calculate tumor volumes using the formula:

tumor volume=π÷6(length×width×width)

for tumors grown in mice. Antitumor effects are expressed as percent delta tumor growth inhibition (TGI), which was calculated as follows:

percent delta TGI=1−(dT÷dC)×100,

where dT=change in mean tumor volume in treatment group compared to the value at staging, and dC=change in mean tumor volume in control group compared to the value at staging.

In both models, while treatment with either cetuximab or 2C2-YTE delayed tumor growth, combination therapy with 2C2-YTE and cetuximab yielded the greatest delay in tumor growth (FIGS. 5A and 5B).

NRG Expression is Strongly Associated with TGF-α and AREG Expression in SCCHN Tumor Samples

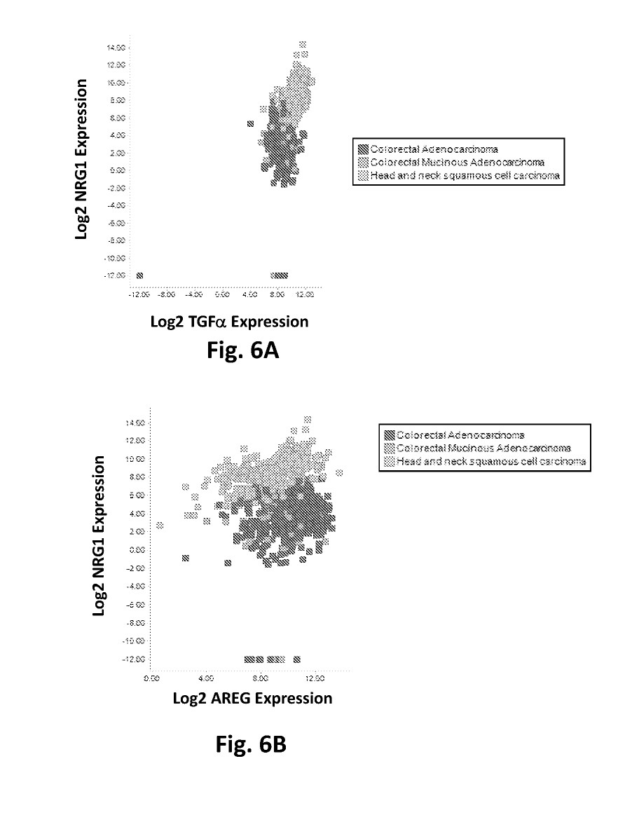

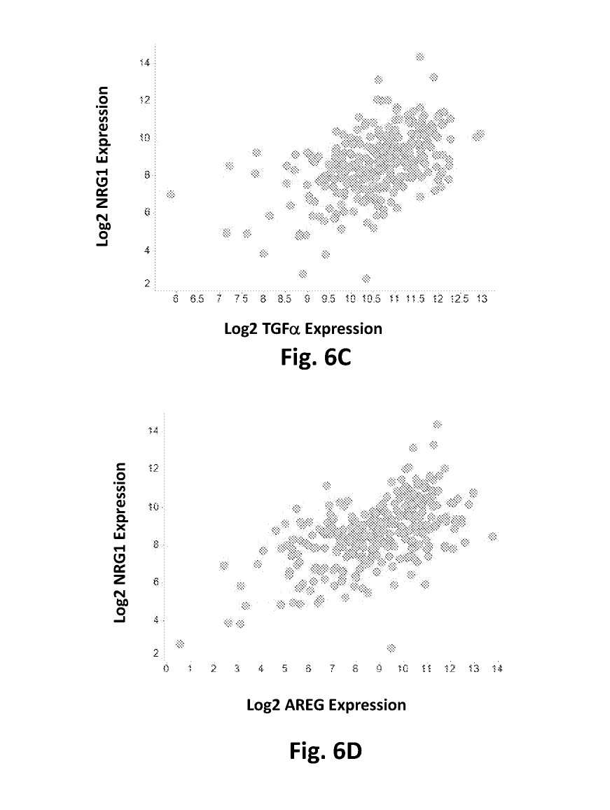

Given the prevalence of overexpression of NRG in SCCHN cancer samples (FIG. 1), the expression levels of the EGFR ligands TGF-α and amphiregulin (“AREG”) were evaluated in colorectal adenocarcinoma samples, colorectal mucinous adenocarcinoma samples, and SCCHN samples (FIG. 6). RNAseq data were upper quartile-normalized within (rather than across) cancer types. Log 2-transformed NRG expression data were plotted as a function of Log 2-transformed expression data for each of the EGFR ligands. Statistical analysis was generated using linear regression after patients with Log 2 expression values of −12 were removed. Correlates with p-values <0.05 and r2>0.25 were deemed positive.

NRG mRNA levels were strongly associated with TGF-α and AREG mRNA levels in SCCHN samples, but not in the colorectal adenocarcinoma and colorectal mucinous adenocarcinoma samples tested (FIGS. 6A and 6B, and Table 7). FIGS. 6C and 6D show the association in SCCHN samples alone. In contrast, NRG mRNA expression was not strongly associated with other EGFR ligands, EGF, EREG, HB-EGF, Epigen, or BTC levels in either the SCCHN or colorectal cancer (CRC) samples tested (Table 7).

Table 7 provides the association between the indicated EGFR ligands and NRG in either SCCHN or CRC samples.

| EGFR ligand | SCCHN (n = 303) | CRC (n = 537) |
|---|---|---|
| AREG | P = 8.53E−28, r² = 0.328 | P = 6.26E−7, r² = 0.045 |
| TGF-α | P = 2.87E−20, r² = 0.247 | P = 1.77E−5, r² = 0.034 |
| EGF | P = 0.319, r² = 0.003 | P = 0.161, r² = 0.004 |
| EREG | P = 7.21E−17, r² = 0.207 | P = 3.28E−13, r² = 0.094 |
| HB-EGF | P = 1.85E−6, r² = 0.073 | P = 2.32E−4, r² = 0.025 |
| Epigen | P = 8.37E−11, r² = 0.131 | P = 0.742, r² = 0.000 |
| BTC | P = 1.79E−4, r² = 0.046 | P = 0.186, r² = 0.003 |

### SCCHN Cell Lines Sensitive to 2C2-YTE and Cetuximab Secrete High Levels of TGF-α and AREG

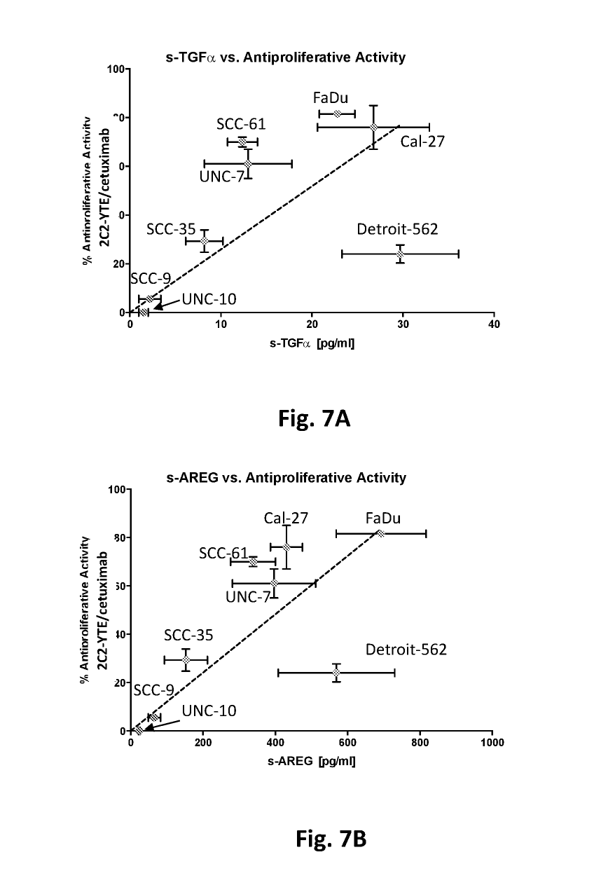

The levels of secreted TGF-α and AREG protein (FIGS. 7A and 7B, respectively) in SCCHN cell lines were quantified by ELISA after a 48 hour incubation in serum free media. SCCHN cell lines were incubated with 100 nM cetuximab for 48 hours to promote accumulation (and prevent internalization and degradation) of the secreted forms of TGF-α and AREG in the cell growth media. Ligand levels were quantified from conditioned media using the Human DuoSet ELISA assay for TGF-α and AREG (R&D). The ELISA was performed according to the manufacturer's instructions. Data were plotted as a function of the combined anti-proliferative activity of 2C2-YTE in combination with cetuximab.

SCCHN cell lines sensitive to treatment with 2C2-YTE and cetuximab (SCC-61, FaDu, UNC-7, and Cal-27) secreted high levels of both TGF-α and AREG (FIGS. 7A and 7B). These data indicate that the levels of TGF-α and AREG can be utilized to determine the sensitivity of cells, e.g., SCCHN tumor cells, to Her3 inhibitors such as 2C2-YTE and/or to EGFR inhibitors, such as cetuximab.

### Example 2: Association of ErbB/HER Biomarkers with Antitumor Activity of the Anti-Erbb3/HER3 Monoclonal Antibody 2C2-YTE in SCCHN

A proximity-dependent dual-antibody immunoassay (VeraTag®) was used to quantify total receptor levels (HER1/EGFR, HER2 and HER3) and dimerization/activation (EGFR homodimer, HER2-HER3 heterodimer, HER3-PI3 kinase (p85) and phospho-HER3) in formalin-fixed paraffin-embedded (FFPE) SSCHN tumors, and in cell lines and xenografts treated with 2C2-YTE. Elevated levels of phosphorylated HER3 were found to be predictive of 2C2-YTE activity. In a mouse xenograft model of SCCHN, 2C2-YTE reduced tumor volume by 85% and correlated with both phosphorylated HER3 (p=0.0063) and HER3-PI3K (p=0.0220).

### Evaluation of NRG Expression in a Cohort of Human SCCHN Tumor Samples

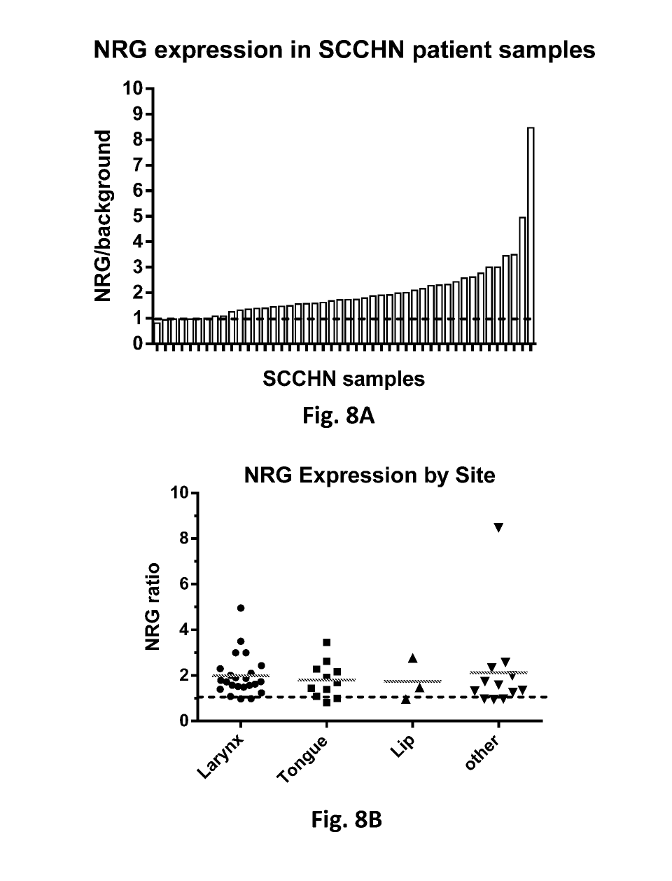

The results in Example 1 demonstrated that NRG is frequently overexpressed in SCCHN cancer cells. For this study, forty-eight human SCCHN formalin-fixed paraffin embedded (“FFPE”) tumor samples were analyzed for NRG expression using the^{RNAscope}® in situ hybridization assay (Advanced Cell Diagnostics; Hayward Calif.) and AQUA (Automated Quantitative Analysis; Genoptix; Carlsbad Calif.) quantitation. Background was defined as the AQUA score of a non-specific probe. As with the samples evaluated in Example 1, the majority of SCCHN tumor samples evaluated for this study express NRG (FIG. 8A), with NRG expression occurring in larynx, tongue, lip, and other sample locations (FIG. 8B).

### Characterization of ErbB Expression Profile in a Cohort of Human SCCHN FFPE Tumor Samples Using Veratag® Assays

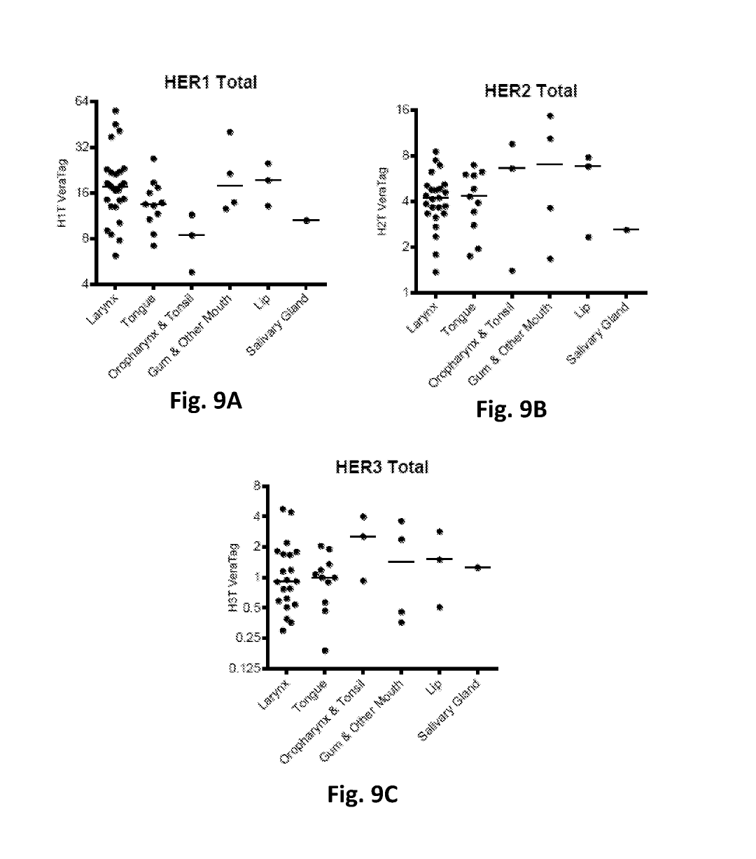

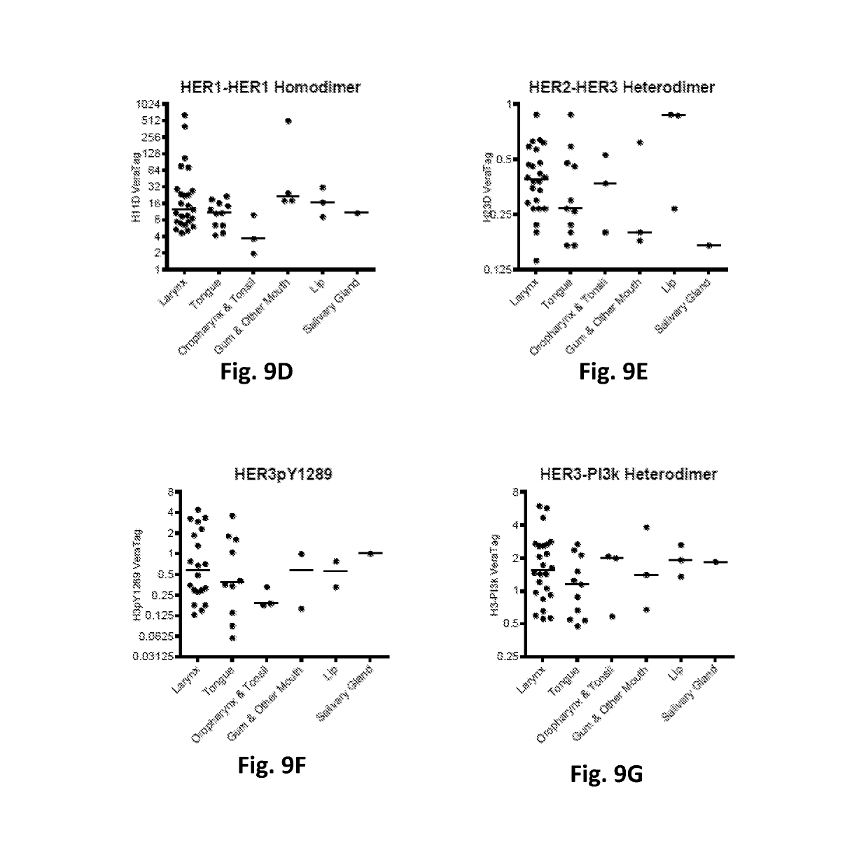

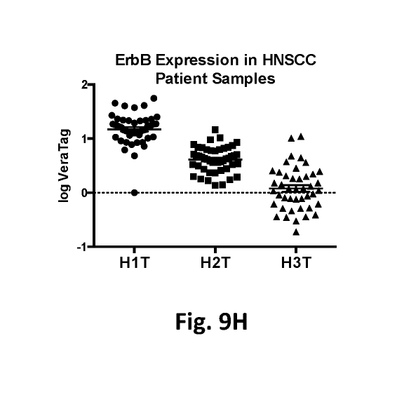

SCCHN FFPE tumor samples from a variety of anatomical locations were evaluated for expression of EGFR (H1T), ErbB2 (H2T), ErbB3 (H3T), EGFR homodimers (H11D), ErbB2/ErbB3 heterodimers (H23D), activated ErbB3 (ErbB3pY1289, H3pY1289), and ErbB3-PI3k heterodimers (H3PI3k) (FIGS. 9A-9G, respectively) using the VeraTag® assay. FIG. 9H compares the expression of EGFR (H1T), ErbB2 (H2T), and ErbB3 (H3T) amongst all SCCHN patient samples. This analysis indicated that these receptors, and receptor dimers are broadly expressed in SCCHN tumors from different anatomical locations.

VeraTag® assays rely on the proximity of two antibodies, one conjugated to biotin, and one conjugated to a fluorescent reporter dye. Streptavidin-functionalized sensitizer dye binds to Ab-biotin. 670 nm light effects singlet oxygen release, which in turn induces cleavage and release of the VeraTag® reporter molecule into illumination buffer. Illumination buffer is collected and analyzed by capillary electrophoresis. FFPE blocks, antibody conjugates and molecular scissors were prepared for the analyses as detailed in previous publications (DeFazio et al., 2011, Breast Cancer Research, 2011, 13:R44; Mukherjee et al., 2011, PLoS One 6:e16443).

Pairwise comparison of Veratag® ErbB assays in human SCCHN FFPE tumor samples (Table 8) revealed a positive correlation between total ErbB3 (H3T) and ErbB2 (H2T) expression. In addition, EGFR homodimer (H11D) expression levels showed a negative correlation with ErbB3 expression and a positive correlation with total EGFR (H1T) expression levels. Finally, PI3K-bond ErbB3 (H3-PI3K) showed a positive correlation with phosphorylated ErbB3 (H3pY1289).

Table 8 depicts pairwise comparison of Veratag® ErbB assays in a cohort of human SCCHN FFPE tumor samples.

| Spearman r | | | | | | |
|---|---|---|---|---|---|---|
| (p value) | H2T | H3T | H11D | H23D | H3pY1289 | H3-PI3k |
| H1T | 0.2056 | −0.2333 | 0.7900 | 0.1756 | −0.0584) | −0.0930 |
| | (0.1704) | (0.1321) | (<0.0001) | (0.2487) | (0.2487) | (0.5431) |
| H2T | | 0.4314 | 0.1306 | 0.7218 | −0.0648 | 0.1186 |
| | | (0.0049) | (0.3923) | (<0.0001) | (0.7069) | (0.4489) |
| H3T | | | −0.3580 | 0.4031 | −0.1113 | 0.0067 |
| | | | (0.0184) | (0.0081) | (0.5183) | (0.9663) |
| H11D | | | | 0.0674 | −0.0076 | −0.0489 |
| | | | | (0.6599) | (0.9636) | (0.7495) |
| H23D | | | | | 0.1377 | 0.2480 |
| | | | | | (0.4163) | (0.1046) |
| H3pY1289 | | | | | | 0.5508 |
| | | | | | | (0.0003) |

NRG Expression does not Correlate with ErbB Expression or ErbB3 Activation in SCCHN Tumor Samples

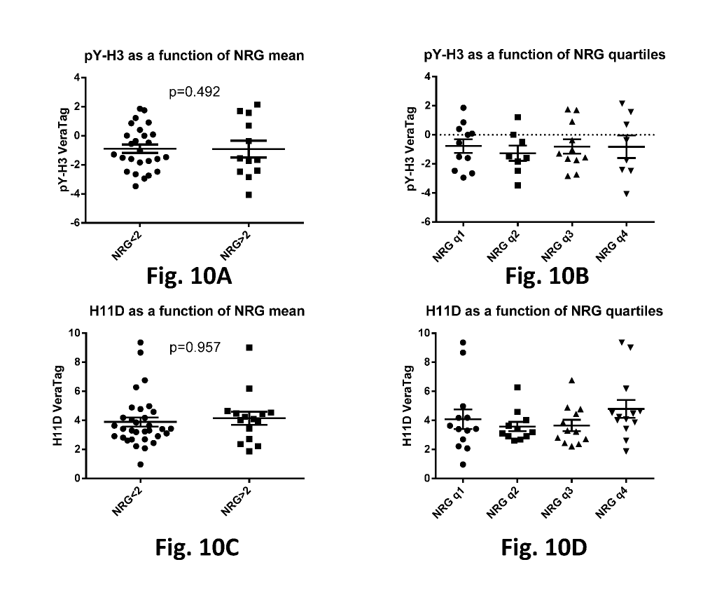

EGFR homodimers (H11D) and activated ErbB3 (pY-H3) were evaluated as a function of the level of NRG in SCCHN cell lines. FIGS. 10A and 10C show pY-H3 and H11D levels, respectively, in cell lines above and below the mean of NRG expression. FIGS. 10B and 10D show pY-H3 and H11D levels in cell lines expressing NRG levels in the first, second, third, and fourth quartiles of NRG expression. NRG expression was not found to correlate with EGFR expression or HER3 activation. Table 9, below, depicts p-values for the data in FIGS. 10B and 10D.

Table 9 depicts the p-values for the NRG quartile data shown in FIG. 10B-10D.

| | Q1 | Q2 | Q3 | Q4 |
|---|---|---|---|---|
| pY-H3 | | | | |
| (p-value) | | | | |
| Q1 | — | 0.450 | 0.766 | 0.787 |
| Q2 | 0.450 | — | 0.340 | 0.414 |
| Q3 | 0.766 | 0.340 | — | 0.990 |
| H11D | | | | |
| (p-value) | | | | |
| Q1 | — | 0.236 | 0.241 | 0.844 |
| Q2 | 0.236 | — | 0.723 | 0.177 |
| Q3 | 0.241 | 0.723 | — | 0.177 |

### 2C2-YTE Anti-Proliferative Activity and High Levels of EGFR Homodimers and Phosphorylated ErbB3 in SCCHN Cells

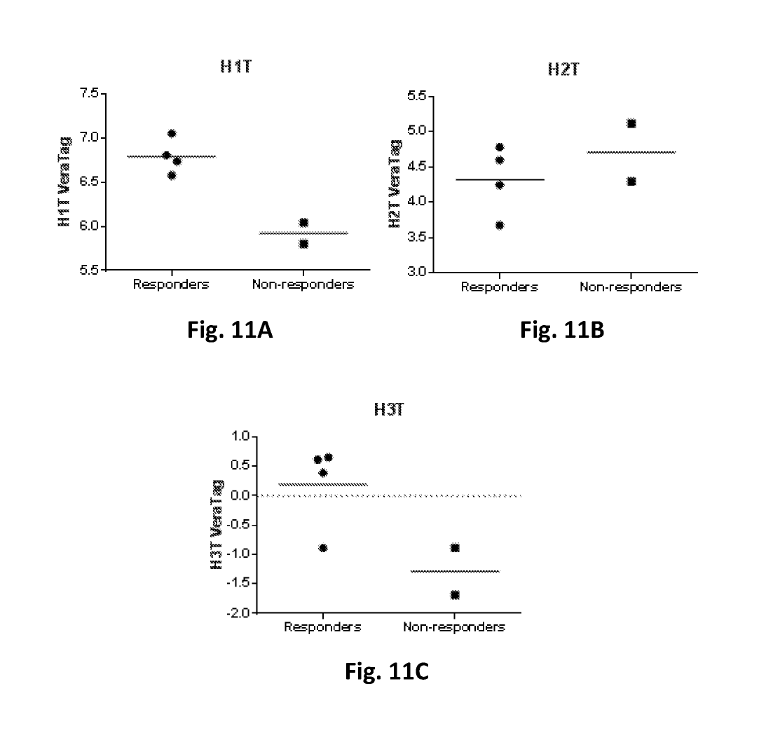

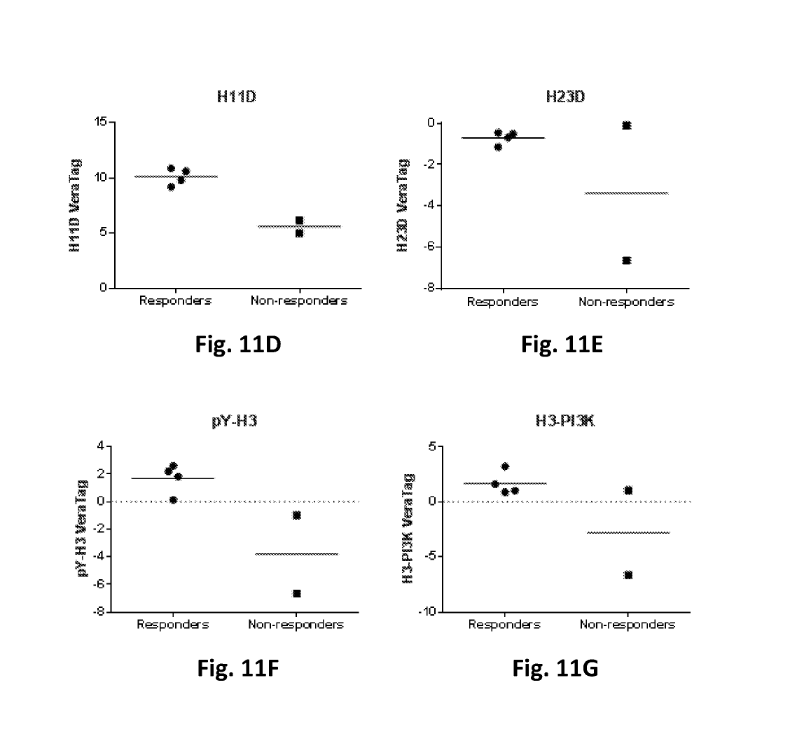

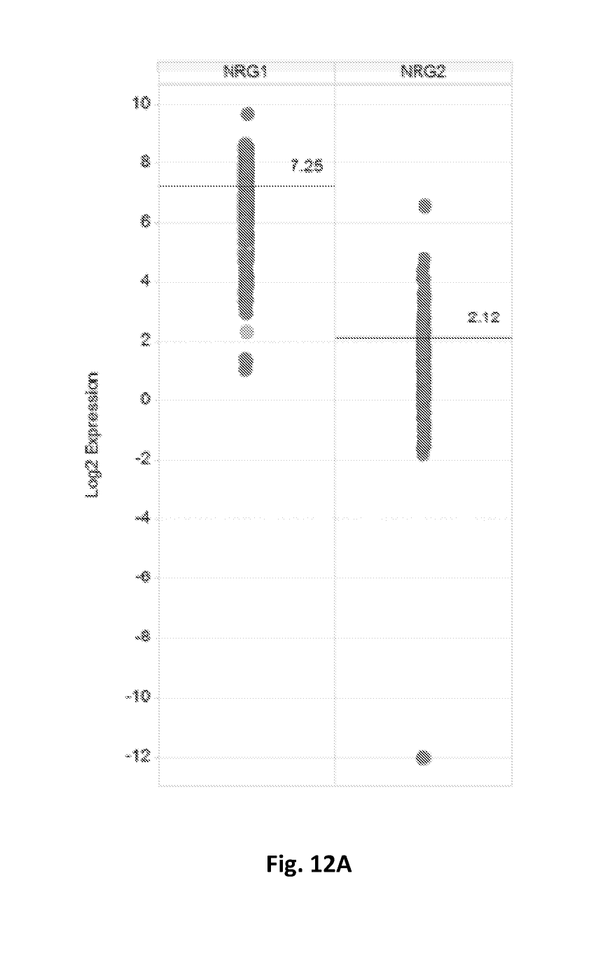

Next, correlation between 2C2-YTE anti-proliferative activity and (i) EGFR homodimers (H11D), (ii) phosphorylated ErbB3 (pY-H3) was evaluated, as was the pY-H3 pharmacodynamic ratio, the results of which are shown in FIG. 11 and summarized in Table 10, below. High levels (basal; pre-treatment with 2C2-YTE) of both EGFR homodimers (H11D) and phosphorylated ErbB3 (pY-H3) were found to correlate with 2C2-YTE antitumor activity. These data indicate that the levels of EGFR homodimer and the levels of phosphorylated ErbB3 can be utilized to determine the sensitivity of cells, e.g., SCCHN tumor cells, to Her3 inhibitors such as 2C2-YTE. In addition, especially in view of the results presented above regarding the effects of the EGFR inhibitor cetuximab on the cell surface expression of ErbB3, these data also support the use of the levels of EGFR homodimer and the levels of phosphorylated ErbB3 in determining the sensitivity of cells, e.g., SCCHN tumor cells, to treatment with a combination of HER3 inhibitors, such as 2C2-YTE, and EGFR inhibitors, such as cetuximab. Moreover, these data also indicate that phosphorylated ErbB3 can act as a pharmacodynamic marker for 2C2-YTE treatment and antitumor activity.

TABLE 10

| SCCHN | 2C2-YTE | H11D | pY-H3 | pY-H3 |
|---|---|---|---|---|
| Cell | Antiproliferative | (control*/ | (control*/ | pharmacodynamic |
| line | Activity | 2C2-YTE) | 2C2-YTE) | (PD) ratio |
| SCC61 | ++ | 1880/1200 | 6.1/0.6 | 10.6 |
| CAL27 | ++ | 1560/1270 | 4.6/0.6 | 7.5 |
| UNC7 | ++ | 581/538 | 3.5/0.4 | 8.4 |
| SCC35 | + | 890/933 | 1.1/0.4 | 2.8 |
| UNC10 | − | 32/30 | 0 | 0 |
| 4213 | − | 71/74 | 0.5/0.6 | 1.0 |
| (non- | | | | |
| cancerous | | | | |
| skin cell | | | | |
| line) | | | | |

*Control IgG1 treatment.

The level of EGFR homodimers (H11D), total EGFR (H1T), ErbB3-PI3k, and NRG compared to tumor growth inhibition mediated by 2C2-YTE were then evaluated primary SCCHN NRG1-expressing tumor samples (FIG. 11A-11G; Table 11). Treatment of the majority of primary NRG-expressing SCCHN tumors with 2C2-YTE resulted in an antitumor effect (≥30% TGI; 8/9). While not statistically significant, possibly due to small sample size, the results of the study demonstrate a trend toward high levels of EGFR homodimers correlating with 2C2-YTE antitumor activity.

Table 11 provides the level of tumor growth inhibition in a panel of NRG-expressing primary human SCCHN tumors treated with 2C2-YTE.

| Tumor | H1T | H11D | HER3-PI3k | H3T | pY-H3 | NRG | % TGI |
|---|---|---|---|---|---|---|---|
| 1 | 50.39 | 1720.12 | 2.42 | 0.38 | 0.58 | 3-4 | 78 |
| 2 | 9.02 | 16.06 | 2.25 | 0.54 | 0.73 | 3-4 | 11 |
| 3 | 18.46 | 649.34 | 2.47 | 0.88 | 0.87 | 3-4 | 40 |
| 4 | 28.06 | 1222.11 | 2.01 | 1.07 | 0.79 | ND | 50 |
| 5 | 16.18 | 10.22 | 3.33 | 5.46 | 0.89 | 1 | 30 |
| 6 | 15.81 | 16.80 | 3.07 | 1.62 | 1.10 | 2 | 37 |
| 7 | 5.08 | 14.35 | 3.11 | 0.65 | 0.71 | 1 | ND |
| 8 | 6.87 | 162.66 | 1.44 | 0.88 | 0.83 | 3-4 | 59 |
| 9 | 26.82 | 1400.49 | 0.90 | 1.83 | 0.63 | 3-4 | 35 |
| 10 | 15.74 | 81.58 | 1.76 | 0.52 | 0.60 | 1 | 63 |
| 11 | 2.76 | 3.45 | 2.60 | 1.50 | 0.91 | 1 | ND |

### Example 3: Expression of NRG1 and NRG2 in B-Raf Mutated Thyroid Cancer and Melanoma

Tumor types with a high prevalence of BRAF mutation express NRG1 and/or NRG2.

### Normalization of Mined Expression Data

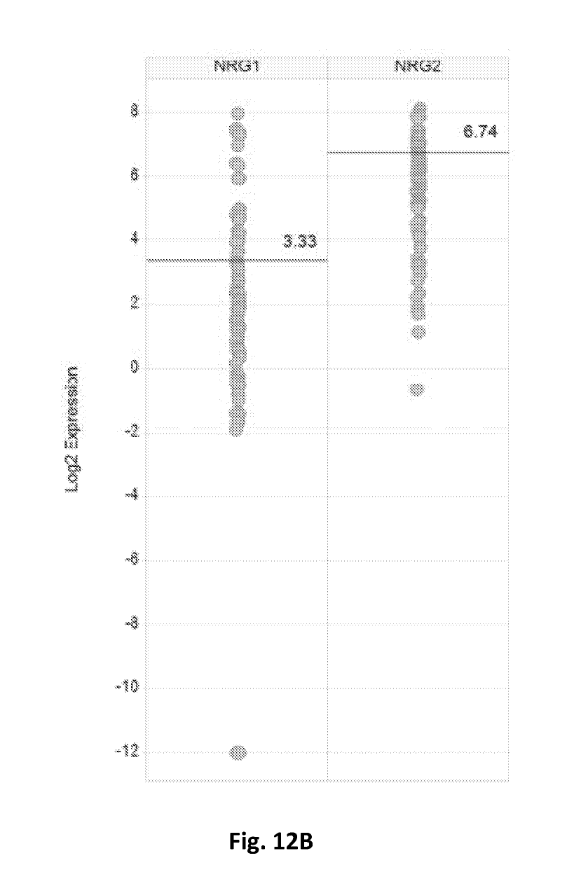

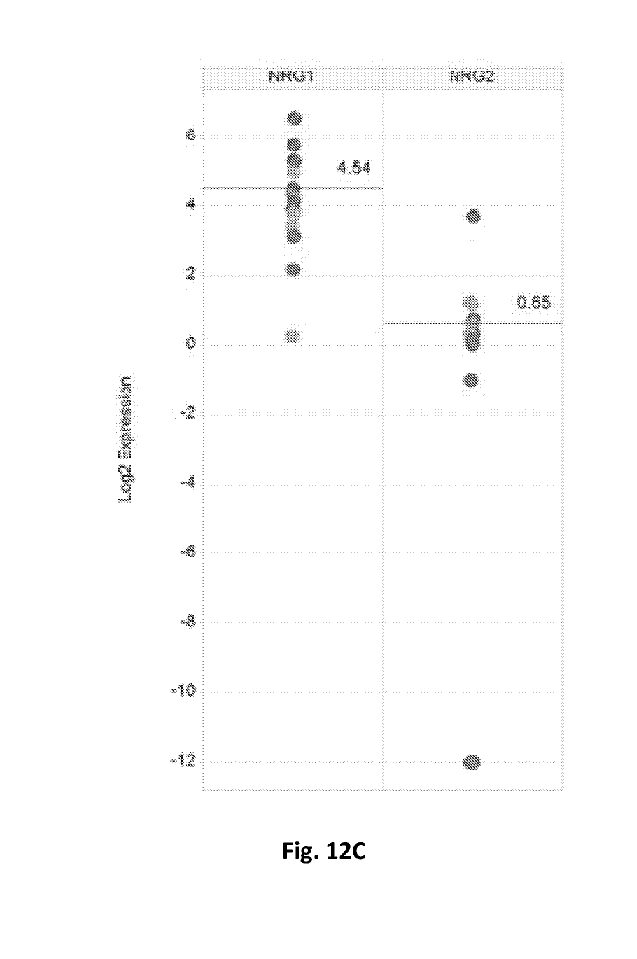

The collection of all log 2 median-centered data from each of Affymetrix U133A (FIG. 12A), Affymetrix U133 2.0 (FIG. 12B), and Affymetrix U133 Plus 2.0 (FIG. 12C) platforms from ONCOMINE® was extracted and quantile normalized. ONCOMINE® refers to a cancer microarray database and web-based data-mining platform aimed at facilitating discovery from genome-wide expression analyses (see, e.g., Rhodes et al., 2004, Neoplasia 6(1):1-6). For each gene, a single “best” reporter was chosen by the greatest variance and the largest expression. Expression values were ranked and the average value for each rank across all datasets established a reference distribution. The reference distribution was mapped to original dataset values creating an identical distribution across all samples. Individual quantile normalized datasets were then aggregated into a single meta-dataset.

### Overexpression of NRG1 and NRG2 in Selected Cancer Types

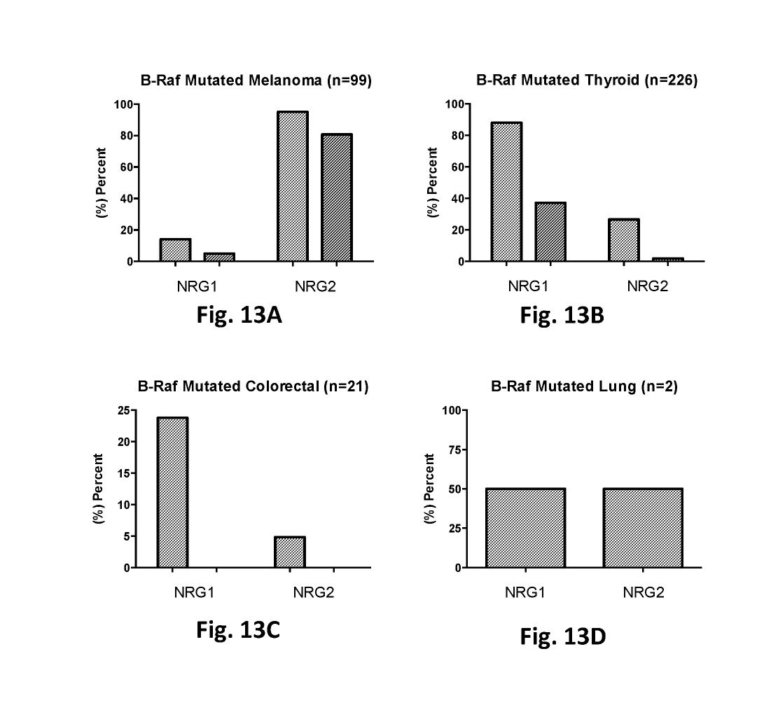

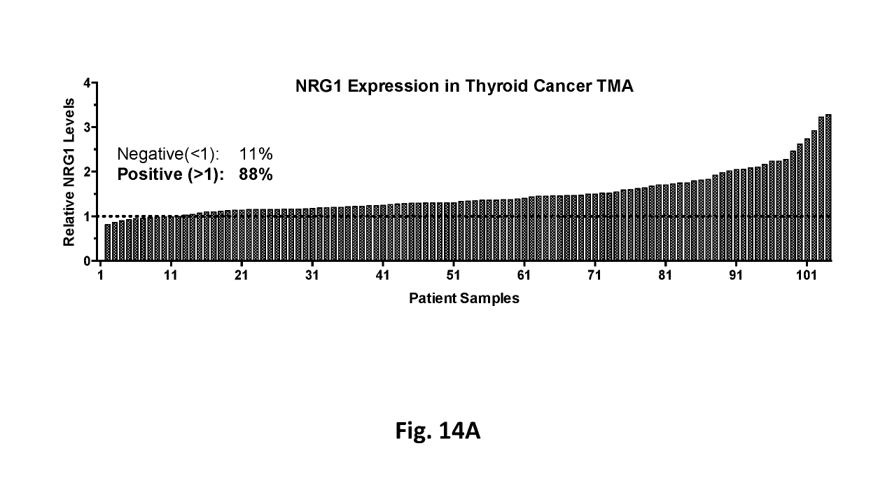

Using the mined expression data described above, the frequency of overexpression of NRG1 and NRG2 within cancer types was calculated by counting the number of samples and dividing by the total number of samples for each cancer type, then multiplying by 100. In FIGS. 13A-C, the left-hand bars for NRG1 and NRG2 indicate percentage of samples with expression above the median, and the right-hand bars for NRG1 and NRG2 indicate the percentage of samples with expression above the third quartile. In FIG. 13D, the bars indicate percentage of samples with expression above the median. NRG1 was found to be highly expressed in B-Raf mutated thyroid cancer (FIG. 13B), and NRG2 was found to be highly expressed in B-Raf mutated melanoma (FIG. 13A). B-Raf mutated colorectal cancer and B-Raf mutated lung expression levels for NRG1 and NRG2 were also calculated (FIGS. 13C-D).

### Further Characterization of NRG1 and NRG2 Expression in Selected Cancer Types

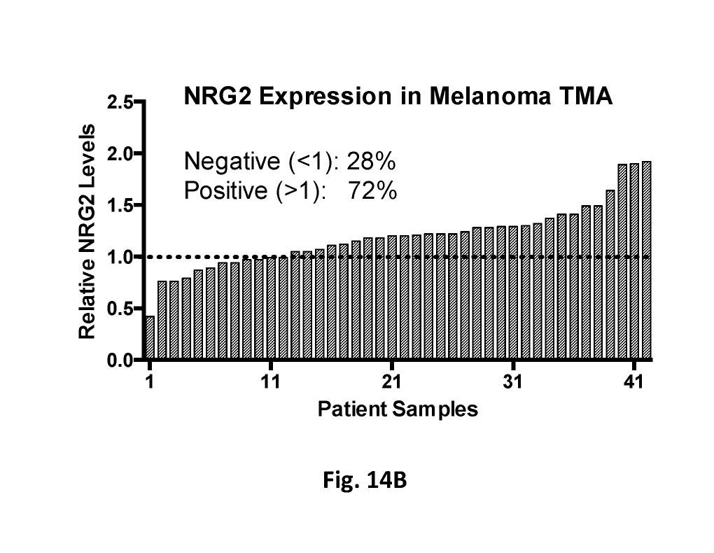

FIG. 14A depicts the level of NRG1 mRNA over background mRNA levels in individual thyroid cancer samples. Each bar represents an individual thyroid cancer patient sample. The frequency of B-Raf mutated tumors in the thyroid samples was 40-50%. FIG. 14B depicts the level of NRG2 mRNA over background mRNA levels in individual melanoma samples. Each bar represents an melanoma patient sample. The frequency of B-Raf mutated tumors in the melanoma samples was about 50%. Background is defined as AQUA (Genoptix; Carlsbad Calif.) score of a non-specific probe, and is indicated by the dotted line. NRG1 and NRG2 were found to be highly prevalent in thyroid cancer and melanoma, respectively, with 88% of thyroid cancers tested expressing NRG1, and 72% of melanomas tested expressing NRG2.

### Example 4: Potency of NRG Isoforms

Genes encoding the full extracellular domains of each NRG gene and isoform (NRG1α, NRG1β, NRG2α, and NRG2β) were modified to encode a signal peptide at their 5′ end, in order to induce secretion, and a His-tag at their 3′ end. Constructs were transfected into HEK293 cells and conditioned media was treated with NaCl to a final concentration of 0.5 M. NRG-containing media was purified by NiNTA chromatography and eluted with stepwise increases in imidazole in a phosphate buffer containing 0.5 M NaCl. NRG-containing fractions were pooled and further purified by size-exclusion chromatography with a mobile phase consisting of a phosphate-based buffer with 0.5 M NaCl. Purified NRG proteins were titrated on T47D breast cancer cells (which naturally express all ErbB receptors) for 10 minutes at 37° C. ErbB3 phosphorylation was measured by ELISA.

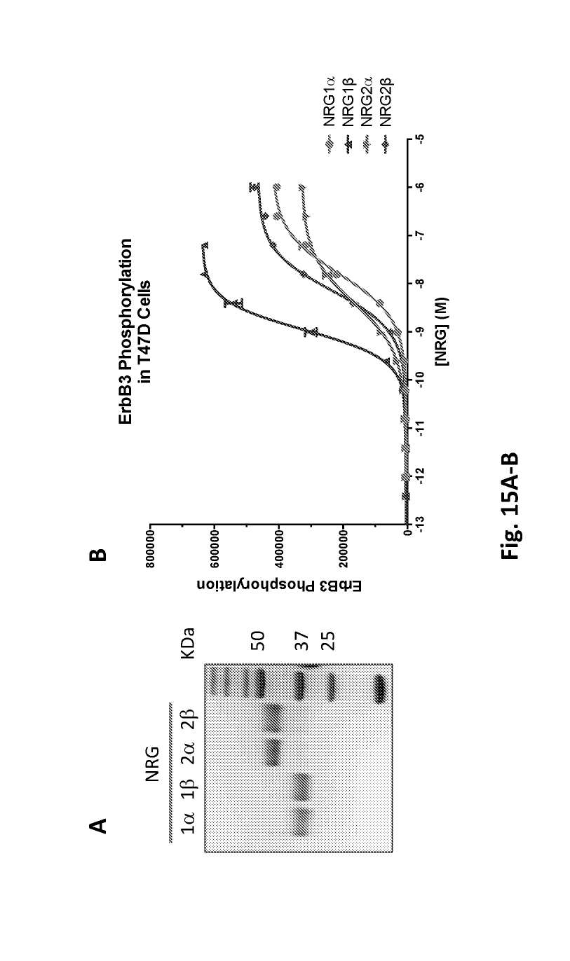

FIG. 15A shows a protein gel of the purified NRG isoforms after NiNTA and size exclusion chromatography. FIG. 15B shows the titration of the purified NRG proteins on T47D cells. All of the isoforms were shown to stimulate ErbB3 phosphorylation in T47D cells. NRG1β activated ErbB3 with an approximately 10-fold greater potency than the other NRG isoforms (FIG. 15B).

### Example 5: 2C2-YTE Activity Correlates with High Levels of EGFR Homodimers

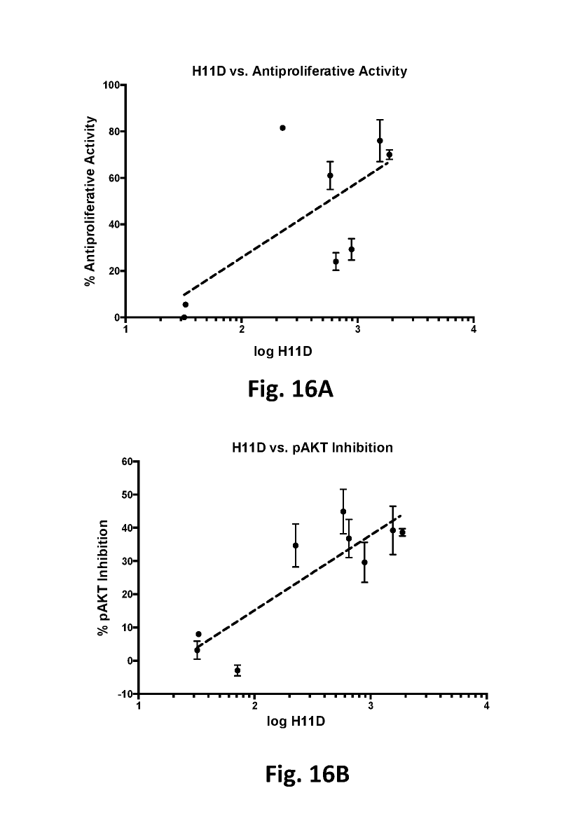

FIGS. 16A-16B depict the correlation of high levels of EGFR homodimer (H11D) with the percent antiproliferative activity (FIG. 16A), and percent Akt phosphorylation (FIG. 16B) upon treatment with 2C2-YTE. A linear regression was performed on the VeraTag data (x-axis of 16A and B) and antiproliferation data (y-axis of 16A), and pAKT inhibition data from cell lines (y-axis of 16B). pAKT data was generated as follows:

Phospho-AKT antibody pairs were purchased from R&D Systems. 30,000 cells were plated in complete growth media and allowed to grow for 2 days. Cells were then treated with or without 100 nM 2C2-YTE in quadruplicate for 2 hours at 37° C. Cells were lysed with a buffer containing 50 mM Tris pH 7.4, 150 mM NaCl, 1% Triton X-100, 0.25% deoxycholate, 1 mM EDTA, 5 mM NaF, 2 mM sodium orthovanadate, and protease inhibitor tablets (Roche). Lysates and phospho-AKT standards (R&D) were added to a Meso Scale Discovery (MSD) 96-well ELISA plate that was coated with a capture AKT antibody and blocked with 1% bovine serum albumin in PBS. Lysates were incubated overnight at 4° C. with shaking, and the plates were subsequently washed with PBS with 0.05% Tween-20, and incubated with an AKT detection antibody for 2 hours at room temperature. After washing, plates were incubated with streptavidin-sulfotag (MSD) for 1 hour at room temperature. Plates were washed, incubated with read buffer (MSD) and read in a Meso Scale Discovery QuickPlex SQ 120 reader. 2C2-YTE-mediated inhibition was calculated by obtaining the ratio of pAKT (in pg/mL) in treated versus the control cells. High levels of EGFR homodimers were found to enrich for 2C2-YTE activity.

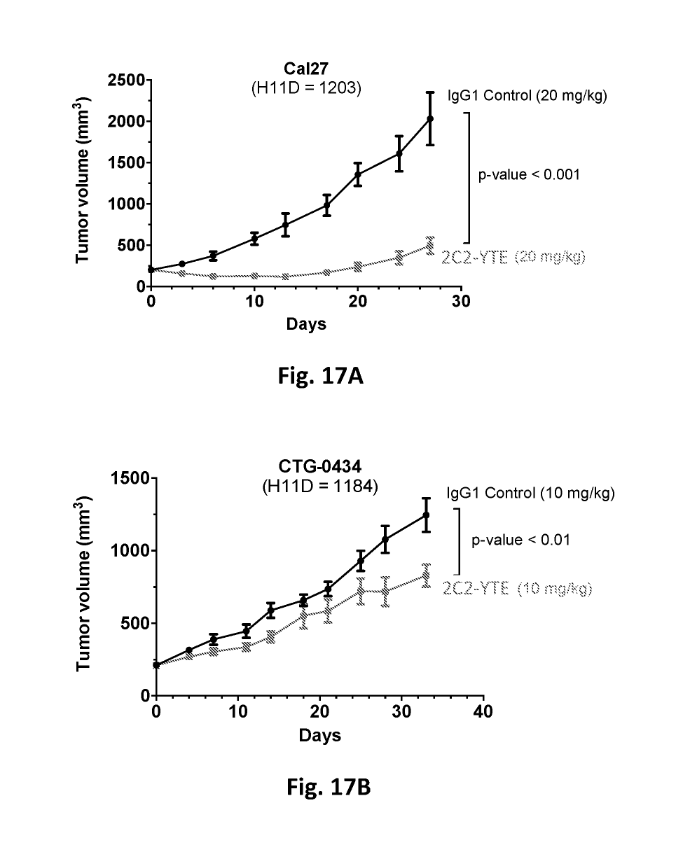

FIGS. 17A-17B depict the reduction in tumor volume over time (days) for 2C2-YTE in comparison with an IgG1 control antibody in Cal27 cells (FIG. 17A), and CTG-0434 cells (FIG. 17B). Models were pre-selected for NRG positivity and high H11D (the VeraTag output is indicated below the cell line/tumor fragment). 1×10⁷ Cal27 cells were implanted in a 1:1 mixture with matrigel (FIG. 17A). Patient-derived xenograft CTG0434 tumor fragments were implanted subcutaneously in NCR nude female mice, and the study was conducted by Champions Oncology (FIG. 17B). Antibodies were dosed twice weekly intraperitoneally. Percent tumor growth inhibition (TGI) was determined by the formula % TGI=[(Ct−C0)−(Tt−T0)]/(Ct−C0)×100 where Tt=treated arm tumor volume at time t; T0=treated arm tumor volume at the start of dosing; Ct=control arm tumor volume at time t; C0=control tumor volume at the start of dosing. All agents were formulated in PBS. Statistical significance was by performing a student's t-test using GraphPad Prism.

### INCORPORATION BY REFERENCE

All of the references cited above, as well as all references cited herein, are incorporated herein by reference in their entireties.

All publications, including patent application publications, and patents mentioned herein are hereby incorporated by reference in their entirety as if each individual publication or patent was specifically and individually indicated to be incorporated by reference.

What is claimed is:

1. A method of treating a cancer, comprising administering to a patient diagnosed with said cancer a therapeutically effective amount of a HER3 inhibitor, an EGFR inhibitor, or a combination of a HER3 inhibitor and an EGFR inhibitor, wherein the patient has a tumor that has been characterized in that cells from the tumor: i) express a neuregulin, and ii) express high levels of one or more of amphiregulin (AREG), TGF-α, and EGFR homodimer.

2. The method of claim 1, wherein cells from the tumor express high levels of amphiregulin.

3. The method of claim 1 or claim 2, wherein cells from the tumor express high levels of TGF-α.

4. The method of any one of claims 1-3, wherein cells from the tumor express high levels of EGFR homodimer.

5. The method of any one of claims 1-4, wherein cells from the tumor express high levels of a neuregulin.

6. The method of any one of claims 1-5, wherein the neuregulin is neuregulin 1 (NRG1).

7. The method of claim 6, wherein the neuregulin is NRG1α.

8. The method of claim 6, wherein the neuregulin is NRG1β.

9. The method of any one of claims 1-8, wherein the neuregulin is neuregulin 2 (NRG2).

10. The method of claim 9, wherein the neuregulin is NRG2α.

11. The method of claim 9, wherein the neuregulin is NRG2β.

12. The method of any one of claims 1-11, comprising administering a therapeutically effective amount of a combination of a HER3 inhibitor and an EGFR inhibitor.

13. The method of any one of claims 1-11, comprising administering a therapeutically effective amount of a HER3 inhibitor.

14. The method of any one of claims 1-13, wherein the HER3 inhibitor is an anti-HER3 antibody or antigen-binding fragment thereof.

15. The method of claim 14, wherein the anti-HER3 antibody or antigen-binding fragment thereof specifically binds to the same HER3 epitope as an antibody or antigen-binding fragment thereof comprising the heavy chain variable region (VH) and light chain variable region (VL) of CL16 or 2C2.

16. The method of claim 15, wherein the VH and VL of CL16 comprise SEQ ID NOs: 2 and 1, respectively, and the VH and VL of 2C2 comprise SEQ ID NOs: 2 and 3, respectively.

17. The method of any one of claims 14-16, wherein the anti-HER3 antibody or antigen-binding fragment thereof is affinity matured.

18. The method of claim 16 or 17, wherein the anti-HER3 antibody or antigen-binding fragment thereof comprises an antibody VL and an antibody VH, wherein the VL comprises the amino acid sequence:
[FW1]X₁GSX₂SNIGLNYVS[FW2]RNNQRPS[FW3]AAWDDX₃X₄X₅GEX₆
[FW4]
wherein [FW1], [FW2], [FW3] and [FW4] represent VL framework regions, and wherein
(a) X₁ represents amino acid residues Arginine (R) or Serine (S),
(b) X₂ represents amino acid residues Serine (S) or Leucine (L),
(c) X₃ represents amino acid residues Serine (S) or Glycine (G),
(d) X₄ represents amino acid residues Leucine (L) or Proline (P),
(e) X₅ represents amino acid residues Arginine (R), Isoleucine (I), Proline (P) or Serine (S), and
(f) X₆ represents amino acid residues Valine (V) or Alanine (A), and
wherein the VH comprises the amino acid sequence:
[FW5]YYYMQ[FW6]X₇IGSSGGVTNYADSVKG[FW7]VGLGDAFDI[FW8]
wherein [FW5], [FW6],[FW7] and [FW8] represent VH framework regions, and wherein X₇ represents amino acid residues Tyrosine (Y), Isoleucine (I) or Valine (V).

19. The method of claim 17, wherein FW1 comprises SEQ ID NO: 40 or 44, FW2 comprises SEQ ID NO: 41, FW3 comprises SEQ ID NO: 42, FW4 comprises SEQ ID NO: 43, FW5 comprises SEQ ID NO: 36, FW6 comprises SEQ ID NO: 37, FW7 comprises SEQ ID NO: 38, and FW8 comprises SEQ ID NO: 39.

20. The method of claim 14, wherein the anti-HER3 antibody or antigen-binding fragment thereof comprises a VL and a VH comprising VL-CDR1, VL-CRD2, VL-CDR3, VH-CDR1, VH-CDR2, and VH-CDR3 amino acid sequences identical or identical except for four, three, two, or one amino acid substitutions in one or more CDRs to: SEQ ID NOs: 18, 21, 22, 31, 32, and 35, SEQ ID NOs: 18, 21, 26, 31, 32 and 35, SEQ ID NOs: 18, 21, 27, 31, 32 and 35, SEQ ID NOs: 20, 21, 22, 31, 32 and 35, SEQ ID NOs: 19, 21, 22, 31, 32 and 35, SEQ ID NOs: 18, 21, 25, 31, 32 and 35, SEQ ID NOs: 18, 21, 28, 31, 32 and 35, SEQ ID NOs: 18, 21, 29, 31, 32 and 35, SEQ ID NOs: 18, 21, 30, 31, 32 and 35, SEQ ID NOs: 18, 21, 23, 31, 32 and 35, SEQ ID NOs: 19, 21, 23, 31, 32 and 35, SEQ ID NOs: 20, 21, 23, 31, 32 and 35, SEQ ID NOs: 18, 21, 24, 31, 32 and 35, or SEQ ID NOs: 18, 21, 25, 31, 32 and 35, respectively.

21. The method of claim 14, wherein the anti-HER3 antibody or antigen-binding fragment thereof comprises an antibody VL and an antibody VH, wherein the VL comprises an amino acid sequence at least about 90% to about 100% identical to a reference amino acid sequence selected from the group consisting of SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 14, SEQ ID NO: 15, SEQ ID NO: 16, and SEQ ID NO: 17.

22. The method of claim 14, wherein the anti-HER3 antibody or antigen-binding fragment thereof comprises an antibody VL and an antibody VH, wherein the VH comprises an amino acid sequence at least about 90% to about 100% identical to a reference amino acid sequence selected from the group consisting of SEQ ID NO: 2, SEQ ID NO: 12 and SEQ ID NO: 13.

23. The method of claim 14, wherein the anti-HER3 antibody or antigen-binding fragment thereof comprises a VL comprising a sequence at least about 90% to about 100% identical to a reference amino acid sequence selected from the group consisting of SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 14, SEQ ID NO: 15, SEQ ID NO: 16, and SEQ ID NO: 17, and wherein the anti-HER3 antibody or antigen-binding fragment comprises a VH comprising a sequence at least about 90% to about 100% identical to a reference amino acid sequence selected from the group consisting of SEQ ID NO: 2, SEQ ID NO: 12 and SEQ ID NO: 13.

24. The method of claim 14, wherein the anti-HER3 antibody or antigen-binding fragment thereof comprises a VL comprising the VL consensus sequence provided in Table 3 and a VH comprising the VH consensus sequence provided in Table 3.

25. The method of claim 14, wherein the anti-HER3 antibody or antigen-binding fragment thereof comprises a VL-CDR1, a VL-CDR2 and a VL-CDR3 comprising SEQ ID NO: 19, SEQ ID NO: 21 and SEQ ID NO: 23, respectively, and a VH-CDR1, a VH-CDR2, and a VH-CDR3 comprising SEQ ID NO: 31, SEQ ID NO: 32, and SEQ ID NO: 35, respectively.

26. The method of claim 14, wherein the anti-HER3 antibody or antigen-binding fragment thereof comprises a VL comprising SEQ ID NO: 3 and a VH comprising SEQ ID NO: 2.

27. The method of any one of claims 18-26, wherein the anti-HER3 antibody or antigen-binding fragment thereof comprises a human heavy chain constant region or fragment thereof.

28. The method of claim 27, wherein the heavy chain constant region or fragment thereof is an IgG constant region.

29. The method of claim 28, wherein the IgG constant region is selected from an IgG1 constant region, an IgG2 constant region, an IgG3 constant region and an IgG4 constant region.

30. The method of claim 28, wherein the IgG constant region is an IgG1 constant region.

31. The method of any one of claims 18-30, wherein the anti-HER3 antibody or antigen-binding fragment comprises a light chain constant region selected from the group consisting of a human kappa constant region and a human lambda constant region.

32. The method of claim 31, wherein the anti-HER3 antibody or antigen-binding fragment comprises a human lambda constant region.

33. The method of any one of claims 27-32, wherein the IgG constant domain comprises one or more amino acid substitutions relative to a wild-type IgG constant domain wherein the modified IgG has an increased half-life compared to the half-life of an IgG having the wild-type IgG constant domain.

34. The method of any one of claims 28-33, wherein the IgG constant domain comprises one or more amino acid substitutions of amino acid residues at positions 251-257, 285-290, 308-314, 385-389, and 428-436, wherein the numbering is according to the EU index as set forth in Kabat.

35. The method of claim 34, wherein at least one IgG constant domain amino acid substitution is selected from the group consisting of:
(a) substitution of the amino acid at position 252 with Tyrosine (Y), Phenylalanine (F), Tryptophan (W), or Threonine (T),
(b) substitution of the amino acid at position 254 with Threonine (T),
(c) substitution of the amino acid at position 256 with Serine (S), Arginine (R), Glutamine (Q), Glutamic acid (E), Aspartic acid (D), or Threonine (T),
(d) substitution of the amino acid at position 257 with Leucine (L),
(e) substitution of the amino acid at position 309 with Proline (P),
(f) substitution of the amino acid at position 311 with Serine (S),
(g) substitution of the amino acid at position 428 with Threonine (T), Leucine (L), Phenylalanine (F), or Serine (S),
(h) substitution of the amino acid at position 433 with Arginine (R), Serine (S), Isoleucine (I), Proline (P), or Glutamine (Q),
(i) substitution of the amino acid at position 434 with Tryptophan (W), Methionine (M), Serine (S), Histidine (H), Phenylalanine (F), or Tyrosine, and
(j) a combination of two or more of said substitutions, wherein the numbering is according to the EU index as set forth in Kabat.

36. The method of claim 34, wherein the human IgG constant domain comprises amino acid substitutions relative to a wild-type human IgG constant domain at positions 252, 254, and 256, wherein
(a) the amino acid at position 252 is substituted with Tyrosine (Y),
(b) the amino acid at position 254 is substituted with Threonine (T), and
(c) the amino acid at position 256 is substituted with Glutamic acid (E),
wherein the numbering is according to the EU index as set forth in Kabat.

37. The method of any one of claims 27-32, wherein the human IgG constant domain comprises amino acid substitutions relative to a wild-type human IgG constant domain at positions 252, 254, and 256, wherein
(a) the amino acid at position 252 is substituted with Tyrosine (Y),
(b) the amino acid at position 254 is substituted with Threonine (T), and
(c) the amino acid at position 256 is substituted with Glutamic acid (E),
wherein the numbering is according to the EU index as set forth in Kabat.

38. The method of any one of claims 34-37, wherein the amino acid at position 434 is substituted with an amino acid selected from the group consisting of Tryptophan (W), Methionine (M), Tyrosine (Y), and Serine (S), and wherein the numbering is according to the EU index as set forth in Kabat.

39. The method of claim 38, wherein the amino acid at position 428 is substituted with an amino acid selected from the group consisting of Threonine (T), Leucine (L), Phenylalanine (F), and Serine (S), and wherein the numbering is according to the EU index as set forth in Kabat.

40. The method of claim 38, wherein the amino acid at position 257 is substituted with Leucine (L), and the amino acid at Kabat position 434 is substituted with Tyrosine (Y), and wherein the numbering is according to the EU index as set forth in Kabat.

41. The method of claim 9 wherein the amino acid at Kabat position 428 is substituted with Leucine (L), and the amino acid at Kabat position 434 is substituted with Serine (S).

42. The method of claim 14, wherein the anti-HER3 antibody or antigen-binding fragment thereof comprises an antibody VL of SEQ ID NO:3, an antibody VH of SEQ ID NO: 2, and an IgG1 constant region of SEQ ID 46.

43. The method of claim 41, wherein the human IgG constant region comprises amino acid substitutions relative to a wild-type human IgG constant domain at positions 252, 254, and 256, wherein the numbering is according to the EU index as set forth in Kabat, and wherein
(a) the amino acid at position 252 is substituted with Tyrosine (Y),
(b) the amino acid at position 254 is substituted with Threonine (T), and
(c) the amino acid at position 256 is substituted with Glutamic acid (E).

44. The method of claim 25, wherein the anti-HER3 antibody or antigen-binding fragment thereof comprises a human IgG1 constant region and a human lambda constant region.

45. The method of claim 44, wherein the IgG1 constant region comprises amino acid substitutions relative to a wild-type human IgG constant domain at positions 252, 254, and 256, wherein the numbering is according to the EU index as set forth in Kabat, and wherein
(a) the amino acid at position 252 is substituted with Tyrosine (Y),
(b) the amino acid at position 254 is substituted with Threonine (T), and
(c) the amino acid at position 256 is substituted with Glutamic acid (E).

46. The method of claim 26, wherein the anti-HER3 antibody or antigen-binding fragment thereof comprises a human IgG1 constant region and a human lambda constant region.

47. The method of claim 46, wherein the IgG1 constant region comprises amino acid substitutions relative to a wild-type human IgG constant domain at positions 252, 254, and 256, wherein the numbering is according to the EU index as set forth in Kabat, and wherein
(a) the amino acid at position 252 is substituted with Tyrosine (Y),
(b) the amino acid at position 254 is substituted with Threonine (T), and
(c) the amino acid at position 256 is substituted with Glutamic acid (E).

48. The method of any one of claims 14-47, wherein the anti-HER3 antibody is a human antibody, a humanized antibody, a chimeric antibody, a monoclonal antibody, a polyclonal antibody, a recombinant antibody, a multispecific antibody, or an antigen-binding fragment thereof.

49. The method of claim 48, wherein the anti-HER3 antibody is a human antibody.

50. The method of any one of claims 14-49, which antigen-binding fragment is Fv, Fab, F(ab′)2, Fab′, dsFv, scFv, and sc(Fv)2.

51. The method of any one of claims 14-50, which anti-HER3 antibody or antigen-binding fragment is conjugated to at least one heterologous agent.

52. The method of any one of claims 1-11, comprising administering an EGFR inhibitor.

53. The method of claim 12 or 52, wherein the EGFR inhibitor is an anti-EGFR antibody or an antigen-binding fragment thereof.

54. The method of claim 53, wherein the anti-EGFR antibody is cetuximab.

55. The method of any one of claims 1-54, wherein the cancer is a squamous cell carcinoma of the head and neck (SCCHN).

56. The method of claim 1, wherein the cancer is a SCCHN, comprising administering to a patient diagnosed with said SCCHN a therapeutically effective amount of a HER3 inhibitor and an EGFR inhibitor, wherein the HER3 inhibitor is an antibody comprising a VL that comprises SEQ ID NO: 3, a human lambda constant region, a VH that comprises SEQ ID NO: 2, and a human IgG1 constant region comprising amino acid substitutions relative to a wild-type human IgG constant domain at positions 252, 254, and 256, wherein
(a) the amino acid at position 252 is substituted with Tyrosine (Y),
(b) the amino acid at position 254 is substituted with Threonine (T), and
(c) the amino acid at position 256 is substituted with Glutamic acid (E),
wherein the numbering is according to the EU index as set forth in Kabat; and wherein the EGFR inhibitor is cetuximab.

57. A method of determining whether a patient diagnosed with cancer is indicated as likely to be responsive to treatment with a HER3 inhibitor, an EGFR inhibitor, or a combination of a HER3 inhibitor and an EGFR inhibitor, comprising measuring the expression of one or more of AREG, TGF-α, and EGFR homodimer in a sample from the patient, wherein the sample comprises tumor cells and a neuregulin, and wherein high levels of one or more of AREG, TGF-α, and EGFR homodimer in the sample indicates that the patient is likely to be responsive to treatment with a HER3 inhibitor, an EGFR inhibitor, or a combination of a HER3 inhibitor and an EGFR inhibitor.

58. A method of determining whether a patient diagnosed with cancer is indicated as likely to be responsive to treatment with a HER3 inhibitor, an EGFR inhibitor, or a combination of a HER3 inhibitor and an EGFR inhibitor, comprising measuring the expression of a neuregulin, and at least one of AREG, TGF-α, and EGFR homodimer in a sample from the patient, wherein the sample comprises tumor cells, and wherein the presence of the neuregulin and high levels of one or more of AREG, TGF-α, and EGFR homodimer in the sample indicates that the patient is likely to be responsive to treatment with a HER3 inhibitor, an EGFR inhibitor, or a combination of a HER3 inhibitor and an EGFR inhibitor.

59. The method of claim 57 or 58, wherein the method comprises measuring the expression of AREG in the sample.

60. The method of any one of claims 57-59, wherein the method comprises measuring the expression of TGF-α in the sample.

61. The method of any one of claims 57-59, wherein the method comprises measuring the expression of EGFR homodimer in the sample.

62. The method of any one of claims 57-60, wherein the level of neuregulin in the sample indicates a high level of neuregulin.

63. The method of any one of claims 57-62, wherein the neuregulin is NRG1.

64. The method of claim 63, wherein the neuregulin is NRG1α.

65. The method of claim 63, wherein the neuregulin is NRG1β.

66. The method of any one of claims 57-65, wherein the neuregulin is NRG2.

67. The method of claim 66, wherein the neuregulin is NRG2α.

68. The method of claim 66, wherein the neuregulin is NRG2β.

69. The method of any one of claims 57-68, which is a method of determining whether a patient diagnosed with cancer is indicated as likely to be responsive to treatment with a HER3 inhibitor and an EGFR inhibitor.

70. The method of any one of claims 57-68, which is a method of determining whether a patient diagnosed with cancer is indicated as likely to be responsive to treatment with a HER3 inhibitor.

71. The method of any one of claims 57-60, wherein the HER3 inhibitor is an anti-HER3 antibody or an antigen-binding fragment thereof.

72. The method of claim 71, wherein the anti-HER3 antibody or antigen-binding fragment thereof specifically binds to the same HER3 epitope as an antibody or antigen-binding fragment thereof comprising the heavy chain variable region (VH) and light chain variable region (VL) of CL16 or 2C2.

73. The method of claim 72, wherein the VH and VL of CL16 comprise SEQ ID NOs: 2 and 1, respectively, and the VH and VL of 2C2 comprise SEQ ID NOs: 2 and 3, respectively.

74. The method of any one of claims 71-73, wherein the anti-HER3 antibody or antigen-binding fragment thereof is affinity matured.

75. The method of any one of claims 71-74, wherein the anti-HER3 antibody or antigen-binding fragment thereof comprises an antibody VL and an antibody VH, wherein the VL comprises the amino acid sequence:
[FW1]X₁GSX₂SNIGLNYVS[FW2]RNNQRPS[FW3]AAWDDX₃X₄X₅GEX₆
[FW4]
wherein [FW1], [FW2], [FW3] and [FW4] represent VL framework regions, and wherein
(a) X₁ represents amino acid residues Arginine (R) or Serine (S),
(b) X₂ represents amino acid residues Serine (S) or Leucine (L),
(c) X₃ represents amino acid residues Serine (S) or Glycine (G),
(d) X₄ represents amino acid residues Leucine (L) or Proline (P),
(e) X₅ represents amino acid residues Arginine (R), Isoleucine (I), Proline (P) or Serine (S), and
(f) X₆ represents amino acid residues Valine (V) or Alanine (A), and
wherein the VH comprises the amino acid sequence:
[FW5]YYYMQ[FW6]X₇IGSSGGVTNYADSVKG[FW7]VGLGDAFDI[FW8]
wherein [FW5], [FW6],[FW7] and [FW8] represent VH framework regions, and wherein X₇ represents amino acid residues Tyrosine (Y), Isoleucine (I) or Valine (V).

76. The method of claim 75, wherein FW1 comprises SEQ ID NO: 40 or 44, FW2 comprises SEQ ID NO: 41, FW3 comprises SEQ ID NO: 42, FW4 comprises SEQ ID NO: 43, FW5 comprises SEQ ID NO: 36, FW6 comprises SEQ ID NO: 37, FW7 comprises SEQ ID NO: 38, and FW8 comprises SEQ ID NO: 39.

77. The method of claim 71, wherein the anti-HER3 antibody or antigen-binding fragment thereof comprises a VL and a VH comprising VL-CDR1, VL-CRD2, VL-CDR3, VH-CDR1, VH-CDR2, and VH-CDR3 amino acid sequences identical or identical except for four, three, two, or one amino acid substitutions in one or more CDRs to: SEQ ID NOs: 18, 21, 22, 31, 32, and 35, SEQ ID NOs: 18, 21, 26, 31, 32 and 35, SEQ ID NOs: 18, 21, 27, 31, 32 and 35, SEQ ID NOs: 20, 21, 22, 31, 32 and 35, SEQ ID NOs: 19, 21, 22, 31, 32 and 35, SEQ ID NOs: 18, 21, 25, 31, 32 and 35, SEQ ID NOs: 18, 21, 28, 31, 32 and 35, SEQ ID NOs: 18, 21, 29, 31, 32 and 35, SEQ ID NOs: 18, 21, 30, 31, 32 and 35, SEQ ID NOs: 18, 21, 23, 31, 32 and 35, SEQ ID NOs: 19, 21, 23, 31, 32 and 35, SEQ ID NOs: 20, 21, 23, 31, 32 and 35, SEQ ID NOs: 18, 21, 24, 31, 32 and 35, or SEQ ID NOs: 18, 21, 25, 31, 32 and 35, respectively.

78. The method of claim 71, wherein the anti-HER3 antibody or antigen-binding fragment thereof comprises an antibody VL and an antibody VH, wherein the VL comprises an amino acid sequence at least about 90% to about 100% identical to a reference amino acid sequence selected from the group consisting of SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 14, SEQ ID NO: 15, SEQ ID NO: 16, and SEQ ID NO: 17.

79. The method of claim 71, wherein the anti-HER3 antibody or antigen-binding fragment thereof comprises an antibody VL and an antibody VH, wherein the VH comprises an amino acid sequence at least about 90% to about 100% identical to a reference amino acid sequence selected from the group consisting of SEQ ID NO: 2, SEQ ID NO: 12 and SEQ ID NO: 13.

80. The method of claim 71, wherein the anti-HER3 antibody or antigen-binding fragment thereof comprises a VL comprising a sequence at least about 90% to about 100% identical to a reference amino acid sequence selected from the group consisting of SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 14, SEQ ID NO: 15, SEQ ID NO: 16, and SEQ ID NO: 17, and wherein the anti-HER3 antibody or antigen-binding fragment comprises a VH comprising a sequence at least about 90% to about 100% identical to a reference amino acid sequence selected from the group consisting of SEQ ID NO: 2, SEQ ID NO: 12 and SEQ ID NO: 13.

81. The method of claim 71, wherein the anti-HER3 antibody or antigen-binding fragment thereof comprises a VL comprising the VL consensus sequence provided in Table 3 and a VH comprising the VH consensus sequence provided in Table 3.

82. The method of claim 71, wherein the anti-HER3 antibody or antigen-binding fragment thereof comprises a VL-CDR1, a VL-CDR2 and a VL-CDR3 comprising SEQ ID NO: 19, SEQ ID NO: 21 and SEQ ID NO: 23, respectively, and a VH-CDR1, a VH-CDR2, and a VH-CDR3 comprising SEQ ID NO: 31, SEQ ID NO: 32, and SEQ ID NO: 35, respectively.

83. The method of claim 71, wherein the anti-HER3 antibody or antigen-binding fragment thereof comprises a VL comprising SEQ ID NO: 3 and a VH comprising SEQ ID NO: 2.

84. The method of any one of claims 75-83, wherein the anti-HER3 antibody or antigen-binding fragment thereof comprises a human heavy chain constant region or fragment thereof.

85. The method of claim 84, wherein the heavy chain constant region or fragment thereof is an IgG constant region.

86. The method of claim 85, wherein the IgG constant region is selected from an IgG1 constant region, an IgG2 constant region, an IgG3 constant region and an IgG4 constant region.

87. The method of claim 85, wherein the IgG constant region is an IgG1 constant region.

88. The method of any one of claims 75-87, wherein the anti-HER3 antibody or antigen-binding fragment comprises a light chain constant region selected from the group consisting of a human kappa constant region and a human lambda constant region.

89. The method of claim 88, wherein the anti-HER3 antibody or antigen-binding fragment comprises a human lambda constant region.

90. The method of any one of claims 84-89, wherein the IgG constant domain comprises one or more amino acid substitutions relative to a wild-type IgG constant domain wherein the modified IgG has an increased half-life compared to the half-life of an IgG having the wild-type IgG constant domain.

91. The method of any one of claims 85-90, wherein the IgG constant domain comprises one or more amino acid substitutions of amino acid residues at positions 251-257, 285-290, 308-314, 385-389, and 428-436, wherein the numbering is according to the EU index as set forth in Kabat.

92. The method of claim 91, wherein at least one IgG constant domain amino acid substitution is selected from the group consisting of:
(a) substitution of the amino acid at position 252 with Tyrosine (Y), Phenylalanine (F), Tryptophan (W), or Threonine (T),
(b) substitution of the amino acid at position 254 with Threonine (T),
(c) substitution of the amino acid at position 256 with Serine (S), Arginine (R), Glutamine (Q), Glutamic acid (E), Aspartic acid (D), or Threonine (T),
(d) substitution of the amino acid at position 257 with Leucine (L),
(e) substitution of the amino acid at position 309 with Proline (P),
(f) substitution of the amino acid at position 311 with Serine (S),
(g) substitution of the amino acid at position 428 with Threonine (T), Leucine (L), Phenylalanine (F), or Serine (S),
(h) substitution of the amino acid at position 433 with Arginine (R), Serine (S), Isoleucine (I), Proline (P), or Glutamine (Q),
(i) substitution of the amino acid at position 434 with Tryptophan (W), Methionine (M), Serine (S), Histidine (H), Phenylalanine (F), or Tyrosine, and
(j) a combination of two or more of said substitutions, wherein the numbering is according to the EU index as set forth in Kabat.

93. The method of claim 91, wherein the human IgG constant domain comprises amino acid substitutions relative to a wild-type human IgG constant domain at positions 252, 254, and 256, wherein
(a) the amino acid at position 252 is substituted with Tyrosine (Y),
(b) the amino acid at position 254 is substituted with Threonine (T), and
(c) the amino acid at position 256 is substituted with Glutamic acid (E),
wherein the numbering is according to the EU index as set forth in Kabat.

94. The method of any one of claims 84-89, wherein the human IgG constant domain comprises amino acid substitutions relative to a wild-type human IgG constant domain at positions 252, 254, and 256, wherein
(a) the amino acid at position 252 is substituted with Tyrosine (Y),
(b) the amino acid at position 254 is substituted with Threonine (T), and
(c) the amino acid at position 256 is substituted with Glutamic acid (E),
wherein the numbering is according to the EU index as set forth in Kabat.

95. The method of any one of claims 91-94, wherein the amino acid at position 434 is substituted with an amino acid selected from the group consisting of Tryptophan (W), Methionine (M), Tyrosine (Y), and Serine (S), and wherein the numbering is according to the EU index as set forth in Kabat.

96. The method of claim 95, wherein the amino acid at position 428 is substituted with an amino acid selected from the group consisting of Threonine (T), Leucine (L), Phenylalanine (F), and Serine (S), and wherein the numbering is according to the EU index as set forth in Kabat.

97. The method of claim 95, wherein the amino acid at position 257 is substituted with Leucine (L), and the amino acid at Kabat position 434 is substituted with Tyrosine (Y), and wherein the numbering is according to the EU index as set forth in Kabat.

98. The method of claim 96, wherein the amino acid at Kabat position 428 is substituted with Leucine (L), and the amino acid at Kabat position 434 is substituted with Serine (S).

99. The method of claim 71, wherein the anti-HER3 antibody or antigen-binding fragment thereof comprises an antibody VL of SEQ ID NO:3, an antibody VH of SEQ ID NO: 2, and an IgG1 constant region of SEQ ID 46.

100. The method of claim 99, wherein the human IgG constant region comprises amino acid substitutions relative to a wild-type human IgG constant domain at positions 252, 254, and 256, wherein the numbering is according to the EU index as set forth in Kabat, and wherein
(a) the amino acid at position 252 is substituted with Tyrosine (Y),
(b) the amino acid at position 254 is substituted with Threonine (T), and
(c) the amino acid at position 256 is substituted with Glutamic acid (E).

101. The method of any one of claims 71-100, wherein the HER3 inhibitor is an anti-HER3 antibody.

102. The method of claim 82, wherein the anti-HER3 antibody or antigen-binding fragment thereof comprises a human IgG1 constant region and a human lambda constant region.

103. The method of claim 102, wherein the IgG1 constant region comprises amino acid substitutions relative to a wild-type human IgG constant domain at positions 252, 254, and 256, wherein the numbering is according to the EU index as set forth in Kabat, and wherein
(a) the amino acid at position 252 is substituted with Tyrosine (Y),
(b) the amino acid at position 254 is substituted with Threonine (T), and
(c) the amino acid at position 256 is substituted with Glutamic acid (E).

104. The method of claim 83, wherein the anti-HER3 antibody or antigen-binding fragment thereof comprises a human IgG1 constant region and a human lambda constant region.

105. The method of claim 104, wherein the IgG1 constant region comprises amino acid substitutions relative to a wild-type human IgG constant domain at positions 252, 254, and 256, wherein the numbering is according to the EU index as set forth in Kabat, and wherein
(a) the amino acid at position 252 is substituted with Tyrosine (Y),
(b) the amino acid at position 254 is substituted with Threonine (T), and
(c) the amino acid at position 256 is substituted with Glutamic acid (E).

106. The method of any one of claims 71-105, wherein the anti-HER3 antibody is a human antibody, a humanized antibody, a chimeric antibody, a monoclonal antibody, a polyclonal antibody, a recombinant antibody, a multispecific antibody, or an antigen-binding fragment thereof.

107. The method of claim 106, wherein the anti-HER3 antibody is a human antibody.

108. The method of any one of claims 71-107, which antigen-binding fragment is Fv, Fab, F(ab′)2, Fab′, dsFv, scFv, and sc(Fv)2.

109. The method of any one of claims 71-108, which anti-HER3 antibody or antigen-binding fragment is conjugated to at least one heterologous agent.

110. The method of any one of claims 57-68, which is a method of determining whether a patient diagnosed with cancer is indicated as likely to be responsive to treatment with a EGFR inhibitor.

111. The method of claim 69 or 110, wherein the EGFR inhibitor is an anti-EGFR antibody or an antigen-binding fragment thereof.

112. The method of claim 111, wherein the anti-EGFR antibody is cetuximab.

113. The method of any one of claims 57-112, wherein the cancer is a squamous cell carcinoma of the head and neck (SCCHN).

114. The method of any one of claims 57-113, wherein the method comprises a first step of obtaining the sample from a tumor from the patient.

115. The method of any one of claims 57-69, wherein, if the patient diagnosed with cancer is indicated as likely to be responsive to treatment with a HER3 inhibitor and an EGFR inhibitor, the method comprises an additional step of administering to the patient a therapeutically effective amount of a HER3 inhibitor and a therapeutically effective amount of an EGFR inhibitor.

116. The method of claim 115, wherein the HER1 inhibitor is an anti-HER3 antibody or an antigen-binding fragment thereof as in any one of claims 14-51.

117. The method of claim 115 or 116, wherein the EGFR inhibitor is cetuximab.

118. A kit comprising components for performing the method of any one of claims 57-117.

119. The method of any one of claims 57-118, wherein the step of measuring the expression of EGFR homodimer comprises measuring the level of protein-protein interaction between EGFR monomers in the sample.

120. The method of claim 58, wherein, if the patient diagnosed with cancer is indicated as likely to be responsive to treatment with a HER3 inhibitor or an EGFR inhibitor and expression of neuregulin is present in the sample, the method comprises an additional step of measuring the expression of AREG in the sample, wherein high expression of AREG in the sample indicates that the patient is likely to be responsive to treatment with a HER3 inhibitor or an EGFR inhibitor.

121. The method of claim 58 or claim 120, wherein, if the patient diagnosed with cancer is indicated as likely to be responsive to treatment with a HER3 inhibitor or an EGFR inhibitor and expression of neuregulin is present in the sample, the method comprises an additional step of measuring the expression of TGF-α in the sample, wherein high expression of TGF-α in the sample indicates that the patient is likely to be responsive to treatment with a HER3 inhibitor or an EGFR inhibitor.

122. The method of claim 58, 120, or 121, wherein, if the patient diagnosed with cancer is indicated as likely to be responsive to treatment with a HER3 inhibitor or an EGFR inhibitor and expression of neuregulin is present in the sample, the method comprises an additional step of measuring the expression of EGFR homodimer in the sample, wherein high expression of EGFR homodimer in the sample indicates that the patient is likely to be responsive to treatment with a HER3 inhibitor or an EGFR inhibitor.

123. The method of claim 57, which is a method of determining whether a patient diagnosed with SCCHN is indicated as likely to be responsive to treatment with a HER3 inhibitor and an EGFR inhibitor, wherein the HER3 inhibitor is an antibody comprising a VL that comprises SEQ ID NO: 3, a human lambda constant region, a VH that comprises SEQ ID NO: 2, and a human IgG1 constant region comprising amino acid substitutions relative to a wild-type human IgG constant domain at positions 252, 254, and 256, wherein
(a) the amino acid at position 252 is substituted with Tyrosine (Y),
(b) the amino acid at position 254 is substituted with Threonine (T), and
(c) the amino acid at position 256 is substituted with Glutamic acid (E),
wherein the numbering is according to the EU index as set forth in Kabat; and wherein the EGFR inhibitor is cetuximab.

124. The method of claim 123, wherein, if the patient diagnosed with cancer is indicated as likely to be responsive to treatment with the HER3 inhibitor and the EGFR inhibitor, the method comprises an additional step of administering to the patient a therapeutically effective amount of the HER3 inhibitor and a therapeutically effective amount of the EGFR inhibitor.

125. A method of treating a cancer, comprising administering to a patient diagnosed with said cancer a therapeutically effective amount of a HER3 inhibitor, an EGFR inhibitor, or a combination of a HER3 inhibitor and an EGFR inhibitor, wherein the patient has a tumor that has been characterized in that cells from the tumor express high levels of EGFR homodimer.

126. The method of claim 125, wherein cells from the tumor express high levels of amphiregulin.

127. The method of claim 125 or claim 126, wherein cells from the tumor express high levels of TGF-α.

128. The method of any one of claims 125-127, comprising administering a therapeutically effective amount of a combination of a HER3 inhibitor and an EGFR inhibitor.

129. The method of any one of claims 125-127, comprising administering a therapeutically effective amount of a HER3 inhibitor.

130. The method of any one of claims 125-129, wherein the HER3 inhibitor is an anti-HER3 antibody or antigen-binding fragment thereof.

131. The method of claim 130, wherein the anti-HER3 antibody or antigen-binding fragment thereof specifically binds to the same HER3 epitope as an antibody or antigen-binding fragment thereof comprising the heavy chain variable region (VH) and light chain variable region (VL) of CL16 or 2C2.

132. The method of claim 131, wherein the VH and VL of CL16 comprise SEQ ID NOs: 2 and 1, respectively, and the VH and VL of 2C2 comprise SEQ ID NOs: 2 and 3, respectively.

133. The method of any one of claims 130-132, wherein the anti-HER3 antibody or antigen-binding fragment thereof is affinity matured.

134. The method of any one of claims 130-133, wherein the anti-HER3 antibody or antigen-binding fragment thereof comprises an antibody VL and an antibody VH, wherein the VL comprises the amino acid sequence:
[FW1]X₁GSX₂SNIGLNYVS[FW2]RNNQRPS[FW3]AAWDDX₃X₄X₅GEX₆
[FW4]
wherein [FW1], [FW2], [FW3] and [FW4] represent VL framework regions, and wherein
(a) X₁ represents amino acid residues Arginine (R) or Serine (S),
(b) X₂ represents amino acid residues Serine (S) or Leucine (L),
(c) X₃ represents amino acid residues Serine (S) or Glycine (G),
(d) X₄ represents amino acid residues Leucine (L) or Proline (P),
(e) X₅ represents amino acid residues Arginine (R), Isoleucine (I), Proline (P) or Serine (S), and
(f) X₆ represents amino acid residues Valine (V) or Alanine (A), and
wherein the VH comprises the amino acid sequence:
[FW5]YYYMQ[FW6]X₇IGSSGGVTNYADSVKG[FW7]VGLGDAFDI[FW8]
wherein [FW5], [FW6],[FW7] and [FW8] represent VH framework regions, and wherein X₇ represents amino acid residues Tyrosine (Y), Isoleucine (I) or Valine (V).

135. The method of claim 134, wherein FW1 comprises SEQ ID NO: 40 or 44, FW2 comprises SEQ ID NO: 41, FW3 comprises SEQ ID NO: 42, FW4 comprises SEQ ID NO: 43, FW5 comprises SEQ ID NO: 36, FW6 comprises SEQ ID NO: 37, FW7 comprises SEQ ID NO: 38, and FW8 comprises SEQ ID NO: 39.

136. The method of claim 130, wherein the anti-HER3 antibody or antigen-binding fragment thereof comprises a VL and a VH comprising VL-CDR1, VL-CRD2, VL-CDR3, VH-CDR1, VH-CDR2, and VH-CDR3 amino acid sequences identical or identical except for four, three, two, or one amino acid substitutions in one or more CDRs to: SEQ ID NOs: 18, 21, 22, 31, 32, and 35, SEQ ID NOs: 18, 21, 26, 31, 32 and 35, SEQ ID NOs: 18, 21, 27, 31, 32 and 35, SEQ ID NOs: 20, 21, 22, 31, 32 and 35, SEQ ID NOs: 19, 21, 22, 31, 32 and 35, SEQ ID NOs: 18, 21, 25, 31, 32 and 35, SEQ ID NOs: 18, 21, 28, 31, 32 and 35, SEQ ID NOs: 18, 21, 29, 31, 32 and 35, SEQ ID NOs: 18, 21, 30, 31, 32 and 35, SEQ ID NOs: 18, 21, 23, 31, 32 and 35, SEQ ID NOs: 19, 21, 23, 31, 32 and 35, SEQ ID NOs: 20, 21, 23, 31, 32 and 35, SEQ ID NOs: 18, 21, 24, 31, 32 and 35, or SEQ ID NOs: 18, 21, 25, 31, 32 and 35, respectively.

137. The method of claim 130, wherein the anti-HER3 antibody or antigen-binding fragment thereof comprises an antibody VL and an antibody VH, wherein the VL comprises an amino acid sequence at least about 90% to about 100% identical to a reference amino acid sequence selected from the group consisting of SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 14, SEQ ID NO: 15, SEQ ID NO: 16, and SEQ ID NO: 17.

138. The method of claim 130, wherein the anti-HER3 antibody or antigen-binding fragment thereof comprises an antibody VL and an antibody VH, wherein the VH comprises an amino acid sequence at least about 90% to about 100% identical to a reference amino acid sequence selected from the group consisting of SEQ ID NO: 2, SEQ ID NO: 12 and SEQ ID NO: 13.

139. The method of claim 130, wherein the anti-HER3 antibody or antigen-binding fragment thereof comprises a VL comprising a sequence at least about 90% to about 100% identical to a reference amino acid sequence selected from the group consisting of SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 14, SEQ ID NO: 15, SEQ ID NO: 16, and SEQ ID NO: 17, and wherein the anti-HER3 antibody or antigen-binding fragment comprises a VH comprising a sequence at least about 90% to about 100% identical to a reference amino acid sequence selected from the group consisting of SEQ ID NO: 2, SEQ ID NO: 12 and SEQ ID NO: 13.

140. The method of claim 130, wherein the anti-HER3 antibody or antigen-binding fragment thereof comprises a VL comprising the VL consensus sequence provided in Table 3 and a VH comprising the VH consensus sequence provided in Table 3.

141. The method of claim 130, wherein the anti-HER3 antibody or antigen-binding fragment thereof comprises a VL-CDR1, a VL-CDR2 and a VL-CDR3 comprising SEQ ID NO: 19, SEQ ID NO: 21 and SEQ ID NO: 23, respectively, and a VH-CDR1, a VH-CDR2, and a VH-CDR3 comprising SEQ ID NO: 31, SEQ ID NO: 32, and SEQ ID NO: 35, respectively.

142. The method of claim 130, wherein the anti-HER3 antibody or antigen-binding fragment thereof comprises a VL comprising SEQ ID NO: 3 and a VH comprising SEQ ID NO: 2.

143. The method of any one of claims 134-142, wherein the anti-HER3 antibody or antigen-binding fragment thereof comprises a human heavy chain constant region or fragment thereof.

144. The method of claim 143, wherein the heavy chain constant region or fragment thereof is an IgG constant region.

145. The method of claim 144, wherein the IgG constant region is selected from an IgG1 constant region, an IgG2 constant region, an IgG3 constant region and an IgG4 constant region.

146. The method of claim 144, wherein the IgG constant region is an IgG1 constant region.

147. The method of any one of claims 134-146, wherein the anti-HER3 antibody or antigen-binding fragment comprises a light chain constant region selected from the group consisting of a human kappa constant region and a human lambda constant region.

148. The method of claim 147, wherein the anti-HER3 antibody or antigen-binding fragment comprises a human lambda constant region.

149. The method of any one of claims 143-148, wherein the IgG constant domain comprises one or more amino acid substitutions relative to a wild-type IgG constant domain wherein the modified IgG has an increased half-life compared to the half-life of an IgG having the wild-type IgG constant domain.

150. The method of any one of claims 144-149, wherein the IgG constant domain comprises one or more amino acid substitutions of amino acid residues at positions 251-257, 285-290, 308-314, 385-389, and 428-436, wherein the numbering is according to the EU index as set forth in Kabat.

151. The method of claim 150, wherein at least one IgG constant domain amino acid substitution is selected from the group consisting of:
(a) substitution of the amino acid at position 252 with Tyrosine (Y), Phenylalanine (F), Tryptophan (W), or Threonine (T),
(b) substitution of the amino acid at position 254 with Threonine (T),
(c) substitution of the amino acid at position 256 with Serine (S), Arginine (R), Glutamine (Q), Glutamic acid (E), Aspartic acid (D), or Threonine (T),
(d) substitution of the amino acid at position 257 with Leucine (L),
(e) substitution of the amino acid at position 309 with Proline (P),
(f) substitution of the amino acid at position 311 with Serine (S),
(g) substitution of the amino acid at position 428 with Threonine (T), Leucine (L), Phenylalanine (F), or Serine (S),
(h) substitution of the amino acid at position 433 with Arginine (R), Serine (S), Isoleucine (I), Proline (P), or Glutamine (Q),
(i) substitution of the amino acid at position 434 with Tryptophan (W), Methionine (M), Serine (S), Histidine (H), Phenylalanine (F), or Tyrosine, and
(j) a combination of two or more of said substitutions, wherein the numbering is according to the EU index as set forth in Kabat.

152. The method of claim 150, wherein the human IgG constant domain comprises amino acid substitutions relative to a wild-type human IgG constant domain at positions 252, 254, and 256, wherein
(a) the amino acid at position 252 is substituted with Tyrosine (Y),
(b) the amino acid at position 254 is substituted with Threonine (T), and
(c) the amino acid at position 256 is substituted with Glutamic acid (E),
wherein the numbering is according to the EU index as set forth in Kabat.

153. The method of any one of claims 142-148, wherein the human IgG constant domain comprises amino acid substitutions relative to a wild-type human IgG constant domain at positions 252, 254, and 256, wherein
(a) the amino acid at position 252 is substituted with Tyrosine (Y),
(b) the amino acid at position 254 is substituted with Threonine (T), and
(c) the amino acid at position 256 is substituted with Glutamic acid (E),
wherein the numbering is according to the EU index as set forth in Kabat.

154. The method of any one of claims 150 to 153, wherein the amino acid at position 434 is substituted with an amino acid selected from the group consisting of Tryptophan (W), Methionine (M), Tyrosine (Y), and Serine (S), and wherein the numbering is according to the EU index as set forth in Kabat.

155. The method of claim 154, wherein the amino acid at position 428 is substituted with an amino acid selected from the group consisting of Threonine (T), Leucine (L), Phenylalanine (F), and Serine (S), and wherein the numbering is according to the EU index as set forth in Kabat.

156. The method of claim 154, wherein the amino acid at position 257 is substituted with Leucine (L), and the amino acid at Kabat position 434 is substituted with Tyrosine (Y), and wherein the numbering is according to the EU index as set forth in Kabat.

157. The method of claim 155, wherein the amino acid at Kabat position 428 is substituted with Leucine (L), and the amino acid at Kabat position 434 is substituted with Serine (S).

158. The method of claim 130, wherein the anti-HER3 antibody or antigen-binding fragment thereof comprises an antibody VL of SEQ ID NO:3, an antibody VH of SEQ ID NO: 2, and an IgG1 constant region of SEQ ID 46.

159. The method of claim 158, wherein the human IgG constant region comprises amino acid substitutions relative to a wild-type human IgG constant domain at positions 252, 254, and 256, wherein the numbering is according to the EU index as set forth in Kabat, and wherein
(a) the amino acid at position 252 is substituted with Tyrosine (Y),
(b) the amino acid at position 254 is substituted with Threonine (T), and
(c) the amino acid at position 256 is substituted with Glutamic acid (E).

160. The method of any one of claims 107-135, wherein the anti-HER3 antibody is a human antibody, a humanized antibody, a chimeric antibody, a monoclonal antibody, a polyclonal antibody, a recombinant antibody, a multispecific antibody, or an antigen-binding fragment thereof.

161. The method of claim 160, wherein the anti-HER3 antibody is a human antibody.

162. The method of any one of claims 130-161, which antigen-binding fragment is Fv, Fab, F(ab′)2, Fab′, dsFv, scFv, and sc(Fv)2.

163. The method of any one of claims 130-161, which anti-HER3 antibody or antigen-binding fragment is conjugated to at least one heterologous agent.

164. The method of any one of claims 125-127, comprising administering a therapeutically effective amount of an EGFR inhibitor.

165. The method of claim 128 or 164, wherein the EGFR inhibitor is an anti-EGFR antibody or an antigen-binding fragment thereof.

166. The method of claim 165, wherein the anti-EGFR antibody is cetuximab.

167. The method of any one of claims 125-166, wherein the cancer is a squamous cell carcinoma of the head and neck (SCCHN).

168. A method of determining whether a patient diagnosed with cancer is indicated as likely to be responsive to treatment with a HER3 inhibitor, an EGFR inhibitor, or a combination of a HER3 inhibitor and an EGFR inhibitor, comprising measuring the expression of at least one of AREG, TGF-α, and EGFR homodimer in a sample from the patient, wherein the sample comprises tumor cells, and high levels of one or more of AREG, TGF-α, and EGFR homodimer in the sample indicates that the patient is likely to be responsive to treatment with a HER3 inhibitor, an EGFR inhibitor, or a combination of a HER3 inhibitor and an EGFR inhibitor.

169. The method of claim 168, wherein the method comprises measuring the expression of AREG in the sample.

170. The method of claim 168 or 169, wherein the method comprises measuring the expression of TGF-α in the sample.

171. The method of any one of claims 168-170, wherein the method comprises measuring the expression of EGFR homodimer in the sample.

172. The method of any one of claims 168-171, which is a method of determining whether a patient diagnosed with cancer is indicated as likely to be responsive to treatment with a HER3 inhibitor and an EGFR inhibitor.

173. The method of any one of claims 168-171, which is a method of determining whether a patient diagnosed with cancer is indicated as likely to be responsive to treatment with a HER3 inhibitor.

174. The method of any one of claims 168-173, wherein the HER3 inhibitor is an anti-HER3 antibody or an antigen-binding fragment thereof.

175. The method of claim 174, wherein the anti-HER3 antibody or antigen-binding fragment thereof specifically binds to the same HER3 epitope as an antibody or antigen-binding fragment thereof comprising the heavy chain variable region (VH) and light chain variable region (VL) of CL16 or 2C2.

176. The method of claim 175, wherein the VH and VL of CL16 comprise SEQ ID NOs: 2 and 1, respectively, and the VH and VL of 2C2 comprise SEQ ID NOs: 2 and 3, respectively.

177. The method of any one of claims 174-176, wherein the anti-HER3 antibody or antigen-binding fragment thereof is affinity matured.

178. The method of any one of claims 174-177, wherein the anti-HER3 antibody or antigen-binding fragment thereof comprises an antibody VL and an antibody VH, wherein the VL comprises the amino acid sequence:
[FW1]X₁GSX₂SNIGLNYVS[FW2]RNNQRPS[FW3]AAWDDX₃X₄X₅GEX₆
[FW4]
wherein [FW1], [FW2], [FW3] and [FW4] represent VL framework regions, and wherein
(a) X₁ represents amino acid residues Arginine (R) or Serine (S),
(b) X₂ represents amino acid residues Serine (S) or Leucine (L),
(c) X₃ represents amino acid residues Serine (S) or Glycine (G),
(d) X₄ represents amino acid residues Leucine (L) or Proline (P),
(e) X₅ represents amino acid residues Arginine (R), Isoleucine (I), Proline (P) or Serine (S), and
(f) X₆ represents amino acid residues Valine (V) or Alanine (A), and wherein the VH comprises the amino acid sequence:
[FW5]YYYMQ[FW6]X₇IGSSGGVTNYADSVKG[FW7]VGLGDAFDI[FW8]
wherein [FW5], [FW6],[FW7] and [FW8] represent VH framework regions, and wherein X₇ represents amino acid residues Tyrosine (Y), Isoleucine (I) or Valine (V).

179. The method of claim 178, wherein FW1 comprises SEQ ID NO: 40 or 44, FW2 comprises SEQ ID NO: 41, FW3 comprises SEQ ID NO: 42, FW4 comprises SEQ ID NO: 43, FW5 comprises SEQ ID NO: 36, FW6 comprises SEQ ID NO: 37, FW7 comprises SEQ ID NO: 38, and FW8 comprises SEQ ID NO: 39.

180. The method of claim 174, wherein the anti-HER3 antibody or antigen-binding fragment thereof comprises a VL and a VH comprising VL-CDR1, VL-CRD2, VL-CDR3, VH-CDR1, VH-CDR2, and VH-CDR3 amino acid sequences identical or identical except for four, three, two, or one amino acid substitutions in one or more CDRs to: SEQ ID NOs: 18, 21, 22, 31, 32, and 35, SEQ ID NOs: 18, 21, 26, 31, 32 and 35, SEQ ID NOs: 18, 21, 27, 31, 32 and 35, SEQ ID NOs: 20, 21, 22, 31, 32 and 35, SEQ ID NOs: 19, 21, 22, 31, 32 and 35, SEQ ID NOs: 18, 21, 25, 31, 32 and 35, SEQ ID NOs: 18, 21, 28, 31, 32 and 35, SEQ ID NOs: 18, 21, 29, 31, 32 and 35, SEQ ID NOs: 18, 21, 30, 31, 32 and 35, SEQ ID NOs: 18, 21, 23, 31, 32 and 35, SEQ ID NOs: 19, 21, 23, 31, 32 and 35, SEQ ID NOs: 20, 21, 23, 31, 32 and 35, SEQ ID NOs: 18, 21, 24, 31, 32 and 35, or SEQ ID NOs: 18, 21, 25, 31, 32 and 35, respectively.

181. The method of claim 174, wherein the anti-HER3 antibody or antigen-binding fragment thereof comprises an antibody VL and an antibody VH, wherein the VL comprises an amino acid sequence at least about 90% to about 100% identical to a reference amino acid sequence selected from the group consisting of SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 14, SEQ ID NO: 15, SEQ ID NO: 16, and SEQ ID NO: 17.

182. The method of claim 174, wherein the anti-HER3 antibody or antigen-binding fragment thereof comprises an antibody VL and an antibody VH, wherein the VH comprises an amino acid sequence at least about 90% to about 100% identical to a reference amino acid sequence selected from the group consisting of SEQ ID NO: 2, SEQ ID NO: 12 and SEQ ID NO: 13.

183. The method of claim 174, wherein the anti-HER3 antibody or antigen-binding fragment thereof comprises a VL comprising a sequence at least about 90% to about 100% identical to a reference amino acid sequence selected from the group consisting of SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 14, SEQ ID NO: 15, SEQ ID NO: 16, and SEQ ID NO: 17, and wherein the anti-HER3 antibody or antigen-binding fragment comprises a VH comprising a sequence at least about 90% to about 100% identical to a reference amino acid sequence selected from the group consisting of SEQ ID NO: 2, SEQ ID NO: 12 and SEQ ID NO: 13.

184. The method of claim 174, wherein the anti-HER3 antibody or antigen-binding fragment thereof comprises a VL comprising the VL consensus sequence provided in Table 3 and a VH comprising the VH consensus sequence provided in Table 3.

185. The method of claim 174, wherein the anti-HER3 antibody or antigen-binding fragment thereof comprises a VL-CDR1, a VL-CDR2 and a VL-CDR3 comprising SEQ ID NO: 19, SEQ ID NO: 21 and SEQ ID NO: 23, respectively, and a VH-CDR1, a VH-CDR2, and a VH-CDR3 comprising SEQ ID NO: 31, SEQ ID NO: 32, and SEQ ID NO: 35, respectively.

186. The method of claim 174, wherein the anti-HER3 antibody or antigen-binding fragment thereof comprises a VL comprising SEQ ID NO: 3 and a VH comprising SEQ ID NO: 2.

187. The method of any one of claims 178-186, wherein the anti-HER3 antibody or antigen-binding fragment thereof comprises a human heavy chain constant region or fragment thereof.

188. The method of claim 187, wherein the heavy chain constant region or fragment thereof is an IgG constant region.

189. The method of claim 188, wherein the IgG constant region is selected from an IgG1 constant region, an IgG2 constant region, an IgG3 constant region and an IgG4 constant region.

190. The method of claim 188, wherein the IgG constant region is an IgG1 constant region.

191. The method of any one of claims 178-189, wherein the anti-HER3 antibody or antigen-binding fragment comprises a light chain constant region selected from the group consisting of a human kappa constant region and a human lambda constant region.

192. The method of claim 191, wherein the anti-HER3 antibody or antigen-binding fragment comprises a human lambda constant region.

193. The method of any one of claims 187-192, wherein the IgG constant domain comprises one or more amino acid substitutions relative to a wild-type IgG constant domain wherein the modified IgG has an increased half-life compared to the half-life of an IgG having the wild-type IgG constant domain.

194. The method of any one of claims claim 188-193, wherein the IgG constant domain comprises one or more amino acid substitutions of amino acid residues at positions 251-257, 285-290, 308-314, 385-389, and 428-436, wherein the numbering is according to the EU index as set forth in Kabat.

195. The method of claim 194, wherein at least one IgG constant domain amino acid substitution is selected from the group consisting of:
(a) substitution of the amino acid at position 252 with Tyrosine (Y), Phenylalanine (F), Tryptophan (W), or Threonine (T),
(b) substitution of the amino acid at position 254 with Threonine (T),
(c) substitution of the amino acid at position 256 with Serine (S), Arginine (R), Glutamine (Q), Glutamic acid (E), Aspartic acid (D), or Threonine (T),
(d) substitution of the amino acid at position 257 with Leucine (L),
(e) substitution of the amino acid at position 309 with Proline (P),
(f) substitution of the amino acid at position 311 with Serine (S),
(g) substitution of the amino acid at position 428 with Threonine (T), Leucine (L), Phenylalanine (F), or Serine (S),
(h) substitution of the amino acid at position 433 with Arginine (R), Serine (S), Isoleucine (I), Proline (P), or Glutamine (Q),
(i) substitution of the amino acid at position 434 with Tryptophan (W), Methionine (M), Serine (S), Histidine (H), Phenylalanine (F), or Tyrosine, and
(j) a combination of two or more of said substitutions, wherein the numbering is according to the EU index as set forth in Kabat.

196. The method of claim 194, wherein the human IgG constant domain comprises amino acid substitutions relative to a wild-type human IgG constant domain at positions 252, 254, and 256, wherein
(a) the amino acid at position 252 is substituted with Tyrosine (Y),
(b) the amino acid at position 254 is substituted with Threonine (T), and
(c) the amino acid at position 256 is substituted with Glutamic acid (E),
wherein the numbering is according to the EU index as set forth in Kabat.

197. The method of any one of claims 187-192, wherein the human IgG constant domain comprises amino acid substitutions relative to a wild-type human IgG constant domain at positions 252, 254, and 256, wherein
(a) the amino acid at position 252 is substituted with Tyrosine (Y),
(b) the amino acid at position 254 is substituted with Threonine (T), and
(c) the amino acid at position 256 is substituted with Glutamic acid (E),
wherein the numbering is according to the EU index as set forth in Kabat.

198. The method of any one of claims 194-197, wherein the amino acid at position 434 is substituted with an amino acid selected from the group consisting of Tryptophan (W), Methionine (M), Tyrosine (Y), and Serine (S), and wherein the numbering is according to the EU index as set forth in Kabat.

199. The method of claim 198, wherein the amino acid at position 428 is substituted with an amino acid selected from the group consisting of Threonine (T), Leucine (L), Phenylalanine (F), and Serine (S), and wherein the numbering is according to the EU index as set forth in Kabat.

200. The method of claim 198, wherein the amino acid at position 257 is substituted with Leucine (L), and the amino acid at Kabat position 434 is substituted with Tyrosine (Y), and wherein the numbering is according to the EU index as set forth in Kabat.

201. The method of claim 199, wherein the amino acid at Kabat position 428 is substituted with Leucine (L), and the amino acid at Kabat position 434 is substituted with Serine (S).

202. The method of claim 164, wherein the anti-HER3 antibody or antigen-binding fragment thereof comprises an antibody VL of SEQ ID NO:3, an antibody VH of SEQ ID NO: 2, and an IgG1 constant region of SEQ ID 46.

203. The method of claim 202, wherein the human IgG constant region, comprises amino acid substitutions relative to a wild-type human IgG constant domain at positions 252, 254, and 256, wherein the numbering is according to the EU index as set forth in Kabat, and wherein
(a) the amino acid at position 252 is substituted with Tyrosine (Y),
(b) the amino acid at position 254 is substituted with Threonine (T), and
(c) the amino acid at position 256 is substituted with Glutamic acid (E).

204. The method of claim 185, wherein the anti-HER3 antibody or antigen-binding fragment thereof comprises a human IgG1 constant region and a human lambda constant region.

205. The method of claim 204, wherein the IgG1 constant region comprises amino acid substitutions relative to a wild-type human IgG constant domain at positions 252, 254, and 256, wherein the numbering is according to the EU index as set forth in Kabat, and wherein
(a) the amino acid at position 252 is substituted with Tyrosine (Y),
(b) the amino acid at position 254 is substituted with Threonine (T), and
(c) the amino acid at position 256 is substituted with Glutamic acid (E).

206. The method of claim 186, wherein the anti-HER3 antibody or antigen-binding fragment thereof comprises a human IgG1 constant region and a human lambda constant region.

207. The method of claim 206, wherein the IgG1 constant region comprises amino acid substitutions relative to a wild-type human IgG constant domain at positions 252, 254, and 256, wherein the numbering is according to the EU index as set forth in Kabat, and wherein
(a) the amino acid at position 252 is substituted with Tyrosine (Y),
(b) the amino acid at position 254 is substituted with Threonine (T), and
(c) the amino acid at position 256 is substituted with Glutamic acid (E).

208. The method of any one of claims 164-207, wherein the HER3 inhibitor is an anti-HER3 antibody.

209. The method of any one of claims 164-208, wherein the anti-HER3 antibody is a human antibody, a humanized antibody, a chimeric antibody, a monoclonal antibody, a polyclonal antibody, a recombinant antibody, a multispecific antibody, or an antigen-binding fragment thereof.

210. The method of claim 209, wherein the anti-HER3 antibody is a human antibody.

211. The method of any one of claims 164-210, which antigen-binding fragment is Fv, Fab, F(ab′)2, Fab′, dsFv, scFv, and sc(Fv)2.

212. The method of any one of claims 164-211, which anti-HER3 antibody or antigen-binding fragment is conjugated to at least one heterologous agent.

213. The method of any one of claims 168-173, which is a method of determining whether a patient diagnosed with cancer is indicated as likely to be responsive to treatment with an EGFR inhibitor.

214. The method of claim 172 or 213, wherein the EGFR inhibitor is an anti-EGFR antibody or an antigen-binding fragment thereof.

215. The method of claim 214, wherein the anti-EGFR antibody is cetuximab.

216. The method of any one of claims 168-215, wherein the cancer is a squamous cell carcinoma of the head and neck (SCCHN).

217. The method of any one of claims 168-216, wherein the method comprises a first step of obtaining the sample from a tumor from the patient.

218. The method of any one of claims 168-172, wherein, if the patient diagnosed with cancer is indicated as likely to be responsive to treatment with a HER3 inhibitor and an EGFR inhibitor, the method comprises an additional step of administering to the patient a therapeutically effective amount of a HER3 inhibitor and a therapeutically effective amount of an EGFR inhibitor.

219. A kit comprising components for performing the method of any one of claims 168-217.

220. The method of any one of claims 171-219, wherein the step of measuring the expression of EGFR homodimer comprises measuring the level of protein-protein interaction between EGFR monomers in the sample.

221. The method of claim 168, wherein, if the patient diagnosed with cancer is indicated as likely to be responsive to treatment with a HER3 inhibitor and an EGFR inhibitor and expression of EGFR homodimer is high in the sample, the method comprises an additional step of measuring the expression of AREG in the sample, wherein high expression of AREG in the sample indicates that the patient is likely to be responsive to treatment with a HER3 inhibitor and an EGFR inhibitor.

222. The method of claim 168 or claim 221, wherein, if the patient diagnosed with cancer is indicated as likely to be responsive to treatment with a HER3 inhibitor and an EGFR inhibitor and expression of EGFR homodimer is high in the sample, the method comprises an additional step of measuring the expression of TGF-α in the sample, wherein high expression of TGF-α in the sample indicates that the patient is likely to be responsive to treatment with a HER3 inhibitor and an EGFR inhibitor.

223. The method of claim 55, wherein the cancer is human papillomavirus (HPV) positive.

224. The method of claim 55, wherein the cancer is HPV negative.

225. The method of claim 113, wherein the cancer is HPV positive.

226. The method of claim 113, wherein the cancer is HPV negative.

227. The method of claim 167, wherein the cancer is HPV positive.

228. The method of claim 167, wherein the cancer is HPV negative.

229. The method of claim 206, wherein the cancer is HPV positive.

230. The method of claim 206, wherein the cancer is HPV negative.

231. A method of treating a cancer, comprising administering to a patient diagnosed with said cancer a therapeutically effective amount of (i) a HER3 inhibitor, (ii) a combination of a HER3 inhibitor and a B-Raf inhibitor, (iii) a combination of a HER3 inhibitor and a MEK inhibitor, or (iv) a combination of a HER3 inhibitor, a B-Raf inhibitor, and a MEK inhibitor, wherein the patient has a tumor that has been characterized in that cells from the tumor express a neuregulin.

232. The method of claim 231, wherein cells from the tumor express NRG1.

233. The method of claim 232, wherein cells from the tumor express NRG1α.

234. The method of claim 232, wherein cells from the tumor express NRG1β.

235. The method of any one of claims 231-234, wherein cells from the tumor express NRG2.

236. The method of claim 235, wherein cells from the tumor express NRG2α.

237. The method of claim 235, wherein cells from the tumor express NRG2β.

238. The method of any one of claims 231-237, wherein cells from the tumor express high levels of NRG1.

239. The method of any one of claims 231-238, wherein cells from the tumor express high levels of NRG2.

240. The method of any one of claims 231-239, comprising administering a therapeutically effective amount of a combination of a HER3 inhibitor and a B-Raf inhibitor.

241. The method of any one of claims 231-239, comprising administering a therapeutically effective amount of a HER3 inhibitor.

242. The method of any one of claims 231-239, comprising administering a therapeutically effective amount of a combination of a HER3 inhibitor and a MEK inhibitor.

243. The method of any one of claims 231-239, comprising administering a therapeutically effective amount of a combination of a HER3 inhibitor, a B-Raf inhibitor, and a MEK inhibitor.

244. The method of any one of claims 231-243, wherein the HER3 inhibitor is an anti-HER3 antibody or antigen-binding fragment thereof.

245. The method of claim 245, wherein the anti-HER3 antibody or antigen-binding fragment thereof specifically binds to the same HER3 epitope as an antibody or antigen-binding fragment thereof comprising the heavy chain variable region (VH) and light chain variable region (VL) of CL16 or 2C2.

246. The method of claim 245, wherein the VH and VL of CL16 comprise SEQ ID NOs: 2 and 1, respectively, and the VH and VL of 2C2 comprise SEQ ID NOs: 2 and 3, respectively.

247. The method of any one of claims 244-246, wherein the anti-HER3 antibody or antigen-binding fragment thereof is affinity matured.

248. The method of claim 246 or 247, wherein the anti-HER3 antibody or antigen-binding fragment thereof comprises an antibody VL and an antibody VH, wherein the VL comprises the amino acid sequence:
[FW1]X₁GSX₂SNIGLNYVS[FW2]RNNQRPS[FW3]AAWDDX₃X₄X₅GEX₆
[FW4]
wherein [FW1], [FW2], [FW3] and [FW4] represent VL framework regions, and wherein
(a) X₁ represents amino acid residues Arginine (R) or Serine (S),
(b) X₂ represents amino acid residues Serine (S) or Leucine (L),
(c) X₃ represents amino acid residues Serine (S) or Glycine (G),
(d) X₄ represents amino acid residues Leucine (L) or Proline (P),
(e) X₅ represents amino acid residues Arginine (R), Isoleucine (I), Proline (P) or Serine (S), and
(f) X₆ represents amino acid residues Valine (V) or Alanine (A), and
wherein the VH comprises the amino acid sequence:
[FW5]YYYMQ[FW6]X₇IGSSGGVTNYADSVKG[FW7]VGLGDAFDI[FW8]
wherein [FW5], [FW6],[FW7] and [FW8] represent VH framework regions, and wherein X₇ represents amino acid residues Tyrosine (Y), Isoleucine (I) or Valine (V).

249. The method of claim 248, wherein FW1 comprises SEQ ID NO: 40 or 44, FW2 comprises SEQ ID NO: 41, FW3 comprises SEQ ID NO: 42, FW4 comprises SEQ ID NO: 43, FW5 comprises SEQ ID NO: 36, FW6 comprises SEQ ID NO: 37, FW7 comprises SEQ ID NO: 38, and FW8 comprises SEQ ID NO: 39.

250. The method of claim 244, wherein the anti-HER3 antibody or antigen-binding fragment thereof comprises a VL and a VH comprising VL-CDR1, VL-CRD2, VL-CDR3, VH-CDR1, VH-CDR2, and VH-CDR3 amino acid sequences identical or identical except for four, three, two, or one amino acid substitutions in one or more CDRs to: SEQ ID NOs: 18, 21, 22, 31, 32, and 35, SEQ ID NOs: 18, 21, 26, 31, 32 and 35, SEQ ID NOs: 18, 21, 27, 31, 32 and 35, SEQ ID NOs: 20, 21, 22, 31, 32 and 35, SEQ ID NOs: 19, 21, 22, 31, 32 and 35, SEQ ID NOs: 18, 21, 25, 31, 32 and 35, SEQ ID NOs: 18, 21, 28, 31, 32 and 35, SEQ ID NOs: 18, 21, 29, 31, 32 and 35, SEQ ID NOs: 18, 21, 30, 31, 32 and 35, SEQ ID NOs: 18, 21, 23, 31, 32 and 35, SEQ ID NOs: 19, 21, 23, 31, 32 and 35, SEQ ID NOs: 20, 21, 23, 31, 32 and 35, SEQ ID NOs: 18, 21, 24, 31, 32 and 35, or SEQ ID NOs: 18, 21, 25, 31, 32 and 35, respectively.

251. The method of claim 244, wherein the anti-HER3 antibody or antigen-binding fragment thereof comprises an antibody VL and an antibody VH, wherein the VL comprises an amino acid sequence at least about 90% to about 100% identical to a reference amino acid sequence selected from the group consisting of SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 14, SEQ ID NO: 15, SEQ ID NO: 16, and SEQ ID NO: 17.

252. The method of claim 244, wherein the anti-HER3 antibody or antigen-binding fragment thereof comprises an antibody VL and an antibody VH, wherein the VH comprises an amino acid sequence at least about 90% to about 100% identical to a reference amino acid sequence selected from the group consisting of SEQ ID NO: 2, SEQ ID NO: 12 and SEQ ID NO: 13.

253. The method of claim 244, wherein the anti-HER3 antibody or antigen-binding fragment thereof comprises a VL comprising a sequence at least about 90% to about 100% identical to a reference amino acid sequence selected from the group consisting of SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 14, SEQ ID NO: 15, SEQ ID NO: 16, and SEQ ID NO: 17, and wherein the anti-HER3 antibody or antigen-binding fragment comprises a VH comprising a sequence at least about 90% to about 100% identical to a reference amino acid sequence selected from the group consisting of SEQ ID NO: 2, SEQ ID NO: 12 and SEQ ID NO: 13.

254. The method of claim 244, wherein the anti-HER3 antibody or antigen-binding fragment thereof comprises a VL comprising the VL consensus sequence provided in Table 3 and a VH comprising the VH consensus sequence provided in Table 3.

255. The method of claim 244, wherein the anti-HER3 antibody or antigen-binding fragment thereof comprises a VL-CDR1, a VL-CDR2 and a VL-CDR3 comprising SEQ ID NO: 19, SEQ ID NO: 21 and SEQ ID NO: 23, respectively, and a VH-CDR1, a VH-CDR2, and a VH-CDR3 comprising SEQ ID NO: 31, SEQ ID NO: 32, and SEQ ID NO: 35, respectively.

256. The method of claim 244, wherein the anti-HER3 antibody or antigen-binding fragment thereof comprises a VL comprising SEQ ID NO: 3 and a VH comprising SEQ ID NO: 2.

257. The method of any one of claims 248-256, wherein the anti-HER3 antibody or antigen-binding fragment thereof comprises a human heavy chain constant region or fragment thereof.

258. The method of claim 257, wherein the heavy chain constant region or fragment thereof is an IgG constant region.

259. The method of claim 258, wherein the IgG constant region is selected from an IgG1 constant region, an IgG2 constant region, an IgG3 constant region and an IgG4 constant region.

260. The method of claim 258, wherein the IgG constant region is an IgG1 constant region.

261. The method of any one of claims 248-260, wherein the anti-HER3 antibody or antigen-binding fragment comprises a light chain constant region selected from the group consisting of a human kappa constant region and a human lambda constant region.

262. The method of claim 261, wherein the anti-HER3 antibody or antigen-binding fragment comprises a human lambda constant region.

263. The method of any one of claims 254-262, wherein the IgG constant domain comprises one or more amino acid substitutions relative to a wild-type IgG constant domain wherein the modified IgG has an increased half-life compared to the half-life of an IgG having the wild-type IgG constant domain.

264. The method of any one of claims 258-263, wherein the IgG constant domain comprises one or more amino acid substitutions of amino acid residues at positions 251-257, 285-290, 308-314, 385-389, and 428-436, wherein the numbering is according to the EU index as set forth in Kabat.

265. The method of claim 264, wherein at least one IgG constant domain amino acid substitution is selected from the group consisting of:
(a) substitution of the amino acid at position 252 with Tyrosine (Y), Phenylalanine (F), Tryptophan (W), or Threonine (T),
(b) substitution of the amino acid at position 254 with Threonine (T),
(c) substitution of the amino acid at position 256 with Serine (S), Arginine (R), Glutamine (Q), Glutamic acid (E), Aspartic acid (D), or Threonine (T),
(d) substitution of the amino acid at position 257 with Leucine (L),
(e) substitution of the amino acid at position 309 with Proline (P),
(f) substitution of the amino acid at position 311 with Serine (S),
(g) substitution of the amino acid at position 428 with Threonine (T), Leucine (L), Phenylalanine (F), or Serine (S),
(h) substitution of the amino acid at position 433 with Arginine (R), Serine (S), Isoleucine (I), Proline (P), or Glutamine (Q),
(i) substitution of the amino acid at position 434 with Tryptophan (W), Methionine (M), Serine (S), Histidine (H), Phenylalanine (F), or Tyrosine, and
(j) a combination of two or more of said substitutions,
wherein the numbering is according to the EU index as set forth in Kabat.

266. The method of claim 264, wherein the human IgG constant domain comprises amino acid substitutions relative to a wild-type human IgG constant domain at positions 252, 254, and 256, wherein
(a) the amino acid at position 252 is substituted with Tyrosine (Y),
(b) the amino acid at position 254 is substituted with Threonine (T), and
(c) the amino acid at position 256 is substituted with Glutamic acid (E),
wherein the numbering is according to the EU index as set forth in Kabat.

267. The method of any one of claims 257-262, wherein the human IgG constant domain comprises amino acid substitutions relative to a wild-type human IgG constant domain at positions 252, 254, and 256, wherein
(a) the amino acid at position 252 is substituted with Tyrosine (Y),
(b) the amino acid at position 254 is substituted with Threonine (T), and
(c) the amino acid at position 256 is substituted with Glutamic acid (E),
wherein the numbering is according to the EU index as set forth in Kabat.

268. The method of any one of claims 254-267, wherein the amino acid at position 434 is substituted with an amino acid selected from the group consisting of Tryptophan (W), Methionine (M), Tyrosine (Y), and Serine (S), and wherein the numbering is according to the EU index as set forth in Kabat.

269. The method of claim 268, wherein the amino acid at position 428 is substituted with an amino acid selected from the group consisting of Threonine (T), Leucine (L), Phenylalanine (F), and Serine (S), and wherein the numbering is according to the EU index as set forth in Kabat.

270. The method of claim 268, wherein the amino acid at position 257 is substituted with Leucine (L), and the amino acid at Kabat position 434 is substituted with Tyrosine (Y), and wherein the numbering is according to the EU index as set forth in Kabat.

271. The method of claim 269, wherein the amino acid at Kabat position 428 is substituted with Leucine (L), and the amino acid at Kabat position 434 is substituted with Serine (S).

272. The method of claim 244, wherein the anti-HER3 antibody or antigen-binding fragment thereof comprises an antibody VL of SEQ ID NO:3, an antibody VH of SEQ ID NO: 2, and an IgG1 constant region of SEQ ID 46.

273. The method of claim 272, wherein the human IgG constant region comprises amino acid substitutions relative to a wild-type human IgG constant domain at positions 252, 254, and 256, wherein the numbering is according to the EU index as set forth in Kabat, and wherein
(a) the amino acid at position 252 is substituted with Tyrosine (Y),
(b) the amino acid at position 254 is substituted with Threonine (T), and
(c) the amino acid at position 256 is substituted with Glutamic acid (E).

274. The method of claim 255, wherein the anti-HER3 antibody or antigen-binding fragment thereof comprises a human IgG1 constant region and a human lambda constant region.

275. The method of claim 274, wherein the IgG1 constant region comprises amino acid substitutions relative to a wild-type human IgG constant domain at positions 252, 254, and 256, wherein the numbering is according to the EU index as set forth in Kabat, and wherein
(a) the amino acid at position 252 is substituted with Tyrosine (Y),
(b) the amino acid at position 254 is substituted with Threonine (T), and
(c) the amino acid at position 256 is substituted with Glutamic acid (E).

276. The method of claim 256, wherein the anti-HER3 antibody or antigen-binding fragment thereof comprises a human IgG1 constant region and a human lambda constant region.

277. The method of claim 276, wherein the IgG1 constant region comprises amino acid substitutions relative to a wild-type human IgG constant domain at positions 252, 254, and 256, wherein the numbering is according to the EU index as set forth in Kabat, and wherein
(a) the amino acid at position 252 is substituted with Tyrosine (Y),
(b) the amino acid at position 254 is substituted with Threonine (T), and
(c) the amino acid at position 256 is substituted with Glutamic acid (E).

278. The method of any one of claims 244-263, wherein the anti-HER3 antibody is a human antibody, a humanized antibody, a chimeric antibody, a monoclonal antibody, a polyclonal antibody, a recombinant antibody, a multispecific antibody, or an antigen-binding fragment thereof.

279. The method of claim 264, wherein the anti-HER3 antibody is a human antibody.

280. The method of any one of claims 244-279, which antigen-binding fragment is Fv, Fab, F(ab′)2, Fab′, dsFv, scFv, and sc(Fv)2.

281. The method of any one of claims 244-280, which anti-HER3 antibody or antigen-binding fragment is conjugated to at least one heterologous agent.

282. The method of any one of claims 231-281, wherein the B-Raf inhibitor is vemurafenib.

283. The method of any one of claims 231-281, wherein the B-Raf inhibitor is dabrafenib.

284. The method of any one of claims 231-283, wherein the MEK inhibitor is selumetinib.

285. The method of any one of claims 231-283, wherein the MEK inhibitor is trametinib.

286. The method of any one of claims 231-285, wherein the cancer is characterized by a BRAF mutation.

287. The method of any one of claims 231-286, wherein the cancer is resistant to treatment with a BRAF inhibitor.

288. The method of any one of claims 231-287, wherein the cancer is resistant to treatment with a MEK inhibitor

289. The method of any one of claims 231-288, wherein the cancer is resistant to treatment with a BRAF inhibitor and a MEK inhibitor.

290. The method of any one of claims 231-289, wherein the cancer is melanoma.

291. The method of claim 290, wherein the cancer is B-Raf mutated melanoma.

292. The method of any one of claims 231-289, wherein the cancer is thyroid cancer.

293. The method of claim 292, wherein the thyroid cancer is B-Raf mutated thyroid cancer.

294. The method of any one of claims 231-289, wherein the cancer is colorectal cancer.

295. The method of claim 294, wherein the colorectal cancer is B-Raf mutated colorectal cancer.

296. The method of any one of claims 231-289, wherein the cancer is lung cancer.

297. The method of claim 296, wherein the lung cancer is B-Raf mutated lung cancer.

298. The method of claim 296 or claim 297, wherein the lung cancer is non small cell lung carcinoma.

299. The method of any one of claims 231-289, wherein the cancer is hairy cell leukemia.

300. The method of claim 299, wherein the hairy cell leukemia is B-Raf mutated hairy cell leukemia.

301. The method of any one of claims 231-289, wherein the cancer is a squamous cell carcinoma of the head and neck (SCCHN), ovarian cancer, pancreatic cancer, bladder cancer, testicular cancer, endometrial cancer, hepatocellular carcinoma, breast cancer, gastric cancer, or prostate cancer.

302. The method of any one of claims 231-301, wherein the method comprises a step of measuring the expression of the neuregulin in the cells from the tumor.

303. A method of determining whether a patient diagnosed with cancer is indicated as likely to be responsive to treatment with (i) a HER3 inhibitor, (ii) a combination of a HER3 inhibitor and a B-Raf inhibitor, (iii) a combination of a HER3 inhibitor and a MEK inhibitor, or (iv) a combination of a HER3 inhibitor, a B-Raf inhibitor, and a MEK inhibitor, comprising measuring the expression of a neuregulin, wherein high levels of the neuregulin in the sample indicates that the patient is likely to be responsive to treatment with (i) a HER3 inhibitor, (ii) a combination of a HER3 inhibitor and a B-Raf inhibitor, (iii) a combination of a HER3 inhibitor and a MEK inhibitor, or (iv) a combination of a HER3 inhibitor, a B-Raf inhibitor, and a MEK inhibitor.

304. The method of claim 303, wherein the neuregulin is NRG1.

305. The method of claim 304, wherein the neuregulin is NRG1α.

306. The method of claim 304, wherein the neuregulin is NRG1β.

307. The method of any one of claims 303-306, wherein the neuregulin is NRG2.

308. The method of claim 307, wherein the neuregulin is NRG2α.

309. The method of claim 307, wherein the neuregulin is NRG2β.

310. The method of any one of claims 303-309, which is a method of determining whether a patient diagnosed with cancer is indicated as likely to be responsive to treatment with a HER3 inhibitor and a B-Raf inhibitor.

311. The method of any one of claims 303-309, which is a method of determining whether a patient diagnosed with cancer is indicated as likely to be responsive to treatment with a HER3 inhibitor.

312. The method of any one of claims 303-309, which is a method of determining whether a patient diagnosed with cancer is indicated as likely to be responsive to treatment with a HER3 inhibitor and a MEK inhibitor.

313. The method of any one of claims 303-309, which is a method of determining whether a patient diagnosed with cancer is indicated as likely to be responsive to treatment with a HER3 inhibitor, a B-Raf inhibitor, and a MEK inhibitor.

314. The method of any one of claims 303-313, wherein the HER3 inhibitor is an anti-HER3 antibody or an antigen-binding fragment thereof.

315. The method of claim 314, wherein the anti-HER3 antibody or antigen-binding fragment thereof specifically binds to the same HER3 epitope as an antibody or antigen-binding fragment thereof comprising the heavy chain variable region (VH) and light chain variable region (VL) of CL16 or 2C2.

316. The method of claim 315, wherein the VH and VL of CL16 comprise SEQ ID NOs: 2 and 1, respectively, and the VH and VL of 2C2 comprise SEQ ID NOs: 2 and 3, respectively.

317. The method of any one of claims 314-316, wherein the anti-HER3 antibody or antigen-binding fragment thereof is affinity matured.

318. The method of any one of claims 314-317, wherein the anti-HER3 antibody or antigen-binding fragment thereof comprises an antibody VL and an antibody VH, wherein the VL comprises the amino acid sequence:
[FW1]X₁GSX₂SNIGLNYVS[FW2]RNNQRPS[FW3]AAWDDX₃X₄X₅GEX₆
[FW4]
wherein [FW1], [FW2], [FW3] and [FW4] represent VL framework regions, and wherein
(a) X₁ represents amino acid residues Arginine (R) or Serine (S),
(b) X₂ represents amino acid residues Serine (S) or Leucine (L), (c) X₃ represents amino acid residues Serine (S) or Glycine (G), (d) X₄ represents amino acid residues Leucine (L) or Proline (P),
(e) X₅ represents amino acid residues Arginine (R), Isoleucine (I), Proline (P) or Serine (S), and
(f) X₆ represents amino acid residues Valine (V) or Alanine (A), and
wherein the VH comprises the amino acid sequence:
[FW5]YYYMQ[FW6]X₇IGSSGGVTNYADSVKG[FW7]VGLGDAFDI[FW8]
wherein [FW5], [FW6],[FW7] and [FW8] represent VH framework regions, and wherein X₇ represents amino acid residues Tyrosine (Y), Isoleucine (I) or Valine (V).

319. The method of claim 318, wherein FW1 comprises SEQ ID NO: 40 or 44, FW2 comprises SEQ ID NO: 41, FW3 comprises SEQ ID NO: 42, FW4 comprises SEQ ID NO: 43, FW5 comprises SEQ ID NO: 36, FW6 comprises SEQ ID NO: 37, FW7 comprises SEQ ID NO:
38, and FW8 comprises SEQ ID NO: 39.

320. The method of claim 314, wherein the anti-HER3 antibody or antigen-binding fragment thereof comprises a VL and a VH comprising VL-CDR1, VL-CRD2, VL-CDR3, VH-CDR1, VH-CDR2, and VH-CDR3 amino acid sequences identical or identical except for four, three, two, or one amino acid substitutions in one or more CDRs to: SEQ ID NOs: 18, 21, 22, 31, 32, and 35, SEQ ID NOs: 18, 21, 26, 31, 32 and 35, SEQ ID NOs: 18, 21, 27, 31, 32 and 35, SEQ ID NOs: 20, 21, 22, 31, 32 and 35, SEQ ID NOs: 19, 21, 22, 31, 32 and 35, SEQ ID NOs: 18, 21, 25, 31, 32 and 35, SEQ ID NOs: 18, 21, 28, 31, 32 and 35, SEQ ID NOs: 18, 21, 29, 31, 32 and 35, SEQ ID NOs: 18, 21, 30, 31, 32 and 35, SEQ ID NOs: 18, 21, 23, 31, 32 and 35, SEQ ID NOs: 19, 21, 23, 31, 32 and 35, SEQ ID NOs: 20, 21, 23, 31, 32 and 35, SEQ ID NOs: 18, 21, 24, 31, 32 and 35, or SEQ ID NOs: 18, 21, 25, 31, 32 and 35, respectively.

321. The method of claim 314, wherein the anti-HER3 antibody or antigen-binding fragment thereof comprises an antibody VL and an antibody VH, wherein the VL comprises an amino acid sequence at least about 90% to about 100% identical to a reference amino acid sequence selected from the group consisting of SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 14, SEQ ID NO: 15, SEQ ID NO: 16, and SEQ ID NO: 17.

322. The method of claim 314, wherein the anti-HER3 antibody or antigen-binding fragment thereof comprises an antibody VL and an antibody VH, wherein the VH comprises an amino acid sequence at least about 90% to about 100% identical to a reference amino acid sequence selected from the group consisting of SEQ ID NO: 2, SEQ ID NO: 12 and SEQ ID NO: 13.

323. The method of claim 314, wherein the anti-HER3 antibody or antigen-binding fragment thereof comprises a VL comprising a sequence at least about 90% to about 100% identical to a reference amino acid sequence selected from the group consisting of SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 14, SEQ ID NO: 15, SEQ ID NO: 16, and SEQ ID NO: 17, and wherein the anti-HER3 antibody or antigen-binding fragment comprises a VH comprising a sequence at least about 90% to about 100% identical to a reference amino acid sequence selected from the group consisting of SEQ ID NO: 2, SEQ ID NO: 12 and SEQ ID NO: 13.

324. The method of claim 314, wherein the anti-HER3 antibody or antigen-binding fragment thereof comprises a VL comprising the VL consensus sequence provided in Table 3 and a VH comprising the VH consensus sequence provided in Table 3.

325. The method of claim 314, wherein the anti-HER3 antibody or antigen-binding fragment thereof comprises a VL-CDR1, a VL-CDR2 and a VL-CDR3 comprising SEQ ID NO: 19, SEQ ID NO: 21 and SEQ ID NO: 23, respectively, and a VH-CDR1, a VH-CDR2, and a VH-CDR3 comprising SEQ ID NO: 31, SEQ ID NO: 32, and SEQ ID NO: 35, respectively.

326. The method of claim 314, wherein the anti-HER3 antibody or antigen-binding fragment thereof comprises a VL comprising SEQ ID NO: 3 and a VH comprising SEQ ID NO: 2.

327. The method of any one of claims 318-326, wherein the anti-HER3 antibody or antigen-binding fragment thereof comprises a human heavy chain constant region or fragment thereof.

328. The method of claim 327, wherein the heavy chain constant region or fragment thereof is an IgG constant region.

329. The method of claim 327, wherein the IgG constant region is selected from an IgG1 constant region, an IgG2 constant region, an IgG3 constant region and an IgG4 constant region.

330. The method of claim 327, wherein the IgG constant region is an IgG1 constant region.

331. The method of any one of claims 318-330, wherein the anti-HER3 antibody or antigen-binding fragment comprises a light chain constant region selected from the group consisting of a human kappa constant region and a human lambda constant region.

332. The method of claim 331, wherein the anti-HER3 antibody or antigen-binding fragment comprises a human lambda constant region.

333. The method of any one of claims 324-332, wherein the IgG constant domain comprises one or more amino acid substitutions relative to a wild-type IgG constant domain wherein the modified IgG has an increased half-life compared to the half-life of an IgG having the wild-type IgG constant domain.

334. The method of any one of claims 328-333, wherein the IgG constant domain comprises one or more amino acid substitutions of amino acid residues at positions 251-257, 285-290, 308-314, 385-389, and 428-436, wherein the numbering is according to the EU index as set forth in Kabat.

335. The method of claim 334, wherein at least one IgG constant domain amino acid substitution is selected from the group consisting of:
(a) substitution of the amino acid at position 252 with Tyrosine (Y), Phenylalanine (F), Tryptophan (W), or Threonine (T),
(b) substitution of the amino acid at position 254 with Threonine (T),
(c) substitution of the amino acid at position 256 with Serine (S), Arginine (R), Glutamine (Q), Glutamic acid (E), Aspartic acid (D), or Threonine (T),
(d) substitution of the amino acid at position 257 with Leucine (L),
(e) substitution of the amino acid at position 309 with Proline (P),
(f) substitution of the amino acid at position 311 with Serine (S),
(g) substitution of the amino acid at position 428 with Threonine (T), Leucine (L), Phenylalanine (F), or Serine (S),
(h) substitution of the amino acid at position 433 with Arginine (R), Serine (S), Isoleucine (I), Proline (P), or Glutamine (Q),
(i) substitution of the amino acid at position 434 with Tryptophan (W), Methionine (M), Serine (S), Histidine (H), Phenylalanine (F), or Tyrosine, and
(j) a combination of two or more of said substitutions, wherein the numbering is according to the EU index as set forth in Kabat.

336. The method of claim 334, wherein the human IgG constant domain comprises amino acid substitutions relative to a wild-type human IgG constant domain at positions 252, 254, and 256, wherein
(a) the amino acid at position 252 is substituted with Tyrosine (Y),
(b) the amino acid at position 254 is substituted with Threonine (T), and
(c) the amino acid at position 256 is substituted with Glutamic acid (E),
wherein the numbering is according to the EU index as set forth in Kabat.

337. The method of any one of claims 324-332, wherein the human IgG constant domain comprises amino acid substitutions relative to a wild-type human IgG constant domain at positions 252, 254, and 256, wherein
(a) the amino acid at position 252 is substituted with Tyrosine (Y),
(b) the amino acid at position 254 is substituted with Threonine (T), and
(c) the amino acid at position 256 is substituted with Glutamic acid (E),
wherein the numbering is according to the EU index as set forth in Kabat.

338. The method of any one of claims 334-337, wherein the amino acid at position 434 is substituted with an amino acid selected from the group consisting of Tryptophan (W), Methionine (M), Tyrosine (Y), and Serine (S), and wherein the numbering is according to the EU index as set forth in Kabat.

339. The method of claim 338, wherein the amino acid at position 428 is substituted with an amino acid selected from the group consisting of Threonine (T), Leucine (L), Phenylalanine (F), and Serine (S), and wherein the numbering is according to the EU index as set forth in Kabat.

340. The method of claim 338, wherein the amino acid at position 257 is substituted with Leucine (L), and the amino acid at Kabat position 434 is substituted with Tyrosine (Y), and wherein the numbering is according to the EU index as set forth in Kabat.

341. The method of claim 339, wherein the amino acid at Kabat position 428 is substituted with Leucine (L), and the amino acid at Kabat position 434 is substituted with Serine (S).

342. The method of claim 314, wherein the anti-HER3 antibody or antigen-binding fragment thereof comprises an antibody VL of SEQ ID NO:3, an antibody VH of SEQ ID NO: 2, and an IgG1 constant region of SEQ ID 46.

343. The method of claim 341, wherein the human IgG constant region comprises amino acid substitutions relative to a wild-type human IgG constant domain at positions 252, 254, and 256, wherein the numbering is according to the EU index as set forth in Kabat, and wherein
(a) the amino acid at position 252 is substituted with Tyrosine (Y),
(b) the amino acid at position 254 is substituted with Threonine (T), and
(c) the amino acid at position 256 is substituted with Glutamic acid (E).

344. The method of claim 325, wherein the anti-HER3 antibody or antigen-binding fragment thereof comprises a human IgG1 constant region and a human lambda constant region.

345. The method of claim 344, wherein the IgG1 constant region comprises amino acid substitutions relative to a wild-type human IgG constant domain at positions 252, 254, and 256, wherein the numbering is according to the EU index as set forth in Kabat, and wherein
(a) the amino acid at position 252 is substituted with Tyrosine (Y),
(b) the amino acid at position 254 is substituted with Threonine (T), and
(c) the amino acid at position 256 is substituted with Glutamic acid (E).

346. The method of claim 326, wherein the anti-HER3 antibody or antigen-binding fragment thereof comprises a human IgG1 constant region and a human lambda constant region.

347. The method of claim 346, wherein the IgG1 constant region comprises amino acid substitutions relative to a wild-type human IgG constant domain at positions 252, 254, and 256, wherein the numbering is according to the EU index as set forth in Kabat, and wherein
(a) the amino acid at position 252 is substituted with Tyrosine (Y),
(b) the amino acid at position 254 is substituted with Threonine (T), and
(c) the amino acid at position 256 is substituted with Glutamic acid (E).

348. The method of any one of claims 314-347, wherein the HER3 inhibitor is an anti-HER3 antibody.

349. The method of any one of claims 314-347, wherein the anti-HER3 antibody is a human antibody, a humanized antibody, a chimeric antibody, a monoclonal antibody, a polyclonal antibody, a recombinant antibody, a multispecific antibody, or an antigen-binding fragment thereof.

350. The method of claim 349, wherein the anti-HER3 antibody is a human antibody.

351. The method of any one of claims 314-350, which antigen-binding fragment is Fv, Fab, F(ab′)2, Fab′, dsFv, scFv, and sc(Fv)2.

352. The method of any one of claims 314-351, which anti-HER3 antibody or antigen-binding fragment is conjugated to at least one heterologous agent.

353. The method of any one of claims 303-352, wherein the B-Raf inhibitor is vemurafenib.

354. The method of any one of claims 303-352, wherein the B-Raf inhibitor is dabrafenib.

355. The method of any one of claims 303-354, wherein the MEK inhibitor is trametinib.

356. The method of any one of claims 303-354, wherein the MEK inhibitor is selumetinib.

357. The method of any one of claims 303-356, wherein the cancer is characterized by a BRAF mutation.

358. The method of any one of claims 303-357, wherein the cancer is resistant to treatment with a BRAF inhibitor.

359. The method of any one of claims 303-358, wherein the cancer is resistant to treatment with a MEK inhibitor

360. The method of any one of claims 303-359, wherein the cancer is resistant to treatment with a BRAF inhibitor and a MEK inhibitor.

361. The method of any one of claims 303-360, wherein the cancer is melanoma.

362. The method of claim 361, wherein the cancer is B-Raf mutated melanoma.

363. The method of any one of claims 303-360, wherein the cancer is thyroid cancer.

364. The method of claim 363, wherein the thyroid cancer is B-Raf mutated thyroid cancer.

365. The method of any one of claims 303-360, wherein the cancer is colorectal cancer.

366. The method of claim 365, wherein the colorectal cancer is B-Raf mutated colorectal cancer.

367. The method of any one of claims 303-360, wherein the cancer is lung cancer.

368. The method of claim 367, wherein the lung cancer is B-Raf mutated lung cancer.

369. The method of claim 367 or claim 368, wherein the lung cancer is non small cell lung carcinoma.

370. The method of any one of claims 303-360, wherein the cancer is hairy cell leukemia.

371. The method of claim 360, wherein the hairy cell leukemia is B-Raf mutated hairy cell leukemia.

372. The method of any one of claims 303-360, wherein the cancer is a squamous cell carcinoma of the head and neck (SCCHN), ovarian cancer, pancreatic cancer, bladder cancer, testicular cancer, endometrial cancer, hepatocellular carcinoma, breast cancer, gastric cancer, or prostate cancer.

373. The method of claim 372, wherein the cancer is SCCHN.

374. The method of any one of claims 303-373, wherein the method comprises a first step of obtaining the sample from a tumor from the patient.

375. The method of any one of claims 303-374, wherein, if the patient diagnosed with cancer is indicated as likely to be responsive to treatment with a HER3 inhibitor and a B-Raf inhibitor, the method comprises an additional step of administering to the patient a therapeutically effective amount of a HER3 inhibitor and a therapeutically effective amount of a B-Raf inhibitor.

376. The method of any one of claims 303-374, wherein, if the patient diagnosed with cancer is indicated as likely to be responsive to treatment with a HER3 inhibitor, the method comprises an additional step of administering to the patient a therapeutically effective amount of a HER3 inhibitor.

377. The method of any one of claims 303-374, wherein, if the patient diagnosed with cancer is indicated as likely to be responsive to treatment with a HER3 inhibitor and a MEK inhibitor, the method comprises an additional step of administering to the patient a therapeutically effective amount of a HER3 inhibitor and a therapeutically effective amount of a MEK inhibitor.

378. The method of any one of claims 303-374, wherein, if the patient diagnosed with cancer is indicated as likely to be responsive to treatment with a HER3 inhibitor, a B-Raf inhibitor, and a MEK inhibitor, the method comprises an additional step of administering to the patient a therapeutically effective amount of a HER3 inhibitor, a B-Raf inhibitor, and a MEK inhibitor.

379. A kit comprising components for performing the method of any one of claims 303-378.


SEQUENCE LISTING (48 sequences)

SEQ ID NO: 1 - protein, 110 aa, Artificial Sequence
  Synthetic sequence
QSVLTQPPSASGTPGQRVTISCSGSSSNIGLNYVSWYQQLPGTAPKLLISRNNQRPSGVPDRFSGSKSGTSASLAISGLRSEDEADYYCAAWDDSLSGEVFGGGTKLTVL

SEQ ID NO: 2 - protein, 118 aa, Artificial Sequence
  Synthetic sequence
EVQLLESGGGLVQPGGSLRLSCAASGFTFSYYYMQWVRQAPGKGLEWVSYIGSSGGVTNYADSVKGRFTISRDNSKNTLYLQMNSLRAEDTAVYYCARVGLGDAFDIWGQGTMVTVSS

SEQ ID NO: 3 - protein, 110 aa, Artificial Sequence
  Synthetic sequence
QSVLTQPPSASGTPGQRVTISCSGSLSNIGLNYVSWYQQLPGTAPKLLISRNNQRPSGVPDRFSGSKSGTSASLAISGLRSEDEADYYCAAWDDSPPGEAFGGGTKLTVL

SEQ ID NO: 4 - protein, 110 aa, Artificial Sequence
  Synthetic sequence
QSVLTQPPSASGTPGQRVTISCSGSSSNIGLNYVSWYQQLPGTAPKLLISRNNQRPSGVPDRFSGSKSGTSASLAISGLRSEDEADYYCAAWDDGLPGEVFGGGTKLTVL

SEQ ID NO: 5 - protein, 110 aa, Artificial Sequence
  Synthetic sequence
QSVLTQPPSASGTPGQRVTISCSGSSSNIGLNYVSWYQQLPGTAPKLLISRNNQRPSGVPDRFSGSKSGTSASLAISGLRSEDEADYYCAAWDDSLIGEVFGGGTKLTVL

SEQ ID NO: 6 - protein, 110 aa, Artificial Sequence
  Synthetic sequence
QSVLTQPPSASGTPGQRVTISCRGSSSNIGLNYVSWYQQLPGTAPKLLISRNNQRPSGVPDRFSGSKSGTSASLAISGLRSEDEADYYCAAWDDSLSGEVFGGGTKLTVL

SEQ ID NO: 7 - protein, 110 aa, Artificial Sequence
  Synthetic sequence
QSVLTQPPSASGTPGQRVTISCSGSLSNIGLNYVSWYQQLPGTAPKLLISRNNQRPSGVPDRFSGSKSGTSASLAISGLRSEDEADYYCAAWDDSLSGEVFGGGTKLTVL

SEQ ID NO: 8 - protein, 110 aa, Artificial Sequence
  Synthetic sequence
QSVLTQPPSASGTPGQRVTISCSGSSSNIGLNYVSWYQQLPGTAPKLLISRNNQRPSGVPDRFSGSKSGTSASLAISGLRSEDEADYYCAAWDDSLPGEVFGGGTKLTVL

SEQ ID NO: 9 - protein, 110 aa, Artificial Sequence
  Synthetic sequence
QSVLTQPPSASGTPGQRVTISCSGSSSNIGLNYVSWYQQLPGTAPKLLISRNNQRPSGVPDRFSGSKSGTSASLAISGLRSEDEADYYCAAWDDSLSGEAFGGGTKLTVL

SEQ ID NO: 10 - protein, 110 aa, Artificial Sequence
  Synthetic sequence
QSVLTQPPSASGTPGQRVTISCSGSSSNIGLNYVSWYQQLPGTAPKLLISRNNQRPSGVPDRFSGSKSGTSASLAISGLRSEDEADYYCAAWDDSPSGEVFGGGTKLTVL

SEQ ID NO: 11 - protein, 110 aa, Artificial Sequence
  Synthetic sequence
QSVLTQPPSASGTPGQRVTISCSGSSSNIGLNYVSWYQQLPGTAPKLLISRNNQRPSGVPDRFSGSKSGTSASLAISGLRSEDEADYYCAAWDDSLRGEVFGGGTKLTVL

SEQ ID NO: 12 - protein, 118 aa, Artificial Sequence
  Synthetic sequence
EVQLLESGGGLVQPGGSLRLSCAASGFTFSYYYMQWVRQAPGKGLEWVSIIGSSGGVTNYADSVKGRFTISRDNSKNTLYLQMNSLRAEDTAVYYCARVGLGDAFDIWGQGTMVTVSS

SEQ ID NO: 13 - protein, 118 aa, Artificial Sequence
  Synthetic sequence
EVQLLESGGGLVQPGGSLRLSCAASGFTFSYYYMQWVRQAPGKGLEWVSVIGSSGGVTNYADSVKGRFTISRDNSKNTLYLQMNSLRAEDTAVYYCARVGLGDAFDIWGQGTMVTVSS

SEQ ID NO: 14 - protein, 110 aa, Artificial Sequence
  Synthetic sequence
QSVLTQPPSASGTPGQRVTISCSGSSSNIGLNYVSWYQQLPGTAPKLLISRNNQRPSGVPDRFSGSKSGTSASLAISGLRSEDEADYYCAAWDDSPPGEAFGGGTKLTVL

SEQ ID NO: 15 - protein, 110 aa, Artificial Sequence
  Synthetic sequence
QSVLTQPPSASGTPGQRVTISCRGSSSNIGLNYVSWYQQLPGTAPKLLISRNNQRPSGVPDRFSGSKSGTSASLAISGLRSEDEADYYCAAWDDSPPGEAFGGGTKLTVL

SEQ ID NO: 16 - protein, 110 aa, Artificial Sequence
  Synthetic sequence
QSVLTQPPSASGTPGQRVTISCSGSSSNIGLNYVSWYQQLPGTAPKLLISRNNQRPSGVPDRFSGSKSGTSASLAISGLRSEDEADYYCAAWDDSPSGEAFGGGTKLTVL

SEQ ID NO: 17 - protein, 110 aa, Artificial Sequence
  Synthetic sequence
QYELTQPPSASGTPGQRVTMSCSGSSSNIGLNYVSWYQQLPGTAPKLLISRNNQRPSGVPDRFSGSKSGTSASLAISGLRSEDEADYYCAAWDDSLSGEVFGGGTKLTVL

SEQ ID NO: 18 - protein, 13 aa, Artificial Sequence
  Synthetic sequence
SGSSSNIGLNYVS

SEQ ID NO: 19 - protein, 13 aa, Artificial Sequence
  Synthetic sequence
SGSLSNIGLNYVS

SEQ ID NO: 20 - protein, 13 aa, Artificial Sequence
  Synthetic sequence
RGSSSNIGLNYVS

SEQ ID NO: 21 - protein, 7 aa, Artificial Sequence
  Synthetic sequence
RNNQRPS

SEQ ID NO: 22 - protein, 11 aa, Artificial Sequence
  Synthetic sequence
AAWDDSLSGEV

SEQ ID NO: 23 - protein, 11 aa, Artificial Sequence
  Synthetic sequence
AAWDDSPPGEA

SEQ ID NO: 24 - protein, 11 aa, Artificial Sequence
  Synthetic sequence
AAWDDSPSGEA

SEQ ID NO: 25 - protein, 11 aa, Artificial Sequence
  Synthetic sequence
AAWDDSLPGEV

SEQ ID NO: 26 - protein, 11 aa, Artificial Sequence
  Synthetic sequence
AAWDDGLPGEV

SEQ ID NO: 27 - protein, 11 aa, Artificial Sequence
  Synthetic sequence
AAWDDSLIGEV

SEQ ID NO: 28 - protein, 11 aa, Artificial Sequence
  Synthetic sequence
AAWDDSLSGEA

SEQ ID NO: 29 - protein, 11 aa, Artificial Sequence
  Synthetic sequence
AAWDDSPSGEV

SEQ ID NO: 30 - protein, 11 aa, Artificial Sequence
  Synthetic sequence
AAWDDSLRGEV

SEQ ID NO: 31 - protein, 5 aa, Artificial Sequence
  Synthetic sequence
YYYMQ

SEQ ID NO: 32 - protein, 17 aa, Artificial Sequence
  Synthetic sequence
YIGSSGGVTNYADSVKG

SEQ ID NO: 33 - protein, 17 aa, Artificial Sequence
  Synthetic sequence
IIGSSGGVTNYADSVKG

SEQ ID NO: 34 - protein, 17 aa, Artificial Sequence
  Synthetic sequence
VIGSSGGVTNYADSVKG

SEQ ID NO: 35 - protein, 9 aa, Artificial Sequence
  Synthetic sequence
VGLGDAFDI

SEQ ID NO: 36 - protein, 30 aa, Artificial Sequence
  Synthetic sequence
EVQLLESGGGLVQPGGSLRLSCAASGFTFS

SEQ ID NO: 37 - protein, 14 aa, Artificial Sequence
  Synthetic sequence
WVRQAPGKGLEWVS

SEQ ID NO: 38 - protein, 32 aa, Artificial Sequence
  Synthetic sequence
RFTISRDNSKNTLYLQMNSLRAEDTAVYYCAR

SEQ ID NO: 39 - protein, 11 aa, Artificial Sequence
  Synthetic sequence
WGQGTMVTVSS

SEQ ID NO: 40 - protein, 22 aa, Artificial Sequence
  Synthetic sequence
QSVLTQPPSASGTPGQRVTISC

SEQ ID NO: 41 - protein, 15 aa, Artificial Sequence
  Synthetic sequence
WYQQLPGTAPKLLIS

SEQ ID NO: 42 - protein, 32 aa, Artificial Sequence
  Synthetic sequence
GVPDRFSGSKSGTSASLAISGLRSEDEADYYC

SEQ ID NO: 43 - protein, 10 aa, Artificial Sequence
  Synthetic sequence
FGGGTKLTVL

SEQ ID NO: 44 - protein, 22 aa, Artificial Sequence
  Synthetic sequence
QYELTQPPSASGTPGQRVTMSC

SEQ ID NO: 45 - protein, 330 aa, Homo sapiens [sequence omitted]

SEQ ID NO: 46 - protein, 330 aa, Artificial Sequence
  Synthetic sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - DNA, 648 bp, Artificial Sequence
  Synthetic sequence
cagtacgaattgactcagccaccctcagcgtctgggacccccgggcagagtgtcaccatgtcttgttctgggagcagctccaatatcggacttaattatgtatcctggtaccagcacctcccaggaacggcccccaaactcctcatctctaggaataatcagcggccctcaggggtccctgaccgattctctggctccaagtctggcacctcagcctccctggccatcagtgggctccggtccgaagatgaggctgattattattgtgcagcatgggatgacagcctgagtggtgaggtattcggcggagggaccaagctgaccgtcctcggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctgcagaatgctct

SEQ ID NO: 48 - DNA, 356 bp, Artificial Sequence
  Synthetic sequence
gaagttcaattgttagagtctggtggcggtcttgttcagcctggtggttctttacgtctttcttgcgctgcttccggattcactttctcttattactatatgcagtgggttcgccaagctcctggtaaaggtttggagtgggtttcttatatcggttcttctggtggcgttactaattatgctgactccgttaaaggtcgcttcactatctctagagacaactctaagaatactctctacttgcagatgaacagcttaagggctgaggacacggccgtgtattactgtgcgagagtaggtcttggggatgcttttgatatctggggccaagggacaatggtcaccgtctcaagcgc